SEQUENCE LISTING (2 sequences)

SEQ ID NO: 1 - DNA, 69 bp, Artificial Sequence
  primer for cloning human VEGFR2 gene
aattaagtcgacatggactacaaggatgacgatgacaagaagcgggccaatggaggggaactgaagaca

SEQ ID NO: 2 - DNA, 42 bp, Artificial Sequence
  primer for cloning human VEGFR2 gene
aattaagcatgcttaaacaggaggagagctcagtgtggtccc

(19) United States
(12) Patent Application Publication
(10) Pub. No.: US 2009/0137580 A1
(43) Pub. Date: May 28, 2009

(54) Fused Heterocyclic Derivatives and Use Thereof

(75) Inventors: Shinichi Imamura, Tsukuba-shi (JP); Yuya Oguro, Tsukuba-shi (JP)

Correspondence Address: HAMRE, SCHUMANN, MUELLER & LARSON, P.C., P.O. BOX 2902, MINNEAPOLIS, MN, 55402-0902, US

(73) Assignee: Takeda Pharmaceutical Company Limited, Osaki-shi (JP)

(21) Appl. No.: 11/922,310

(22) PCT Filed: Jul. 5, 2006
(86) PCT No.: PCT/JP2006/313815

§ 371 Date: Dec. 14, 2007

(30) Foreign Application Priority Data
Jul. 5, 2005 (JP) 2005-196866
Feb. 28, 2006 (JP) 2006-054102

(51) Int. Cl. C07D 487/04; A61K 31/44; A61K 31/519; A61K 31/55; A61K 31/5377; A61P 35/04

(52) U.S. Cl. 514/234.2; 544/280; 514/265.1; 544/117; 514/252.16

(57) ABSTRACT

The present invention provides a fused heterocyclic derivative showing a potent kinase inhibitory activity and use thereof. A compound represented by the formula:

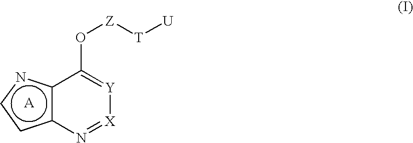

wherein ring A is an optionally substituted pyrrole ring, X is an optionally substituted CH, Y is an optionally substituted CH or nitrogen atom, Z is an optionally substituted divalent hydrocarbon group or optionally substituted divalent heterocyclic group, T is a single bond or an optionally substituted C₁₋₃ alkylene group, and U is an optionally substituted amido group, an optionally substituted sulfonamido group, an optionally substituted ureido group, an optionally substituted carbamoyl group or an optionally substituted thioureido group, or a salt thereof, and a pharmaceutical agent containing the compound or a prodrug thereof, which is a kinase (VEGFR, VEGFR2, PDGFR, TIE2) inhibitor, an angiogenesis inhibitor, an agent for the prophylaxis or treatment of cancer, an agent for inhibiting growth of cancer or an agent for suppressing metastasis of cancer.

### TECHNICAL FIELD

The present invention relates to fused heterocyclic derivatives and use thereof. More particularly, the present invention relates to pyrrolo[3,2-d]pyrimidine derivative and pyrrolo[3,2-b]pyridine derivatives having potent kinase inhibitory activity and useful for the prophylaxis or treatment of cancer, and use thereof.

### BACKGROUND ART

For a solid tumor to grow to a size greater than a certain level, formation of new blood vessels to supply sufficient nutrition and oxygen to the cancer cells is necessary (e.g., see New England Journal of Medicine (1971) Vol. 285, No. 21, pages 1182-1186). As one of the important factors causing angiogenesis in tumors, vascular endothelial growth factors (VEGF) are known, where VEGFs bind to vascular endothelial growth factor receptors (VEGFR) that express on vascular endothelial cells, and transduce cell growth signals (e.g., see Endocrine Reviews (1997) Vol. 18, No. 1, pages 4-25). Accordingly, inhibition of the VEGF-VEGFR signal transduction system is considered to inhibit angiogenesis and tumor growth (e.g., see Drug Discovery Today (2001) Vol. 6, No. 19, pages 1005-1024). Since tumor blood vessels are also involved in hematogenous metastasis of cancer, moreover, inhibition of angiogenesis is considered to be also effective for the inhibition of cancer metastasis.

As compounds inhibiting receptor tyrosine kinases including VEGFR, phthalazine derivatives (e.g., see WO98/35958), pyrrole substituted 2-indolinone derivatives (e.g., see WO01/60814), quinazoline derivatives (e.g., see WO01/32651), ω-carboxyaryl substituted diphenylurea derivatives (e.g., see WO00/42012), quinoline derivatives and quinazoline derivatives (e.g., see WO00/43366), nitrogen-containing aromatic ring derivatives (e.g., see WO02/32872) and the like are known. However, no VEGFR inhibitor has ever been placed on the market as a therapeutic drug for cancer.

As the pyrrolo[3,2-d]pyrimidine derivatives, WO98/08847 describes a compound represented by the formula:

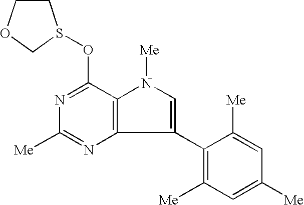

As the pyrrolo[3,2-b]pyridine derivatives, Journal of Organic Chemistry (2002) Vol. 67, No. 7, pages 2345-2347 describes a compound represented by the formula:

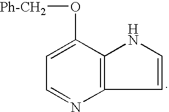

### DISCLOSURE OF THE INVENTION

### Problems to be Solved by the Invention

A kinase inhibitor superior in the affinity for kinases, efficacy expression, pharmacokinetic, solubility, interaction with other pharmaceutical products, safety and stability is expected to show a superior therapeutic effect. However, as the situation stands, one sufficiently satisfactory in terms of affinity for kinases, efficacy expression, pharmacokinetic, solubility, interaction with other pharmaceutical products, safety and stability has not been found. Thus, there is a demand for the development of a compound having superior kinase inhibitory activity and sufficiently satisfactory as a pharmaceutical product. Accordingly, it is an object of the present invention to provide a compound, which has superior kinase inhibitory activity, shows lower toxicity and is sufficiently satisfactory as a pharmaceutical product.

### Means of Solving the Problems

The present inventors have conducted intensive studies in an attempt to solve the above-mentioned problems and found that the compounds represented by the following formulas (I)-(III) and salts thereof (sometimes to be referred to as compounds (I)-(III) in the present specification) have superior kinase inhibitory activity, which resulted in the completion of the present invention.

Accordingly, the present invention provides the following.

(1) A compound represented by the formula:

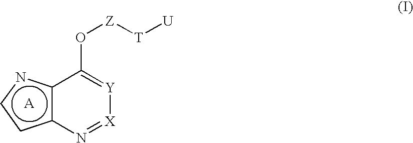

wherein ring A is an optionally substituted pyrrole ring, X is an optionally substituted CH, Y is an optionally substituted CH or nitrogen atom, Z is an optionally substituted divalent hydrocarbon group or an optionally substituted divalent heterocyclic group, T is a single bond or an optionally substituted C₁₋₃ alkylene group, and U is an optionally substituted amido group, an optionally substituted sulfonamido group, an optionally substituted ureido group, an optionally substituted carbamoyl group or an optionally substituted thioureido group, or a salt thereof.
(2) The compound of the above-mentioned (1), wherein X is CH, or a salt thereof.
(3) The compound of the above-mentioned (1), wherein Y is a nitrogen atom, or a salt thereof.
(4) The compound of the above-mentioned (1), wherein U is an optionally substituted ureido group, or a salt thereof.
(5) The compound of the above-mentioned (1), wherein T is a single bond, or a salt thereof.
(6) The compound of the above-mentioned (1), wherein ring A is an unsubstituted pyrrole ring or a pyrrole ring having substituent(s) on a ring nitrogen atom, or a salt thereof.
(7) The compound of the above-mentioned (1), which is represented by the formula:

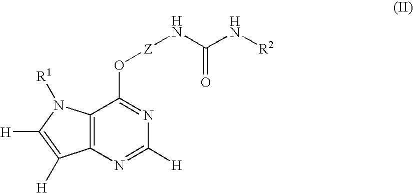

wherein R¹ is a hydrogen atom, an optionally substituted hydrocarbon group, an optionally substituted heterocyclic group or an acyl group, Z is an optionally substituted divalent hydrocarbon group or an optionally substituted divalent heterocyclic group, and R² is a hydrogen atom, an optionally substituted hydrocarbon group, an optionally substituted heterocyclic group, an optionally substituted amino group, an optionally substituted hydroxy group, an optionally substituted sulfanyl group or an acyl group, or a salt thereof.
(8) The compound of the above-mentioned (7), wherein R¹ is a hydrogen atom or an optionally substituted hydrocarbon group, or a salt thereof.
(9) The compound of the above-mentioned (7), wherein Z is an optionally substituted C₆₋₁₄ arylene group or an optionally substituted divalent heterocyclic group, or a salt thereof.
(10) The compound of the above-mentioned (7), wherein Z is a C₆₋₁₄ arylene group substituted by a halogen atom, or a salt thereof.
(11) The compound of the above-mentioned (7), wherein R² is an optionally substituted C₆₋₁₄ aryl group or an optionally substituted heterocyclic group, or a salt thereof.
(12) The compound of the above-mentioned (1), which is represented by the formula:

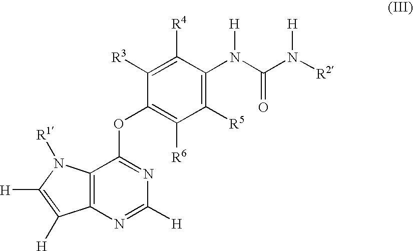

wherein R^{1′} is a hydrogen atom or an optionally substituted hydrocarbon group, R^{2′} is an optionally substituted phenyl group or an optionally substituted heterocyclic group, and R³, R⁴, R⁵ and R⁶ are each independently a hydrogen atom, a halogen atom, a cyano group, an optionally substituted hydrocarbon group, an optionally substituted amino group, an optionally substituted hydroxy group, an optionally substituted sulfanyl group or an acyl group or a salt thereof.
(13) The compound of the above-mentioned (12), wherein R⁴ is a halogen atom and R^{1′} is an optionally substituted hydrocarbon group, or a salt thereof.
(14)(i) N-{2-Chloro-4-[(5-methyl-5H-pyrrolo[3,2-d]pyrimidin-4-yl)oxy]phenyl}-N′-[3-(trifluoromethyl)phenyl]urea,
(ii) N-{2-chloro-4-[(5-methyl-5H-pyrrolo[3,2-d]pyrimidin-4-yl)oxy]phenyl}-N′-[3-(trifluoromethoxy)phenyl]urea,
(iii) N-{2-fluoro-4-[(5-methyl-5H-pyrrolo[3,2-d]pyrimidin-4-yl)oxy]phenyl}-N′-[3-(trifluoromethyl)phenyl]urea,
(iv) N-{2-chloro-4-[(5-methyl-5H-pyrrolo[3,2-d]pyrimidin-4-yl)oxy]phenyl}-N′-[4-(trifluoromethyl)pyridin-2-yl]urea,
(v) N-[2-chloro-4-(5H-pyrrolo[3,2-d]pyrimidin-4-yloxy)phenyl]-N′-[3-(trifluoromethyl)phenyl]urea,
(vi) N-{2-chloro-4-[(5-methyl-5H-pyrrolo[3,2-d]pyrimidin-4-yl)oxy]phenyl}-N′-[2-fluoro-5-(trifluoromethyl)phenyl]urea,
(vii) N-{2-chloro-4-[(5,6-dimethyl-5H-pyrrolo[3,2-d]pyrimidin-4-yl)oxy]phenyl}-N′-[3-(trifluoromethyl)phenyl]urea, or a salt of any thereof.
   (15) A prodrug of the compound of the above-mentioned (1).
   (16) A pharmaceutical agent comprising the compound of the above-mentioned (1) or a prodrug thereof.
   (17) The pharmaceutical agent of the above-mentioned (16), which is a kinase inhibitor.
   (18) The pharmaceutical agent of the above-mentioned (17), wherein the kinase is a vascular endothelial growth factor receptor (VEGFR).
   (19) The pharmaceutical agent of the above-mentioned (17), wherein the kinase is vascular endothelial growth factor receptor (VEGFR) 2.
   (20) The pharmaceutical agent of the above-mentioned (17), wherein the kinase is a platelet-derived growth factor receptor (PDGFR).
   (21) The pharmaceutical agent of the above-mentioned (17), wherein the kinase is a tyrosine kinase with Ig and EGF homology domains2 (TIE2).
   (22) The pharmaceutical agent of the above-mentioned (16), which is an angiogenesis inhibitor.
   (23) The pharmaceutical agent of the above-mentioned (16), which is an agent for the prophylaxis or treatment of cancer.
   (24) The pharmaceutical agent of the above-mentioned (16), which is an agent for inhibiting growth of cancer.
   (25) The pharmaceutical agent of the above-mentioned (16), which is an agent for suppressing metastasis of cancer.
   (26) A method for the prophylaxis or treatment of cancer, which comprises administering an effective amount of the compound of the above-mentioned (1) or a prodrug thereof to a mammal.
   (27) Use of the compound of the above-mentioned (1) or a prodrug thereof for the production of an agent for the prophylaxis or treatment of cancer.
   (28) The compound of the above-mentioned (12) wherein

(1) R¹ is a C₁₋₈ alkyl group optionally substituted by —(CH₂)ₘ-Q, —(CH₂)ₘ-Z¹-optionally substituted C₁₋₄ alkyl, —(CH₂)ₘ-Z²-(CH₂)ₙ-Q or —(CH₂)ₘ-Z²-(CH₂)ₙ-Z¹-optionally halogenated C₁₋₄ alkyl (preferably Q is hydroxy or —CONH₂, m is 0, Z¹ is —NH—CO— or —NH—CO₂—, or, Z¹ and Z² are each —O—) (the C₁₋₈ alkyl group is particularly methyl or ethyl),

(2) R^{2′} is a C₁₋₈ alkyl group (particularly methyl and propyl), a C₃₋₈ cycloalkyl group (particularly cyclopropyl), a C₆₋₁₈ aryl-C₁₋₄ alkyl group (particularly benzyl or phenylethyl), a C₆₋₁₄ aryl group (particularly phenyl, naphthyl, biphenylyl, tetrahydronaphthyl), aromatic monocyclic heterocyclic group (particularly pyridyl, oxazolyl, isoxazolyl, pyrimidinyl, pyrazolyl and thiadiazolyl), a non-aromatic (aliphatic) heterocyclic group (particularly piperidinyl), an aromatic fused heterocyclic group (particularly quinolyl, isoquinolyl and benzothiazolyl), or an aliphatic fused heterocyclic group (particularly benzodioxinyl or tetrahydroisoquinolyl), which is optionally substituted by substituent(s) selected from a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO-(optionally substituted alkyl group, alkoxy group, optionally substituted heterocyclic group or optionally substituted amino group), a C₆₋₁₈ aryl group, a heterocyclic group, an optionally substituted alkylthio group and an optionally substituted alkylsulfinyl group, and

(3) R³, R⁴, R⁵ and R⁶ are each independently a hydrogen atom or a halogen atom (preferably R⁴ is a halogen atom), or a salt thereof.

(29) The compound of the above-mentioned (12), wherein

(1) R^{1′} is a C₁₋₈ alkyl group optionally substituted by substituent(s) selected from hydroxy, —O-optionally halogenated C₁₋₄ alkyl, —O—(CH₂)ₙ-hydroxy and —O—(CH₂)ₙ—O-optionally halogenated C₁₋₄ alkyl (C₁₋₈ alkyl group is particularly methyl or ethyl),

(2) R^{2′} is a phenyl group, a 5- or 6-membered aromatic monocyclic heterocyclic group having 1 to 3 hetero atoms selected from a nitrogen atom, an oxygen atom and an optionally oxidized sulfur atom (particularly, pyridyl, oxazolyl, isoxazolyl or thiadiazolyl) or a 8- to 12-membered aliphatic fused heterocyclic group having 1 to 3 hetero atoms selected from a nitrogen atom, an oxygen atom and an optionally oxidized sulfur atom (particularly, benzodioxinyl), which is optionally substituted by substituent(s) selected from a halogen atom, an optionally halogenated C₁₋₄ alkyl group, an optionally halogenated C₁₋₄ alkyl-oxy group, a C₆₋₁₈ aryl-oxy group and a 5- to 8-membered heterocyclic group having 1 to 3 hetero atoms selected from a nitrogen atom, an oxygen atom and an optionally oxidized sulfur atom, and

(3) R³, R⁴, R⁵ and R⁶ are all hydrogen atoms, or one of R³, R⁴, R⁵ and R⁶ is a halogen atom and the rest are hydrogen atoms (preferably R⁴ is a halogen atom and R³, R⁵ and R⁶ are hydrogen atoms), or a salt thereof.

(30) The compound of the above-mentioned (12), wherein

(1) R^{1′} is methyl, ethyl, 2-hydroxyethoxyethyl, 2-methoxyethoxyethyl, 2-methoxyethyl or 2-hydroxyethyl,

(2) R^{2′} is methyl, n-propyl, benzyl, phenyl, trifluoromethylphenyl, chlorophenyl, methoxyphenyl, bromophenyl, fluorophenyl, methylphenyl, trifluoromethoxyphenyl, phenoxyphenyl, t-butylphenyl, chlorotrifluorophenyl, tetrafluoroethoxyphenyl, imidazolylphenyl, tetrafluorobenzodioxinyl, methylisoxazolyl, trifluoromethylthiadiazolyl, trifluoromethyloxazolyl or trifluoromethylpyridyl, and

(3) R³, R⁴, R⁵ and R⁶ are all hydrogen atoms, or one of R³, R⁴, R⁵ and R⁶ is a fluorine atom or a chlorine atom and the rest are hydrogen atoms, or a salt thereof.

(31) A production method of a compound represented by the formula:

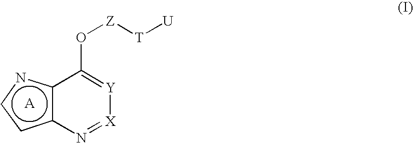

wherein each symbol is as defined in the above-mentioned (1), or a salt thereof, which comprises reacting a compound represented by the formula:

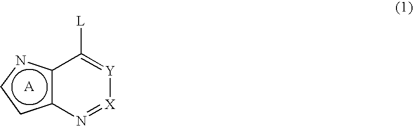

wherein L is a leaving group, and other symbols are as defined in the above-mentioned (1), or a salt thereof, with a compound represented by the formula:

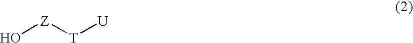

wherein each symbol is as defined in the above-mentioned (1), or a salt thereof.
(32) A production method of a compound represented by the formula:

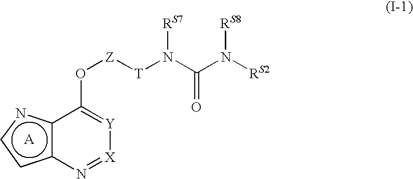

wherein R^{S7} is a hydrogen atom, an optionally substituted hydrocarbon group or an optionally substituted heterocyclic group, R^{S8} is a hydrogen atom, and R^{S2} is a hydrogen atom, an optionally substituted hydrocarbon group, an optionally substituted heterocyclic group, an optionally substituted amino group, an optionally substituted hydroxy group, an optionally substituted sulfanyl group or an acyl group, or a salt thereof, which comprises reacting a compound represented by the formula:

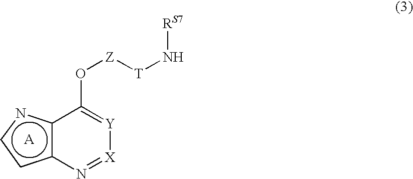

wherein R^{S7} is as defined above, and other symbols are as defined in the above-mentioned (1), or a salt thereof, with an isocyanate derivative represented by the formula: R^{S2}NCO wherein R^{S2} is as defined above.
(33) A production method of a compound represented by the formula:

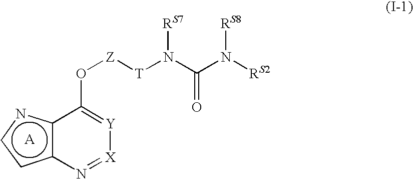

wherein R^{S7} is a hydrogen atom, an optionally substituted hydrocarbon group or an optionally substituted heterocyclic group, R^{S8} is a hydrogen atom, an optionally substituted hydrocarbon group or an optionally substituted heterocyclic group, R^{S2} is a hydrogen atom, an optionally substituted hydrocarbon group, an optionally substituted heterocyclic group, an optionally substituted amino group, an optionally substituted hydroxy group, an optionally substituted sulfanyl group or an acyl group, and other symbols are as defined in the above-mentioned (1), or a salt thereof, which comprises reacting a compound represented by the formula:

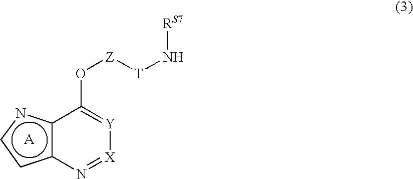

wherein R^{S7} is as defined above, and other symbols are as defined in the above-mentioned (1), or a salt thereof, with a compound represented by the formula: R^{S2}R^{S8}NC(O)L¹ wherein R^{S2} and R^{S8} are as defined above, and L¹ is a leaving group.
(34) A production method of a compound represented by the formula:

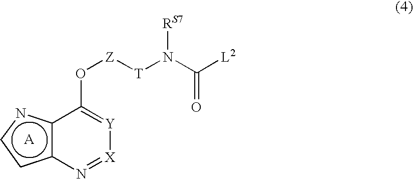

wherein R^{S7} is a hydrogen atom, an optionally substituted hydrocarbon group or an optionally substituted heterocyclic group, L² is a leaving groups and other symbols are as defined in the above-mentioned (1), or a salt thereof, which comprises reacting a compound represented by the formula:

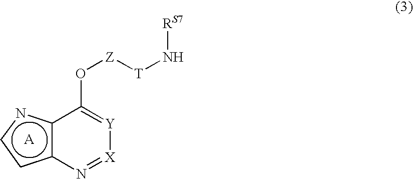

wherein R^{S7} is as defined above and other symbols are as defined in the above-mentioned (1), or a salt thereof, with a compound represented by formula: L¹C(O)L² wherein L¹ and L² are leaving groups.
(35) A production method of a compound represented by the formula:

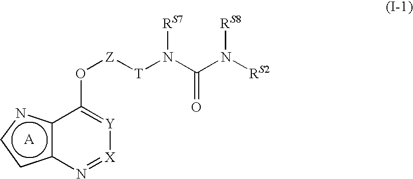

wherein R^{S7} is a hydrogen atom, an optionally substituted hydrocarbon group or an optionally substituted heterocyclic group, R^{S8} is a hydrogen atom, an optionally substituted hydrocarbon group or an optionally substituted heterocyclic group, R^{S2} is a hydrogen atom, an optionally substituted hydrocarbon group, an optionally substituted heterocyclic group, an optionally substituted amino group, an optionally substituted hydroxy group, an optionally substituted sulfanyl group or an acyl group and other symbols are as defined in the above-mentioned (1), or a salt thereof, which comprises reacting a compound represented by the formula:

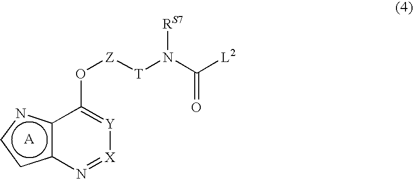

wherein R^{S7} is as defined above, L² is a leaving group and other symbols are as defined in the above-mentioned (1), or a salt thereof, with an amine derivative represented by the formula: R^{S2}R^{S8}NH wherein R^{S2} and R^{S8} are as defined above.
(36) A production method of a compound represented by the formula:

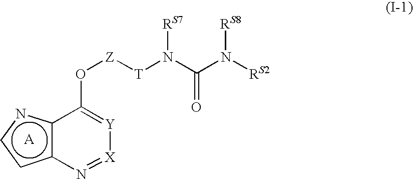

wherein R^{S7} is a hydrogen atom, an optionally substituted hydrocarbon group or an optionally substituted heterocyclic group, R^{S8} is a hydrogen atom, an optionally substituted hydrocarbon group or an optionally substituted heterocyclic group, R^{S2} is a hydrogen atom, an optionally substituted hydrocarbon group, an optionally substituted heterocyclic group, an optionally substituted amino group, an optionally substituted hydroxy group, an optionally substituted sulfanyl group or an acyl group and other symbols are as defined in the above-mentioned (1), or a salt thereof, which comprises reacting a compound represented by the formula:

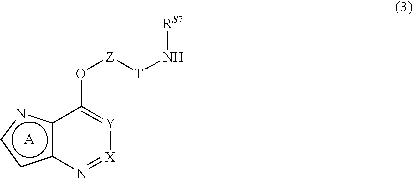

wherein R^{S7} is as defined above, and other symbols are as defined in the above-mentioned (1), or a salt thereof, with a compound represented by formula: L¹C(O)L² wherein L¹ and L² are leaving groups, and reacting the obtained compound represented by the formula:

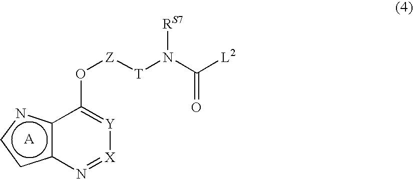

wherein each symbol is as defined above, or a salt thereof, with an amine derivative represented by the formula: R^{S2}R^{S8} NH wherein R^{S2} and R^{S8} are as defined above.

The present invention further provides the following.

(37) A compound represented by the formula:

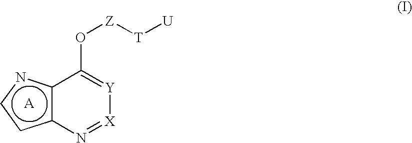

wherein ring A is an optionally substituted pyrrole ring, X is an optionally substituted CH, Y is an optionally substituted CH or nitrogen atom, Z is an optionally substituted divalent hydrocarbon group or an optionally substituted divalent heterocyclic group, T is a single bond or an optionally substituted C₁₋₃ alkylene group, and U is an optionally substituted amido group, an optionally substituted sulfonamido group or an optionally substituted ureido group, or a salt thereof.
(38) The compound of the above-mentioned (37), wherein X is CH, or a salt thereof.
(39) The compound of the above-mentioned (37), wherein Y is a nitrogen atom, or a salt thereof.
(40) The compound of the above-mentioned (37), wherein U is an optionally substituted ureido group, or a salt thereof.
(41) The compound of the above-mentioned (37), wherein T is a single bond, or a salt thereof.
(42) The compound of the above-mentioned (37) represented by the formula:

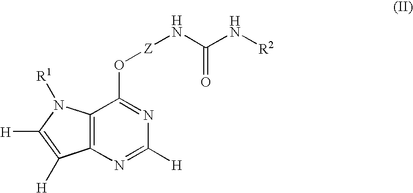

wherein R¹ is a hydrogen atom, an optionally substituted hydrocarbon group, an optionally substituted heterocyclic group or an acyl group, Z is an optionally substituted divalent hydrocarbon group or an optionally substituted divalent heterocyclic group, and R² is a hydrogen atom, an optionally substituted hydrocarbon group, an optionally substituted heterocyclic group, an optionally substituted amino group, an optionally substituted hydroxy group, an optionally substituted sulfanyl group or an acyl group, or a salt thereof.
(43) The compound of the above-mentioned (42), wherein Z is an optionally substituted C₆₋₁₄ arylene group or an optionally substituted divalent heterocyclic group, or a salt thereof.
(44) The compound of the above-mentioned (42), wherein Z is a C₆₋₁₄ arylene group substituted by a halogen atom, or a salt thereof.
(45) The compound of the above-mentioned (42), wherein R² is an optionally substituted C₆₋₁₄ aryl group or an optionally substituted heterocyclic group, or a salt thereof.
(46) The compound of the above-mentioned (37), which is represented by the formula:

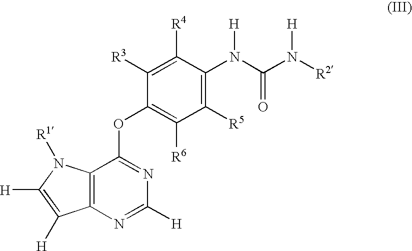

wherein R^{1′} is a hydrogen atom or an optionally substituted hydrocarbon group, R^{2′} is an optionally substituted phenyl group or an optionally substituted heterocyclic group, and R³, R⁴, R⁵ and R⁶ are each independently a hydrogen atom, a halogen atom, a cyano group, an optionally substituted hydrocarbon group, an optionally substituted amino group, an optionally substituted hydroxy group, an optionally substituted sulfanyl group or an acyl group, or a salt thereof.
(47) The compound of the above-mentioned (46), wherein R⁴ is a halogen atom, or a salt thereof.
(48) A prodrug of the compound of the above-mentioned (37).
(49) A pharmaceutical agent comprising the compound of the above-mentioned (37) or a prodrug thereof.
(50) The pharmaceutical agent of the above-mentioned (49), which is a kinase (phosphoenzyme) inhibitor.
(51) The pharmaceutical agent of the above-mentioned (50), wherein the kinase is a vascular endothelial growth factor receptor (VEGFR).
(52) The pharmaceutical agent of the above-mentioned (50), wherein the kinase is vascular endothelial growth factor receptor (VEGFR) 2.
(53) The pharmaceutical agent of the above-mentioned (50), wherein the kinase is a platelet-derived growth factor receptor (PDGFR).
(54) The pharmaceutical agent of the above-mentioned (49), which is an angiogenesis inhibitor.
(55) The pharmaceutical agent of the above-mentioned (49), which is an agent for the prophylaxis or treatment of cancer.
(56) The pharmaceutical agent of the above-mentioned (49), which is an agent for inhibiting growth of cancer.
(57) The pharmaceutical agent of the above-mentioned (49), which is an agent for suppressing metastasis of cancer. (58) A method for the prophylaxis or treatment of cancer, which comprises administering an effective amount of the compound of the above-mentioned (37) or a salt thereof, or a prodrug thereof to a mammal.
(59) Use of the compound of the above-mentioned (37) or a salt thereof, or a prodrug thereof for the production of an agent for the prophylaxis or treatment of cancer.
(60) A pharmaceutical agent comprising the compound of the above-mentioned (1) or a salt thereof, or a prodrug thereof.
(61) A method for the prophylaxis or treatment of cancer, which comprises administering an effective amount of the compound of the above-mentioned (1) or a salt thereof, or a prodrug thereof to a mammal.
(62) Use of the compound of the above-mentioned (1) or a salt thereof, or a prodrug thereof for the production of an agent for the prophylaxis or treatment of cancer.

### EFFECT OF THE INVENTION

Since the compounds (I)-(III) of the present invention, salts thereof and prodrugs thereof show superior inhibitory action on kinases such as vascular endothelial growth factor receptors and the like, clinically useful agents for the prophylaxis or treatment of diseases (e.g., cancer and the like) associated with the action of the vascular endothelial growth factors in living organisms can be provided. In addition, the compounds (I)-(III) of the present invention, salts thereof and prodrugs thereof are also superior in efficacy expression, pharmacokinetic, solubility, interaction with other pharmaceutical products, safety and stability, they are useful as pharmaceutical agents.

Since the compounds (I)-(III) of the present invention, salts thereof and prodrugs thereof show potent inhibitory action against kinases such as vascular endothelial growth factor receptors, platelet-derived-growth factor receptors, angiopoietin receptors and the like, and also show potent angiogenesis inhibitory action, they can provide clinically useful agents for the prophylaxis or treatment of cancer, cancer growth inhibitors, cancer metastasis inhibitors. Furthermore, the compounds (I)-(III) of the present invention, salts thereof and prodrugs thereof can provide clinically useful agents for the prophylaxis or treatment of diseases other than cancer, such as chronic rheumatoid arthritis, diabetic retinopathy and the like, and are superior in terms of efficacy expression, pharmacokinetic, solubility, interaction with other pharmaceutical products, safety and stability.

### BEST MODE FOR EMBODYING THE INVENTION

The present invention is explained in detail in the following.

In compound (I), the pyrrole ring of the “optionally substituted pyrrole ring” for ring A may have 1 to 3 substituents at substitutable position(s). When the ring has plural substituents, the substituents may be the same or different. As such substituent, for example,

(i) a halogen atom,
(ii) a cyano group,
(iii) a nitro group,
(iv) an optionally substituted hydrocarbon group
(C₁₋₈ alkyl group, C₂₋₈ alkenyl group, C₂₋₈ alkynyl group, C₃₋₈ cycloalkyl group, C₃₋₈ cycloalkenyl group, C₃₋₈ cycloalkyl-C₁₋₄ alkyl group, C₃₋₈ cycloalkenyl-C₁₋₄ alkyl group, C₆₋₁₈ aryl group, C₆₋₁₈ aryl-C₁₋₄ alkyl group and the like),
(v) a hydroxy group,
(vi) an optionally substituted hydrocarbon-oxy group
(C₁₋₈ alkyl-oxy group, C₂₋₈ alkenyl-oxy group, C₂₋₈ alkynyl-oxy group, C₃₋₈ cycloalkyl-oxy group, C₆₋₁₈ aryl-oxy group, C₆₋₁₈ aryl-C₁₋₄ alkyl-oxy group and the like),
(vii) an optionally substituted heterocycle-oxy group,
(viii) an optionally substituted carbamoyl group,
(ix) an acyl group
(optionally substituted C₁₋₈ alkyl-carbonyl group, optionally substituted-C₆₁₈ aryl-carbonyl group, optionally substituted C₆₋₁₈ aryl-C₁₋₄ alkyl-carbonyl group, optionally substituted C₁₋₈ alkylsulfonyl group, optionally substituted C₆₋₁₈ aryl-sulfonyl group, optionally substituted heterocycle-carbonyl group, optionally substituted heterocyclic sulfonyl group and the like),
(x) an optionally substituted amino group,
(xi) an optionally substituted sulfanyl group,
(xii) an optionally substituted heterocyclic group, and the like (hereinafter to be referred to as substituent group (1)) can be mentioned.

As the “halogen atom”, fluorine, chlorine, bromine and iodine can be mentioned.

As the “C₁₋₈ alkyl group”, for example, methyl, ethyl, n-propyl, i-propyl, n-butyl, i-butyl, s-butyl, t-butyl, n-pentyl, i-pentyl, t-pentyl, neopentyl, n-hexyl, i-hexyl, n-heptyl and n-octyl and the like can be mentioned, with preference given to a C₁₋₆ alkyl group. As the “C₁₋₄ alkyl group”, for example, methyl, ethyl, n-propyl, i-propyl, n-butyl and i-butyl can be mentioned.

As the “C₂₋₈ alkenyl group”, for example, ethenyl, propenyl, butenyl, pentenyl, hexenyl, heptenyl, octenyl, butadienyl and the like can be mentioned, with preference given to a C₂₋₄ alkenyl group.

As the “C₂₋₈ alkynyl group”, for example, ethynyl, propynyl, butynyl, pentynyl, hexynyl, heptynyl, octynyl and the like can be mentioned, with preference given to a C₂₋₄ alkynyl group.

As the “C₃₋₈ cycloalkyl group”, for example, cyclopropyl, cyclobutyl, cyclopentyl, cyclohexyl, cycloheptyl and cyclooctyl and the like can be mentioned, with preference given to a C₃₋₆ cycloalkyl group.

As the “C₃₋₈ cycloalkenyl group”, for example, cyclopropenyl, cyclobutenyl, cyclopentenyl, cyclohexenyl, cycloheptenyl, cyclooctenyl and the like can be mentioned, with preference given to a C₃₋₆ cycloalkenyl group.

As the “C₃₋₈ cycloalkyl-C₁₋₄ alkyl group”, for example, cyclopropylmethyl, cyclopropylethyl, cyclobutylmethyl, cyclopentylmethyl, cyclohexylmethyl, cyclohexylethyl and the like can be mentioned.

As the “C₃₋₈ cycloalkenyl-C₁₋₄ alkyl group”, for example, cyclopentenylmethyl, cyclohexenylmethyl, cyclohexenylethyl, cyclohexenylpropyl, cycloheptenylmethyl, cycloheptenylethyl and the like can be mentioned.

As the “C₆₋₁₈ aryl group”, for example, phenyl, 1-naphthyl, 2-naphthyl, biphenylyl, 2-anthryl, phenanthryl, acenaphthylenyl and the like can be mentioned.

As the “C₆₋₁₈ aryl-C₁₋₄ alkyl group”, for example, phenylmethyl(benzyl), phenylethyl(phenethyl) and the like can be mentioned.

As the “C₁₋₈ alkyl-oxy group”, for example, methyloxy(methoxy), ethyloxy(ethoxy), propyloxy(propoxy), isopropyloxy(isopropoxy), butyloxy(butoxy), isobutyloxy(isobutoxy), pentyloxy(pentoxy), hexyloxy(hexoxy) and the like can be mentioned.

As the “C₂₋₈ alkenyl-oxy group”, for example, ethenyloxy, propenyloxy, butenyloxy, pentenyloxy, hexenyloxy, heptenyloxy, octenyloxy and the like can be mentioned.

As the “C₂₋₈ alkynyl-oxy group”, for example, ethynyloxy, propynyloxy, butynyloxy, pentynyloxy, hexynyloxy, heptynyloxy, octynyloxy and the like can be mentioned.

As the “C₃₋₈ cycloalkyl-oxy group”, for example, cyclopropyloxy, cyclobutyloxy, cyclopentyloxy, cyclohexyloxy, cycloheptyloxy, cyclooctyloxy and the like can be mentioned.

As the “C₆₋₁₈ aryl-oxy group”, for example, phenyloxy(phenoxy), naphthyloxy(naphthoxy) and the like can be mentioned.

As the “C₆₋₁₈ aryl-C₁₋₄ alkyl-oxy group”, for example, phenylmethyloxy(benzyloxy), phenylethyloxy(phenethyloxy) and the like can be mentioned.

As the “C₁₋₈ alkyl-carbonyl group”, for example, methylcarbonyl(acetyl), ethylcarbonyl(propionyl), propylcarbonyl(butyryl), butylcarbonyl(pentanoyl), pentylcarbonyl, hexylcarbonyl, heptylcarbonyl, octylcarbonyl and the like can be mentioned.

As the “C₆₋₁₈ aryl-carbonyl group”, for example, phenylcarbonyl(benzoyl), naphthylcarbonyl(naphthoyl), anthrylcarbonyl, phenanthrylcarbonyl, acenaphthylenylcarbonyl and the like can be mentioned.

As the “C₆₋₁₈ aryl-C₁₋₄ alkyl-carbonyl group”, for example, phenylacetyl(benzylcarbonyl), phenylpropionyl(phenethylcarbonyl), phenylbutyryl, phenylpentanoyl and the like can be mentioned.

As the “C₁₋₈ alkylsulfonyl group”, for example, methylsulfonyl, ethylsulfonyl, propylsulfonyl, butylsulfonyl, pentylsulfonyl, hexylsulfonyl, heptylsulfonyl, octylsulfonyl and the like can be mentioned.

As the “C₆₋₁₈ aryl-sulfonyl group”, for example, phenylsulfonyl, naphthylsulfonyl and the like can be mentioned.

As the “heterocyclic group” or “heterocycle” of the heterocycle-oxy group, heterocycle-carbonyl group or heterocyclic sulfonyl group, for example, 5- to 12-membered “aromatic heterocyclic group” (aromatic monocyclic heterocyclic group or aromatic fused heterocyclic group etc.) or “saturated or unsaturated aliphatic heterocyclic group” having, as a ring-constituting atom (ring atom), one or more (preferably 1 to 4, more preferably 1 or 2) of and 1 to 3 kinds (preferably one or two kinds) of hetero atoms selected from an oxygen atom, an optionally oxidized sulfur atom, a nitrogen atom etc. (preferably, an oxygen atom, a sulfur atom, a nitrogen atom etc.) can be mentioned. Specifically, a 5- or 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl and the like, a 8- to 12-membered aromatic fused heterocyclic group (preferably, a heterocyclic group wherein the aforementioned 5- or 6-membered aromatic monocyclic heterocyclic group is condensed with a benzene ring or a heterocyclic group wherein same or different two heterocycles of the aforementioned 5- or 6-membered aromatic monocyclic heterocyclic group are condensed) such as benzofuranyl, isobenzofuranyl, benzothienyl, isobenzothienyl, indolyl, isoindolyl, 1H-indazolyl, benzimidazolyl, benzoxazolyl, 1,2-benzoisoxazolyl, benzothiazolyl, 1,2-benzoisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acrydinyl, phenoxazinyl, phenothiazinyl, phenazinyl, phenoxathiinyl, thianthrenyl, phenanthridinyl, phenanthrolyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like, a 3- to 8-membered (preferably 5- or 6-membered) saturated or unsaturated (preferably saturated) non-aromatic heterocyclic group (aliphatic heterocyclic group) such as oxiranyl, azetidinyl, oxetanyl, thietanyl, pyrrolidinyl, tetrahydrofuryl, thioranyl, piperidinyl, tetrahydropyranyl, thianyl, morpholinyl, thiomorpholinyl, piperazinyl, azepanyl, oxepanyl, thiepanyl, oxazepanyl, thiazepanyl, azocanyl, oxocanyl, thiocanyl, oxazocanyl, thiazocanyl, dioxinyl and the like, and the like can be mentioned. These non-aromatic heterocyclic groups may be oxo-substituted and, for example, 2-oxoazetidinyl, 2-oxopyrrolidinyl, 2-oxopiperidinyl, 2-oxoazepanyl, 2-oxoazocanyl, 2-oxotetrahydrofuryl, 2-oxotetrahydropyranyl, 2-oxothioranyl, 2-oxothianyl, 2-oxopiperazinyl, 2-oxoxepanyl, 2-oxoxazepanyl, 2-oxothiepanyl, 2-oxothiazepanyl, 2-oxoxocanyl, 2-oxothiocanyl, 2-oxoxazocanyl, 2-oxothiazocanyl, 2-oxodioxinyl and the like can be mentioned. These aliphatic heterocyclic groups may be condensed with a benzene ring or the above-mentioned aromatic monocyclic heterocyclic group to form an aliphatic fused heterocyclic group. As the aliphatic fused heterocyclic group wherein an aliphatic heterocyclic group is condensed with a benzene ring, for example, benzodioxinyl, tetrahydroisoquinolyl and the like can be mentioned.

These hydrocarbon groups (C₁₋₈ alkyl group, C₂₋₈ alkenyl group, C₂₋₈ alkynyl group, C₃₋₈ cycloalkyl group, C₃₋₈ cycloalkenyl group, C₃₋₈ cycloalkyl-C₁₋₄ alkyl group, C₃₋₈ cycloalkenyl-C₁₋₄ alkyl group, C₆₋₁₈ aryl group, C₆₋₁₈ aryl-C₁₋₄ alkyl group and the like), hydrocarbon-oxy group (C₁₋₈ alkyl-oxy group, C₂₋₈ alkenyl-oxy group, C₂₋₈ alkynyl-oxy group, C₃₋₈ cycloalkyl-oxy group, C₆₋₁₈ aryl-oxy group, C₆₋₁₈ aryl-C₁₋₄ alkyl-oxy group and the like), heterocycle-oxy group, carbamoyl group, C₁₋₈ alkyl-carbonyl group, C₆₋₁₈ aryl-carbonyl group, C₆₋₁₈ aryl-C₁₋₄ alkyl-carbonyl group, C₁₋₈ alkylsulfonyl group, C₆₋₁₈ aryl-sulfonyl group, heterocycle-carbonyl group, heterocyclic sulfonyl group, amino group, sulfanyl group and heterocyclic group may have one to the maximum acceptable number of substituents at any substitutable position(s), and when two or more substituents are contained, the substituents may be the same or different. As such substituents, for example,

(i) a halogen atom,
(ii) oxo,
(iii) optionally halogenated C₁₋₄ alkyl,
(iv) an optionally substituted heterocyclic group
(preferably, a 5- to 8-membered heterocyclic group having 1 to 3 hetero atoms selected from a nitrogen atom, an oxygen atom and an optionally oxidized sulfur atom and, as the substituent, halogen atom, oxo, optionally halogenated C₁₋₄ alkyl, optionally halogenated C₁₋₄ alkoxy, formyl, C₁₋₄ alkyl-carbonyl, amino, mono- or di-C₁₋₄ alkylamino and the like can be used),
(v) —(CH₂)ₘ-Q,
(vi) —(CH₂)ₘ-Z¹-optionally substituted C₁₋₄ alkyl
(as the substituent, halogen atom, hydroxy, C₁₋₄ alkylsulfonyl, optionally halogenated C₁₋₄ alkyl, C₁₋₄ alkoxy, formyl, C₁₋₄ alkyl-carbonyl, amino, mono or di-C₁₋₄ alkylamino and the like can be used),
(vii) —(CH₂)ₘ-Z¹-optionally substituted C₃₋₈ cycloalkyl
(as the substituent, halogen atom, oxo, optionally halogenated C₁₋₄ alkyl, C₁₋₄ alkoxy, formyl, C₁₋₄ alkyl-carbonyl, amino, mono or di-C₁₋₄ alkylamino and the like can be used),
(viii) —(CH₂)ₘ-Z¹-optionally substituted C₆₋₁₈ aryl
(as the substituent, halogen atom, oxo, optionally halogenated C₁₋₄ alkyl, C₁₋₄ alkoxy, formyl, C₁₋₄ alkyl-carbonyl, amino, mono or di-C₁₋₄ alkylamino and the like can be used),
(ix) —(CH₂)ₘ-Z²-(CH₂)ₙ-Q,
(x) —(CH₂)ₘ-Z²-(CH₂)ₙ-Z¹-optionally halogenated C₁₋₄ alkyl,
(xi) —(CH₂)ₘ-Z²-(CH₂)ₙ-Z¹-optionally substituted C₃₋₈ cycloalkyl
(as the substituent, halogen atom, oxo, optionally halogenated C₁₋₄ alkyl, C₁₋₄ alkoxy, formyl, C₁₋₄ alkyl-carbonyl, amino, mono or di-C₁₋₄ alkylamino and the like can be used),
(xii) —(CH₂)ₘ-Z²-(CH₂)ₙ-Z¹-optionally substituted C₆₋₁₈ aryl
(as the substituent, halogen atom, oxo, optionally halogenated C₁₋₄ alkyl, C₁₋₄ alkoxy, formyl, C₁₋₄ alkyl-carbonyl, amino, mono or di-C₁₋₄ alkylamino and the like can be used),
(xiii) —(CH₂)ₘ-Z¹-optionally substituted heterocyclic group
(as the heterocyclic group, preferred is a 5- to 8-membered heterocyclic group having 1 to 3 hetero atoms selected from a nitrogen atom, an oxygen atom and an optionally oxidized sulfur atom and, as the substituent, halogen atom, oxo, optionally halogenated C₁₋₄ alkyl, C₁₋₄ alkoxy, formyl, C₁₋₄ alkyl-carbonyl, amino, mono or di-C₁₋₄ alkylamino and the like can be used),
(xiv) —(CH₂)ₘ-Z¹-optionally halogenated C₁₋₄ alkoxy,
(xv) —(CH₂)ₘ-Z²-(CH₂)ₙ-Z¹-(CH₂)ₙ-Z¹-optionally halogenated C₁₋₄ alkyl
(m is an integer of 0 to 4, n is an integer of 1 to 4, Q is hydroxy, carboxy, cyano, nitro, —NR⁷R⁸, —CONR⁷R⁸, —OCONH₂ or —SO₂NR⁷R⁸, Z¹ is —O—, —CO—, —C(OH)R⁹—, —C(═N—OR⁹)—, —S—, —SO—, —SO₂—, —N(COR⁹)—, —N(CO₂R¹⁰)—, —N(SO₂R¹⁰)—, —CO—O—, —O—CO—, —CO—NR⁹—, —NR⁹—CO—, —NR⁹—CO₂—, —NR⁹—CO—NH—, —NR⁹—SO₂— or —NR⁹—C(═NH)—NH—, and Z² is —O—, —CO—, —C(OH)R⁹—, —C(═N—OR⁹)—, —S—, —SO—, —SO₂—, —NR⁹—, —N(COR⁹)—, —N(CO₂R¹⁰)—, —N(SO₂R¹⁰)—, —CO—O—, —O—CO—, —CO—NR⁹—, —NR⁹—CO—, —NR⁹—CO₂—, —NR⁹—CO—NH—, —NR⁹—C(═NH)—NH—, —NR⁹—SO₂— or —SO₂—NR⁹—. (CH₂)ₘ and (CH₂), may be substituted by 1 to 5 substituents selected from halogen, optionally halogenated C₁₋₄ alkyl and hydroxy, and when m or n is not less than 2, a subset of —CH₂CH₂— of (CH₂)ₘ and (CH₂), may be replaced with —CH═CH— or —C≡C—. R⁷ and R⁸ are the same or different and each is a hydrogen atom or a C₁₋₄ alkyl group, or R⁷ and R⁸ are bonded to form, together with a nitrogen atom, a 3- to 8-membered saturated or unsaturated aliphatic heterocyclic group. R⁹ is a hydrogen atom or a C₁₋₄ alkyl group, and R¹⁰ is a C₁₋₄ alkyl group, and the like (hereinafter to be referred to as substituent group (2)) can be mentioned.

In compound (I), as the substituent that CH of the “optionally substituted CH” for X and Y can have, substituent(s) selected from the above-mentioned substituent group (1) can be mentioned. When the substituent(s) selected from substituent group (1) is(are) substituted by two or more substituents, the substituents may be the same or different.

In compound (I), as the “hydrocarbon” of the “optionally substituted divalent hydrocarbon group” for Z, for example, saturated or unsaturated chain (linear or branched) aliphatic hydrocarbon, saturated or unsaturated cyclic aliphatic hydrocarbon (alicyclic hydrocarbon), monocyclic and condensed polycyclic aromatic hydrocarbon and the like can be mentioned. As the chain aliphatic hydrocarbon, for example, C₁₋₈ alkane, C₂₋₈ alkene and C₂₋₈ alkyne can be mentioned and, as the cyclic aliphatic hydrocarbon, for example, C₃₋₈ cycloalkane and C₃₋₈ cycloalkene can be mentioned. As the monocyclic and condensed polycyclic aromatic hydrocarbon, for example, C₆₋₁₈ arene can be mentioned. The positions of the two bonds from the “hydrocarbon” of the “optionally substituted divalent hydrocarbon group” are not particularly limited, as long as they are realizable.

As the C₁₋₈ alkane of the “optionally substituted divalent hydrocarbon group” for Z, for example, methane, ethane, propane, butane, pentane, hexane, heptane, octane and the like can be mentioned.

As the “C₂₋₈ alkene” of the “optionally substituted divalent hydrocarbon group” for Z, for example, ethene, propene, butene, pentene, hexene, heptene, octene and the like can be mentioned.

As the “C₂₋₈ alkyne” of the “optionally substituted divalent hydrocarbon group” for Z, for example, ethyne, propyne, butyne, pentyne, hexyne, heptyne, octyne and the like can be mentioned.

As the “C₃₋₈ cycloalkane” of the “optionally substituted divalent hydrocarbon group” for Z, for example, cyclopropane, cyclobutane, cyclopentane, cyclohexane, cycloheptane, cyclooctane and the like can be mentioned.

As the “C₃₋₈ cycloalkene” of the “optionally substituted divalent hydrocarbon group” for Z, for example, cyclopropene, cyclobutene, cyclopentene, cyclohexene, cycloheptene, cyclooctene and the like can be mentioned.

As the “C₆₋₁₈ arene” of the “optionally substituted divalent hydrocarbon group” for Z, for example, benzene, biphenyl, naphthalene, anthracene, phenanthrene, acenaphthylene, methylbenzene and the like can be mentioned.

In compound (I), the “hydrocarbon group” of the “optionally substituted divalent hydrocarbon group” for Z may have one to the maximum acceptable number of substituents at any substitutable position(s), and when two or more substituents are contained, the substituents may be the same or different. As such substituents, substituents selected from the above-mentioned substituent group (1) can be mentioned, with preference given to a halogen atom.

In compound (I), as the “heterocycle” of the “optionally substituted divalent heterocyclic group” for Z, for example, a 5- to 12-membered “aromatic heterocycle” or “saturated or unsaturated aliphatic heterocycle” having, as a ring-constituting atom (ring atom), one or more (preferably 1 to 4, more preferably 1 or 2) of 1 to 3 kinds (preferably one or two kinds) of hetero atoms selected from an oxygen atom, an optionally oxidized sulfur atom, a nitrogen atom etc. (preferably, an oxygen atom, a sulfur atom, a nitrogen atom etc.) can be mentioned. As the “aromatic heterocycle”, an aromatic monocyclic heterocycle or aromatic fused heterocycle and the like can be mentioned. The positions of the two bonds from the “heterocycle” of the “optionally substituted divalent heterocyclic group” are not particularly limited, as long as they are realizable.

As the “aromatic monocyclic heterocycle”, for example, a 5- or 6-membered aromatic monocyclic heterocycle such as furan, thiophene, pyrrole, oxazole, isoxazole, thiazole, isothiazole, imidazole, pyrazole, 1,2,3-oxadiazole, 1,2,4-oxadiazole, 1,3,4-oxadiazole, furazan, 1,2,3-thiadiazole, 1,2,4-thiadiazole, 1,3,4-thiadiazole, 1,2,3-triazole, 1,2,4-triazole, tetrazole, pyridine, pyridazine, pyrimidine, pyrazine, triazine and the like, and the like can be mentioned.

As the “aromatic fused heterocycle”, for example, a 8- to 12-membered aromatic fused heterocycle such as benzofuran, isobenzofuran, benzothiophene, isobenzothiophene, indole, isoindole, 1H-indazole, benzimidazole, benzoxazole, 1,2-benzoisoxazole, benzothiazole, 1,2-benzoisothiazole, 1H-benzotriazole, quinoline, isoquinoline, cinnoline, quinazoline, quinoxaline, phthalazine, naphthyridine, purine, pteridine, carbazole, carboline, acridine, phenoxazine, phenothiazine, phenazine, phenoxathine, thianthrene, phenanthridine, phenanthroline, indolizine, pyrrolo[1,2-b]pyridazine, pyrazolo[1,5-a]pyridine, imidazo[1,2-a]pyridine, imidazo[1,5-a]pyridine, imidazo[1,2-b]pyridazine, imidazo[1,2-a]pyrimidine, 1,2,4-triazolo[4,3-a]pyridine, 1,2,4-triazolo[4,3-b]pyridazine and the like, and the like can be mentioned. As the aromatic fused heterocycle, a heterocycle wherein the aforementioned 5- or 6-membered aromatic monocyclic heterocycle is condensed with a benzene ring and a heterocycle wherein same or different two heterocycles of the aforementioned 5- or 6-membered aromatic monocyclic heterocycle are condensed are preferable.

As the “saturated or unsaturated aliphatic heterocycle.”, for example, a 3- to 8-membered (preferably 5- or 6-membered) saturated or unsaturated (preferably saturated) aliphatic heterocycle (non-aromatic heterocycle) such as oxirane, azetidine, oxetane, thietane, pyrrolidine, tetrahydrofuran, tetrahydrothiophene, piperidine, tetrahydropyran, tetrahydrothiopyran, morpholine, thiomorpholine, piperazine, azepane, oxepane, thien, oxazepane, thiazepane, azocane, oxocane, thiocane, oxazocane, thiazocane and the like, and the like can be mentioned. These may be substituted by oxo and may be, for example, 2-oxoazetidine, 2-oxopyrrolidine, 2-oxopiperidine, 2-oxoazepane, 2-oxoazocane, 2-oxotetrahydrofuran, 2-oxotetrahydropyran, 2-oxotetrahydrothiophene, 2-oxothiane, 2-oxopiperazine, 2-oxooxepane, 2-oxooxazepane, 2-oxothiepane, 2-oxothiazepane, 2-oxooxocane, 2-oxothiocane, 2-oxooxazocane, 2-oxothiazocane and the like. In addition, these aliphatic heterocycles may be condensed with a benzene ring or the above-mentioned aromatic monocyclic heterocycle to form an aliphatic fused heterocycle.

In compound (I), the heterocyclic group of the “optionally substituted divalent heterocyclic group” for Z may have one to the maximum acceptable number of substituents at any substitutable position(s), and when two or more substituents are contained, the substituents may be the same or different. As such substituent, substituents selected from the above-mentioned substituent group (1) can be mentioned.

In compound (I), as the C₁₋₃ alkylene group of the “optionally substituted C₁₋₃ alkylene group” for T, a methylene group, an ethylene group, a methylethylene group and a propylene group can be mentioned. These C₁₋₃ alkylene groups may have one to the maximum acceptable number of substituents at any substitutable position(s), and when two or more substituents are contained, the substituents may be the same or different. As such substituent, substituents selected from the above-mentioned substituent group (1) can be mentioned.

In compound (I), the amido group of the “optionally substituted amido group”, the sulfonamido group of the “optionally substituted sulfonamido group”, the ureido group of the “optionally substituted ureido group”, the carbamoyl group of the “optionally substituted carbamoyl group”, and the thioureido group of the “optionally substituted thioureido group” for U may have one to the maximum acceptable number of substituents at any substitutable position(s), and when plural substituents are contained, the substituents may be the same or different. As such substituent, substituents selected from the above-mentioned substituent group (1) can be mentioned.

In compound (I), ring A is preferably unsubstituted pyrrole ring or a pyrrole ring having substituent(s) on a ring nitrogen atom. The substituent on the ring nitrogen atom is preferably the optionally substituted hydrocarbon group, the optionally substituted heterocyclic group or the acyl group in the substituent group (1), more preferably the optionally substituted hydrocarbon group, further preferably the optionally substituted C₁₋₈ alkyl group (C₁₋₈ alkyl group is preferably methyl or ethyl). The substituent of the substituent(s) (particularly C₁₋₈ alkyl group) on a ring nitrogen atom is preferably —(CH₂)ₘ-Q, —(CH₂)ₘ-Z¹-optionally substituted C₁₋₄ alkyl, —(CH₂)ₘ-Z² (CH₂)ₙ-Q, or —(CH₂)ₘ-Z² (CH₂)ₙ-Z¹-optionally halogenated C₁₋₄ alkyl, more preferably Q is hydroxy or —CONH₂, m is 0, Z¹ is —NH—CO— or —NH—CO₂— or Z¹ and Z² are —O—. As the specific substituent(s) on a ring nitrogen atom, methyl, ethyl, isopropyl, 2-hydroxyethoxyethyl, 2-methoxyethoxyethyl, 2-methoxyethyl, 2-hydroxyethyl, t-butoxycarbonylaminomethyl, t-butoxycarbonylaminoethyl, 2-hydroxy-2-methylpropylcarbonylaminomethyl, 2-hydroxy-2-methylpropylcarbonylaminoethyl, 1-methylsulfonyl-1-methylethylcarbonylaminoethyl, carbamoylmethyl and the like can be mentioned.

In compound (I), X is preferably CH, Y is preferably a nitrogen atom, and T is preferably a single bond.

In compound (I), Z is preferably an optionally substituted C₃₋₈ cycloalkanediyl group, an optionally substituted C₆₋₁₄ arylene group or an optionally substituted divalent heterocyclic group, more preferably an optionally substituted cyclohexanediyl group, an optionally substituted phenylene group, a phenylenemethylene group or a naphthalenediyl group, or an optionally substituted divalent aromatic fused heterocyclic group (the divalent aromatic fused heterocyclic group is preferably quinolinediyl). When Z is substituted, the substituent is preferably halogen atom, cyano group, optionally substituted hydrocarbon group, optionally substituted hydrocarbon-oxy group, optionally substituted amino group, optionally substituted hydroxy group, optionally substituted sulfanyl group or acyl group, more preferably halogen atom, exemplified for the above-mentioned substituent group (1). As specific Z, cyclohexanediyl, phenylene, methylphenylene, methoxyphenylene, hydroxymethylphenylene, fluorophenylene, chlorophenylene, naphthalenediyl, quinolinediyl, phenylenemethylene and the like can be mentioned.

In compound (I), U is preferably an “optionally substituted amido group”, an “optionally substituted sulfonamido group”, an “optionally substituted ureido group”, an “optionally substituted carbamoyl group” or an “optionally substituted thioureido group”, by substituent(s) selected from the optionally substituted hydrocarbon group, the optionally substituted heterocyclic group, the optionally substituted amino group, the optionally substituted hydroxy group, the optionally substituted sulfanyl group and the acyl group exemplified for the above-mentioned substituent group (1).

More preferably U is an “optionally substituted ureido group” by substituent(s) selected from the optionally substituted hydrocarbon group, the optionally substituted heterocyclic group, the optionally substituted amino group, the optionally substituted hydroxy group, the optionally substituted sulfanyl group and the acyl group exemplified for the above-mentioned substituent group (1), more preferably, an “optionally substituted ureido group” by the substituent(s) selected from a C₆₋₁₄ aryl group optionally substituted by the substituent(s) exemplified for the above-mentioned substituent group (2) and the optionally substituted heterocyclic group exemplified for the above-mentioned substituent group (1), and still more preferably, an “optionally substituted ureido group” by the substituent(s) selected from a phenyl group optionally substituted by the substituent(s) exemplified for the above-mentioned substituent group (2) and the optionally substituted heterocyclic group exemplified for the above-mentioned substituent group (1).

As preferable examples of compound (I), for example, the following compounds can be mentioned.

### Compound (I-1)

Compound (I) wherein U is an optionally substituted (having substituents R^{S2}, R^{S7} and R^{S8}) ureido group

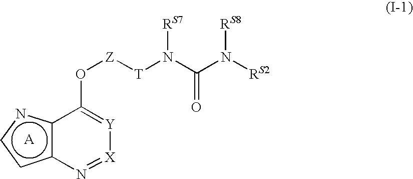

wherein R^{S2} is a hydrogen atom, an optionally substituted hydrocarbon group, an optionally substituted heterocyclic group, an optionally substituted amino group, an optionally substituted hydroxy group, an optionally substituted sulfanyl group or an acyl group, and R^{S7} and R^{S8} are each a hydrogen atom, an optionally substituted hydrocarbon group or an optionally substituted heterocyclic group.

In compound (I-1), preferable ring A, X, Y, Z and T are as explained above.

In compound (I-1), as the optionally substituted hydrocarbon group, optionally substituted heterocyclic group, optionally substituted amino group, optionally substituted hydroxy group, optionally substituted sulfanyl group and acyl group for R^{S2}, those similar to the optionally substituted hydrocarbon group, optionally substituted heterocyclic group, optionally substituted amino group, optionally substituted hydroxy group, optionally substituted sulfanyl group and acyl group, respectively exemplified for the above-mentioned substituent group (1), can be used.

As the optionally substituted hydrocarbon group and optionally substituted heterocyclic group for R^{S7} and R^{S8}, those similar to the optionally substituted hydrocarbon group and optionally substituted heterocyclic group, respectively exemplified for the above-mentioned substituent group (1), can be used.

R^{S7} and R^{S8} are each preferably a hydrogen atom.

R^{S2} is preferably a C₁₋₈ alkyl group (the C₁₋₈ alkyl group is preferably methyl or propyl) optionally substituted by the substituent(s) exemplified for the above-mentioned substituent group (2), a C₃₋₈ cycloalkyl group (the C₃₋₈ cycloalkyl group is preferably cyclopropyl) optionally substituted by the substituent(s) exemplified for the above-mentioned substituent group (2), a C₆₋₁₈ aryl-C₁₋₄ alkyl group (the C₆₋₁₈ aryl-C₁₋₄ alkyl group is preferably benzyl or phenylethyl) optionally substituted by the substituent(s) exemplified for the above-mentioned substituent group (2), a C₆₋₁₄ aryl group (the C₆₋₁₄ aryl group is preferably phenyl, naphthyl, biphenylyl or tetrahydronaphthyl) optionally substituted by the substituent(s) exemplified for the above-mentioned substituent group (2) or a heterocyclic group optionally substituted by the substituent(s) exemplified for the above-mentioned substituent group (2) (more preferably optionally substituted aromatic monocyclic heterocyclic group (the aromatic monocyclic heterocyclic group is preferably pyridyl, oxazolyl, isoxazolyl, pyrimidinyl, pyrazolyl or thiadiazolyl), an optionally substituted non-aromatic (aliphatic) heterocyclic group (the non-aromatic heterocyclic group is preferably piperidinyl), an optionally substituted aromatic fused heterocyclic group (the aromatic fused heterocyclic group is preferably quinolyl, isoquinolyl or benzothiazolyl), or an optionally substituted aliphatic fused heterocyclic group (the aliphatic fused heterocyclic group is preferably benzodioxinyl or tetrahydroisoquinolyl)).

The substituents exemplified for the above-mentioned substituent group (2) preferably include a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO— (optionally substituted alkyl group, alkoxy group, optionally substituted heterocyclic group or optionally substituted amino group), a C₆₋₁₈ aryl group, a heterocyclic group, an optionally substituted alkylthio group and an optionally substituted alkylsulfinyl group, more preferably halogen atom, a C₁₋₄ alkyl group optionally substituted by halogen atom or Q, a C₁₋₄ alkyl oxy group optionally substituted by halogen atom or Q, a C₆₋₁₈ aryl-oxy group optionally substituted by halogen atom or Q, and an optionally substituted heterocyclic group, more preferably, a halogen atom, an optionally halogenated C₁₋₄ alkyl group, an optionally halogenated C₁₋₄ alkyl-oxy group, a C₆₋₁₈ aryl-oxy group, and a heterocyclic group (preferably a 5- to 8-membered heterocyclic group having 1 to 3 hetero atoms selected from a nitrogen atom, an oxygen atom and an optionally oxidized sulfur atom).

As specific R^{S2}, methyl, n-propyl, cyclopropyl, cyclopropylmethyl, phenylethyl, trifluoropropyl, benzyl, phenyl, naphthyl, biphenylyl, ethynylphenyl, trifluoromethylphenyl, chlorophenyl, methoxyphenyl, bromophenyl, fluorophenyl, methylphenyl, dimethoxyphenyl, trifluoromethoxyphenyl, phenoxyphenyl, t-butylphenyl, methyl(trifluoromethyl)phenyl, hydroxy(trifluoromethyl)phenyl, {trifluoro(hydroxy)ethyl}phenyl, {trifluoro(methyl)(hydroxy)ethyl}phenyl, fluoro{trifluoro(methyl)(hydroxy)ethyl}phenyl, fluoro(trifluoromethyl)phenyl, chloro(trifluoromethyl)phenyl, cyano(trifluoromethyl)phenyl, {trifluoro(methoxy)ethyl}phenyl, methoxy(trifluoromethyl)phenyl, trifluoromethyl(methoxycarbonyl)phenyl, trifluoromethyl(benzyloxy)phenyl, trifluoromethyl(morpholinocarbonyl)phenyl, trifluoromethyl(morpholinomethyl)phenyl, trifluoromethyl(N-methylpiperazinylcarbonyl)phenyl, trifluoromethyl(N-methylpiperidinyloxy)phenyl, chlorotrifluorophenyl, difluoromethoxyphenyl, trifluoromethylthiophenyl, trifluoromethylsulfinylphenyl, trifluoromethyl(imidazolyl)phenyl, trifluoromethyl(morpholino)phenyl, trifluoromethyl(methylcarbamoyl)phenyl, trifluoromethyl(N-methylpiperazinylmethyl)phenyl, trifluoromethyl(hydroxypiperazinylmethyl)phenyl, acetylphenyl, methoxycarbonylphenyl, tetrafluoroethoxyphenyl, imidazolylphenyl, tetrafluorobenzodioxinyl, methylisoxazolyl, methyl(phenyl)isoxazolyl, t-butylisoxazolyl, trifluoromethylthiadiazolyl, trifluoromethyloxazolyl, pyridyl, trifluoromethylpyridyl, trifluoromethylpyridyl(N-oxide), chloropyridyl, methylpyridyl, dimethylpyridyl, quinolyl, isoquinolyl, tetrahydroisoquinolyl, N-methyl-tetrahydroisoquinolyl, N-trifluoromethylcarbonyl-tetrahydroisoquinolyl, methoxypyrimidinyl, N-t-butoxycarbonylpiperidinyl, N-methyl-t-butylpyrazolyl, N-trifluoroethylpyrazolyl, N-methyl-{trifluoro(hydroxy)ethyl}pyrazolyl, hydroxy(trifluoromethyl)tetrahydronaphthyl, benzothiazolyl and the like can be mentioned.

### Compound (I-2)

Compound (I) wherein U is an optionally substituted (having substituents R^{S7} and R^{S9}) amido group

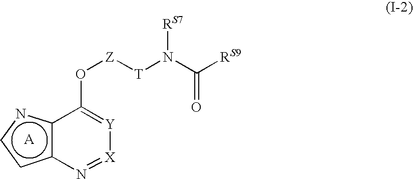

wherein R^{S9} is a hydrogen atom, an optionally substituted hydrocarbon group or an optionally substituted heterocyclic group and R^{S7} is as defined above.

In compound (I-2), preferable ring A, X, Y, Z and T are as explained above.

As the optionally substituted hydrocarbon group and optionally substituted heterocyclic group for R^{S9}, those similar to the optionally substituted hydrocarbon group and optionally substituted heterocyclic group, respectively exemplified for the above-mentioned substituent group (1) can be used.

R^{S7} is preferably a hydrogen atom.

As specific R^{S9}, a hydrogen atom, phenyl, benzyl and the like can be mentioned.

### Compound (I-3)

Compound (I) wherein U is an optionally substituted (having substituents R^{S11} and R^{S12}) carbamoyl group

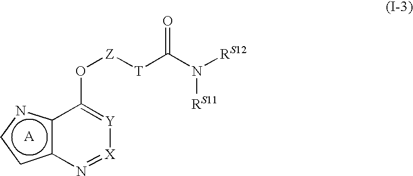

wherein R^{S11} and R^{S12} are each a hydrogen atom, an optionally substituted hydrocarbon group, an optionally substituted heterocyclic group, an optionally substituted amino group, an optionally substituted hydroxy group, an optionally substituted sulfanyl group or an acyl group.

In compound (I-3), preferable ring A, X, Y, Z and T are as explained above.

As the optionally substituted hydrocarbon group, optionally substituted heterocyclic group, optionally substituted amino group, optionally substituted hydroxy group, optionally substituted sulfanyl group and acyl group for R^{S11} and R^{S12}, those similar to the optionally substituted hydrocarbon group, optionally substituted heterocyclic group, optionally substituted amino group, optionally substituted hydroxy group, optionally substituted sulfanyl group and acyl group, respectively exemplified for the above-mentioned substituent group (1), can be used.

Preferably, one of R^{S11} and R^{S12} is a hydrogen atom.

As specific R^{S11} and R^{S12}, a hydrogen atom, methyl, phenyl and the like can be mentioned.

### Compound (I-4)

Compound (I) wherein U is an optionally substituted (having substituents R^{S7} and R^{s13}) sulfonamido group

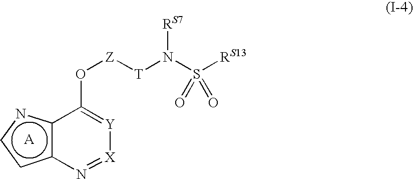

wherein R^{S13} is a hydroxy group, an optionally substituted hydrocarbon group or an optionally substituted heterocyclic group, and R^{S7} is as defined above.

In compound (I-4), preferable ring A, X, Y, Z and T are as explained above.

As the optionally substituted hydrocarbon group and optionally substituted-heterocyclic group for R^{S13}, those similar to the optionally substituted hydrocarbon group and optionally substituted heterocyclic group, respectively exemplified for the above-mentioned substituent group (1), can be used.

### Compound (I-5)

Compound (I) wherein U is an optionally substituted (having substituents R^{S14}, R^{S15} and R^{S16}) thioureido group

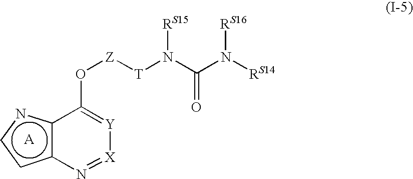

wherein R^{S14} is a hydrogen atom, an optionally substituted hydrocarbon group, an optionally substituted heterocyclic group, an optionally substituted amino group, an optionally substituted hydroxy group, an optionally substituted sulfanyl group or an acyl group, and R^{S15} and R^{S16} are each a hydrogen atom, an optionally substituted hydrocarbon group or an optionally substituted heterocyclic group.

In compound (I-5), preferable ring A, X, Y, Z and T are as explained above.

In compound (I-5), as the optionally substituted hydrocarbon group, optionally substituted heterocyclic group, optionally substituted amino group, optionally substituted hydroxy group, optionally substituted sulfanyl group and acyl group for R^{S14}, those similar to the optionally substituted hydrocarbon group, optionally substituted heterocyclic group, optionally substituted amino group, optionally substituted hydroxy group, optionally substituted sulfanyl group and acyl group, respectively exemplified for the above-mentioned substituent group (1), can be used.

As the optionally substituted hydrocarbon group and optionally substituted heterocyclic group for R^{S15} and R^{S16}, those similar to the optionally substituted hydrocarbon group and optionally substituted heterocyclic group, respectively exemplified for the above-mentioned substituent group (1), can be used.

R^{S15} and R^{S16} are each preferably hydrogen atom.

R^{S14} is preferably a C₁₋₈ alkyl group (the C₁₋₈ alkyl group is preferably methyl or propyl) optionally substituted by the substituent(s) exemplified for the above-mentioned substituent group (2), a C₃₋₈ cycloalkyl group (the C₃₋₈ cycloalkyl group is preferably cyclopropyl) optionally substituted by the substituent(s) exemplified for the above-mentioned substituent group (2), a C₆₋₁₈ aryl-C₁₋₄ alkyl group (the C₆₋₁₈ aryl-C₁₋₄ alkyl group is preferably benzyl or phenylethyl) optionally substituted by the substituent(s) exemplified for the above-mentioned substituent group (2), a C₆₋₁₄ aryl group (the C₆₋₁₄ aryl group is preferably phenyl, naphthyl, biphenylyl or tetrahydronaphthyl) optionally substituted by the substituent(s) exemplified for the above-mentioned substituent group (2) or a heterocyclic group optionally substituted by the substituent(s) exemplified for the above-mentioned substituent group (2) (more preferably, an optionally substituted aromatic monocyclic heterocyclic group (the aromatic monocyclic heterocyclic group is preferably pyridyl, oxazolyl, isoxazolyl, pyrimidinyl, pyrazolyl or thiadiazolyl), an optionally substituted non-aromatic (aliphatic) heterocyclic group (the non-aromatic heterocyclic group is preferably piperidinyl), an optionally substituted aromatic fused heterocyclic group (the aromatic fused heterocyclic group is preferably quinolyl, isoquinolyl or benzothiazolyl), or an optionally substituted aliphatic fused heterocyclic group (the aliphatic fused heterocyclic group is preferably benzodioxinyl or tetrahydroisoquinolyl)).

The substituents exemplified for the above-mentioned substituent group (2) preferably include a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO— (optionally substituted alkyl group, alkoxy group, optionally substituted heterocyclic group or optionally substituted amino group), a C₆₋₁₈ aryl group, a heterocyclic group, an optionally substituted alkylthio group, or an optionally substituted alkylsulfinyl group, more preferably a halogen atom, a C₁₋₄ alkyl group optionally substituted by halogen atom or Q, a C₁₋₄ alkyl-oxy group optionally substituted by halogen atom or Q, or a C₆₋₁₈ aryl-oxy group optionally substituted by halogen atom or Q, optionally substituted heterocyclic group, more preferably, a halogen atom, an optionally halogenated Cl₁₋₄ alkyl group, an optionally halogenated C₁₋₄ alkyl-oxy group, a C₆₋₁₈ aryl-oxy group, or a heterocyclic group (preferably, a 5- to 8-membered heterocyclic group having 1 to 3 hetero atoms selected from a nitrogen atom, an oxygen atom and an optionally oxidized sulfur atom).

As specific R^{S14}, phenyl and the like can be mentioned.

A more preferable example of compound (I) is, for example, the following compound.

Compound (I) wherein

(1) ring A is a pyrrole ring having, on a ring nitrogen atom, a C₁₋₈ alkyl group (the C₁₋₈ alkyl group is particularly methyl or ethyl) optionally substituted by substituent(s) selected from —(CH₂)ₘ-Q, —(CH₂)ₘ-Z¹-optionally substituted C₁₋₄ alkyl, —(CH₂)ₘ-Z²-(CH₂)ₙ-Q and —(CH₂)ₘ-Z²-(CH₂)ₙ-Z¹-optionally halogenated C₁₋₄ alkyl (preferably Q is hydroxy or —CONH₂, m is 0, Z¹ is —NH—CO— or —NH—CO₂—, or Z¹ and Z² are each —O—),

(2) X is CH,

(3) Y is a nitrogen atom,

(4) Z is an optionally substituted C₃₋₈ cycloalkanediyl group (preferably optionally substituted cyclohexanediyl group), an optionally substituted C₆₋₁₄ arylene group (preferably optionally substituted phenylene group or naphthalenediyl group) or an optionally substituted divalent heterocyclic group (preferably optionally substituted divalent aromatic fused heterocyclic group (the divalent aromatic fused heterocyclic group is preferably quinolinediyl)),

(5) T is a single bond, and

(6) U is a ureido group substituted by a C₁₋₈ alkyl group (particularly methyl or propyl), a C₃₋₈ cycloalkyl group (particularly cyclopropyl), a C₆₋₁₈ aryl-C₁₋₄ alkyl group (particularly benzyl or phenylethyl), a C₆₋₁₄ aryl group (particularly phenyl, naphthyl, biphenylyl or tetrahydronaphthyl), an aromatic monocyclic heterocyclic group (particularly pyridyl, oxazolyl, isoxazolyl, pyrimidinyl, pyrazolyl or thiadiazolyl), a non-aromatic (aliphatic) heterocyclic group (particularly piperidinyl), an aromatic fused heterocyclic group (particularly quinolyl, isoquinolyl or benzothiazolyl), or an aliphatic fused heterocyclic group (particularly benzodioxinyl or tetrahydroisoquinolyl), each of which may be substituted by substituent(s) selected from a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO— (optionally substituted alkyl group, alkoxy group, optionally substituted heterocyclic group or optionally substituted amino group), a C₆₋₁₈ aryl group, a heterocyclic group, an optionally substituted alkylthio group and an optionally substituted alkylsulfinyl group.

A more preferable example of compound (I) is, for example, the following compound.

Compound (I) wherein

(1) ring A is a pyrrole ring having, on a ring nitrogen atom, a C₁₋₈ alkyl group (the C₁₋₈ alkyl group is particularly methyl or ethyl) optionally substituted by substituent(s) selected from hydroxy, —O-optionally halogenated C₁₋₄ alkyl, —O —(CH₂)ₙ-hydroxy and —O—(CH₂)ₙ—O-optionally halogenated C₁₋₄ alkyl,

(2) X is CH,

(3) Y is a nitrogen atom,

(4) Z is a C₆₋₁₄ arylene group (particularly, phenylene group or naphthalenediyl group) or a 5- to 12-membered divalent heterocyclic group having 1 to 3 hetero atoms selected from a nitrogen atom, an oxygen atom and an optionally oxidized sulfur atom (preferably a 8- to 12-membered divalent aromatic fused heterocyclic group having 1 to 3 hetero atoms selected from a nitrogen atom, an oxygen atom and an optionally oxidized sulfur atom, particularly quinolinediyl), each optionally substituted by a halogen atom, preferably a C₆₋₁₄ arylene group (particularly phenylene group) substituted by a halogen atom,

(5) T is a single bond, and

(6) U is a ureido group substituted by a C₁₋₈ alkyl group (particularly methyl or propyl), a C₆₋₁₈ aryl-C₁₋₄ alkyl group (particularly benzyl), a C₆₋₁₄ aryl group (particularly phenyl), a 5- or 6-membered aromatic monocyclic heterocyclic group having 1 to 3 hetero atoms selected from a nitrogen atom, an oxygen atom and an optionally oxidized sulfur atom (particularly pyridyl, oxazolyl, isoxazolyl or thiadiazolyl) or a 8- to 12-membered aliphatic fused heterocyclic group having 1 to 3 hetero atoms selected from a nitrogen atom, an oxygen atom and an optionally oxidized sulfur atom (particularly benzodioxinyl), each of which may be substituted by substituent(s) selected from a halogen atom, an optionally halogenated C₁₋₄ alkyl group, an optionally halogenated C₁₋₄ alkyl-oxy group, a C₆₋₁₈ aryl-oxy group and a 5- to 8-membered heterocyclic group having 1 to 3 hetero atoms selected from a nitrogen atom, an oxygen atom and an optionally oxidized sulfur atom.

A particularly preferable example of compound (I) is, for example, the following compound.

Compound (I) wherein

(1) ring A is a pyrrole ring having, on a ring nitrogen atom, methyl, ethyl, 2-hydroxyethoxyethyl, 2-methoxyethoxyethyl, 2-methoxyethyl or 2-hydroxyethyl,

(2) X is CH,

(3) Y is a nitrogen atom,

(4) Z is phenylene, fluorophenylene, chlorophenylene, naphthalenediyl or quinolinediyl,

(5) T is a single bond, and

(6) U is a ureido group substituted by methyl, n-propyl, benzyl, phenyl, trifluoromethylphenyl, chlorophenyl, methoxyphenyl, bromophenyl, fluorophenyl, methylphenyl, trifluoromethoxyphenyl, phenoxyphenyl, t-butylphenyl, chlorotrifluorophenyl, tetrafluoroethoxyphenyl, imidazolylphenyl, tetrafluorobenzodioxinyl, methylisoxazolyl, trifluoromethylthiadiazolyl, trifluoromethyloxazolyl or trifluoromethylpyridyl.

Compound (II) in the present invention is a preferable compound of compound (I), and corresponds to compound (I) wherein ring A is an optionally substituted pyrrole ring (having substituent R¹ on a ring nitrogen atom), X is CH, Y is a nitrogen atom, T is a single bond and U is a ureido group having substituent R².

In compound (II), as the “optionally substituted hydrocarbon group”, “optionally substituted heterocyclic group” and “acyl group” for R¹, those similar to the “optionally substituted hydrocarbon group”, “optionally substituted heterocyclic group” and “acyl group” exemplified for the above-mentioned substituent group (1) can be used.

In compound (II), as the “optionally substituted hydrocarbon group”, “optionally substituted heterocyclic group”, “optionally substituted amino group”, “optionally substituted hydroxy group”, “optionally substituted sulfanyl group” and “acyl group” for R², those similar to the “optionally substituted hydrocarbon group”, “optionally substituted heterocyclic group”, “optionally substituted amino group”, “optionally substituted hydroxy group”, “optionally substituted sulfanyl group” and “acyl group” exemplified for the above-mentioned substituent group (1) can be used.

In compound (II), R¹ is preferably a hydrogen atom or an optionally substituted hydrocarbon group, more preferably an optionally substituted hydrocarbon group, more preferably a C₁₋₈ alkyl group (the C₁₋₈ alkyl group is preferably methyl or ethyl) optionally substituted by the substituent(s) exemplified for the above-mentioned substituent group (2).

When R¹ is substituted, the substituent is preferably —(CH₂)ₘ-Q, —(CH₂)ₘ-Z¹-optionally substituted C₁₋₄ alkyl, —(CH₂)ₘ-Z²-(CH₂)ₙ-Q, or —(CH₂)ₘ-Z²-(CH₂)ₙ-Z¹-optionally halogenated C₁₋₄ alkyl, more preferably Q is hydroxy or —CONH₂, m is 0, Z¹ is NH—CO— or —NH—CO₂—, or Z¹ and Z² are each —O—. As specific R¹, methyl, ethyl, isopropyl, 2-hydroxyethoxyethyl, 2-methoxyethoxyethyl, 2-methoxyethyl, 2-hydroxyethyl, t-butoxycarbonylaminomethyl, t-butoxycarbonylaminoethyl, 2-hydroxy-2-methylpropylcarbonylaminomethyl, 2-hydroxy-2-methylpropylcarbonylaminoethyl, 1-methylsulfonyl-1-methylethylcarbonylaminoethyl, carbamoylmethyl and the like can be mentioned.

In compound (II), Z is preferably a C₃₋₈ cycloalkanediyl group optionally substituted by substituent(s) exemplified for the above-mentioned substituent group (1), a C₆₋₁₄ arylene group optionally substituted by substituent(s) exemplified for the above-mentioned substituent group (1) or a 5- to 12-membered divalent heterocyclic group having, as a ring-constituting atom (ring atom), 1 to 3 kinds of 1 to 4 hetero atoms selected from an oxygen atom, an optionally oxidized sulfur atom, a nitrogen atom and the like, which is optionally substituted by substituent(s) exemplified for the above-mentioned substituent group (1), more preferably a cyclohexanediyl group optionally substituted by substituent(s) exemplified for the above-mentioned substituent group (1), a phenylene group optionally substituted by substituent(s) exemplified for the above-mentioned substituent group (1), a phenylenemethylene group or a naphthalenediyl group, or a 8- to 12-membered divalent aromatic fused heterocyclic group (the divalent aromatic fused heterocyclic group is preferably quinolinediyl) having, as a ring-constituting atom (ring atom), 1 to 3 kinds of 1 to 4 hetero atoms selected from an oxygen atom, an optionally oxidized sulfur atom, a nitrogen atom and the like, which is optionally substituted by substituent(s) exemplified for the above-mentioned substituent group (1).

When Z is substituted, the substituent is preferably a halogen atom, a cyano group, an optionally substituted hydrocarbon group, an optionally substituted hydrocarbon-oxy group, an optionally substituted amino group, an optionally substituted hydroxy group, an optionally substituted sulfanyl group or an acyl group, more preferably a halogen atom. As specific Z, cyclohexanediyl, phenylene, methylphenylene, methoxyphenylene, hydroxymethylphenylene, fluorophenylene, chlorophenylene, naphthalenediyl, quinolinediyl, phenylenemethylene and the like can be mentioned.

In compound (II), R² is preferably an optionally substituted C₁₋₈ alkyl group (the C₁₋₈ alkyl group is preferably methyl or propyl), an optionally substituted C₃₋₈ cycloalkyl group (the C₃₋₈ cycloalkyl group is preferably cyclopropyl), an optionally substituted C₆₋₁₈ aryl-C₁₋₄ alkyl group (the C₆₋₁₈ aryl-C₁₋₄ alkyl group is preferably benzyl or phenylethyl), an optionally substituted C₆₋₁₄ aryl group (the C₆₋₁₄ aryl group is preferably phenyl, naphthyl, biphenylyl or tetrahydronaphthyl) or an optionally substituted heterocyclic group (more preferably, an optionally substituted aromatic monocyclic heterocyclic group (the aromatic monocyclic heterocyclic group is preferably pyridyl, oxazolyl, isoxazolyl, pyrimidinyl, pyrazolyl or thiadiazolyl), an optionally substituted non-aromatic (aliphatic) heterocyclic group (the non-aromatic heterocyclic group is preferably piperidinyl), an optionally substituted aromatic fused heterocyclic group (the aromatic fused heterocyclic group is preferably quinolyl, isoquinolyl or benzothiazolyl), or an optionally substituted aliphatic fused heterocyclic group (the aliphatic fused heterocyclic group is preferably benzodioxinyl or tetrahydroisoquinolyl)).

When R² is substituted, the substituent is preferably a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO-(optionally substituted alkyl group, alkoxy group, optionally substituted heterocyclic group or optionally substituted amino group), a C₆₋₁₈ aryl group, a heterocyclic group, an optionally substituted alkylthio group, or an optionally substituted alkylsulfinyl group, more preferably, a halogen atom, an optionally substituted C₁₋₈ alkyl group, an optionally substituted C₁₋₈ alkyl-oxy group, an optionally substituted C₆₋₁₈ aryl-oxy group, or an optionally substituted heterocyclic group, more preferably, a halogen atom, a C₁₋₈ alkyl group optionally substituted by a halogen atom, a C₁₋₈ alkyl-oxy group optionally substituted by a halogen atom, a C₆₋₁₈ aryl-oxy group or a heterocyclic group.

As specific R², methyl, n-propyl, cyclopropyl, cyclopropylmethyl, phenylethyl, trifluoropropyl, benzyl, phenyl, naphthyl, biphenylyl, ethynylphenyl, trifluoromethylphenyl, chlorophenyl, methoxyphenyl, bromophenyl, fluorophenyl, methylphenyl, dimethoxyphenyl, trifluoromethoxyphenyl, phenoxyphenyl, t-butylphenyl, methyl(trifluoromethyl)phenyl, hydroxy(trifluoromethyl)phenyl, {trifluoro(hydroxy)ethyl}phenyl, {trifluoro(methyl)(hydroxy)ethyl}phenyl, fluoro{trifluoro(methyl)(hydroxy)ethyl}phenyl, fluoro(trifluoromethyl)phenyl, chloro(trifluoromethyl)phenyl, cyano(trifluoromethyl)phenyl, {trifluoro(methoxy)ethyl}phenyl, methoxy(trifluoromethyl)phenyl, trifluoromethyl(methoxycarbonyl)phenyl, trifluoromethyl(benzyloxy)phenyl, trifluoromethyl(morpholinocarbonyl)phenyl, trifluoromethyl(morpholinomethyl)phenyl, trifluoromethyl(N-methylpiperazinylcarbonyl)phenyl, trifluoromethyl(N-methylpiperidinyloxy)phenyl, chlorotrifluorophenyl, difluoromethoxyphenyl, trifluoromethylthiophenyl, trifluoromethylsulfinylphenyl, trifluoromethyl(imidazolyl)phenyl, trifluoromethyl(morpholino)phenyl, trifluoromethyl(methylcarbamoyl)phenyl, trifluoromethyl(N-methylpiperazinylmethyl)phenyl, trifluoromethyl(hydroxypiperazinylmethyl)phenyl, acetylphenyl, methoxycarbonylphenyl, tetrafluoroethoxyphenyl, imidazolylphenyl, tetrafluorobenzodioxinyl, methylisoxazolyl, methyl(phenyl)isoxazolyl, t-butylisoxazolyl, trifluoromethylthiadiazolyl, trifluoromethyloxazolyl, pyridyl, trifluoromethylpyridyl, trifluoromethylpyridyl(N-oxide), chloropyridyl, methylpyridyl, dimethylpyridyl, quinolyl, isoquinolyl, tetrahydroisoquinolyl, N-methyl-tetrahydroisoquinolyl, N-trifluoromethylcarbonyl-tetrahydroisoquinolyl, methoxypyrimidinyl, N-t-butoxycarbonylpiperidinyl, N-methyl-t-butylpyrazolyl, N-trifluoroethylpyrazolyl, N-methyl-{trifluoro(hydroxy)ethyl}pyrazolyl, hydroxy(trifluoromethyl)tetrahydronaphthyl, benzothiazolyl and the like can be mentioned.

A preferable example of compound (II) is, for example, the following compound.

Compound (II) wherein

(1) R¹ is a C₁₋₈ alkyl group (the C₁₋₈ alkyl group is particularly methyl or ethyl) optionally substituted by —(CH₂)ₘ-Q, —(CH₂)ₘ-Z¹-optionally substituted C₁₋₄ alkyl, —(CH₂)ₘ-Z²-(CH₂)ₙ-Q or —(CH₂)ₘ-Z²-(CH₂)ₙ-Z¹-optionally halogenated C₁₋₄ alkyl (preferably Q is hydroxy or —CONH₂, m is 0, Z¹ is —NH—CO— or —NH—CO₂—, or Z¹ and Z² are each —O—),

(2) Z is an optionally substituted C₃₋₈ cycloalkanediyl group (preferably optionally substituted cyclohexanediyl group), an optionally substituted C₆₋₁₄ arylene group (preferably optionally substituted phenylene group or naphthalenediyl group) or an optionally substituted divalent heterocyclic group (preferably optionally substituted divalent aromatic fused heterocyclic group (the divalent aromatic fused heterocyclic group is preferably quinolinediyl)), and

(3) R² is a C₁₋₈ alkyl group (particularly methyl and propyl), a C₃₋₈ cycloalkyl group (particularly cyclopropyl), a C₆₋₁₈ aryl-C₁₋₄ alkyl group (particularly benzyl or phenylethyl), a C₆₋₁₄ aryl group (particularly phenyl, naphthyl, biphenylyl, tetrahydronaphthyl), an aromatic monocyclic heterocyclic group (particularly pyridyl, oxazolyl, isoxazolyl, pyrimidinyl, pyrazolyl and thiadiazolyl), a non-aromatic (aliphatic)heterocyclic group (particularly piperidinyl), an aromatic fused heterocyclic group (particularly quinolyl, isoquinolyl or benzothiazolyl), or an aliphatic fused heterocyclic group (particularly benzodioxinyl or tetrahydroisoquinolyl), which is optionally substituted by substituent(s) selected from a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO-(optionally substituted alkyl group, alkoxy group, optionally substituted heterocyclic group or optionally substituted amino group), a C₆₋₁₈ aryl group, a heterocyclic group, an optionally substituted alkylthio group, and an optionally substituted alkylsulfinyl group.

A more preferable example of compound (II) is, for example, the following compound.

Compound (II) wherein

(1) R¹ is a C₁₋₈ alkyl group (the C₁₋₈ alkyl group is particularly methyl or ethyl) optionally substituted by hydroxy, —O-optionally halogenated C₁₋₄ alkyl, —O—(CH₂)ₙ-hydroxy or —O—(CH₂)ₙ—O-optionally halogenated C₁₋₄ alkyl,

(2) Z is a C₆₋₁₄ arylene group (particularly, a phenylene group or a naphthalenediyl group) or a 5- to 12-membered divalent heterocyclic group having 1 to 3 hetero atoms selected from a nitrogen atom, an oxygen atom and an optionally oxidized sulfur atom (preferably, a 8- to 12-membered divalent aromatic fused heterocyclic group having 1 to 3 hetero atoms selected from a nitrogen atom, an oxygen atom and an optionally oxidized sulfur atom (particularly, quinolinediyl), each optionally substituted by a halogen atom, preferably a C₆₋₁₄ arylene group (particularly, a phenylene group) substituted by a halogen atom, and

(3) R² is a C₁₋₈ alkyl group (particularly, methyl or propyl), a C₆₋₁₈ aryl-C₁₋₄ alkyl group (particularly, benzyl), a C₆₋₁₄ aryl group (particularly, phenyl), a 5- or 6-membered aromatic monocyclic heterocyclic group having 1 to 3 hetero atoms selected from a nitrogen atom, an oxygen atom and an optionally oxidized sulfur atom (particularly, pyridyl, oxazolyl, isoxazolyl or thiadiazolyl) or an aliphatic fused heterocyclic group (particularly, benzodioxinyl), which is optionally substituted by substituent(s) selected from a halogen atom, an optionally halogenated C₁₋₄ alkyl group, an optionally halogenated C₁₋₄ alkyl-oxy group, a C₆₋₁₈ aryl-oxy group and a 5- to 8-membered heterocyclic group having 1 to 3 hetero atoms selected from a nitrogen atom, an oxygen atom and an optionally oxidized sulfur atom.

A particularly preferable example of compound (II) is, for example, the following compound.

Compound (II) wherein

(1). R¹ is methyl, ethyl, 2-hydroxyethoxyethyl, 2-methoxyethoxyethyl, 2-methoxyethyl or 2-hydroxyethyl,

(2) Z is phenylene, fluorophenylene, chlorophenylene, naphthalenediyl or quinolinediyl, and

(3) R² is methyl, n-propyl, benzyl, phenyl, trifluoromethylphenyl, chlorophenyl, methoxyphenyl, bromophenyl, fluorophenyl, methylphenyl, trifluoromethoxyphenyl, phenoxyphenyl, t-butylphenyl, chlorotrifluorophenyl, tetrafluoroethoxyphenyl, imidazolylphenyl, tetrafluorobenzodioxinyl, methylisoxazolyl, trifluoromethylthiadiazolyl, trifluoromethyloxazolyl or trifluoromethylpyridyl.

As specific examples of compound (II), for example, the compounds of Examples 1-150, 153, 156, 159 and 163 can be mentioned.

Compound (III) in the present invention is a preferable compound of compound (II). It corresponds to compound (II) wherein R¹ is a hydrogen atom or an optionally substituted hydrocarbon group, Z is a 1,4-phenylene group having substituents R³, R⁴, R⁵ and R⁶, R² is an optionally substituted hydrocarbon group (specifically, an optionally substituted phenyl group) or an optionally substituted heterocyclic group.

In other words, compound (III) in the present invention is a more preferable compound (I). It corresponds to compound (I) wherein ring A is an optionally substituted (having substituent R¹, which is a hydrogen atom or an optionally substituted hydrocarbon group, on a ring nitrogen atom) pyrrole ring, X is CH, Y is a nitrogen atom, Z is a 1,4-phenylene group having substituents R³, R⁴, R⁵ and R⁶, T is a 2′ single bond, and U is a substituted (having substituent R^{2′} which is an optionally substituted phenyl group or an optionally substituted heterocyclic group) ureido group.

As the substituent of the “optionally substituted phenyl group” for R^{2′} in compound (III), those exemplified for the above-mentioned substituent group (2) can be used.

The “optionally substituted heterocyclic group” for R^{2′} in compound (III) is as explained in the above as to the “optionally substituted heterocyclic group” for R² in compound (II).

As the “optionally substituted hydrocarbon group”, “optionally substituted amino group”, “optionally substituted hydroxy group”, “optionally substituted sulfanyl group” and “acyl group” for R³, R⁴, R⁵ and R⁶ in compound (III), those similar to the “optionally substituted hydrocarbon group”, “optionally substituted amino group”, “optionally substituted hydroxy group”, “optionally substituted sulfanyl group” and “acyl group” respectively exemplified for the above-mentioned substituent group (1) can be used.

In compound (III), R^{1′} is preferably an optionally substituted hydrocarbon group, more preferably an optionally substituted C₁₋₈ alkyl group (the C₁₋₈ alkyl group is preferably methyl or ethyl). When R^{1′} is substituted, the substituent is preferably —(CH₂)ₘ-Q, —(CH₂)ₘ-Z¹-optionally substituted C₁₋₄ alkyl, —(CH₂)ₘZ²-(CH₂)ₙ-Q, or —(CH₂)ₘ-Z¹-(CH₂)ₙ-Z¹-optionally halogenated C₁₋₄ alkyl, more preferably Q is hydroxy or —CONH₂, m is 0, Z¹ is —NH—CO— or —NH—CO₂—, or Z¹ and Z² are each —O—. As specific R¹, methyl, ethyl, isopropyl, 2-hydroxyethoxyethyl, 2-methoxyethoxyethyl, 2-methoxyethyl, 2-hydroxyethyl, t-butoxycarbonylaminomethyl, t-butoxycarbonylaminoethyl, 2-hydroxy-2-methylpropylcarbonylaminomethyl, 2-hydroxy-2-methylpropylcarbonylaminoethyl, 1-methylsulfonyl-1-methylethylcarbonylaminoethyl, carbamoylmethyl and the like can be mentioned.

In compound (III), R² is preferably a C₁₋₈ alkyl group (preferably methyl or propyl), a C₃₋₈ cycloalkyl group (preferably cyclopropyl), a C₆₋₁₈ aryl-C₁₋₄ alkyl group (preferably benzyl or phenylethyl), a C₆₋₁₄ aryl group (preferably phenyl, naphthyl, biphenylyl or tetrahydronaphthyl) or a heterocyclic group (more preferably, an aromatic monocyclic heterocyclic group (preferably pyridyl, oxazolyl, isoxazolyl, pyrimidinyl, pyrazolyl or thiadiazolyl), a non-aromatic (aliphatic) heterocyclic group (preferably piperidinyl), an aromatic fused heterocyclic group (preferably quinolyl, isoquinolyl or benzothiazolyl), or an aliphatic fused heterocyclic group (preferably benzodioxinyl or tetrahydroisoquinolyl)), which is optionally substituted by substituent(s) selected from a halogen atom, an optionally substituted C₁₋₈ alkyl group, an optionally substituted C₁₋₈ alkyl-oxy group, an optionally substituted C₆₋₁₈ aryl-oxy group and an optionally substituted heterocyclic group, more preferably, a phenyl group or heterocyclic group optionally substituted by substituent(s) selected from a halogen atom, an optionally substituted C₁₋₈ alkyl group, an optionally substituted C₁₋₈ alkyl-oxy group, an optionally substituted C₆₋₁₈ aryl-oxy group and an optionally substituted heterocyclic group. More preferably, it is a phenyl group, an aromatic monocyclic heterocyclic group (the aromatic monocyclic heterocyclic group is preferably pyridyl, oxazolyl, isoxazolyl or thiadiazolyl) or an aliphatic fused heterocyclic group (the aliphatic fused heterocyclic group is preferably benzodioxinyl), which is optionally substituted by substituent(s) selected from a halogen atom, a C₁₋₈ alkyl group optionally substituted by a halogen atom, a C₁₋₈ alkyl-oxy group optionally substituted by a halogen atom, a C₆₋₁₈ aryl-oxy group and a heterocyclic group.

As specific R^{2′}, methyl, n-propyl, cyclopropyl, cyclopropylmethyl, phenylethyl, trifluoropropyl, benzyl, phenyl, naphthyl, biphenylyl, ethynylphenyl, trifluoromethylphenyl, chlorophenyl, methoxyphenyl, bromophenyl, fluorophenyl, methylphenyl, dimethoxyphenyl, trifluoromethoxyphenyl, phenoxyphenyl, t-butylphenyl, methyl(trifluoromethyl)phenyl, hydroxy(trifluoromethyl)phenyl, {trifluoro(hydroxy)ethyl}phenyl, {trifluoro(methyl)(hydroxy)ethyl}phenyl, fluoro{trifluoro(methyl)(hydroxy)ethyl}phenyl, fluoro(trifluoromethyl)phenyl, chloro(trifluoromethyl)phenyl, cyano(trifluoromethyl)phenyl, {trifluoro(methoxy)ethyl}phenyl, methoxy(trifluoromethyl)phenyl, trifluoromethyl(methoxycarbonyl)phenyl, trifluoromethyl(benzyloxy)phenyl, trifluoromethyl(morpholinocarbonyl)phenyl, trifluoromethyl(morpholinomethyl)phenyl, trifluoromethyl(N-methylpiperazinylcarbonyl)phenyl, trifluoromethyl(N-methylpiperidinyloxy)phenyl, chlorotrifluorophenyl, difluoromethoxyphenyl, trifluoromethylthiophenyl, trifluoromethylsulfinylphenyl, trifluoromethyl(imidazolyl)phenyl, trifluoromethyl(morpholino)phenyl, trifluoromethyl(methylcarbamoyl)phenyl, trifluoromethyl(N-methylpiperazinylmethyl)phenyl, trifluoromethyl(hydroxypiperazinylmethyl)phenyl, acetylphenyl, methoxycarbonylphenyl, tetrafluoroethoxyphenyl, imidazolylphenyl, tetrafluorobenzodioxinyl, pyridyl, methylisoxazolyl, methyl(phenyl)isoxazolyl, t-butylisoxazolyl, trifluoromethylthiadiazolyl, trifluoromethyloxazolyl, trifluoromethylpyridyl, trifluoromethylpyridyl(N-oxide), chloropyridyl, methylpyridyl, dimethylpyridyl, quinolyl, isoquinolyl, tetrahydroisoquinolyl, N-methyl-tetrahydroisoquinolyl, N-trifluoromethylcarbonyl-tetrahydroisoquinolyl, methoxypyrimidinyl, N-t-butoxycarbonylpiperidinyl, N-methyl-t-butylpyrazolyl, N-trifluoroethylpyrazolyl, N-methyl-{trifluoro(hydroxy)ethyl}pyrazolyl, hydroxy(trifluoromethyl)tetrahydronaphthyl, benzothiazolyl, N-methylpiperidinyl, t-butylpyrazolyl and the like can be mentioned.

In compound (III), R³, R⁴, R⁵ and R⁶ are preferably each independently a hydrogen atom or a halogen atom. More preferably, R³, R⁴, R⁵ and R⁶ are all hydrogen atoms, or one of R³, R⁴, R⁵ and R⁶ is a halogen atom and the rest are hydrogen atoms.

A preferable example of compound (III) is, for example, the following compound.

Compound (III) wherein

(1) R^{1′} is a C₁₋₈ alkyl group (the C₁₋₈ alkyl group is particularly methyl or ethyl) optionally substituted by —(CH₂)ₘ-Q, —(CH₂)ᵣ-Z¹-optionally substituted C₁₋₄ alkyl, —(CH₂)ₘ-Z²-(CH₂)ₙ-Q or —(CH₂)ₘ-Z²-(CH₂)ₙ-Z¹-optionally halogenated C₁₋₄ alkyl (preferably Q is hydroxy or —CONH₂, m is 0, Z¹ is —NH—CO— or —NH—CO₂—, or Z¹ and Z² are each —O—),

(2) R^{2′} is a C₁₋₈ alkyl group (particularly methyl and propyl), a C₃₋₈ cycloalkyl group (particularly cyclopropyl), a C₆₋₁₈ aryl-C₁₋₄ alkyl group (particularly benzyl or phenylethyl), a C₆₋₁₄ aryl group (particularly phenyl, naphthyl, biphenylyl, tetrahydronaphthyl), an aromatic monocyclic heterocyclic group (particularly pyridyl, oxazolyl, isoxazolyl, pyrimidinyl, pyrazolyl and thiadiazolyl), a non-aromatic (aliphatic)heterocyclic group (particularly piperidinyl), an aromatic fused heterocyclic group (particularly quinolyl, isoquinolyl or benzothiazolyl), or an aliphatic fused heterocyclic group (particularly benzodioxinyl or tetrahydroisoquinolyl), which is optionally substituted by substituent(s) selected from a halogen atom, an oxo group, a cyano group, a hydroxy group, an optionally substituted C₁₋₈ alkyl group, an optionally substituted C₁₋₈ alkyl-oxy group, an optionally substituted heterocycle-oxy group, a C₃₋₈ cycloalkyl group, a C₂₋₈ alkynyl group, —CO-(optionally substituted alkyl group, alkoxy group, optionally substituted heterocyclic group or optionally substituted amino group), a C₆₋₁₈ aryl group, a heterocyclic group, an optionally substituted alkylthio group, and an optionally substituted alkylsulfinyl group, and

(3) R³, R⁴, R⁵ and R⁶ are each independently a hydrogen atom or a halogen atom (preferably R⁴ is a halogen atom).

A more preferable example of compound (III) is, for example, the following compound.

Compound (III) wherein

(1) R^{1′} is a C₁₋₈ alkyl group (the C₁₋₈ alkyl group is particularly methyl or ethyl) optionally substituted by substituent(s) selected from hydroxy, —O-optionally halogenated C₁₋₄ alkyl, —O—(CH₂)ₙ-hydroxy and —O— (CH₂)ₙ—O-optionally halogenated C₁₋₄ alkyl,

(2) R^{2′} is a phenyl group, a 5- or 6-membered aromatic monocyclic heterocyclic group having 1 to 3 hetero atoms selected from a nitrogen atom, an oxygen atom and an optionally oxidized sulfur atom (particularly, pyridyl, oxazolyl, isoxazolyl or thiadiazolyl) or a 8- to 12-membered aliphatic fused heterocyclic group having 1 to 3 hetero atoms selected from a nitrogen atom, an oxygen atom and an optionally oxidized sulfur atom (particularly, benzodioxinyl), which is optionally substituted by substituent(s) selected from a halogen atom, an optionally halogenated C₁₋₄ alkyl group, an optionally halogenated C₁₋₄ alkyl-oxy group, a C₆₋₁₈ aryl-oxy group and a 5- to 8-membered heterocyclic group having 1 to 3 hetero atoms selected from a nitrogen atom, an oxygen atom and an optionally oxidized sulfur atom, and

(3) R³, R⁴, R⁵ and R⁶ are all hydrogen atoms, or one of R³, R⁴, R⁵ and R⁶ is a halogen atom and the rest are hydrogen atoms (preferably R⁴ is a halogen atom and R³, R⁵ and R⁶ are hydrogen atoms).

A particularly preferable example of compound (III) is, for example, the following compound.

Compound (III) wherein

(1) R^{1′} is methyl, ethyl, 2-hydroxyethoxyethyl, 2-methoxyethoxyethyl, 2-methoxyethyl or 2-hydroxyethyl,

(2) R^{2′} is methyl, n-propyl, benzyl, phenyl, trifluoromethylphenyl, chlorophenyl, methoxyphenyl, bromophenyl, fluorophenyl, methylphenyl, trifluoromethoxyphenyl, phenoxyphenyl, t-butylphenyl, chlorotrifluorophenyl, tetrafluoroethoxyphenyl, imidazolylphenyl, tetrafluorobenzodioxinyl, methylisoxazolyl, trifluoromethylthiadiazolyl, trifluoromethyloxazolyl or trifluoromethylpyridyl, and

(3) R³, R⁴, R⁵ and R⁶ are all hydrogen atoms, or one of R³, R⁴, R⁵ and R⁶ is a fluorine atom or a chlorine atom and the rest are hydrogen atoms.

As specific examples of compound (III), for example, compounds of Examples 2-9, 11, 12, 14-33, 36-104, 106, 109-150 and 156 can be mentioned.

As a salt of compound (I) (including compound (II) and compound (III)), for example, metal salts, ammonium salts, salts with organic bases, salts with inorganic acids, salts with organic acids, salts with basic or acidic amino acids and the like can be mentioned. As preferable examples of the metal salt, alkali metal salts such as sodium salt, potassium salt and the like; alkaline earth metal salts such as calcium salt, magnesium salt, barium salt and the like; aluminum salt and the like can be mentioned. As preferable examples of the salts with organic bases, salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like can be mentioned. As preferable examples of the salts with inorganic acids, salts with hydrochloric acid, hydrobromic acid, nitric acid, sulfuric acid, phosphoric acid and the like can be mentioned. As preferable examples of the salts with organic acids, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned. As preferable examples of the salts with basic amino acids, salts with arginine, lysine, ornithine and the like can be mentioned. As preferable examples of the salts with acidic amino acids, salts with aspartic acid, glutamic acid and the like can be mentioned.

Of these, pharmaceutically acceptable salts are preferable. For example, when a compound has an acidic functional group therein, alkali metal salts (e.g., sodium salt, potassium salt and the like), alkaline earth metal salts (e.g., calcium salt, magnesium salt and the like) and the like, ammonium salt and the like can be mentioned. When a compound has a basic functional group therein, salts with inorganic acids such as hydrochloric acid, hydrobromic acid, nitric acid, sulfuric acid, phosphoric acid and the like, and salts with organic acids such as acetic acid, phthalic acid, fumaric acid, oxalic acid, tartaric acid, maleic acid, citric acid, succinic acid, methanesulfonic acid, benzenesulfonic acid, p-toluenesulfonic acid and the like can be mentioned.

Now, the production methods of the compound (I) of the present invention are explained.

The compound (I) of the present invention is obtained, for example, by the method shown by the following reaction scheme or a method analogous thereto and the like.

The compound in the formula encompasses one in the form of a salt, and as such salt, for example, those similar to the salts of compound (I) and the like can be used.

While the compound obtained in each step can be used in the form of a reaction mixture or as a crude product for the next reaction, it can also be isolated from the reaction mixture according to a conventional method, and easily purified by a separation means such as recrystallization, distillation, chromatography and the like.

The outline of the reaction schemes is shown in the following, wherein the symbols of the compounds in the schemes are as defined above.

### [Production Method 1]

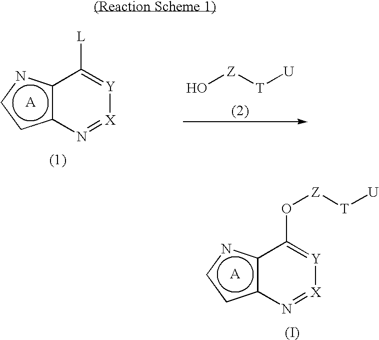

wherein L is a leaving group, and other symbols are as defined above.

As the leaving group for L, for example, a halogen atom, an optionally substituted alkylsulfonyl group, an optionally substituted alkylsulfonyloxy group, an optionally substituted arylsulfonyloxy group and the like can be used.

As the halogen atom, a fluorine atom, a chlorine atom, a bromine atom, an iodine atom and the like can be used.

As the alkylsulfonyl group, for example, a C₁₋₆ alkylsulfonyl group such as methylsulfonyl, ethylsulfonyl and the like, and the like can be used.

As the alkylsulfonyloxy group, for example, a C₁₋₆ alkylsulfonyloxy group such as methylsulfonyloxy, ethylsulfonyloxy and the like, and the like can be used.

As the arylsulfonyloxy group, for example, a C₆₋₁₄ arylsulfonyloxy group such as phenylsulfonyloxy and the like, and the like can be used.

As the substituent of the alkylsulfonyl group, alkylsulfonyloxy group or arylsulfonyloxy group, for example, a halogen atom (e.g., a fluorine atom, a chlorine atom, a bromine atom), an optionally halogenated C₁₋₆ alkyl (e.g., methyl, ethyl, trifluoromethyl), a nitro group and the like can be used.

Compound (I) can be produced by reacting compound (I) with compound (2). Compound (2) is used in an amount of 0.1-10 equivalents, preferably 0.3-3 equivalents, relative to compound (I). Where necessary, a base may be added. As the base, inorganic bases, organic bases and the like can be used. Specifically, for example, sodium hydroxide, potassium hydroxide, sodium carbonate, potassium carbonate, cesium carbonate, sodium hydrogen carbonate, triethylamine, N-ethyldiisopropylamine, N-methylmorpholine, 1,8-diazabicyclo[5.4.0]undeca-7-ene, pyridine, 4-(dimethylamino)pyridine, N,N-dimethylaniline, sodium methoxide, sodium ethoxide, potassium t-butoxide, sodium hydride, sodium amide, lithiumdiisopropylamide and the like can be mentioned. Such base is used in an amount of 1-30 equivalents, preferably 1-10 equivalents, relative to compound (1). This reaction is advantageously carried out in a solvent inert to the reaction. The solvent is not particularly limited as long as the reaction proceeds. For example, alcohols such as methanol, ethanol, 2-propanol, 2-methyl-2-propanol and the like, ethers such as diethyl ether, tetrahydrofuran, 1,4-dioxane, 1,2-dimethoxyethane and the like, hydrocarbons such as benzene, toluene, cyclohexane, hexane and the like, amides such as N,N-dimethylformamide, N,N-dimethylacetamide, 1-methyl-2-pyrrolidone and the like, halogenated hydrocarbons such as dichloromethane, chloroform, carbon tetrachloride, 1,2-dichloroethane and the like, nitrites such as acetonitrile and the like, sulfoxides such as dimethyl sulfoxide and the like, water and the like can be used alone or in a mixture thereof. While the reaction time varies depending on the reagents and solvents to be used, it is generally 10 min-100 hr, preferably 30 min-50 hr. The reaction temperature is generally −78° C. to 200° C., preferably 0° C. to 150° C. As compound (2), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

### [Production Method 2]

In compound (I), when U is an optionally substituted ureido group, for example, the compound can also be produced by the method shown in Reaction Scheme 2.

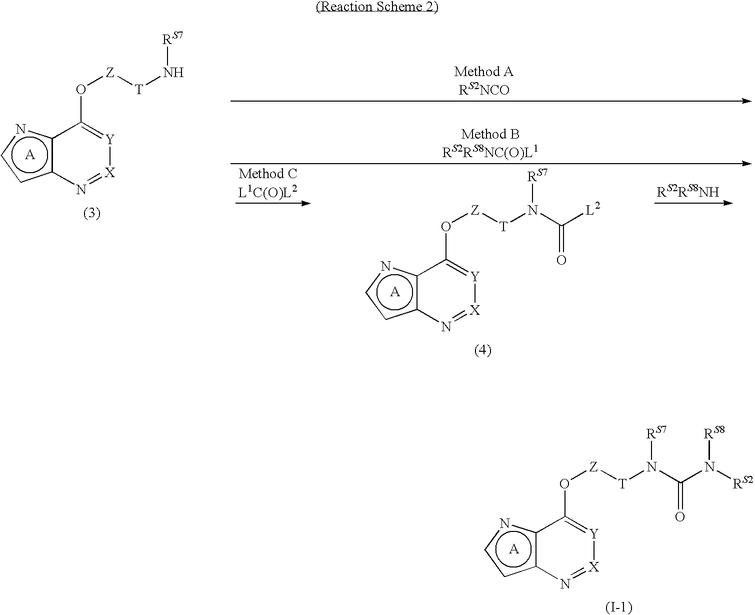

wherein L¹ and L² are leaving groups, and other symbols are as defined above.

As the leaving group for L¹ and L² for example, a halogen atom, an optionally substituted aryloxy group, an optionally substituted alkyloxy group, a 1-imidazolyl group and the like can be used.

As the aryloxy group, for example, a C₆₋₁₄ aryloxy group such as phenyloxy and the like, and the like can be used.

As the alkyloxy group, for example, a C₁₋₆ alkyloxy group such as methyloxy, ethyloxy and the like, and the like can be used.

As the substituent of the aryloxy group or alkyloxy group, for example, a halogen atom (e.g., a fluorine atom, a chlorine atom, a bromine atom), an optionally halogenated C₁₋₆ alkyl (e.g., methyl, ethyl, trifluoromethyl), a nitro group and the like can be used.

According to method A, compound (I-1) is produced by reacting compound (3) with an isocyanate derivative (R^{S2}NCO). The isocyanate derivative (R^{S2}NCO) is used in an amount of 0.1-10 equivalents, preferably 0.3-3 equivalents, relative to compound (3). In addition, a base may be used in an amount of 0.01-10 equivalents, preferably 0.03-5 equivalents, relative to compound (3). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As the isocyanate derivative (R^{S2}NCO), a commercially available one may be used, or the derivative can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

In method B, compound (I-1) is produced by reacting compound (3) with a compound represented by the formula: R^{S2}R^{S8}NC(O)L¹. The compound represented by the formula: R^{S2}R^{S8}NC(O)L¹ is used in an amount of 0.1-10 equivalents, preferably 0.3-3 equivalents, relative to compound (3). In addition, a base may be used in an amount of 0.01-10 equivalents, preferably 0.03-5 equivalents, relative to compound (3). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As the compound represented by R^{S2}R^{S8}NC(O)L¹, a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

In method C, compound (4) is first produced by reacting compound (3) with a compound represented by the formula: L¹C(O)L², and then compound (4) is reacted with amine derivative (R^{S2}R^{S8}NH) to give compound (I-1). The compound represented by the formula: L¹C(O)L² is used in an amount of 0.1-10 equivalents, preferably 0.3-3 equivalents, relative to compound (3). In addition, a base may be used in an amount of 0.01-10 equivalents, preferably 0.03-5 equivalents, relative to compound (3). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As the compound represented by L¹C(O)L², a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto. While the obtained compound (4) can be used in the form of a reaction mixture or as a crude product for the next reaction, it can also be isolated and purified from the reaction mixture according to a conventional method and used for the next reaction. The amine derivative (R^{S2}R^{S8}NH) is used in an amount of 0.1-10 equivalents, preferably 0.3-3 equivalents, relative to compound (4). In addition, a base may be used in an amount of 0.01-10 equivalents, preferably 0.03-5 equivalents, relative to compound (4). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr. preferably 30 min-50 hr. The reaction temperature is generally −78° C. to 200° C., preferably 0° C. to 150° C. As the amine derivative (R^{S2}R^{S8}NH), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

Compound (3) shown in Reaction Scheme 2 can be produced, for example, by the method shown in the following Scheme. Compound (3-a) is encompassed in compound (3).

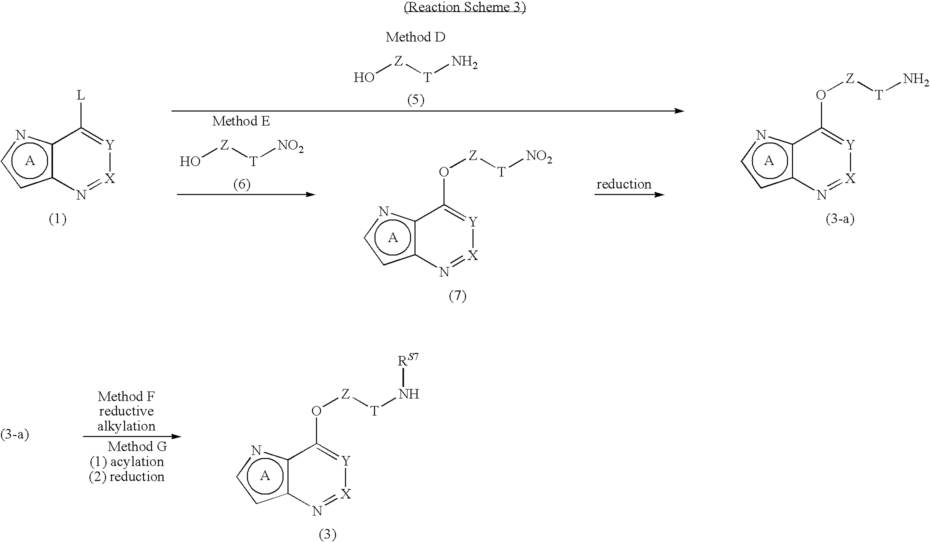

wherein each symbol is as defined above.

In method D, compound (3-a) is produced by reacting compound (I) with compound (5). Compound (5) is used in an amount of 0.1-10 equivalents, preferably 0.3-3 equivalents, relative to compound (I). Where necessary, a base may be added. As the base, those similar to the base exemplified for Reaction Scheme 1 can be used. The base is used in an amount of 1-30 equivalents, preferably 1-10 equivalents, relative to compound (I). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As compound (5), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

In method E, compound (7) is produced by first reacting compound (1) with compound (6), and then the nitro group of compound (7) is reduced to give compound (3-a). Compound (6) is used in an amount of 0.1-10 equivalents, preferably 0.3-3 equivalents, relative to compound (I). In addition, a base may be used in an amount of 1-30 equivalents, preferably 1-10 equivalents, relative to compound (3). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As compound (6), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto. For example, a method using a reducing agent such as a metal such as zinc, iron, tin and the like, a metal salt such as stannous chloride and the like, a metal hydrogen complex compound such as lithium aluminum hydride and the like, and the like, a contact hydrogenation method using a catalyst such as palladium carbon, platinum oxide, Raney-nickel and the like, and the like can be used. In the method using a reducing agent, the reducing agent is used in an amount of 1-500 equivalents, preferably 1-100 equivalents, relative to compound (7). Where necessary, an acidic substance (hydrochloric acid, acetic acid, ammonium chloride and the like) or a basic substance (sodium hydroxide and the like) may be added. In the contact hydrogenation method, a catalyst is used in an amount of 5-1000 wt %, preferably 10-500 wt %, relative to compound (7) and the hydrogen pressure is generally 1-100 atm. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C.

In method F, compound (3-a) is subjected to a reductive alkylation using an aldehyde derivative or a ketone derivative to give compound (3). The reductive alkylation can be carried out according to a method known per se, for example, the methods described in Advanced Organic Chemistry, 4th Ed., Jerry March, Comprehensive Organic Transformations, 2nd Ed., Richard C. Larock and the like, or a method analogous thereto.

In method G, compound (3-a) is acylated to give an amide form, and the amide group is reduced to give compound (3). The acylation and reduction of the amide group can be carried out according to a method known per se, for example, the methods described in Advanced Organic Chemistry, 4th Ed., Jerry March, Comprehensive Organic Transformations, 2nd Ed., Richard C. Larock and the like, or a method analogous thereto.

### [Production Method 3]

In compound (I), when U is an optionally substituted amido group, for example, the compound can also be produced by the method shown in Reaction Scheme 4.

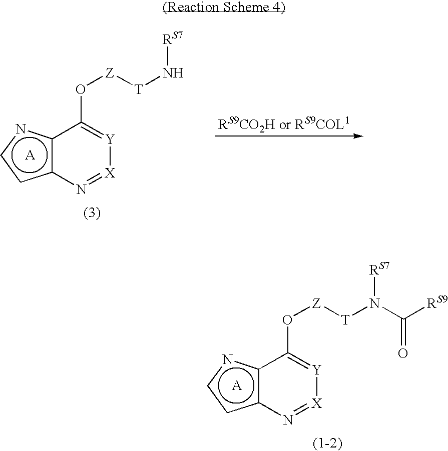

wherein each symbol is as defined above.

As the optionally substituted hydrocarbon group and optionally substituted heterocyclic group for R^{S9}, those similar to the aforementioned optionally substituted hydrocarbon group and optionally substituted heterocyclic group exemplified for R² can be used.

In this method, compound (3) is reacted with carboxylic acid (R^{S9}CO₂H) in the presence of a condensation agent, or compound (3) is reacted with a reactive derivative (R^{S9}COL¹) of carboxylic acid to give compound (I-2).

For reaction of compound (3) with carboxylic acid (R^{S9}CO₂H) in the presence of a condensation agent, carboxylic acid (R^{S9}CO₂H) is used in an amount of 0.1-10 equivalents, preferably 0.3-3 equivalents, relative to compound (3). As the condensation agent, for example, 1-ethyl-1-(3-dimethylaminopropyl)carbodiimide hydrochloride, 1,3-dicyclohexylcarbodiimide, diethyl cyanophosphate, diphenylphosphoryl azide, 1,1′-carbonyldiimidazole, benzotriazol-1-yloxytripyrrolidinophosphonium hexafluorophosphate and the like can be used. The condensation agent is used in an amount of 1-10 equivalents, preferably 1-5 equivalents, relative to compound (3). Where necessary, a suitable condensation promoter (e.g., 1-hydroxybenzotriazole, N-hydroxysuccinimide and the like) can be used. The condensation promoter is used in an amount of 0.1-10 equivalents, preferably 0.3-3 equivalents, relative to compound (3). This reaction may proceed more smoothly by the addition of a base. As the base, those similar to the base exemplified for Reaction Scheme 1 can be used. The base is used in an amount of 0.01-10 equivalents, preferably 0.03-5 equivalents, relative to compound (3). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As carboxylic acid (R^{S9}CO₂H), a commercially available one may be used, or the acid can be produced according to a method known per se, for example, the methods described in Advanced Organic Chemistry, 4th Ed., Jerry March, Comprehensive Organic Transformations, 2nd Ed., Richard C . . . . Larock and the like, or a method analogous thereto.

For reaction of compound (3) with a reactive derivative (R^{S9}COL¹) of carboxylic acid, the reactive derivative (R^{S9}COL¹) of carboxylic acid is used in an amount of 0.1-10 equivalents, preferably 0.3-3 equivalents, relative to compound (3). In this method, while the reaction is generally carried out in the presence of a base, the presence of a base is not necessarily essential. As the base, those similar to the base exemplified for Reaction Scheme 1 can be used. The base is used in an amount of 0.01-10 equivalents, preferably 0.03-5 equivalents, relative to compound (3). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As the reactive derivative (R^{S9}COL¹) of carboxylic acid, a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

### [Production Method 4]

In compound (I), when U is an optionally substituted carbamoyl group, for example, the compound can also be produced by a method shown in Reaction Scheme 5.

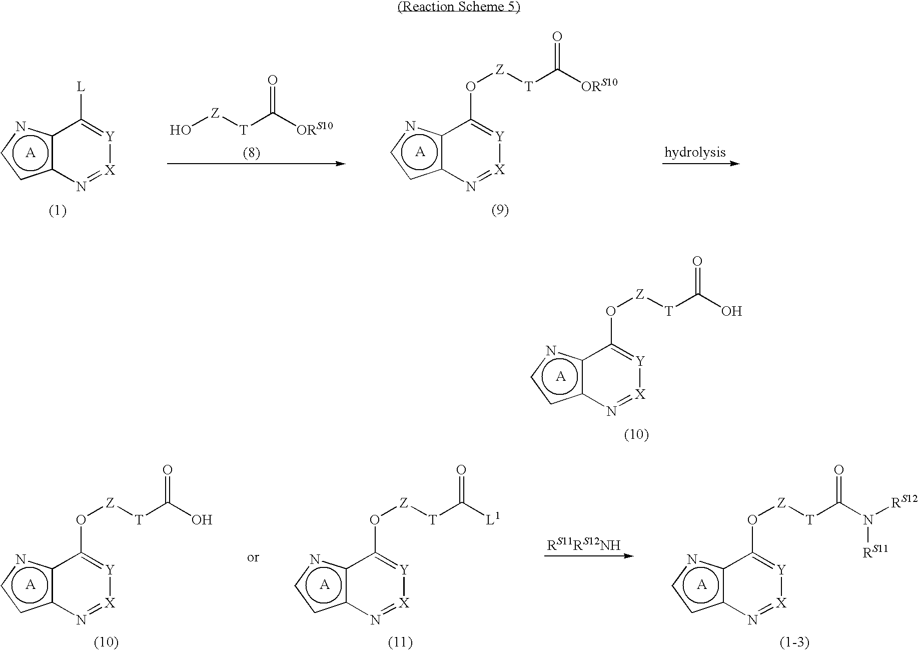

wherein R^{S10} is an alkyl group, and other symbols are as defined above.

As the alkyl group for R^{S10}, for example, a C₁₋₆ alkyl group such as methyl, ethyl, t-butyl and the like, and the like can be used.

Compound (9) can be produced by reacting compound (I) with compound (8). Compound (8) is used in an amount of 0.1-10 equivalents, preferably 0.3-3 equivalents, relative to compound (1). Where necessary, a base may be added. As the base, those similar to the base exemplified for Reaction Scheme 1 can be used. The base is used in an amount of 1-30 equivalents, preferably 1-10 equivalents, relative to compound (1). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As compound (8), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

Compound (10) can be produced by subjecting compound (9) to hydrolysis. This reaction is carried out according to a conventional method in the presence of an acid or base in a water-containing solvent. As the acid, for example, hydrochloric acid, sulfuric acid, acetic acid, hydrobromic acid and the like can be mentioned. As the base, for example, sodium hydroxide, potassium hydroxide, lithium hydroxide, barium hydroxide, calcium hydroxide, potassium carbonate, sodium carbonate, cesium carbonate, sodium methoxide and the like can be mentioned. The acid or base is used in an amount of 1-50 equivalents, preferably 1-10 equivalents, relative to compound (9). As the water-containing solvent, for example, a mixed solvent of one or more kinds selected from methanol, ethanol, tetrahydrofuran, 1,4-dioxane and the like and water and the like can be mentioned. When hydrolysis is performed using an acid, an excess acid may be used as a solvent. The reaction time is generally 10 min-100 hr, preferably 30 min-50 hr. The reaction temperature is generally −78° C. to 200° C., preferably 0° C. to 150° C.

Compound (I-3) can be produced by reacting compound (10) with an amine derivative (R^{S11}R^{S12}NH) in the presence of a condensation agent, or by reacting a reactive derivative (11) of compound (10) with an amine derivative (R^{S11}R^{S12}NH). When a condensation agent is used, the amine derivative (R^{S11}R^{S12}NH) is used in an amount of 0.1-10 equivalents, preferably 0.3-3 equivalents, relative to compound (10). As the condensation agent, those similar to the condensation agents exemplified in Reaction Scheme 4 can be used. The condensation agent is used in an amount of 1-10 equivalents, preferably 1-5 equivalents, relative to compound (10). Where necessary, the condensation promoter exemplified for Reaction Scheme 4 can be used. The condensation promoter is used in an amount of 0.1-10 equivalents, preferably 0.3-3 equivalents, relative to compound (10). This reaction may proceed more smoothly by the addition of a base. As the base, those similar to the base exemplified for Reaction Scheme 1 can be used. The base is used in an amount of 0.01-10 equivalents, preferably 0.03-5 equivalents, relative to compound (10). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As amine derivative (R^{S11}R^{S12}NH), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto. For reaction of reactive derivative (11) with an amine derivative (R^{S11}R^{S12}NH), the amine derivative (R^{S11}R^{S11}NH) is used in an amount of 0.1-10 equivalents, preferably 0.3-3 equivalents, relative to reactive derivative (11). While this reaction is generally carried out in the presence of a base, the presence of a base is not necessarily essential. As the base, those similar to the base exemplified for Reaction Scheme 1 can be used. The base is used in an amount of 0.01-10 equivalents, preferably 0.03-5 equivalents, relative to reactive derivative (11). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. Reactive derivative (11) can be produced according to a method known per se, for example, the methods described in Advanced Organic Chemistry, 4th Ed., Jerry March, Comprehensive Organic Transformations, 2nd Ed., Richard C. Larock and the like, or a method analogous thereto from compound (10).

### [Production Method 5]

In compound (I), when U is an optionally substituted sulfonamido group, for example, the compound can also be produced by a method shown in Reaction Scheme 6.

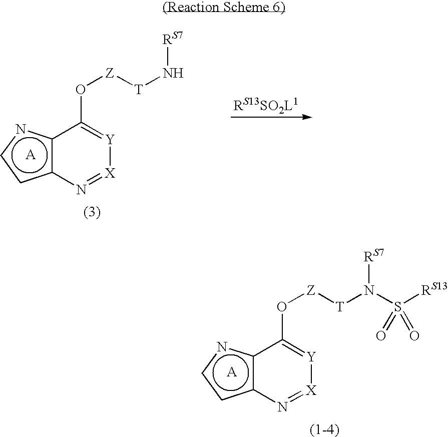

wherein each symbol is as defined above.

Compound (I-4) can be produced by reacting compound (3) with a reactive derivative (R^{S13} SO₂L¹) of sulfonic acid. The reactive derivative (R^{S13}SO₂L¹) of sulfonic acid is used in an amount of 0.1-10 equivalents, preferably 0.3-3 equivalents, relative to compound (3). In this method, while the reaction is generally carried out in the presence of a base, the presence of a base is not necessarily essential. As the base, those similar to the base exemplified for Reaction Scheme 1 can be used. The base is used in an amount of 0.01-10 equivalents, preferably 0.03-5 equivalents, relative to compound (3). This reaction is advantageously carried out in a solvent inert to the reaction. As the solvent, those similar to the solvent exemplified for Reaction Scheme 1 can be used. The reaction time is generally 10 min-100 hr, preferably 30 min-24 hr. The reaction temperature is generally −78° C. to 200° C., preferably 0° C. to 150° C. As the reactive derivative (R^{S13}SO₂L¹) of sulfonic acid, a commercially available one may be used, or the derivative can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

### [Production Method 6]

In compound (I), when U is an optionally substituted thioureido group, for example, the compound can also be produced by a method shown in Reaction Scheme 7.

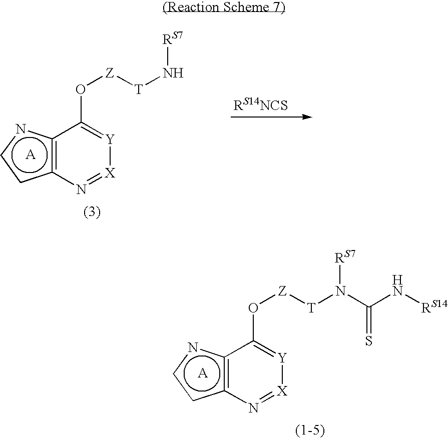

wherein each symbol is as defined above.

Compound (I-5) can be produced by reacting compound (3) with a thioisocyanate derivative (R^{S14}NCS). The thioisocyanate derivative (R^{S14}NCS) is used in an amount of 0.1-10 equivalents, preferably 0.3-3 equivalents, relative to compound (3). In addition, a base may be used in an amount of 0.01-10 equivalents, preferably 0.03-5 equivalents, relative to compound (3). As the base, those similar to the base exemplified for Reaction Scheme 1 can be used. This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As the thioisocyanate derivative (R^{S14}NCS), a commercially available one may be used, or the derivative can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

The aforementioned compound (I) can be produced according to a method known per se, for example, the methods described in Journal of Medicinal Chemistry, vol. 43, pages 4288-4312 (2000), Journal of Organic Chemistry, vol. 67, pages 2345-2347 (2002) and the like, or a method analogous thereto.

The aforementioned compound (I) can also be produced, for example, by a method shown in Reaction Scheme 8. Compound, (1-a) and compound (I-b) are encompassed in compound (I) Compound (12) can be produced according to the method described in Journal of Organic Chemistry, vol. 64, pages 8411-8412 (1999), or a method analogous thereto.

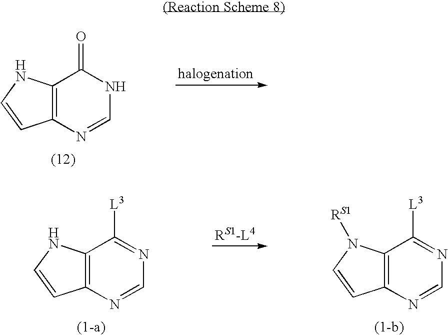

wherein R^{S1} is a hydrogen atom, an optionally substituted hydrocarbon group, an optionally substituted heterocyclic group or an optionally substituted acyl group, L³ is a halogen atom, L⁴ is a leaving group, and other symbols are as defined 1 above.

As the optionally substituted hydrocarbon group and optionally substituted heterocyclic group and acyl group for R^{S1}, those similar to the optionally substituted hydrocarbon group, optionally substituted heterocyclic group and acyl group, respectively exemplified for the above-mentioned substituent group (1), can be used.

As the halogen atom for L³, a fluorine atom, a chlorine atom, a bromine atom, an iodine atom and the like can be used.

As the leaving group for L⁴, those similar to the aforementioned leaving group exemplified for L can be used.

Compound (1-a) can be produced by reacting compound (12) with a halogenating agent. As the halogenating agent, for example, 1-500 equivalents of phosphorus oxychloride, phosphorus pentachloride, phosphorus trichloride, thionyl chloride, sulfuryl chloride, phosphorus tribromide and the like, relative to compound (12), can be used. Where necessary, the reaction may be carried out in the presence of a base such as N,N-diethylaniline, N,N-dimethylaniline, pyridine and the like. While the reaction may be carried out without solvent, as the reaction solvent, for example, dichloromethane, chloroform, carbon tetrachloride, 1,2-dichloroethane, benzene, toluene, xylene, diethyl ether, tetrahydrofuran, 1,4-dioxane and the like may be used. The reaction time is generally 10 min-100 hr, preferably 30 min-50 hr. The reaction temperature is generally −78° C. to 200° C., preferably 0° C. to 150° C.

Compound (1-b) can be produced by reacting compound (1-a) with compound (R^{S1}-L⁴). The compound (R^{S1}-L⁴) is used in an amount of 0.1-10 equivalents, preferably 0.3-3 equivalents, relative to compound (1-a). Where necessary, a base may be added. As the base, those similar to the base exemplified for Reaction Scheme 1 can be used. The base is used in an amount of 1-30 equivalents, preferably 1-10 equivalents, relative to compound (5). This reaction is advantageously carried out in a solvent inert to the reaction. As the solvent, those similar to the solvent exemplified for Reaction Scheme 1 can be used. The reaction time is generally 10 min-100 hr, preferably 30 min-50 hr. The reaction temperature is generally −78° C. to 200° C., preferably 0° C. to 150° C. As compound (R^{S1}-L⁴), a commercially available one may be used, or the compound can be produced according to a method known per se, for example, the methods described in Advanced Organic Chemistry, 4th Ed., Jerry March, Comprehensive Organic Transformations, 2nd Ed., Richard C. Larock and the like, or a method analogous thereto.

In the above respective reactions, when starting compounds have an amino group, a carboxyl group and a hydroxyl group as substituent(s), these groups may be protected by a protecting group generally used in the peptide chemistry and the like. In this case, after the reaction, a desired compound can be obtained by eliminating the protecting groups, as necessary. The introduction and elimination of these protecting groups can be performed according to a method known per se, for example, the method described in Protective Groups in Organic Synthesis, 3rd Ed., Theodora W. Greene, Peter G. M. Wuts, Wiley-Interscience (1999) and the like.

When desired, compound (I) can also be produced by carrying out, in addition to the above-mentioned reactions, known hydrolysis, deprotection, acylation reaction, alkylation reaction, oxidation reaction, cyclization reaction, carbon chain extension reaction and substituent exchanging reaction, solely or in combination of two or more thereof.

Compound (I) can be isolated and purified by a separation means known per se, such as phase transfer, concentration, solvent extraction, fractionation, liquid conversion crystallization, recrystallization, chromatography and the like.

When compound (I) is obtained as a free compound, it can be converted into a desired salt by a method known per se or a modification thereof; conversely, when compound (I) is obtained as a salt, it can be converted into a free form or another desired salt by a method known per se or a modification thereof.

The compound (I) may be used as a prodrug. A prodrug of the compound (I) means a compound which is converted to the compound (I) of the present invention with a reaction due to an enzyme, an gastric acid, etc. under the physiological condition in the living body, that is, a compound which is converted to the compound (I) of the present invention with oxidation, reduction, hydrolysis, etc. according to an enzyme; a compound which is converted to the compound (I) of the present invention by hydrolysis etc. due to gastric acid, and the like.

A prodrug of compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation and tert-butylation, etc.); a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation, dimethylaminomethylcarbonylation, etc.); a compound obtained by subjecting a carboxyl group in compound (I) to an esterification- or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification and methylamidation, etc.) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

When compound (I) has isomers such as optical isomer, stereoisomer, positional isomer, rotational isomer and the like, and any isomers and mixtures are encompassed in the compound (I). For example, when compound (I) has an optical isomer, an optical isomer separated from a racemate is also encompassed in the compound (I). These isomers can be obtained as independent products by a synthesis means or a separation means (concentration, solvent extraction, column chromatography, recrystallization and the like) known per se.

The compound (I) may be a crystal, and both a single crystal and crystal mixtures are encompassed in compound (I). Crystals can be produced by crystallization according to crystallization methods known per se.

The compound (I) may be a solvate (e.g., hydrate etc.) or a non-solvate, both of which are encompassed in compound (I).

A compound labeled with an isotope (e.g., ³H, ¹⁴C, ³⁵S, ¹²⁵I and the like) and the like is also encompassed in compound (I).

The compounds (I)-(III) of the present invention and a prodrug thereof (hereinafter sometimes to be abbreviated as the compound of the present invention) have, for example, a kinase inhibitory action. As the kinase, for example, vascular endothelial growth factor receptor (VEGFR), platelet-derived growth factor receptor (PDGFR), tyrosine kinase with Ig and EGF homology domains2 (TIE2) and the like can be mentioned. As the vascular endothelial growth factor receptor (VEGFR), vascular endothelial growth factor receptor 1 (VEGFR1, Flt-1), vascular endothelial growth factor receptor 2 (VEGFR2, KDR, Flk-1), vascular endothelial growth factor receptor 3 (VEGFR3, Flt-4) and the like can be mentioned. Of these, vascular endothelial growth factor receptor 2 (VEGFR2) is preferable. As the platelet-derived growth factor receptor (PDGFR), platelet-derived growth factor receptor a (PDGFRx), platelet-derived growth factor receptor (PDGFRP) and the like can be mentioned. Particularly, as kinases, vascular endothelial growth factor receptor 2 (VEGFR2), platelet-derived growth factor receptor (PDGFR) and tyrosine kinase with Ig and EGF homology domains2 (TIE2) are preferable. As other kinases, fibroblast growth factor receptor 1 (FGFR1), fibroblast growth factor receptor 2 (FGFR2), fibroblast growth factor receptor 3 (FGFR3), fibroblast growth factor receptor 4 (FGFR4), stem cell factor receptor (c-Kit), Aurora A, Aurora B, CDK, MEK1, MEK2, A-Raf, B-Raf, C-Raf, Akt, ERK, MAPK, Src, MET, epidermal growth factor receptor (EGFR), human epidermal growth factor receptor 2 (HER2), human epidermal growth factor receptor 4 (HER4) and the like can also be mentioned.

For example, the vascular endothelial growth factor receptor 2 inhibitory activity of the compound of the present invention can be determined according to Experimental Example 1, the platelet-derived growth factor receptor inhibitory activity can be determined according to Experimental Example 2 or Experimental Example 3, the Tie2 inhibitory activity can be determined according to Experimental Example 4, the vascular endothelial cell growth inhibitory activity can be determined according to Experimental Example 5, and the antitumor activity can be determined according to Experimental Example 6.

The compound of the present invention particularly shows strong inhibitory activity against vascular endothelial growth factor receptor (VEGFR), and the selectivity for vascular endothelial growth factor receptor 2 (VEGFR2, KDR, Flk-1) is specifically high. Moreover, the compound also shows potent kinase inhibitory activity against VEGFR1, PDGFR and TIE2. In addition, since the compound of the present invention is also superior in the efficacy expression, pharmacokinetics (absorption, distribution, metabolism, excretion etc.), solubility (water-solubility etc.), interaction with other pharmaceutical products, safety (acute toxicity, chronic toxicity, genetic toxicity, reproductive toxicity, cardiotoxicity, carcinogenicity etc.) and stability (chemical stability, stability to enzyme etc.), it is useful as a pharmaceutical agent.

Therefore, the compound of the present invention is useful as a kinase inhibitor, preferably a vascular endothelial growth factor receptor (VEGFR) inhibitor, a platelet-derived growth factor receptor (PDGFR) inhibitor, a tyrosine kinase with Ig and EGF homology domains2 (TIE2) inhibitor, more preferably, a vascular endothelial growth factor receptor 2 (VEGFR2, KDR, Flk-1) inhibitor, for mammals (e.g., mouse, rat, hamster, rabbit, cat, dog, bovine, sheep, monkey, human etc.). The compound of the present invention is used as a pharmaceutical agent such as an angiogenesis inhibitor, a vascular endothelial cell growth inhibitor, or an agent for the prophylaxis or treatment of diseases possibly influenced by vascular endothelial growth factor, such as cancer (e.g., colorectal cancer, lung cancer, mesothelioma, pancreatic cancer, gastric cancer, breast cancer, ovarian cancer, prostate cancer, liver cancer, thyroid cancer, kidney cancer, uterus cancer, cerebral tumor, melanoma, sarcoma, urinary bladder cancer, blood cancer including multiple myeloma and the like), diabetic retinopathy, rheumatoid arthritis, psoriasis, atherosclerosis, Kaposi sarcoma, COPD, pain, asthma, inflammation (e.g., endometriosis, nephritis, osteoarthritis and the like) or hypertension, an agent for inhibiting growth of cancer, an agent for suppressing metastasis of cancer, apoptosis inducer and the like. Particularly, the compound of the present invention is effective for patients having cancer with expression or high expression of vascular endothelial growth factor receptor (VEGFR) and/or platelet-derived growth factor receptor (PDGFR) and/or Tie2, such as colorectal cancer, lung cancer, pancreatic cancer, gastric cancer, breast cancer, ovarian cancer, prostate cancer, liver cancer, thyroid cancer, kidney cancer, cerebral tumor, melanoma, urinary bladder cancer, blood cancer and the like. Of these, the compound of the present invention is effective for patients having, for example, colorectal cancer, ovary cancer, prostate cancer or kidney cancer.

The compound of the present invention can be administered orally or parenterally as it is or after mixing with a pharmacologically acceptable carrier.

The dosage form of the compound of the present invention for oral administration, for example, tablet (including sugar-coated tablet, film-coated tablet), pill, granule, powder, capsule (including soft capsule, microcapsule), syrup, emulsion, suspension and the like, and the dosage form for parenteral administration is, for example, injection, injecting agent, instillation, suppository and the like. In addition, it is effective to make a sustained release preparation by combining with a suitable base (e.g., polymer of butyric acid, polymer of glycolic acid, copolymer of butyric acid-glycolic acid, a mixture of polymer of butyric acid and polymer of glycolic acid, polyglycerol fatty acid ester etc.).

As a method for producing the compound of the present invention in the above-mentioned dosage form, a known production method generally used in the pertinent field can be applied. When the above-mentioned dosage form is produced, suitable amounts of additives such as an excipients, a binder, a disintegrant, a lubricant, a sweetening agent, a surfactant, a suspending agent, an emulsifier and the like generally used in the preparation field are appropriately added as necessary, and produced.

When the compound of the present invention is prepared into a tablet, for example, it can be produced by adding an excipient, a binder, a disintegrant, a lubricant and the like, and when a pill and a granule are to be prepared, they can be produced by adding an excipient, a binder, a disintegrant and the like. When a powder and a capsule are to be prepared, they can be produced by adding an excipient and the like, and when a syrup is to be prepared, it can be produced by adding a sweetener and the like, and when an emulsion or a suspension is to be prepared, it can be produced by adding a suspending agent, a surfactant, an emulsifier and the like.

Examples of the excipient include lactose, sucrose, glucose, starch, sucrose, microcrystalline cellulose, powdered glycyrrhiza, mannitol, sodium hydrogen carbonate, calcium phosphate, calcium sulfate and the like.

Examples of the binder include 5-10 wt % starch liquid paste, 10-20 wt % gum arabic solution or gelatin solution, 1-5 wt % tragacanth solution, carboxymethyl cellulose solution, sodium alginate solution, glycerin and the like.

Examples of the disintegrant include starch, calcium carbonate and the like.

Examples of the lubricant include magnesium stearate, stearic acid, calcium stearate, purified talc and the like.

Examples of the sweetener include glucose, fructose, invert sugar, sorbitol, xylitol, glycerin, simple syrup and the like.

Examples of the surfactant include sodium lauryl sulfate, polysorbate 80, sorbitan monofatty acid ester, polyoxyl stearate 40 and the like.

Examples of the suspending agent include gum arabic, sodium alginate, sodium carboxymethyl cellulose, methyl cellulose, bentonite and the like.

Examples of the emulsifier include gum arabic, tragacanth, gelatin, polysorbate 80 and the like.

Furthermore, when the compound of the present invention is produced in the above-mentioned dosage form, a suitable amount of a coloring agent, a preservative, an aromatic, a corrigent, a stabilizer, viscous agents and the like typically used in the field of preparation can be added on demand.

As the injection, intravenous injection as well as subcutaneous injection, intracutaneous injection, intramuscular injection, instillation and the like are mentioned, and as a sustained release preparation, iontophoresis transdermal agent and the like are mentioned.

Such injections are prepared by methods known per se, or by dissolving, suspending or emulsifying the compound of the present invention in a sterilized aqueous solution or oily liquid. As an aqueous solution for injection, physiological saline, isotonic solutions containing glucose or other auxiliary drugs (e.g., D-sorbitol, D-mannitol, sodium chloride and the like) and the like can be mentioned, and they can be used in combination with suitable dissolution aids, such as alcohols (e.g., ethanol), polyalcohols (e.g., propylene glycol, polyethylene glycol), nonionic surfactants (e.g., polysorbate 8.0, HCO-50) and the like. As an oily liquid, sesame oil, soybean oil and the like can be mentioned, which may be used in combination with dissolution aids such as benzyl benzoate, benzyl alcohol and the like. In addition, buffers (e.g., phosphate buffer, sodium acetate buffer), soothing agents (e.g., benzalkonium chloride, procaine hydrochloride and the like), stabilizers (e.g., human serum albumin, polyethylene glycol and the like), preservatives (e.g., benzyl alcohol, phenol and the like) and the like may be added. A prepared injection is generally filled in an ampoule.

While the content of the compound of the present invention in the pharmaceutical agent of the present invention varies depending on the form of the pharmaceutical preparation, it is generally about 0.01 to 100 wt %, preferably about 2 to 85 wt %, more preferably about 5 to 70 wt %, relative to the entire preparation.

While the content of the additive in the pharmaceutical agent of the present invention varies depending on the form of the pharmaceutical preparation, it is generally about 1 to 99.9 wt %, preferably about 10 to 90 wt %, relative to the entire preparation.

The compound of the present invention is stable and low toxic, and can be used safely. While the daily dose varies depending on the condition and body weight of patients, the kind of compound, administration route and the like, in the case of, for example, oral administration to patients for the treatment of cancer, the daily dose to an adult (body weight about 60 kg) is about 1 to 1000 mg, preferably about 3 to 300 mg, more preferably about 10 to 200 mg, as an active ingredient (the compound of the present invention), which can be given in a single administration or administered in 2 or 3 portions a day.

When the compound of the present invention is administered parenterally, it is generally administered in the form of a liquid (e.g., injection). While the dose varies depending on the subject of administration, target organ, symptom, administration method and the like, it is, for example, about 0.01 mg-about 100 mg, preferably about 0.01-about 50 mg, more preferably about 0.01-about 20 mg, in the form of an injection, relative to 1 kg of body weight, which is preferably given by intravenous injection.

The compound of the present invention can be used concurrently with other drugs. To be specific, the compound of the present invention can be used together with medicaments such as hormonal therapeutic agents, chemotherapeutic agents, immunotherapeutic agents, pharmaceutical agents inhibiting the action of cell growth factors or cell growth factor receptors and the like. In the following, the drugs that can be used in combination with the compound of the present invention are abbreviated as concomitant drugs.

As the “hormonal therapeutic agents”, for example, fosfestrol, diethylstylbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, anti-estrogens (e.g., tamoxifen citrate, toremifene citrate and the like), pill preparations, mepitiostane, testrolactone, aminoglutethimide, LH-RH agonists (e.g., goserelin acetate, buserelin, leuprorelin and the like), droloxifene, epitiostanol, ethinylestradiol sulfonate, aromatase inhibitors (e.g., fadrozole hydrochloride, anastrozole, retrozole, exemestane, vorozole, formestane and the like), anti-androgens (e.g., flutamide, bicartamide, nilutamide and the like), 5α-reductase inhibitors (e.g., finasteride, epristeride and the like), adrenocorticohormone drugs (e.g., dexamethasone, prednisolone, betamethasone, triamcinolone and the like), androgen synthesis inhibitors (e.g., abiraterone and the like), retinoid and drugs that retard retinoid metabolism (e.g., liarozole and the like) and the like can be used.

As the “chemotherapeutic agents”, for example, alkylating agents, metabolic antagonists, antitumor antibiotics, plant-derived antitumor agents and the like can be used.

As the “alkylating agents”, for example, nitrogen mustard, nitrogen mustard-N-oxide hydrochloride, chlorambucil, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin and DDS preparations thereof and the like can be used.

As the “metabolic antagonists”, for example, mercaptopurine, 6-mercaptopurine riboside, thioinosine, methotrexate, pemetrexed, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, Capecitabine and the like), aminopterine, nelzarabine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine, bendamustine and DDS preparations thereof and the like can be used.

As the “antitumor antibiotics”, for example, actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride and DDS preparations thereof and the like can be used.

As the “plant-derived antitumor agents”, for example, etoposide, etoposide phosphate, vinblastine sulfate, vincristine sulfate, vindesine sulfate, teniposide, paclitaxel, docetaxel, vinorelbine and DDS preparations thereof and the like can be used.

As the “immunotherapeutic agents (BRM)”, for example, picibanil, krestin, schizophyllan, lentinan, ubenimex, interferons, interleukins, macrophagecolony stimulating agents, granulocyte colony stimulating factors, erythropoietin, lymphotoxin, BCG vaccines, corynebacterium parvum, levamisole, polysaccharide K, procodazole, anti-CTLA4 antibody and the like can be used.

As the “cell growth factor” of the “pharmaceutical agents inhibiting the action of cell growth factors or cell growth factor receptors”, any substances that promote cell proliferation, which are normally peptides having a molecular weight of not more than 20,000 that are capable of exhibiting their action at low concentrations by binding to a receptor, including (1) EGF (epidermal growth factor) or substances possessing substantially the same activity as it [e.g., EGF, TGFα, heregulin, and the like], (2) insulin or substances possessing substantially the same activity as it [e.g., insulin, IGF (insulin-like growth factor)-1, IGF-2, and the like], (3) FGF (fibroblast growth factor) or substances possessing substantially the same activity as it [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor A), HGF (hepatocyte growth factor), VEGF (vascular endothelial growth factor), and the like], angiopoietin and the like can be used.

The “cell growth factor receptor” may be any receptors capable of binding to the above-mentioned cell growth factor, and specifically, EGF receptor, HER2, insulin receptor-1, insulin receptor-2, IGF receptor, FGF receptor-1 or FGF receptor-2, VEGF receptor, Tie-2, PDGF receptor and the like can be used.

As the “pharmaceutical agents inhibiting the action of cell growth factors or cell growth factor receptors”, EGF inhibitor, TGFα inhibitor, heregulin inhibitor, insulin inhibitor, IGF inhibitor, FGF inhibitor, KGF inhibitor, CSF inhibitor, EPO inhibitor, IL-2 inhibitor, NGF inhibitor, PDGF inhibitor, TGFβ inhibitor, HGF inhibitor, VEGF inhibitor, angiopoietin inhibitor, EGF receptor inhibitor, HER2 inhibitor, HER4 inhibitor, insulin receptor-1 inhibitor, insulin receptor-2 inhibitor, IGF receptor inhibitor, FGF receptor-1 inhibitor, FGF receptor-2 inhibitor, FGF receptor-3 inhibitor, FGF receptor-4 inhibitor, VEGF receptor inhibitor, Tie-2 inhibitor, PDGF receptor inhibitor, Abl inhibitor, Raf inhibitor, FLT3 inhibitor, c-Kit inhibitor, Src inhibitor, PKC inhibitor, Trk inhibitor, Ret inhibitor, mTOR inhibitor, Aurora inhibitor, PLK inhibitor, MEK (MEK1/2) inhibitor, MET inhibitor, CDK inhibitor, Akt inhibitor, ERK inhibitor, and the like can be used. More specifically, anti-VEGF antibody (Bevacizumab etc.), anti-HER2 antibody (Trastuzumab, Pertuzumab etc.), anti-EGFR antibody (Cetuximab, Panitumumab, Matuzumab, Nimotuzumab etc.), anti-VEGFR antibody, Imatinib, Erlotinib, Gefitinib, Sorafenib, Sunitinib, Dasatinib, Lapatinib, Vatalanib, 4-(4-fluoro-2-methyl-1H-indol-5-yloxy)-6-methoxy-7-[3-(1-pyrrolidinyl)propoxy]quinazoline (AZD-2171), Lestaurtinib, Pazopanib, Canertinib, Tandutinib, 3-(4-bromo-2,6-difluorobenzyloxy)-5-[3-[4-(1-pyrrolidinyl)butyl]ureido]isothiazole-4-carboxamide (CP-547632), Axitinib, N-(3,3-dimethyl-2,3-dihydro-1H-indol-6-yl)-2-(pyridin-4-ylmethylamino)pyridine-3-carboxamide (AMG-706), Nilotinib, 6-[4-(4-ethylpiperazin-1-ylmethyl)phenyl]-N-[1(R)— phenylethyl]-7H-pyrrolo[2,3-d]pyrimidin-4-amine (AEE-788), Vandetanib, Temsirolimus, Everolimus, Enzastaurin, N-[4-[4-(4-methylpiperazin-1-yl)-6-(3-methyl-1H-pyrazol-5-ylamino)pyrimidin-2-ylsulfanyl]phenyl]cyclopropanecarboxamide (VX-680), phosphoric acid 2-[N-[3-[4-[5-[N-(3-fluorophenyl)carbamoylmethyl]-1H-pyrazol-3-ylamino]quinazolin-7-yloxy]propyl]-N-ethylamino]ethyl ester (AZD-1152), 4-[9-chloro-7-(2,6-difluorophenyl)-5H-pyrimido[5,4-d][2]benzazepin-2-ylamino]benzoic acid (MLN-8054), N-[2-methoxy-5-[(E)-2-(2,4,6-trimethoxyphenyl)vinylsulfonylmethyl]phenyl]glycine sodium salt (ON-1910Na), 4-[8-cyclopentyl-7(R)-ethyl-5-methyl-6-oxo-5,6,7,8-tetrahydropteridin-2-ylamino]-3-methoxy-N-(1-methylpiperidin-4-yl)benzamide (BI-2536), 5-(4-bromo-2-chlorophenylamino)-4-fluoro-1-methyl-1H-benzimidazole-6-carbohydroxamic acid 2-hydroxyethyl ester (AZD-6244), N-[2(R),3-Dihydroxypropoxy]-3,4-difluoro-2-(2-fluoro-4-iodophenylamino)benzamide (PD-0325901) and the like can be used.

In addition to the aforementioned drugs, L-asparaginase, aceglatone, procarbazine hydrochloride, protoporphyrin-cobalt complex salt, mercuric hematoporphyrin-sodium, topoisomerase I inhibitors (e.g., irinotecan, topotecan, and the like), topoisomerase II inhibitors (e.g., sobuzoxane, and the like), differentiation inducers (e.g., retinoid, vitamin D, and the like), other angiogenesis inhibitors (e.g., fumagillin, shark extract, COX-2 inhibitor and the like), α-blockers (e.g., tamsulosin hydrochloride and the like), bisphosphonic acid (pamidronate, zoledronate etc.), thalidomide, 5 azacytidine, decitabine, bortezomib, antitumor antibody such as anti-CD20 antibody and the like, toxin labeled antibody and the like can also be used.

By combining the compound of the present invention and a concomitant drug, a superior effect such as

(1) the dose can be reduced as compared to single administration of the compound of the present invention or a concomitant drug,
(2) the drug to be combined with the compound of the present invention can be selected according to the condition of patients (mild case, severe case and the like),
(3) the period of treatment can be set longer,
(4) a sustained treatment effect can be designed,
(5) a synergistic effect can be afforded by a combined use of the compound of the present invention and a concomitant drug, and the like, can be achieved.

In the present specification, a pharmaceutical agent for use of the compound of the present invention and a concomitant drug in combination may be referred to as the “combination agent of the present invention”.

When using the combination agent of the present invention, the administration time of the compound of the present invention and the concomitant drug is not restricted, and the compound of the present invention or the concomitant drug can be administered to an administration subject simultaneously, or may be administered at different times. The dosage of the concomitant drug may be determined according to the administration amount clinically used, and can be appropriately selected depending on an administration subject, administration route, disease, combination and the like.

The administration mode of the compound of the present invention and the concomitant drug of the present invention include the following methods:

(1) The compound of the present invention and the concomitant drug are simultaneously produced to give a single preparation for administration. (2) The compound of the present invention and the concomitant drug are separately produced to give two kinds of preparations which are administered simultaneously by the same administration route. (3) The compound of the present invention and the concomitant drug are separately produced to give two kinds of preparations which are administered by the same administration route only at the different times. (4) The compound of the present invention and the concomitant drug are separately produced to give two kinds of preparations which are administered simultaneously by the different administration routes. (5) The compound of the present invention and the concomitant drug are separately produced to give two kinds of preparations which are administered by the different administration routes only at different times (for example, the compound of the present invention and the concomitant drug are administered in this order, or in the reverse order). The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like. When the subject of administration is human, for example, a concomitant drug can be used in 0.01-100 parts by weight relative to 1 part by weight of the compound of the present invention.

A combination agent of the present invention has low toxicity, and for example, the compound of the present invention and/or the above-mentioned concomitant drug can be mixed, according to a method known per se, with a pharmacologically acceptable carrier to give pharmaceutical compositions, such as tablets (including sugar-coated tablet, film-coated tablet), powders, granules, capsules (including soft capsule), solutions, injections, suppositories, sustained release agents and the like, which can be safely administered orally or parenterally (e.g., local, rectum, vein, and the like). An injection can be administered by intravenous, intramuscular, subcutaneous or intra-tissue administration directly to the lesion.

As a pharmacologically acceptable carrier which may be used for preparing a preparation of a combination agent of the present invention, those similar to the aforementioned pharmacologically acceptable carriers that can be used for the production of the pharmaceutical agent of the present invention can be mentioned. Where necessary, the aforementioned additives that can be used for the production of the pharmaceutical agent of the present invention, such as preservatives, antioxidants, coloring agents, sweetening agents, adsorbents, wetting agents and the like can be also used in appropriate amounts.

The mixing ratio of the compound of the present invention to the concomitant drug in the combination agent of the present invention can be appropriately selected depending on an administration subject, administration route, diseases and the like.

For example, the content of the compound of the present invention in the combination agent of the present invention differs depending on the form of a preparation, and is usually from about 0.01 to 100% by weight, preferably from about 0.1 to 50% by weight, further preferably from about 0.5 to 20% by weight, based on the preparation.

The content of the concomitant drug in the combination agent of the present invention differs depending on the form of a preparation, and is usually from about 0.01 to 90% by weight, preferably from about 0.1 to 50% by weight, further preferably from about 0.5 to 20% by weight, based on the preparation.

The content of additives in the combination agent of the present invention differs depending on the form of a preparation, and is usually from about 1 to 99.99% by weight, preferably from about 10 to 90% by weight, based on the preparation.

In the case when the compound of the present invention and the concomitant drug are separately prepared respectively, the same contents may be adopted.

These preparations can be produced by a method known per se usually used in a preparation process.

For example, the compound of the present invention and the concomitant drug can be made into an aqueous injection together with a dispersing agent (e.g., Tween 80 (manufactured by Atlas Powder, US), HCO 60 (manufactured by Nikko Chemicals), polyethylene glycol, carboxymethylcellulose, sodium alginate, hydroxypropylmethylcellulose, dextrin and the like), a stabilizer (e.g., ascorbic acid, sodium pyrosulfite, and the like), a surfactant (e.g., Polysorbate 80, macrogol and the like), a solubilizer (e.g., glycerin, ethanol and the like), a buffer (e.g., phosphoric acid and alkali metal salt thereof, citric acid and alkali metal salt thereof, and the like), an isotonizing agent (e.g., sodium chloride, potassium chloride, mannitol, sorbitol, glucose and the like), a pH regulator (e.g., hydrochloric acid, sodium hydroxide and the like), a preservative (e.g., ethyl p-oxybenzoate, benzoic acid, methylparaben, propylparaben, benzyl alcohol and the like), a dissolving agent (e.g., conc. glycerin, meglumine and the like), a dissolution aid (e.g., propylene glycol, sucrose and the like), a soothing agent (e.g., glucose, benzyl alcohol and the like), and the like, or can be dissolved, suspended or emulsified in a vegetable oil such as olive oil, sesame oil, cotton seed oil, corn oil and the like or a dissolution aid such as propylene glycol and prepared into an oily injection, whereby an injection is afforded.

In addition, an excipient (e.g., lactose, sucrose, starch and the like), a disintegrating agent (e.g., starch, calcium carbonate and the like), a binder (e.g., starch, gum Arabic, carboxymethylcellulose, polyvinylpyrrolidone, hydroxpropylcellulose and the like), a lubricant (e.g., talc, magnesium stearate, polyethylene glycol 6000 and the like) and the like, for example, can be added to the compound of the present invention or the concomitant drug, according to a method known per se, and the mixture can be compression-molded, then if desirable, the molded product can be coated by a method known per se for the purpose of masking of taste, enteric property or durability, to obtain a preparation for oral administration. As this coating agent, for example, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudoragit (methacrylic acid-acrylic acid copolymer, manufactured by Rohm, DE), pigment (e.g., iron oxide red, titanium dioxide, etc.) and the like can be used. The preparation for oral administration may be any of an immediate-release preparation and a sustained release preparation.

Moreover, the compound of the present invention and the concomitant drug can be made into an oily or aqueous solid, semisolid or liquid suppository according to a method known per se, by mixing with an oily substrate, aqueous substrate or aqueous gel substrate. As the above-mentioned oily substrate, for example, glycerides of higher fatty acids [e.g., cacao butter, Witepsols (manufactured by Dynamit Nobel, Germany), etc.], glycerides of medium chain fatty acid [e.g., Miglyols (manufactured by Dynamit Nobel, Germany), etc.], or vegetable oils (e.g., sesame oil, soybean oil, cotton seed oil and the like), and the like are listed. Further, as the aqueous substrate, for example, polyethylene glycols, propylene glycol are listed, and as the aqueous gel substrate, for example, natural gums, cellulose derivatives, vinyl polymers, acrylic acid polymers and the like are listed.

As the above-mentioned sustained release preparation, sustained release microcapsules and the like are listed. The sustained release microcapsule can be produced by a method known per se, such as the method shown in the following. [2].

The compound of the present invention is preferably molded into an oral administration preparation such as a solid preparation (e.g., powder, granule, tablet, capsule) and the like, or molded into a rectal administration preparation such as a suppository. Particularly, an oral administration preparation is preferable.

The concomitant drug can be made into the above-mentioned drug form depending on the kind of the drug.

[1] An injection of the compound of the present invention or the concomitant drug, and preparation thereof, [2] a sustained release preparation or immediate-release preparation of the compound of the present invention or the concomitant drug, and preparation thereof, [3] a sublingual, buccal or intraoral quick integrating agent of the compound of the present invention or the concomitant drug, and preparation thereof, are specifically described in the following.

### [1] Injection and Preparation Thereof.

An injection prepared by dissolving the compound of the present invention or the concomitant drug into water is preferable. This injection may be allowed to contain a benzoate and/or salicylate.

The injection is obtained by dissolving the compound of the present invention or the concomitant drug, and if desirable, a benzoate and/or salicylate, into water.

As the above-mentioned salts of benzoic acid and salicylic acid, for example, salts of alkali metals such as sodium, potassium and the like, salts of alkaline earth metals such as calcium, magnesium and the like, ammonium salts, meglumine salts, salts with organic bases such as tromethamol and the like, etc. can be mentioned.

The concentration of the compound of the present invention or the concomitant drug in an injection is from 0.5 to 50 w/v %, preferably from about 3 to 20 w/v %. The concentration of a benzoate or/and salicylate is from 0.5 to 50 w/v %, preferably from about 3 to 20 w/v %.

Into an injection of the present invention, additives usually used in an injection, for example, a stabilizer (e.g., ascorbic acid, sodium pyrosulfite and the like), a surfactant (e.g., Polysorbate 80, macrogol and the like), a solubilizer (e.g., glycerin, ethanol and the like), a buffer (e.g., phosphoric acid and alkali metal salt thereof, citric acid and alkali metal salt thereof, and the like), an isotonizing agent (e.g., sodium chloride, potassium chloride and the like), a dispersing agent (e.g., hydroxypropylmethylcellulose, dextrin), a pH regulator (e.g., hydrochloric acid, sodium hydroxide and the like), a preservative (e.g., ethyl p-oxybenzoate, benzoic acid and the like), a dissolving agent (e.g., conc. glycerin, meglumine and the like), a dissolution aid (e.g., propylene glycol, sucrose and the like), a soothing agent (e.g., glucose, benzyl alcohol and the like), and the like, can be appropriately blended. These additives are generally blended in a proportion usually used in an injection.

It is advantageous that pH of an injection is controlled from pH 2 to 12, preferably from pH 2.5 to 8.0 by addition of a pH regulator.

An injection is obtained by dissolving the compound of the present invention or the concomitant drug and if desirable, a benzoate and/or a salicylate, and if necessary, the above-mentioned additives into water. These may be dissolved in any order, and can be appropriately dissolved in the same manner as in a conventional method of producing an injection.

An aqueous solution for injection may be advantageously heated, alternatively, for example, filter sterilization, high pressure heat sterilization and the like can be conducted in the same manner as for a usual injection, to provide an injection.

It may be advantageous that an aqueous solution for injection is subjected to high pressure heat sterilization at 100 to 121° C. for 5 to 30 min.

Further, a preparation endowed with an antibacterial property of a solution may also be produced so that it can be used as a preparation which is divided and administered multiple-times.

### [2] Sustained Release Preparation or Immediate-Release Preparation, and Preparation Thereof

A sustained release preparation is preferable which is obtained, if desirable, by coating a nucleus containing the compound of the present invention or the concomitant drug with a film agent such as a water-insoluble substance, swellable polymer and the like. For example, a sustained release preparation for oral administration of once administration per day type is preferable.

As the water-insoluble substance used in a film agent, there are listed, for example, cellulose ethers such as ethylcellulose, butylcellulose and the like, cellulose esters such as cellulose acetate, cellulose propionate and the like, polyvinyl esters such as polyvinyl acetate, polyvinyl butyrate and the like, acrylic acid/methacrylic acid copolymers, methyl methacrylate copolymers, ethoxyethyl methacrylate/cinnamoethyl methacrylate/aminoalkyl methacrylate copolymers, polyacrylic acid, polymethacrylic acid, methacrylic acid alkylamide copolymers, poly(methyl methacrylate), polymethacrylate, polymethacrylamide, aminoalkyl methacrylate copolymers, poly(methacrylic anhydride), glycidyl methacrylate copolymer, particularly, acrylic acid-based polymers such as Eudoragit (Rohm Pharma) such as Eudoragit RS-100, RL-100, RS-30D, RL-30D, RL-PO, RS-PO (ethyl acrylate/methyl methacrylate/trimethylammoniumethyl methacrylate chloride copolymer), Eudoragit NE-30D (methyl methacrylate/ethyl acrylate copolymer), and the like, hydrogenated oils such as hydrogenated castor oil (e.g., Lubri wax (Freund Corporation) and the like), waxes such as carnauba wax, glycerin fatty acid ester, paraffin and the like, polyglycerin fatty esters, and the like.

As the swellable polymer, polymers having an acidic dissociating group and showing pH dependent swell are preferable, and polymers having an acidic dissociating group, which manifest small swelling in acidic regions such as in stomach and large swelling in neutral regions such as in small intestine and large intestine, are preferable.

As such a polymer having an acidic dissociating group and showing pH dependent swell, cross-linkable polyacrylic acid copolymers such as, for example, Carbomer 934P, 940, 941, 974P, 980, 1342 and the like, polycarbophil, calcium polycarbophil (last two are manufactured by BF Goodrich), Hiviswako 103, 104, 105, 304 (all are manufactured by Wako Pure Chemical Industries, Ltd.), and the like, are listed.

The film agent used in a sustained release preparation may further contain a hydrophilic substance.

As the hydrophilic substance, for example, polysaccharides which may contain a sulfate group such as pullulan, dextrin, alkali metal alginate and the like, polysaccharides having a hydroxyalkyl group or carboxyalkyl group such as hydroxypropylcellulose, hydroxypropylmethylcellulose, carboxymethylcellulose sodium and the like, methylcellulose, polyvinylpyrrolidone, polyvinyl alcohol, polyethylene glycol and the like can be mentioned.

The content of a water-insoluble substance in the film agent of a sustained release preparation is from about 30 to about 90% (w/w), preferably from about 35 to about 80% (w/w), further preferably from about 4.0 to about 75% (w/w), the content of a swellable polymer is from about 3 to about 30% (w/w), preferably from about 3 to about 15% (w/w). The film agent may further contain a hydrophilic substance, and in which case, the content of a hydrophilic substance in the film agent is about 50% (w/w) or less, preferably about 5 to 40% (w/w), further preferably from about 5 to 35% (w/w). This % (w/w) indicates % by weight based on a film agent composition which is obtained by removing a solvent (e.g., water, lower alcohols such as methanol, ethanol and the like) from a film agent solution.

The sustained release preparation is produced by preparing a nucleus containing a drugs as exemplified below, then, coating the resulted nucleus with a film agent solution prepared by heat-solving a water-insoluble substance, swellable polymer and the like or by dissolving or dispersing it in a solvent.

### I. Preparation of Nucleus Containing Drug

The form of nucleus containing a drug to be coated with a film agent (hereinafter, sometimes simply referred to as nucleus) is not particularly restricted, and preferably, the nucleus is formed into particles such as a granule or fine particle.

When the nucleus is composed of granules or fine particles, the average particle size thereof is preferably from about 150 to about 2000 μm, further preferably, from about 500 to about 1400 μm.

Preparation of the nucleus can be effected by a usual production method. For example, a suitable excipient, binding agent, disintegrating agent, lubricant, stabilizer and the like are mixed with a drug, and the mixture is subjected to a wet extrusion granulating method, fluidized bed granulating method or the like, to prepare a nucleus.

The content of drugs in a nucleus is from about 0.5 to about 95% (w/w), preferably from about 5.0 to about 80% (w/w), further preferably from about 30 to about 70% (w/w).

As the excipient contained in the nucleus, for example, saccharides such as sucrose, lactose, mannitol, glucose and the like, starch, crystalline cellulose, calcium phosphate, corn starch and the like are used. Among them, crystalline cellulose, corn starch are preferable.

As the binding agent, for example, polyvinyl alcohol, hydroxypropylcellulose, polyethylene glycol, polyvinyl pyrrolidone, Pluronic F68, gum Arabic, gelatin, starch and the like are used. As the disintegrating agent, for example, carboxymethylcellulose calcium (ECG505), croscarmellose sodium (Ac-Di-Sol), crosslinked polyvinylpyrrolidone (Crospovidone), low substituted hydroxypropylcellulose (L-HPC) and the like are used. Among them, hydroxypropylcellulose, polyvinylpyrrolidone, low substituted hydroxypropylcellulose are preferable. As the lubricant and coagulation inhibitor, for example, talc, magnesium stearate and inorganic salts thereof are used, and as the lubricant, polyethylene glycol and the like are used. As the stabilizer, acids such as tartaric acid, citric acid, succinic acid, fumaric acid, maleic acid and the like, are used.

A nucleus can also be prepared by, in addition to the above-mentioned, for example, a rolling granulation method in which a drug or a mixture of a drug with an excipient, lubricant and the like is added portionwise onto an inert carrier particle which is the core of the nucleus while spraying a binder dissolved in a suitable solvent such as water, lower alcohol (e.g., methanol, ethanol and the like) and the like, a pan coating method, a fluidized bed coating method or a melt granulating method. As the inert carrier particle, for example, those made of sucrose, lactose, starch, crystalline cellulose or waxes can be used, and the average particle size thereof is preferably from about 100 μm to about 1500 μm.

For separating a drug contained in a nucleus and a film agent, the surface of the nucleus may be coated with a protective agent. As the protective agent, for example, the above-mentioned hydrophilic substances, water-insoluble substances and the like are used. As the protective agent, preferably polyethylene glycol, and polysaccharides having a hydroxyalkyl group or carboxyalkyl group are used, more preferably, hydroxypropylmethylcellulose and hydroxypropylcellulose are used. The protective agent may contain, as stabilizer, acids such as tartaric acid, citric acid, succinic acid, fumaric acid, maleic acid and the like, and lubricants such as talc and the like. When the protective agent is used, the coating amount is from about 1 to about 15% (w/w), preferably from about 1 to about 10% (w/w), further preferably from about 2 to about 8% (w/w), based on the nucleus.

The protective agent can be coated by a usual coating method, and specifically, the protective agent can be coated by spray-coating the nucleus, for example, by a fluidized bed coating method, pan coating method and the like.

II. Coating of Nucleus with Film Agent

A nucleus obtained in the above-mentioned step I is coated with a film agent solution obtained by heat-solving the above-mentioned water-insoluble substance and pH-dependent swellable polymer, and a hydrophilic substance, or by dissolving or dispersing them in a solvent, to give a sustained release preparation.

As the method for coating a nucleus with a film agent solution, for example, a spray coating method and the like are listed.

The composition ratio of a water-insoluble substance, swellable polymer or hydrophilic substance in a film agent solution is appropriately selected so that the contents of these components in a coated film are the above-mentioned contents, respectively.

The coating amount of a film agent is from about 1 to about 90% (w/w), preferably from about 5 to about 50% (w/w), further preferably from about 5 to about 35% (w/w), based on a nucleus (not including coating amount of protective agent).

As the solvent in a film agent solution, water or an organic solvent can be used alone or in admixture thereof. In the case of use in admixture, the mixing ratio of water to an organic solvent (water/organic solvent: by weight) can be varied in the range from 1 to 100%, and preferably from 1 to about 30%. The organic solvent is not particularly restricted providing it dissolves a water-insoluble substance, and for example, lower alcohols such as methyl alcohol, ethyl alcohol, isopropyl alcohol, n-butyl alcohol and the like, lower alkanone such as acetone and the like, acetonitrile, chloroform, methylene chloride and the like are used. Among them, lower alcohols are preferable, and ethyl alcohol and isopropyl alcohol are particularly preferable. Water, and a mixture of water with an organic solvent are preferably used as a solvent for a film agent. In this case, if necessary, an acid such as tartaric acid, citric acid, succinic acid, fumaric acid, maleic acid and the like may also be added into a film agent solution for stabilizing the film agent solution.

An operation of coating by spray coating can be effected by a usual coating method, and specifically, it can be effected by spray-coating a film agent solution onto a nucleus by a fluidized bed coating method, pan coating method and the like. In this case, if necessary, talc, titanium oxide, magnesium stearate, calcium stearate, light anhydrous silicic acid and the like may also be added as a lubricant, and glycerin fatty acid ester, hydrogenated castor oil, triethyl citrate, cetyl alcohol, stearyl alcohol and the like may also be added as a plasticizer.

After coating with a film agent, if necessary, an antistatic agent such as talc and the like may be mixed.

The immediate-release preparation may be liquid (solution, suspension, emulsion and the like) or solid (particle, pill, tablet and the like). As the immediate-release preparation, oral agents and parenteral agents such as an injection and the like are used, and oral agents are preferable.

The immediate-release preparation, usually, may contain, in addition to an active component drug, also carriers, additives and excipients conventionally used in the preparation field (hereinafter, sometimes abbreviated as excipient). The excipient used is not particularly restricted providing it is an excipient ordinarily used as a preparation excipient. For example, as the excipient for an oral solid preparation, lactose, starch, corn starch, crystalline cellulose (Avicel PH101, manufactured by Asahi Kasei Corporation, and the like), powder sugar, granulated sugar, mannitol, light anhydrous silicic acid, magnesium carbonate, calcium carbonate, L-cysteine and the like are listed, and preferably, corn starch and mannitol and the like are listed. These excipients can be used alone or in combination of two or more. The content of the excipient is, for example, from about 4.5 to about 99.4 w/w %, preferably from about 20 to about 98.5 w/w %, further preferably from about 30 to about 97 w/w %, based on the total amount of the immediate-release preparation.

The content of a drug in the immediate-release preparation can be appropriately selected in the range from about 0.5 to about 95 w/w %, preferably from about 1 to about 60 w/w % based on the total amount of the immediate-release preparation.

When the immediate-release preparation is an oral solid preparation, it usually contains, in addition to the above-mentioned components, also an integrating agent. As this integrating agent, for example, carboxymethylcellulose calcium (ECG-505, manufactured by Gotoku Yakuhin), croscarmellose sodium (for example, Actisol, manufactured by Asahi Kasei Corporation), crospovidone (for example, Kollidon CL, manufactured by BASF), low substituted hydroxypropylcellulose (manufactured by Shin-Etsu Chemical Co., Ltd.), carboxymethylstarch (manufactured by Matsutani Kagaku K.K.), carboxymethylstarch sodium (Exprotab, manufactured by Kimura Sangyo), partially pregelatinized starch (PCS, manufactured by Asahi Kasei Corporation), and the like are used, and for example, those which disintegrate a granule by adsorbing water in contact with water, causing swelling, or making a channel between an effective ingredient constituting the nucleus and an excipient, can be used. These disintegrating agents can be used alone or in combination of two or more. The amount of the disintegrating agent used is appropriately selected depending on the kind and blending amount of a drug used, design of releasing property, and the like, and for example, from about 0.05 to about 30 w/w %, preferably from about 0.5 to about 15 w/w %, based on the total amount of the immediate-release preparation.

When the immediate-release preparation is an oral solid preparation, it may further contain, in addition to the above-mentioned composition, if desired, additives conventional in solid preparations. As such an additive, there are used, for example, a binder (e.g., sucrose, gelatin, gum Arabic powder, methylcellulose, hydroxypropylcellulose, hydroxypropylmethylcellulose, carboxymethylcellulose, polyvinylpyrrolidone, pullulan, dextrin and the like), a lubricant (e.g., polyethylene glycol, magnesium stearate, talc, light anhydrous silicic acid (for example, Aerosil (Nippon Aerosil)), a surfactant (e.g., anionic surfactants such as sodium alkylsulfate and the like, nonionic surfactants such as polyoxyethylene fatty acid ester and polyoxyethylene sorbitan fatty acid ester, polyoxyethylene castor oil derivatives and the like), a coloring agent (e.g., tar coloring matter, caramel, iron oxide red, titanium oxide, riboflavins), if necessary, an appetizing agent (e.g., sweetening agent, flavoring agent and the like), an adsorbent, preservative, wetting agent, antistatic agent, and the like. Further, as the stabilizer, an organic acid such as tartaric acid, citric acid, succinic acid, fumaric acid and the like may also be added.

As the above-mentioned binder, hydroxypropylcellulose, polyethylene glycol and polyvinylpyrrolidone and the like are preferably used.

The immediate-release preparation can be prepared by, based on a usual technology of producing preparations, mixing the above-mentioned components, and if necessary, further kneading the mixture, and molding it. The above-mentioned mixing is conducted by generally used methods, for example, mixing, kneading and the like. Specifically, when an immediate-release preparation is formed, for example, into a particle, it can be prepared, according to the same means as in the above-mentioned method for preparing a nucleus of a sustained release preparation, by mixing the components using a vertical granulator, universal kneader (manufactured by Hata Tekkosho), fluidized bed granulator FD-5S (manufactured by Powrex Corporation), and the like, and then, granulating the mixture by a wet extrusion granulation method, fluidized bed granulation method and the like.

Thus obtained immediate-release preparation and sustained release preparation may be themselves made into products or made into products appropriately together with preparation excipients and the like, separately, by an ordinary method, then, may be administered simultaneously or may be administered in combination at any administration interval, or they may be themselves made into one oral preparation (e.g., granule, fine particle, tablet, capsule and the like) or made into one oral preparation appropriately together with preparation excipients and the like. It may also be permissible that they are made into granules or fine particles, and filled in the same capsule to be used as a preparation for oral administration.

### [3] Sublingual, Buccal or Intraoral Quick Disintegrating Agent and Preparation Thereof.

Sublingual, buccal or intraoral quick disintegrating agents may be a solid preparation such as tablet and the like, or may be an oral mucosa membrane patch (film).

As the sublingual, buccal or intraoral quick disintegrating agent, a preparation containing the compound of the present invention or the concomitant drug and an excipient is preferable. It may contain also auxiliary agents such as a lubricant, isotonizing agent, hydrophilic carrier, water-dispersible polymer, stabilizer and the like. Further, for easy absorption and increased in vivo use efficiency, β-cyclodextrin or β-cyclodextrin derivatives (e.g., hydroxypropyl-β-cyclodextrin and the like) and the like may also be contained.

As the above-mentioned excipient, lactose, sucrose, D-mannitol, starch, crystalline cellulose, light anhydrous silicic acid and the like are listed. As the lubricant, magnesium stearate, calcium stearate, talc, colloidal silica and the like are listed, and particularly, magnesium stearate and colloidal silica are preferable. As the isotonizing agent, sodium chloride, glucose, fructose, mannitol, sorbitol, lactose, saccharose, glycerin, urea and the like are listed, and particularly, mannitol is preferable. As the hydrophilic carrier, swellable hydrophilic carriers such as crystalline cellulose, ethylcellulose, crosslinkable polyvinylpyrrolidone, light anhydrous silicic acid, silicic acid, dicalcium phosphate, calcium carbonate and the like are listed, and particularly, crystalline cellulose (e.g., microcrystalline cellulose and the like) is preferable. As the water-dispersible polymer, gums (e.g., gum tragacanth, acacia gum, cyamoposis gum), alginates (e.g., sodium alginate), cellulose derivatives (e.g., methylcellulose, carboxymethylcellulose, hydroxymethylcellulose, hydroxypropylcellulose, hydroxypropylmethylcellulose), gelatin, water-soluble starch, polyacrylic acids (e.g., Carbomer), polymethacrylic acid, polyvinyl alcohol, polyethylene glycol, polyvinylpyrrolidone, polycarbophil, ascorbate, palmitates and the like are listed, and hydroxypropylmethylcellulose, polyacrylic acid, alginate, gelatin, carboxymethylcellulose, polyvinylpyrrolidone, polyethylene glycol and the like are preferable. Particularly, hydroxypropylmethylcellulose is preferable. As the stabilizer, cysteine, thiosorbitol, tartaric acid, citric acid, sodium carbonate, ascorbic acid, glycine, sodium sulfite and the like are listed, and particularly, citric acid and ascorbic acid are preferable.

The sublingual, buccal or intraoral quick disintegrating agent can be produced by mixing the compound of the present invention or the concomitant drug and an excipient by a method known per se. Further, if desired, auxiliary agents such as a lubricant, isotonizing agent, hydrophilic carrier, water-dispersible polymer, stabilizer, coloring agent, sweetening agent, preservative and the like may be mixed. The sublingual, buccal or intraoral quick disintegrating agent is obtained by mixing the above-mentioned components simultaneously or at a time interval, then subjecting the mixture to tablet-making molding under pressure. For obtaining suitable hardness, it may also be permissible that the materials are moistened by using a solvent such as water, alcohol and the like if desired before and after the tablet making process, and after the molding, the materials are dried, to obtain a product.

In the case of molding into a mucosa membrane patch (film), the compound of the present invention or the concomitant drug and the above-mentioned water-dispersible polymer (preferably, hydroxypropylcellulose, hydroxypropylmethylcellulose), excipient and the like are dissolved in a solvent such as water and the like, and the resulted solution is cast to give a film. Further, additives such as a plasticizer, stabilizer, antioxidant, preservative, coloring agent, buffer, sweetening agent and the like may also be added. For imparting suitable elasticity to the film, glycols such as polyethylene glycol, propylene glycol and the like may be contained, or for enhancing adhesion of the film to an intraoral mucosa membrane lining, a bio-adhesive polymer (e.g., polycarbophil, carbopol) may also be contained. In the casting, a solution is poured on the non-adhesive surface, spread to uniform thickness (preferably, about 10 to 1000 micron) by an application tool such as a doctor blade and the like, then, the solution is dried to form a film. It may be advantageous that thus formed film is dried at room temperature or under heat, and cut into a desired area.

As the preferable intraoral quick disintegrating agent, solid quick scattering dose agents composed of a network body comprising the compound of the present invention or the concomitant drug, and a water-soluble or water-diffusible carrier which is inert to the compound of the present invention or concomitant drug, are listed. This network body is obtained by sublimating a solvent from the solid composition constituted of a solution prepared by dissolving the compound of the present invention or the concomitant drug in a suitable solvent.

It is preferable that the composition of an intraoral quick disintegrating agent contains a matrix forming agent and a secondary component, in addition to the compound of the present invention or the concomitant drug.

Examples of the matrix forming agent include gelatins, dextrins, animal proteins or vegetable proteins such as soybean, wheat and psyllium seed protein and the like; rubber substances such as gum Arabic, guar gum, agar, xanthane gum and the like; polysaccharides; alginic acids; carboxymethylcelluloses; carageenans; dextrans; pectines; synthetic polymers such as polyvinylpyrrolidone and the like; substances derived from a gelatin-gum Arabic complex, and the like. Further, saccharides such as mannitol, dextrose, lactose, galactose, trehalose and the like; cyclic saccharides such as cyclodextrin and the like; inorganic salts such as sodium phosphate, sodium chloride and aluminum silicate and the like; amino acids having 2 to 12 carbon atoms such as glycine, L-alanine, L-aspartic acid, L-glutamic acid, L-hydroxyproline, L-isoleucine, L-leucine, L-phenylalanine and the like, are contained.

One or more of the matrix forming agents can be introduced in a solution or suspension before solidification. Such a matrix forming agent may be present in addition to a surfactant, or may be present while a surfactant being excluded. The matrix forming agents aid to maintain the compound of the present invention or the concomitant drug in the solution or suspension in diffused condition, in addition to formation of the matrix.

The composition may contain secondary components such as a preservative, antioxidant, surfactant, thickening agent, coloring agent, pH controlling agent, flavoring agent, sweetening agent, food taste masking agent and the like. As the suitable coloring agent, there are listed red, black and yellow iron oxides, and FD & C dyes such as FD & C Blue 2, FD & C Red 40 and the like manufactured by Ellis and Everard. Examples of the suitable flavoring agent include mint, raspberry, licorice, orange, lemon, grapefruit, caramel, vanilla, cherry, grape flavor and combinations thereof. Examples of the suitable pH controlling agent include citric acid, tartaric acid, phosphoric acid, hydrochloric acid and maleic acid. Examples of the suitable sweetening agent include aspartame, acesulfame K and thaumatin and the like. Examples of the suitable food taste masking agent include sodium bicarbonate, ion exchange resin, cyclodextrin-inclusion compounds, adsorbent substances and microcapsulated apomorphine.

The preparation contains the compound of the present invention or the concomitant drug in an amount usually from about 0.1 to about 50% by weight, preferably from about 0.1 to about 30% by weight, and preferable are preparations (such as the above-mentioned sublingual agent, buccal and the like) which can dissolve 90% or more of the compound of the present invention or the concomitant drug (into water) within the time range of about 1 to about 60 min, preferably of about 1 to about 15 min, more preferably of about 2 to about 5 min, and intraoral quick disintegrating preparations which are disintegrated within the range of 1 to 60 sec, preferably of 1 to 30 sec, further preferably of 1 to 10 sec, after placed in an oral cavity.

The content of the above-mentioned excipient in the whole preparation is from about 10 to about 99% by weight, preferably from about 30 to about 90% by weight. The content of β-cyclodextrin or β-cyclodextrin derivative in the whole preparation is from 0 to about 30% by weight. The content of the lubricant in the whole preparation is from about 0.01 to about 10% by weight, preferably from about 1 to about 5% by weight. The content of the isotonizing agent in the whole preparation is from about 0.1 to about 90% by weight, preferably, from about 10 to about 70% by weight. The content of the hydrophilic carrier in the whole preparation is from about 0.1 to about 50% by weight, preferably, from about 10 to about 30% by weight. The content of the water-dispersible polymer in the whole preparation is from about 0.1 to about 30% by weight, preferably, from about 10 to about 25% by weight. The content of the stabilizer in the whole preparation is from about 0.1 to about 10% by weight, preferably, from about 1 to 5% by weight. The above-mentioned preparation may further contain additives such as a coloring agent, sweetening agent, preservative and the like, if necessary.

The dosage of a combination agent of the present invention differs depending on the kind of a compound of the present invention, age, body weight, condition, drug form, administration method, administration period and the like, and for example, for one cancer patient (adult, body weight: about 60 kg), the combination agent is administered intravenously, at a dose of about 0.01 to about 1000 mg/kg/day, preferably about 0.01 to about 100 mg/kg/day, more preferably about 0.1 to about 100 mg/kg/day, particularly about 0.1 to about 50 mg/kg/day, especially about 1.5 to about 30 mg/kg/day, in terms of the compound of the present invention or the concomitant drug, respectively, once or several times in division a day. Of course, since the dose as described above varies depending on various conditions, amounts smaller than the above-mentioned dosage may sometimes be sufficient, further, amounts over that range sometimes have to be administered.

The amount of the concomitant drug can be set at any value unless side effects are problematical. The daily dosage in terms of the concomitant drug differs depending on the severity of the symptom, age, sex, body weight, sensitivity difference of the subject, administration period, interval, and nature, pharmacy, kind of the pharmaceutical preparation, kind of effective ingredient, and the like, and not particularly restricted, and the amount of a drug is, in the case of oral administration for example, usually from about 0.001 to 2000 mg, preferably from about 0.01 to 500 mg, further preferably from about 0.1 to 100 mg, per 1 kg of a mammal and this is usually administered once to 4-times in division a day.

In administration of a combination agent of the present invention, the compound of the present invention may be administered after administration of the concomitant drug or the concomitant drug may be administered after administration of the compound of the present invention, though they may be administered simultaneously. When administered at a time interval, the interval differs depending on the effective ingredient to be administered, drug form and administration method, and for example, when the concomitant drug is administered first, a method in which the compound of the present invention is administered within time range of from 1 min to 3 days, preferably from 10 min to 1 day, more preferably from 15 min to 1 hr after administration of the concomitant drug is exemplified. When the compound of the present invention is administered first, a method in which the concomitant drug is administered within time range of from 1 min to 1 day, preferably from 10 min to 6 hrs, more preferably from 15 min to 1 hr after administration of the compound of the present invention is exemplified.

In a preferable administration method, for example, the concomitant drug which has been formed into an oral administration preparation is administered orally at a daily dose of about 0.001 to 200 mg/kg, and about 15 min later, the compound of the present invention which has been formed into an oral administration-preparation is administered orally at a daily dose of about 0.005 to 100 mg/kg.

Furthermore, the compound of the present invention or the combination agent of the present invention can be used concurrently with a non-drug therapy. To be precise, the compound of the present invention and the combination agent of the present invention can be combined with a non-drug therapy such as (1) surgery, (2) hypertensive chemotherapy using angiotensin II etc., (3) gene therapy, (4) thermotherapy, (5) cryotherapy, (6) laser cauterization, (7) radiotherapy, and the like.

For example, use of the compound of the present invention or the concomitant drug of the present invention before or after operation and the like, or before or after a treatment with a combination of two or three kinds thereof provides effects of prevention of resistance expression, extension of Disease-Free Survival, suppression of metastasis or recurrence of cancer, life prolongation and the like.

In addition, a treatment with the compound of the present invention or the concomitant drug of the present invention can also be combined with supportive therapies [(i) administration of antibiotics (e.g., β-lactam such as pansporin and the like, macrolide such as clarithromycin and the like, and the like) for various associated infections, (ii) administration of high-calorie infusion, amino acid preparations, general vitamin preparations for improvement of malnutrition, (iii) administration of morphine for pain relief, (iv) administration of pharmaceutical agents for reducing side effects such as nausea, vomiting, anorexia, diarrhea, leucopenia, thrombocytopenia, low hemoglobin concentration, hair loss, hepatopathy, renopathy, DIC, fever and the like and (v) administration of pharmaceutical agents for suppressing multiple drug resistance in cancer and the like].

Preferably, the compound of the present invention or the combination agent of the present invention is administered orally (including sustained-release preparations), intravenously (including boluses, infusions and clathrates), subcutaneously and intramuscularly (including boluses, infusions and sustained-release preparations), transdermally, intratumorally or proximally before or after the above-described treatment is conducted.

As a period for administering the compound of the present invention or the combination agent of the present invention before the surgery, etc., for example, it can be administrated 1-time about 30 min to 24 hrs before the surgery, etc., or in 1 to 3 cycles about 3 months to 6 months before the surgery, etc. In this way, the surgery, etc. can be conducted easily because, for example, a cancer tissue would be reduced by administering the compound of the present invention or the combination agent of the present invention before the surgery, and the like.

As a period for administering the compound of the present invention or the combination agent of the present invention after the surgery, etc., for example, it can be administrated repeatedly per a few weeks to 3 months, about 30 min to 24 hrs after the surgery, and the like. In this way, it makes an effect of the surgery, etc. increasing by administering the compound of the present invention or the combination agent of the present invention after the surgery, and the like.

Since the compounds (I)-(III) of the present invention, salts thereof and prodrugs thereof show superior inhibitory activity against kinase such as vascular endothelial growth factor receptor and the like, they can provide clinically useful agents for the prophylaxis or treatment of diseases (e.g., cancer and the like) associated with the action of vascular endothelial growth factors in living organisms. Moreover, since the compounds (I)-(III) of the present invention, salts thereof and prodrugs thereof are superior in the efficacy expression, pharmacokinetic, solubility, interaction with other pharmaceutical products, safety and stability, they are useful as pharmaceutical agents.

### EXAMPLES

The present invention is explained in more detail in the following by referring to Reference Examples, Examples, Formulation Examples and Experimental Examples, which are not to be construed as limitative.

The “room temperature” in the following Reference Examples and Examples indicates normally about 10° C. to about 35° C. The “%” shows percentage by weight unless otherwise indicated, and yield shows mol/mol %.

Other abbreviations used in the specification indicate the following meanings:

s: singlet, d: doublet, t: triplet, q: quartet, m: multiplet, br: broad, J: coupling constant

In the following Examples, products that meet the Japanese Pharmacopoeia 14th Edition or Japanese Pharmaceutical Excipients 2003 were used as various additives such as lactose, cornstarch, microcrystalline cellulose and magnesium stearate.

### Reference Example 1

### 4-chloro-5-methyl-5H-pyrrolo[3,2-d]pyrimidine

A mixture of 4-chloro-5H-pyrrolo[3,2-d]pyrimidine (5.01 g, 32.5 mmol), methyl methanesulfonate (3.07 g, 34.2 mmol), cesium carbonate (21.2 g, 65.2 mmol) and N,N-dimethylformamide (50 mL) was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was washed with ethyl acetate/hexane=1/1 solution to give the title compound (4.36 g, 80%) as a yellow solid.

¹H-NMR (DMSO-d₆, 300 MHz) δ 4.09 (3H, s), 6.67-6.68 (1H, m), 7.95 (1H, d, J=3.0 Hz), 8.57 (1H, s).

### Reference Example 2

### 4-[(5-methyl-5H-pyrrolo[3,2-d]pyrimidin-4-yl)oxy]aniline

A mixture of 4-chloro-5-methyl-5H-pyrrolo[3,2-d]pyrimidine (4.00 g, 23.9 mmol), 4-aminophenol (2.86 g, 26.3 mmol), potassium carbonate (9.91 g, 71.7 mmol) and N-methylpyrrolidone (60 mL) was stirred at 110° C. for 3 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=20/80→100/0) to give the title compound (3.47 g, 60%) as a yellow solid.

¹H-NMR (DMSO-d₆, 300 MHz) δ 4.06 (3H, s), 5.04 (2H, br s), 6.53 (1H, d, J=3.0 Hz), 6.58 (2H, d, J=8.7 Hz), 6.91 (2H, d, J=8.7 Hz), 7.70 (1H, d, J=3.0 Hz), 8.21 (1H, s).

### Reference Example 3

### 2-chloro-4-[(5-methyl-5H-pyrrolo[3,2-d]pyrimidin-4-yl)oxy]aniline

A mixture of 4-chloro-5-methyl-5H-pyrrolo[3,2-d]pyrimidine (168 mg, 1.0 mmol), 4-amino-3-chlorophenol (215 mg, 1.5 mmol), potassium carbonate (415 mg, 3.0 mmol) and N-methylpyrrolidone (3 mL) was stirred at 120° C. for 18 hr. The reaction mixture was diluted with water and extracted with ethyl acetate (×3). The organic layer was concentrated under reduced pressure, and the residue was purified by column chromatography (NH silica gel, hexane/ethyl acetate=90/10→0/100) to give the title compound (100 mg, 36%).

¹H-NMR (CDCl₃, 300 MHz) δ 4.05 (2H, br s), 4.13 (3H, s), 6.64 (1H, d, J=3.0 Hz), 6.84 (1H, d, J=8.7 Hz), 7.00 (1H, dd, J=2.4, 8.7 Hz), 7.20 (1H, d, J=2.4 Hz), 7.31 (1H, d, J=3.0 Hz), 8.45 (1H, s).

### Reference Example 4

### 4-chloro-5-ethyl-5H-pyrrolo[3,2-d]pyrimidine

A mixture of 4-chloro-5H-pyrrolo[3,2-d]pyrimidine (1075 mg, 7.0 mmol), bromoethane (915 mg, 8.4 mmol), cesium carbonate (3421 mg, 10.5 mmol) and N,N-dimethylformamide (10 mL) was stirred at room temperature for 18 hr. The reaction mixture was concentrated under reduced pressure, and the residue was diluted with water and extracted with ethyl acetate (×3). The organic layer was concentrated under reduced pressure, and the residue was purified by column chromatography (silica gel, hexane/ethyl acetate=50/50→0/100) to give the title compound (806 mg, 63%) as a yellow solid.

¹H-NMR (DMSO-d₆, 300 MHz) δ 1.41 (3H, t, J=7.1 Hz), 4.53 (2H, q, J=7.1 Hz), 6.74 (1H, d, J=3.2 Hz), 8.08 (1H, d, J=3.2 Hz), 8.62 (1H, s).

### Reference Example 5

### 2-chloro-4-[(5-ethyl-5H-pyrrolo[3,2-d]pyrimidin-4-yl)oxy]aniline

A mixture of 4-chloro-5-ethyl-5H-pyrrolo[3,2-d]pyrimidine (726 mg, 4.0 mmol), 4-amino-3-chlorophenol (861 mg, 6.0 mmol), cesium carbonate (3910 mg, 12.0 mmol) and N-methylpyrrolidone (5 mL) was stirred at 120° C. for 18 hr. The reaction mixture was diluted with water and extracted with ethyl acetate (×3). The organic layer was concentrated under reduced pressure, and the residue was purified by column chromatography (NH silica gel, hexane/ethyl acetate=90/10→0/100) and recrystallized from diisopropyl ether/ethyl acetate to give the title compound (894 mg, 77%).

¹H-NMR (DMSO-d₆, 300 MHz) δ 1.44 (3H, t, J=7.1 Hz), 4.44 (2H, q, J=7.1 Hz), 5.34 (2H, s), 6.59 (1H, d, J=3.0 Hz), 6.85 (1H, d, J=8.7 Hz), 7.00 (1H, dd, J=8.7, 2.6 Hz), 7.23 (1H, d, J=2.6 Hz), 7.84 (1H, d, J=3.0 Hz), 8.27 (1H, s).

### Reference Example 6

### 2-[2-(4-chloro-5H-pyrrolo[3,2-d]pyrimidin-5-yl)ethoxy]ethyl Benzoate

A mixture of 4-chloro-5H-pyrrolo[3,2-d]pyrimidine (2.13 g, 13.9 mmol), 2-(2-{[(4-methylphenyl)sulfonyl]oxy}ethoxy)ethyl benzoate (4.19 g, 14.6 mmol), cesium carbonate (9.02 g, 27.7 mmol) and N,N-dimethylformamide (25 mL) was stirred at 60° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=20/80→100/0) to give the title compound (3.73 g, 78%).

¹H-NMR (DMSO-d₆, 300 MHz) δ 3.66-3.69 (2H, m), 3.82-3.86 (2H, m), 4.27-4.30 (2H, m), 4.65-4.68 (2H, m), 6.60 (1H, d, J=3.0 Hz), 7.46-7.51 (2H, m), 7.61-7.66 (1H, m), 7.80-7.84 (2H, m), 7.95 (1H, d, J=3.0 Hz), 8.56 (1H, s).

### Reference Example 7

### 2-{2-[4-(4-amino-3-chlorophenoxy)-5H-pyrrolo[3,2-d]pyrimidin-5-yl]ethoxy}ethyl Benzoate

A mixture of 2-[2-(4-chloro-5H-pyrrolo[3,2-d]pyrimidin-5-yl)ethoxy]ethyl benzoate (364 mg, 1.05 mmol), 4-amino-3-chlorophenol (150 mg, 1.05 mmol), potassium carbonate (288 mg, 2.08 mmol) and N-methylpyrrolidone (10 mL) was stirred at 110° C. for 1 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=30/70→100/0) to give the title compound (431 mg, 91%).

¹H-NMR (DMSO-d₆, 300 MHz) δ 3.68-3.71 (2H, m), 3.86-3.89 (2H, m), 4.29-4.32 (2H, m), 4.55-4.59 (2H, m), 5.30 (2H, br s), 6.48 (1H, d, J=3.0 Hz), 6.80 (1H, d, J=8.4 Hz), 6.94 (1H, dd, J=8.4, 2.4 Hz), 7.18 (1H, d, J=2.4 Hz), 7.44-7.99 (2H, m), 7.60-7.66 (1H, m), 7.75 (1H, d, J=3.0 Hz), 7.80-7.83 (2H, m), 8.23 (1H, s).

### Reference Example 8

### 4-chloro-5-[2-(2-methoxyethoxy)ethyl]-5H-pyrrolo[3,2-d]pyrimidine

A mixture of 4-chloro-5H-pyrrolo[3,2-d]pyrimidine (768 mg, 5.0 mmol), 1-bromo-2-(2-methoxyethoxy)ethane (90%, 1000 mg, 4.9 mmol), cesium carbonate (2118 mg, 6.5 mmol) and N,N-dimethylformamide (5 mL) was stirred at room temperature for 18 hr. The reaction mixture was diluted with water and extracted with ethyl acetate (×3). The organic layer was concentrated under reduced pressure, and the residue was purified by column chromatography (NH silica gel, hexane/ethyl acetate=90/10→0/100) to give the title compound (1173 mg, 93%) as a pale-yellow oil.

¹H-NMR (CDCl₃, 300 MHz) δ 3.31 (3H, s), 3.40-3.50 (2H, m), 3.50-3.60 (2H, m), 3.88 (2H, t, J=5.1 Hz), 4.74 (2H, t, J=5.1 Hz), 6.86 (1H, d, J=3.3 Hz), 7.74 (1H, d, J=3.3 Hz), 8.76 (1H, s).

### Reference Example 9

### 2-chloro-4-({5-[2-(2-methoxyethoxy)ethyl]-5H-pyrrolo[3,2-d]pyrimidin-4-yl}oxy)aniline

A mixture of 4-chloro-5-[2-(2-methoxyethoxy)ethyl]-5H-pyrrolo[3,2-d]pyrimidine (1135 mg, 4.4 mmol), 4-amino-3-chlorophenol (956 mg, 6.7 mmol), cesium carbonate (4339 mg, 13.3 mmol) and N-methylpyrrolidone (5 mL) was stirred at 120° C. for 18 hr. The reaction mixture was diluted with water and extracted with ethyl acetate (×3). The organic layer was concentrated under reduced pressure, and the residue was purified by column chromatography (NH silica gel, hexane/ethyl acetate=90/10→10/90). The object fraction was concentrated under reduced pressure. The residue was purified by column chromatography (silica gel, ethyl acetate/methanol=0/100→85/15) to give the title compound (971 mg, 60%) as an oil.

¹H-NMR (DMSO-d₆, 300 MHz) δ 3.15 (3H, s), 3.30-3.40 (2H, m), 3.45-3.55 (2H, m), 3.82 (2H, t, J=5.4 Hz), 4.56 (2H, t, J=5.4 Hz), 5.33 (2H, s), 6.59 (1H, d, J=3.3 Hz), 6.85 (1H, d, J=8.7 Hz), 7.00 (1H, dd, J=8.7, 2.7 Hz), 7.22 (1H, d, J=2.7 Hz), 7.79 (1H, d, J=3.3 Hz), 8.28 (1H, s).

### Reference Example 10

### 3-chloro-4-[(5-methyl-5H-pyrrolo[3,2-d]pyrimidin-4-yl)oxy]aniline

Using 4-chloro-5-methyl-5H-pyrrolo[3,2-d]pyrimidine (300 mg, 1.79 mmol), 4-amino-2-chlorophenol (283 mg, 1.97 mmol), potassium carbonate (544 mg, 3.94 mmol) and N-methylpyrrolidone (5 mL), and in the same manner as in Reference Example 3, the title compound (160 mg, 32%) was obtained as a white solid.

¹H-NMR (DMSO-d₆, 300 MHz) δ 4.07 (3H, s), 5.35 (2H, br s), 6.56-6.58 (2H, m), 6.69-6.70 (1H, m), 7.07 (1H, d, J=8.7 Hz), 7.74 (1H, d, J=2.7 Hz), 8.22 (1H, s).

### Reference Example 11

### 4-chloro-5-(2-methoxyethyl)-5H-pyrrolo[3,2-d]pyrimidine

A mixture of 4-chloro-5H-pyrrolo[3,2-d]pyrimidine (923 mg, 6.00 mmol), 2-bromoethylmethylether (877 mg, 6.31 mmol), cesium carbonate (3.91 g, 12.0 mmol) and N,N-dimethylformamide (50 mL) was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was washed with ethyl acetate/hexane=1/1 solution to give the title compound (1.21 g, 96%) as a yellow solid.

¹H-NMR (DMSO-d₆, 300 MHz) δ 3.19 (3H, s), 3.68 (2H, t, J=5.4 Hz), 4.65 (2H, t, J=5.4 Hz), 6.71-6.72 (1H, m), 7.97 (1H, d, J=3.3 Hz), 8.60 (1H, s).

### Reference Example 12

### 2-chloro-4-{[5-(2-methoxyethyl)-5H-pyrrolo[3,2-d]pyrimidin-4-yl]oxy}aniline

A mixture of 4-chloro-5-(2-methoxyethyl)-5H-pyrrolo[3,2-d]pyrimidine (1.06 g, 5.01 mmol), 4-amino-3-chlorophenol (791 mg, 5.51 mmol), potassium carbonate (1.52 g, 11.0 mmol) and N-methylpyrrolidone (10 mL) was stirred at 110° C. for 1 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=30/70→100/0) to give the title compound (901 mg, 56%) as a white solid.

¹H-NMR (DMSO-d₆, 300 MHz) δ 3.19 (3H, s), 3.72 (2H, t, J=5.4 Hz), 4.55 (2H, t, J=5.4 Hz), 5.31 (2H, s), 6.56 (1H, d, J=3.2 Hz), 6.83 (1H, d, J=8.9 Hz), 6.97 (1H, dd, J=8.9, 2.7 Hz), 7.20 (1H, d, J=2.7 Hz), 7.75 (1H, d, J=3.2 Hz), 8.26 (1H, d, J=0.6 Hz).

### Reference Example 13

### 2-(4-chloro-5H-pyrrolo[3,2-d]pyrimidin-5-yl)ethyl Benzoate

Using 4-chloro-5H-pyrrolo[3,2-d]pyrimidine (3.00 g, 19.5 mmol), 2-bromoethyl benzoate (4.70 g, 20.5 mmol), cesium carbonate (9.53 g, 29.3 mmol) and N,N-dimethylformamide (25 mL) as starting materials, and in the same manner as in Reference Example 11, the title compound (5.50 g, 93%) was obtained as a yellow solid.

¹H-NMR (DMSO-d₆, 300 MHz) δ 4.65 (2H, t, J=5.0 Hz), 4.90 (2H, t, J=5.0 Hz), 6.73 (1H, d, J=3.3 Hz), 7.45 (2H, t, J=7.7 Hz), 7.61 (1H, t, J=7.7 Hz), 7.78-7.81 (2H, m), 8.12 (1H, d, J=3.3 Hz) 8.60 (1H, s).

### Reference Example 14

### 2-[4-(4-amino-3-chlorophenoxy)-5H-pyrrolo[3,2-d]pyrimidin-5-yl]ethyl Benzoate

Using 2-(4-chloro-5H-pyrrolo[3,2-d]pyrimidin-5-yl)ethyl benzoate (2.00 g, 6.63 mmol), 4-amino-3-chlorophenol (1.05 g, 7.29 mmol), potassium carbonate (2.02 g, 14.6 mmol) and N-methylpyrrolidone (10 mL) as starting materials, and in the same manner as in Reference Example 12, the title compound (2.15 g, 79%) was obtained as a white solid.

¹H-NMR (DMSO-d₆, 300 MHz) δ 4.69-4.70 (2H, m), 4.78-4.80 (2H, m), 5.31 (2H, br s), 6.61 (1H, d, J=3.2 Hz), 6.79 (1H, d, J=8.9 Hz), 6.87 (1H, dd, J=8.9, 2.6 Hz), 7.01 (1H, d, J=2.6 Hz), 7.45 (2H, t, J=7.8 Hz), 7.60-7.65 (1H, m), 7.76-7.79 (2H, m), 7.91 (1H, d, J=3.2 Hz), 8.26 (1H, s).

### Reference Example 15

### 4-(2-fluoro-4-nitrophenoxy)-5-methyl-5H-pyrrolo[3,2-d]pyrimidine

A mixture of 4-chloro-5-methyl-5H-pyrrolo[3,2-d]pyrimidine (210 mg, 1.25 mmol), 2-fluoro-4-nitrophenol (236 mg, 1.50 mmol) and o-xylene (10 mL) was stirred at 100° C. for 2 days. The reaction mixture was diluted with ethyl acetate, and the organic layer was washed with water and saturated brine, dried over anhydrous magnesium sulfate, and filtrated. The filtrate was concentrated under reduced pressure, and the residue was washed with ethyl acetate-hexane to give the title compound (260 mg, 72%) as a yellow solid.

¹H-NMR (DMSO-d₆, 200 MHz) δ 4.10 (3H, s), 6.64-6.65 (1H, m), 7.81-7.86 (2H, m), 8.20-8.24 (1H, m), 8.30 (1H, d, J=1.2 Hz), 8.36-8.41 (1H, m).

### Reference Example 16

### 3-fluoro-4-[(5-methyl-5H-pyrrolo[3,2-d]pyrimidin-4-yl)oxy]aniline

A mixture of 4-(2-fluoro-4-nitrophenoxy)-5-methyl-5H-pyrrolo[3,2-d]pyrimidine (250 mg, 0.867 mmol), zinc (567 mg, 8.67 mmol), ammonium chloride (186 mg, 3.47 mmol) and methanol (10 mL) was stirred under reflux for 1 hr. After celite filtration, the filtrate was diluted with water and extracted with ethyl acetate. The organic layer was washed with saturated brine, dried over anhydrous magnesium sulfate, and filtrated. The filtrate was concentrated under reduced pressure, and the residue was washed with ethyl acetate-hexane to give the title compound (150 mg, 67%) as a yellow solid.

¹H-NMR (DMSO-d₆, 200 MHz) δ 4.06 (3H, s), 5.36 (2H, s), 6.37-6.49 (2H, m), 6.57 (1H, dd, J=3.0, 1.2 Hz), 7.00-7.06 (1H, m), 7.75 (1H, d, J=3.0 Hz), 8.24 (1H, d, J=1.2 Hz).

### Reference Example 17

### 4-(3-fluoro-4-nitrophenoxy)-5-methyl-5H-pyrrolo[3,2-d]pyrimidine

Using 4-chloro-5-methyl-5H-pyrrolo[3,2-d]pyrimidine (550 mg, 3.28 mmol), 3-fluoro-4-nitrophenol (619 mg, 3.94 mmol) and o-xylene (20 mL) as starting materials, and in the same manner as in Reference Example 15, the title compound (725 mg, 77%) was obtained as a yellow solid.

¹H-NMR (DMSO-d₆, 300 MHz) δ 4.09 (3H, s), 6.66 (1H, d, J=3.2 Hz), 7.46-7.50 (1H, m), 7.78 (1H, dd, J=12.5, 2.6 Hz), 7.86 (1H, d, J=3.2 Hz), 8.31 (1H, t, J=8.9 Hz), 8.38 (1H, s).

### Reference Example 18

### 2-fluoro-4-[(5-methyl-5H-pyrrolo[3,2-d]pyrimidin-4-yl)oxy]aniline

Using 4-(3-fluoro-4-nitrophenoxy)-5-methyl-5H-pyrrolo[3,2-d]pyrimidine (715 mg, 0.867 mmol), zinc (1.62 g, 24.8 mmol), ammonium chloride (531 mg, 9.92 mmol) and methanol (10 mL) as starting materials, and in the same manner as in Reference Example 16, the title compound (256 mg, 40%) was obtained as a yellow solid.

¹H-NMR (DMSO-d₆, 300 MHz) δ 4.08 (3H, s), 5.11 (2H, s), 6.57 (1H, d, J=3.2 Hz), 6.77-6.89 (2H, m), 7.05 (1H, dd, J=12.0, 2.1 Hz), 7.75 (1H, d, J=3.2 Hz), 8.26 (1H, s).

### Reference Example 19

### 4-[(5-methyl-5H-pyrrolo[3,2-d]pyrimidin-4-yl)oxy]naphthalene-1-amine

Using 4-chloro-5-methyl-5H-pyrrolo[3,2-d]pyrimidine (70.0 mg, 0.418 mmol), 4-amino-1-naphthol (100 mg, 0.628 mmol), potassium carbonate (173 mg, 1.25 mmol) and N-methylpyrrolidone (5 mL) as starting materials, and in the same manner as in Reference Example 2, the title compound (29.0 mg, 25%) was obtained as a white solid.

¹H-NMR (DMSO-d₆, 300 MHz) δ 4.12 (3H, s), 5.99 (2H, s), 6.50 (1H, d, J=7.8 Hz), 6.56-6.57 (1H, m), 6.60 (1H, d, J=7.8 Hz), 7.32-7.36 (2H, m), 7.74 (1H, d, J=3.0 Hz), 7.91-7.94 (1H, m), 7.98-8.01 (1H, m), 8.07 (1H, s).

### Reference Example 20

### 8-[(5-methyl-5H-pyrrolo[3,2-d]pyrimidin-4-yl)oxy]quinoline-5-amine

Using 4-chloro-5-methyl-5H-pyrrolo[3,2-d]pyrimidine (209 mg, 1.25 mmol), 5-amino-8-hydroxyquinoline (300 mg, 1.87 mmol), potassium carbonate (777 mg, 5.63 mmol) and N-methylpyrrolidone (10 mL) as starting materials, and in the same manner as in Reference Example 2, the title compound (202 mg, 55%) was obtained as a yellow solid.

¹H-NMR (DMSO-d₆, 300 MHz) δ 4.15 (3H, s), 5.97 (2H, s), 6.55-6.57 (1H, m), 6.72 (1H, d, J=8.4 Hz), 7.34-7.40 (2H, m), 7.74 (1H, d, J=3.0 Hz), 8.05 (1H, d, J=1.2 Hz), 8.54-8.62 (2H, m).

### Reference Example 21

### 1-(3-nitrophenyl)-1H-imidazole

To a solution of sodium hydride (1.42 g, 35.3 mmol) in N,N-dimethylformamide (20 mL) was added dropwise a solution of imidazole (2.00 g, 29.4 mmol) in N,N-dimethylformamide (10 mL) at 0° C., and the mixture was stirred at room temperature for 1 hr. 1-Fluoro-3-nitrobenzene (4.15 g, 29.4 mmol) was added to the reaction mixture and the mixture was stirred at 100° C. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=50/50) to give the title compound (3.05 g, 55%) as a yellow solid.

¹H-NMR (DMSO-d₆, 300 MHz) δ 7.14 (1H, s), 7.79 (1H, t, J=8.4 Hz), 7.94 (1H, s), 8.13-8.19 (2H, m), 8.45 (1H, s), 8.48 (1H, t, J=2.1 Hz).

### Reference Example 22

### 3-(1H-imidazol-1-yl)aniline

To a solution of 1-(3-nitrophenyl)-1H-imidazole (2.00 g, 10.1 mmol) in methanol (20 mL) was added palladium carbon (50% water-containing product, 200 mg), and the mixture was stirred under a hydrogen atmosphere at room temperature for 2 hr. The reaction mixture was filtered through celite. The filtrate was concentrated under reduced pressure, and the residue was purified by column chromatography (NH silica gel, ethyl acetate) to give the title compound (1.50 g, 93%).

¹H-NMR (DMSO-d₆, 300 MHz) δ 5.36 (2H, br s), 6.50-6.54 (1H, m), 6.65-6.70 (2H, m), 7.03-7.04 (1H, m), 7.10 (1H, t, J=7.8 Hz), 7.52-7.53 (1H, m), 8.03 (1H, s).

### Reference Example 23

### 2-chloro-4-(5H-pyrrolo[3,2-d]pyrimidin-4-yloxy)aniline

A mixture of 4-chloro-5H-pyrrolo[3,2-d]pyrimidine (3.07 g, 20 mmol), 4-amino-3-chlorophenol (3.44 g, 24 mmol), potassium carbonate (8.29 g, 60 mmol) and N-methylpyrrolidone (20 mL) was stirred at 120° C. for 18 hr. The reaction mixture was diluted with water and extracted with ethyl acetate. The organic layer was concentrated under reduced pressure, and the residue was purified by column chromatography (silica gel, hexane/ethyl acetate=70/30→0/100) and recrystallized from ethyl acetate to give the title compound (2.31 g, 44%).

¹H-NMR (DMSO-d₆, 300 MHz) δ 5.33 (2H, s), 6.61 (1H, d, J=3.0 Hz), 6.85 (1H, d, J=8.7 Hz), 6.99 (1H, dd, J=8.7, 2.7 Hz), 7.21 (1H, d, J=2.7 Hz), 7.77 (1H, d, J=3.0 Hz), 8.30 (1H, s), 12.26 (1H, br s).

### Reference Example 24

### 2-methyl-4-[(5-methyl-5H-pyrrolo[3,2-d]pyrimidin-4-yl)oxy]aniline

Using 4-chloro-5-methyl-5H-pyrrolo[3,2-d]pyrimidine (1.68 g, 10 mmol), 4-amino-3-methylphenol (1.48 g, 12 mmol), potassium carbonate (4.15 g, 30 mmol) and N-methylpyrrolidone (10 mL) and in the same manner as in Reference Example 23, the title compound (1.24 g, 49%) was obtained.

¹H-NMR (DMSO-d₆, 300 MHz) δ 2.07 (3H, s), 4.08 (3H, s), 4.81 (2H, s), 6.55 (1H, d, J=3.0 Hz), 6.65 (1H, d, J=8.5 Hz), 6.81 (1H, dd, J=8.5, 2.6 Hz), 6.86 (1H, d, J=2.6 Hz), 7.73 (1H, d, J=3.0 Hz), 8.23 (1H, s).

### Reference Example 25

### 4-chloro-5-isopropyl-5H-pyrrolo[3,2-d]pyrimidine

A mixture of 4-chloro-5H-pyrrolo[3,2-d]pyrimidine (2.30 g, 15 mmol), 2-iodopropane (2.81 g, 16.5 mmol), cesium carbonate (9.77 g, 30 mmol) and N,N-dimethylformamide (15 mL) was stirred at room temperature for 3 days. The reaction mixture was concentrated under reduced pressure, and the residue was diluted with water and extracted with ethyl acetate. The organic layer was concentrated under reduced pressure, and the residue was purified by column chromatography (silica gel, hexane/ethyl acetate=90/10→0/100) to give the title compound (2.20 g, 75%) as a white solid.

¹H-NMR (DMSO-d₆, 300 MHz) δ 1.53 (6H, d, J=6.7 Hz), 5.41 (1H, sept, J=6.7 Hz), 6.80 (1H, d, J=3.2 Hz), 8.24 (1H, d, J=3.2 Hz), 8.62 (1H, s).

### Reference Example 26

### 2-chloro-4-[(5-isopropyl-5H-pyrrolo[3,2-d]pyrimidin-4-yl)oxy]aniline

Using 4-chloro-5-isopropyl-5H-pyrrolo[3,2-d]pyrimidine (978 mg, 5.0 mmol), 4-amino-3-chlorophenol (861 mg, 6.0 mmol), potassium carbonate (2073 mg, 15 mmol) and N-methylpyrrolidone (5 mL), and in the same manner as in Reference Example 3, the title compound (1006 mg, 66%) was obtained.

¹H-NMR (DMSO-d₆, 300 MHz) δ 1.53 (6H, d, J=6.8 Hz), 5.17 (1H, sept, J=6.8 Hz), 5.34 (2H, s), 6.63 (1H, d, J=3.2 Hz), 6.85 (1H, d, J=8.7 Hz), 7.00 (1H, dd, J=8.7, 2.4 Hz), 7.22 (1H, d, J=2.4 Hz), 7.98 (1H, d, J=3.2 Hz), 8.27 (1H, s).

### Reference Example 27

### 1-[4-nitro-2-(trifluoromethyl)phenyl]-1H-imidazole

To a solution of sodium hydride (1.42 g, 35.3 mmol) in N,N-dimethylformamide (10 mL) was added dropwise a solution of imidazole (2.00 g, 29.4 mmol) in N,N-dimethylformamide (10 mL) at 0° C., and the mixture was stirred at room temperature for 30 min. 1-Fluoro-4-nitro-2-(trifluoromethyl)benzene (2.73 mL, 29.4 mmol) was added to the reaction mixture and the mixture was stirred at 100° C. for 7 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was filtrated and washed with ethyl acetate-hexane to give the title compound (1.25 g, 17%) as a yellow solid.

¹H-NMR (DMSO-d₆, 300 MHz) δ 7.15 (1H, s), 7.50 (1H, s), 7.90-7.93 (2H, m), 8.63-8.66 (2H, m).

### Reference Example 28

### Phenyl{2-chloro-4-[(5-methyl-5H-pyrrolo[3,2-d]pyrimidin-4-yl)oxy]phenyl}carbamate

To a solution of 2-chloro-4-[(5-methyl-5H-pyrrolo[3,2-d]pyrimidin-4-yl)oxy]aniline (522 mg, 1.90 mmol) and pyridine (460 μL, 5.70 mmol) in N,N-dimethylacetamide (5 mL) was added phenyl chloroformate (252 μL, 2.00 mmol) with stirring under ice-cooling, and the mixture was stirred at room temperature for 1 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was filtrated and washed with ethyl acetate-hexane to give the title compound (536 mg, 71%) as a brown solid.

¹H-NMR (DMSO-d₆, 300 MHz) δ 4.11 (3H, s), 6.62 (1H, d, J=3.0 Hz), 6.73-6.76 (1H, m), 7.13-7.18 (1H, m), 7.23-7.29 (2H, m), 7.35-7.47 (2H, m), 7.63 (1H, d, J=2.4 Hz), 7.69 (1H, d, J=8.7 Hz), 7.81 (1H, d, J=3.0 Hz), 8.31 (1H, s), is 9.83 (1H, s).

### Reference Example 29-1

### N-methyl-3-nitro-5-(trifluoromethyl)benzamide

A mixture of 3-nitro-5-(trifluoromethyl)benzoic acid (1.00 g, 4.25 mmol), 1-ethyl-3-(3-dimethylaminopropyl)carbodiimide hydrochloride (978 mg, 5.10 mmol), 1-hydroxy-1H-benzotriazole (781 mg, 5.10 mmol), triethylamine (1.77 mL, 12.8 mmol), 2M dimethylamine tetrahydrofuran solution (2.55 mL, 5.10 mmol) and N,N-dimethylformamide (5 mL) was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was collected by filtration and washed with ethyl acetate-hexane to give the title compound (760 mg, 72%) as a yellow solid.

¹H-NMR (DMSO-d₆, 300 MHz) δ 2.85 (3H, d, J=4.4 Hz), 8.60 (1H, s), 8.63 (1H, s), 8.90 (1H, s), 9.05 (1H, d, J=4.4 Hz).

### Reference Example 29-2

### 3-amino-N-methyl-5-(trifluoromethyl)benzamide

To a solution of N-methyl-3-nitro-5-(trifluoromethyl)benzamide (750 mg, 3.02 mmol) in methanol (20 mL) was added palladium carbon (50% water-containing product, 75 mg), and the mixture was stirred under a hydrogen atmosphere at room temperature for 3 hr. After celite filtration of the reaction mixture, the filtrate was concentrated under reduced pressure, and the residue was collected by filtration and washed with ethyl acetate-hexane to give the title compound (180 mg, 28%) as a yellow solid.

¹H-NMR (DMSO-d₆, 300 MHz) δ 2.75 (3H, d, J=4.5 Hz), 5.76 (2H, s), 6.95 (1H, s), 7.21 (1H, s), 7.26 (1H, s), 8.44 (1H, d, J=4.5 Hz).

### Reference Example 30-1

### 3-morpholin-4-yl-5-(trifluoromethyl)benzonitrile

To a solution of 3-fluoro-5-(trifluoromethyl)benzonitrile (1.29 g, 6.82 mmol) in dimethylsulfoxide (20 mL) was added morpholine (5.94 g, 68.2 mmol), and the mixture was stirred at 100° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure and dried to give the title compound (1.23 g, 70%) as an oil.

¹H-NMR (DMSO-d₆, 300 MHz) δ 3.28-3.31 (4H, m), 3.71-3.74 (4H, m), 7.48 (1H, s), 7.56 (1H, s), 7.66 (1H, s).

### Reference Example 30-2

### 3-morpholin-4-yl-5-(trifluoromethyl)benzoic Acid

To a solution of 3-morpholin-4-yl-5-(trifluoromethyl)benzonitrile (1.00 g, 3.90 mmol) in ethanol (5 mL) was added 8N aqueous sodium hydroxide solution (10 mL), and the mixture was stirred under reflux for 5 hr. After cooling the reaction solution to 0° C., 6N hydrochloric acid was added to adjust the reaction solution to pH 3. The precipitated solid was collected by filtration and washed with water to give the title compound (1.10 g, 99%) as a white solid.

¹H-NMR (DMSO-d₆, 300 MHz) δ 3.24-3.27 (4H, m), 3.73-3.76 (4H, m), 7.44 (1H, s), 7.55 (1H, s), 7.67 (1H, s), 13.40 (1H, br s).

### Reference Example 31-1

### Methyl 3-nitro-5-(trifluoromethyl)benzoate

To a solution of 3-nitro-5-(trifluoromethyl)benzoic acid (3.00 g, 12.8 mmol) in N,N-dimethylformamide (100 mL) were added potassium carbonate (5.29 g, 38.3 mmol) and iodomethane (1.19 mL, 19.1 mmol), and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was collected by filtration and washed with ethyl acetate-hexane to give the title compound (2.90 g, 92%) as a yellow solid.

¹H-NMR (DMSO-d₆, 300 MHz) δ 3.95 (3H, s), 8.55 (1H, s), 8.75 (1H, s), 8.81 (1H, s).

### Reference Example 31-2

### Methyl 3-amino-5-(trifluoromethyl)benzoate

To a solution of methyl 3-nitro-5-(trifluoromethyl)benzoate (1.50 g, 6.02 mmol) in methanol (20 mL) was added palladium carbon (50% water-containing product, 15 mg), and the mixture was stirred under a hydrogen atmosphere at room temperature for 3 hr. The reaction mixture was filtered through celite. The filtrate was concentrated under reduced pressure and dried to give the title compound (1.27 g, 96%) as a white solid.

¹H-NMR (DMSO-d₆, 300 MHz) δ 3.85 (3H, s), 5.94 (2H, s), 7.08 (1H, s), 7.26 (1H, s), 7.42 (1H, s).

### Reference Example 32

### 4-morpholin-4-yl-3-(trifluoromethyl)aniline

To a solution of 1-fluoro-4-nitro-2-(trifluoromethyl)benzene (564 mg, 2.70 mmol) in dimethylsulfoxide (20 mL) was added morpholine (2.35 g, 27.0 mmol), and the mixture was stirred at 100° C. for 7 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was dissolved in methanol (15 mL). Palladium carbon (50% water-containing product, 15 mg) was added, and the mixture was stirred under a hydrogen atmosphere at room temperature for 3 hr. The reaction mixture was filtered through celite. The filtrate was concentrated under reduced pressure and dried to give the title compound (668 mg, 99%) as a yellow solid.

¹H-NMR (DMSO-d₆, 300 MHz) δ 2.69-2.72 (4H, m), 3.62-3.65 (4H, m), 5.37 (2H, br s), 6.75-6.81 (2H, m), 7.24 (1H, d, J=8.7 Hz).

### Reference Example 33

### 3-(morpholin-4-ylcarbonyl)-5-(trifluoromethyl)aniline

To a solution of 3-nitro-5-(trifluoromethyl)benzoic acid (2.35 g, 10.0 mmol) in dichloromethane (35 mL)/N,N-dimethylformamide (100 μL) was added dropwise oxalyl chloride (4.30 mL, 50.0 mmol) at 0° C., and the mixture was stirred for 1 hr. The reaction solvent and oxalyl chloride were evaporated under reduced pressure, and the residue was dissolved in dichloromethane (20 mL). Morpholine (2.60 mL, 30.0 mmol) was added at 0° C. and the mixture was stirred for 5 hr. The reaction mixture was diluted with ethyl acetate, and the organic layer was washed with water and saturated brine, dried over anhydrous magnesium sulfate, and filtrated. The filtrate was concentrated under reduced pressure, and the residue was dissolved in methanol (15 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure and dried to give the title compound (1.68 g, 61%) as a yellow solid. ¹H-NMR (DMSO-d₆, 300 MHz) δ 3.30-3.70 (8H, m), 5.80 (2H, s), 6.74 (1H, s), 6.78 (1H, s), 6.80 (1H, s).

### Reference Example 34

### 3-(morpholin-4-ylmethyl)-5-(trifluoromethyl)aniline

To a solution (25 mL) of 3-(morpholin-4-ylcarbonyl)-5-(trifluoromethyl)aniline (1.00 g, 3.65 mmol) in tetrahydrofuran was added dropwise 1.9 mol/l dimethylsulfide-borane-tetrahydrofuran solution (5.76 mL, 10.9 mmol) at 0° C., and the mixture was stirred at room temperature for 1 hr and then under reflux for 3 hr. After cooling the reaction solution to room temperature, 6N hydrochloric acid (10 mL) was added. After stirring for 30 min, the mixture was stirred under reflux for 2 hr. After cooling the reaction solution to 0° C., 8N aqueous sodium hydroxide solution (10 mL) was added. The mixture was diluted with water, and extracted with ethyl acetate (×3). The organic layer was washed with saturated brine, dried over anhydrous magnesium sulfate, and filtrated. The filtrate was concentrated under reduced pressure and dried to give the title compound (685 mg, 72%) as a yellow oil.

¹H-NMR (DMSO-d₆, 300 MHz) δ 2.30-2.40 (4H, m), 3.36 (2H, s), 3.50-3.60 (4H, m), 5.54 (2H, s), 6.69 (1H, s), 6.72 (1H, s), 6.77 (1H, s).

### Reference Example 35

### 3-[(4-methylpiperazin-1-yl)carbonyl]-5-(trifluoromethyl)aniline

Using 3-nitro-5-(trifluoromethyl)benzoic acid (2.35 g, 10.0 mmol), dichloromethane (55 mL), N,N-dimethylformamide (100 μL), oxalyl chloride (4.30 mL, 50.0 mmol), 1-methylpiperazine (3.30 mL, 30.0 mmol), methanol (15 mL) and palladium carbon (50% water-containing product, 100 mg) as starting materials, and in the same manner as in Reference Example 33, the title compound (2.15 g, 75%) was obtained as a white solid.

¹H-NMR (DMSO-d₆, 300 MHz) δ 2.21 (3H, s), 2.25-2.40 (4H, m), 3.25-3.40 (4H, m), 5.80 (2H, s), 6.69 (1H, s), 6.76 (1H, s), 6.89 (1H, s).

### Reference Example 36

### 4-[(1-methylpiperidin-4-yl)oxy]-3-(trifluoromethyl)aniline

To a solution of sodium hydride (398 mg, 9.94 mmol) in N,N-dimethylformamide (5 mL) was added dropwise a solution of 1-methylpiperidin-4-ol (1.26 g, 6.03 mmol) in N,N-dimethylformamide (5 mL) at 0° C., and the mixture was stirred at room temperature for 30 min. 1-Fluoro-4-nitro-2-(trifluoromethyl)benzene (1.91 mL, 6.03 mmol) was added to the reaction mixture, and the mixture was stirred at 100° C. for 7 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was dissolved in methanol (15 mL). Palladium carbon (50% water-containing product, 10 mg) was added, and the mixture was stirred under a hydrogen atmosphere at room temperature for 3 hr. The reaction mixture was filtered through celite. The filtrate was concentrated under reduced pressure and dried to give the title compound (998 mg, 60%) as a black solid.

¹H-NMR (DMSO-d₆, 300 MHz) δ 1.56-1.67 (2H, m), 1.81-1.92 (2H, m), 1.99-2.20 (5H, m), 2.50-2.60 (2H, m), 4.24-4.30 (1H, m), 5.02 (2H, s), 6.75 (1H, dd, J=8.5, 2.8 Hz), 6.81 (1H, d, J=2.8 Hz), 6.97 (1H, d, J=8.5 Hz).

### Reference Example 37-1

### 4-[(4-methylpiperazin-1-yl)carbonyl]-3-(trifluoromethyl)aniline

Using 4-nitro-2-(trifluoromethyl)benzoic acid (2.00 g, 8.51 mmol), dichloromethane (55 mL), N,N-dimethylformamide (100 μL), oxalyl chloride (3.65 mL, 42.4 mmol), 1-methylpiperazine (3.20 mL, 22.7 mmol), methanol (15 mL) and palladium carbon (50% water-containing product, 100 mg) as starting materials, and in the same manner as in Reference Example 33, the title compound (2.05 g, 84%) was obtained as a yellow solid.

¹H-NMR (DMSO-d₆, 300 MHz) δ 2.10-2.40 (7H, m), 3.05-3.20 (2H, m), 3.50-3.65 (2H, m), 5.78 (2H, s), 6.75-6.79 (1H, m), 6.88 (1H, d, J=1.8 Hz), 7.01 (1H, d, J=7.8 Hz).

### Reference Example 37-2

### 4-[(4-methylpiperazin-1-yl)methyl]-3-(trifluoromethyl)aniline

Using 4-[(4-methylpiperazin-1-yl)carbonyl]-3-(trifluoromethyl)aniline (1.00 g, 3.48 mmol), tetrahydrofuran (25 mL), 1.9 mol/l dimethylsulfide-borane-tetrahydrofuran solution (9.16 mL, 17.4 mmol), 6N hydrochloric acid (10 mL) and 8N aqueous sodium hydroxide solution (10 mL) as starting materials, and in the same manner as in Reference Example 34, the title compound (1.26 g, 99%) was obtained as a yellow oil.

¹H-NMR (DMSO-d₆, 300 MHz) δ 2.25 (3H, s), 2.50-2.65 (4H, m), 2.90-3.05 (4H, m), 3.45 (2H, s), 5.48 (2H, s), 6.58 (1H, s), 6.72 (1H, d, J=9.0 Hz), 6.85 (1H, d, J=9.0 Hz).

### Reference Example 38-1

### 4-(morpholin-4-ylcarbonyl)-3-(trifluoromethyl)aniline

Using 4-nitro-2-(trifluoromethyl)benzoic acid (2.00 g, 8.51 mmol), dichloromethane (55 mL), N,N-dimethylformamide (100 μL), oxalyl chloride (3.65 mL, 42.4 mmol), morpholine (2.50 mL, 22.7 mmol), methanol (15 mL) and palladium carbon (50% water-containing product, 100 mg) as starting materials, and in the same manner as in Reference Example 33, the title compound (1.68 g, 72%) was obtained as a yellow solid.

¹H-NMR (DMSO-d₆, 300 MHz) δ 3.00-3.20 (2H, m), 3.40-3.70 (6H, m), 5.80 (2H, s), 6.76-6.80 (1H, m), 6.88 (1H, d, J=2.1 Hz), 7.04 (1H, d, J=7.8 Hz).

### Reference Example 38-2

### 4-(morpholin-4-ylmethyl)-3-(trifluoromethyl)aniline

Using 4-(morpholin-4-ylcarbonyl)-3-(trifluoromethyl)aniline (1.00 g, 3.65 mmol), tetrahydrofuran (25 mL), 1.9 mol/l dimethylsulfide-borane-tetrahydrofuran solution (9.60 mL, 18.2 mmol), 6N hydrochloric acid (10 mL) and 8N aqueous sodium hydroxide solution (10 mL) as starting materials, and in the same manner as in Reference Example 34, the title compound (1.88 g, 99%) was obtained as a yellow oil.

¹H-NMR (DMSO-d₆, 300 MHz) δ 2.30-2.40 (4H, m), 3.39 (2H, s), 3.50-3.60 (4H, m), 5.45 (2H, s), 6.72-6.75 (1H, m), 6.85 (1H, d, J=2.1 Hz), 7.30 (1H, d, J=8.4 Hz).

### Reference Example 39

### 3-[(4-methylpiperazin-1-yl)methyl]-5-(trifluoromethyl)aniline

Using 3-[(4-methylpiperazin-1-yl)carbonyl]-5-(trifluoromethyl)aniline (762 mg, 2.79 mmol), tetrahydrofuran (25 mL), 1.9 mol/l dimethylsulfide-borane-tetrahydrofuran solution (4.40 mL, 8.36 mmol), 6N hydrochloric acid (10 mL) and 8N aqueous sodium hydroxide solution (10 mL) as starting materials, and in the same manner as in Reference Example 34, the title compound (380 mg, 50%) was obtained as a yellow oil.

¹H-NMR (DMSO-d₆, 300 MHz) δ 2.28 (3H, s), 2.60-2.70 (4H, m), 2.95-3.05 (4H, m), 3.43 (2H, s), 5.56 (2H, s), 6.66-6.76 (3H, m).

### Reference Example 40-1

### 3-methoxy-4-nitrophenol

To a solution of 5-fluoro-2-nitroanisole (11.0 g, 64.3 mmol) in dimethylsulfoxide (30 mL)/water (5 mL) was added sodium hydroxide (5.36 g, 129 mmol), and the mixture was stirred at 90° C. for 15 hr. The reaction solution was cooled to 0° C., 6N hydrochloric acid was added to adjust to pH 7. The precipitated solid was filtered off. The filtrate was concentrated under reduced pressure, and the residue was purified by silica gel column chromatography (ethyl acetate/hexane=10/90→50/50) and recrystallized from ethyl acetate-hexane to give the title compound (3.05 g, 28%) as a white solid.

¹H-NMR (DMSO-d₆, 300 MHz) δ 3.88 (3H, s), 6.47 (1H, dd, J=9.1, 2.7 Hz), 6.61 (1H, d, J=2.7 Hz), 7.89 (1H, d, J=9.1 Hz), 10.90 (1H, s).

### Reference Example 40-2

### 2-methoxy-4-[(5-methyl-5H-pyrrolo[3,2-d]pyrimidin-4-yl)oxy]aniline

To a solution of 3-methoxy-4-nitrophenol (1.50 g, 8.86 mmol) in methanol (20 mL) was added palladium carbon (50% water-containing product, 150 mg), and the mixture was stirred under a hydrogen atmosphere at room temperature for 3 hr. The reaction mixture was filtered through celite. The filtrate was concentrated under reduced pressure and dried, and the residue was dissolved in N-methylpyrrolidone (3 mL). Potassium carbonate (2.50 g, 17.7 mmol) and 4-chloro-5-methyl-5H-pyrrolo[3,2-d]pyrimidine (990 mg, 5.91 mmol) were added, and the mixture was stirred at 110° C. for 2 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was collected by filtration and washed with ethyl acetate-hexane to give the title compound (970 mg, 6.1%) as a purple solid.

¹H-NMR (DMSO-d₆, 300 MHz) δ 3.74 (3H, s), 4.08 (3H, s), 4.67 (2H, s), 6.55-6.67 (3H, m), 6.78 (1H, d, J=2.1 Hz), 7.73 (1H, d, J=3.0 Hz), 8.29 (1H, s).

### Reference Example 41

### 1-(3-aminophenyl)-2,2,2-trifluoroethanol

To a solution of methyl 3-nitrobenzoate (5.00 g, 27.6 mmol) in toluene (30 mL) were added trifluoromethyltrimethylsilane (5.10 mL, 34.5 mmol) and tetrabutylammoniumfluoride (180 mg, 0.690 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in methanol (15 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure, and the residue was collected by filtration and washed with ethyl acetate-hexane to give the title compound (3.02 g, 39%) as a white solid.

¹H-NMR (DMSO-d₆, 300 MHz) δ 4.80-4.95 (1H, m), 5.12 (2H, s), 6.50-6.59 (3H, m), 6.68 (1H, s), 6.99 (1H, t, J=8.0 Hz).

### Reference Example 42-1

### 1-[3-amino-5-(trifluoromethyl)benzoyl]piperidin-4-ol

To a solution of 3-nitro-5-(trifluoromethyl)benzoic acid (2.57 g, 10.9 mmol) in dichloromethane (35 mL)/N,N-dimethylformamide (100 μL) was added dropwise oxalyl chloride (4.69 mL, 54.6 mmol) at 0° C., and the mixture was stirred for L hr. The reaction solvent and oxalyl chloride were evaporated under reduced pressure, and the residue was dissolved in dichloromethane (20 mL). 4-aminocyclohexanol (5.53 g, 54.7 mmol) was added at 0° C. and the mixture was stirred for 5 hr. The reaction mixture was diluted with ethyl acetate, and the organic layer was washed with water and saturated brine, dried over anhydrous magnesium sulfate, and filtrated. The filtrate was concentrated under reduced pressure, and the residue was dissolved in methanol (15 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure, and the residue was collected by filtration and washed with ethyl acetate-hexane to give the title compound (1.65 g, 52%) as a white solid.

¹H-NMR (DMSO-d₆, 300 MHz) δ 1.20-1.40 (2H, m), 1.60-1.80 (2H, m), 3.05-3.25 (2H, m), 3.40-4.10 (3H, m), 4.80 (1H, d, J=4.2 Hz), 5.79 (2H, s), 6.68 (1H, s), 6.75 (1H, s), 6.87 (1H, s).

### Reference Example 42-2

### 1-[3-amino-5-(trifluoromethyl)benzyl]piperidin-4-ol

Using 1-[3-amino-5-(trifluoromethyl)benzoyl]piperidin-4-ol (1.50 g, 5.20 mmol), tetrahydrofuran (10 mL), 1.9 mol/l dimethylsulfide-borane-tetrahydrofuran solution (8.25 mL, 15.6 mmol), 6N hydrochloric acid (15 mL) and 8N aqueous sodium hydroxide solution (15 mL) as starting materials, and in the same manner as in Reference Example 34, the title compound (1.20 g, 84%) was obtained as a white solid.

¹H-NMR (DMSO-d₆, 300 MHz) δ 1.33-1.43 (2H, m), 1.68-1.71 (2H, m), 1.97-2.04 (2H, m), 2.51-2.65 (2H, m), 3.33-3.45 (3H, m), 4.55 (1H, d, J=3.9 Hz), 5.53 (2H, s), 6.66 (1H, s), 6.70 (1H, s), 6.75 (1H, s).

### Reference Example 43

### N-(trans-4-hydroxycyclohexyl)-N′-[3-(trifluoromethyl)phenyl]urea

To a solution of trans-4-aminocyclohexanol (2.00 g, 17.4 mmol) and triethylamine (7.23 mL, 52.2 mmol) in tetrahydrofuran (20 mL) was added 3-(trifluoromethyl)phenylisocyanate (2.91 mL, 20.8 mmol), and the mixture was stirred at room temperature for 5 hr. The precipitated solid was collected by filtration and washed with ethyl acetate-hexane to give the title compound (3.80 g, 72%) as a white solid.

¹H-NMR (DMSO-d₆, 300 MHz) δ 1.12-1.29 (4H, m), 1.76-1.93 (4H, m), 3.35-3.41 (2H, m), 4.54-4.56 (1H, m), 6.14 (1H, d, J=7.8 Hz), 7.18-7.21 (1H, m), 7.39-7.44 (2H, m), 7.97 (1H, s), 8.67 (1H, s).

### Reference Example 44

### 2-(3-aminophenyl)-1,1,1-trifluoropropan-2-ol

To a solution of 3-nitroacetophenone (5.25 g, 31.8 mmol) in toluene (100 mL) were added trifluoromethyltrimethylsilane (7.05 mL, 47.7 mmol) and tetrabutylammoniumfluoride (83.0 mg, 0.318 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in methanol (20 mL). Palladium carbon (50% water-containing product, 100 mg) was added, and the mixture was stirred under a hydrogen atmosphere at room temperature for 3 hr. After celite filtration of the reaction mixture, 6N hydrochloric acid (10 mL) was added to the filtrate, and the mixture was stirred at room temperature for 3 hr. After neutralization with 8N aqueous sodium hydroxide solution, the mixture was extracted with ethyl acetate (×3). The organic layer was washed with saturated brine, dried over anhydrous magnesium sulfate, and filtrated. The filtrate was concentrated under reduced pressure, and the residue was collected by filtration and washed with ethyl acetate-hexane to give the title compound (5.38 g, 82%) as a yellow solid.

¹H-NMR (DMSO-d₆, 300 MHz) δ 1.60 (3H, s), 5.08 (2H, s), 6.32 (1H, s), 6.50-6.53 (1H, m), 6.67-6.69 (1H, m), 6.81 (1H, s), 7.00 (1H, t, J=7.8 Hz).

### Reference Example 45

### 7-amino-1-(trifluoromethyl)-1,2,3,4-tetrahydronaphthalen-1-ol

To a solution of 7-nitro-3,4-dihydronaphthalen-1(2H)-one (2.14 g, 11.2 mmol) in tetrahydrofuran (20 mL) were added trifluoromethyltrimethylsilane (1.99 mL, 13.4 mmol) and cesium fluoride (182 mg, 1.20 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in methanol (20 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure, and the residue was collected by filtration and washed with ethyl acetate-hexane to give the title compound (1.15 g, 45%) as a white solid.

¹H-NMR (DMSO-d₆, 300 MHz) δ 1.68-2.10 (4H, m), 2.49-2.64 (2H, m), 4.93 (2H, s), 6.19 (1H, d, J=1.9 Hz), 6.49 (1H, dd, J=8.2, 1.9 Hz), 6.78 (1H, d, J=8.2 Hz), 6.87 (1H, s).

### Reference Example 46

### 1-(5-amino-1-methyl-1H-pyrazol-3-yl)-2,2,2-trifluoroethanol

To a solution of methyl 1-methyl-5-nitro-1H-pyrazole-3-carboxylate (1.00 g, 5.40 mmol) in tetrahydrofuran (7 mL) were added trifluoromethyltrimethylsilane (958 μL, 6.48 mmol) and cesium fluoride (42.0 mg, 0.275 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in methanol (20 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure, and the residue was collected by filtration and washed with ethyl acetate-hexane to give the title compound (388 mg, 38%) as a white solid.

¹H-NMR (DMSO-d₆, 300 MHz) δ 3.48 (3H, s), 4.66-4.78 (1H, m), 5.21 (2H, s), 5.35 (1H, s), 6.41 (1H, br s).

### Reference Example 47-1

### Phenyl[4-(trifluoromethyl)pyridin-2-yl]carbamate

To a solution of 2-amino-4-(trifluoromethyl)pyridine (600 mg, 3.7 mmol) and pyridine (1197 μL, 14.8 mmol) in tetrahydrofuran (10 mL) was added phenyl chloroformate (464 μL, 3.7 mmol) under ice-cooling, and the mixture was stirred at room temperature for 2 hr. The reaction mixture was diluted with water and extracted with ethyl acetate. The organic layer was concentrated under reduced pressure, and the residue was purified by column chromatography (silica gel, hexane/ethyl acetate=100/0→60/40) to give the title compound (842 mg, 81%) as a white solid.

¹H-NMR (CDCl₃, 300 MHz) δ 7.19-7.33 (4H, m), 7.40-7.48 (2H, m), 8.34 (1H, s), 8.52 (1H, d, J=5.1 Hz), 8.83 (1H, br s).

### Reference Example 47-2

### Phenyl[1-oxide-4-(trifluoromethyl)pyridin-2-yl]carbamate

To a solution of phenyl[4-(trifluoromethyl)pyridin-2-yl]carbamate (815 mg, 2.9 mmol) in dichloromethane (20 mL) was added m-chloroperbenzoic acid (70%, 783 mg, 3.2 mmol) and the mixture was stirred at room temperature for 18 hr. The reaction mixture was washed with water, and the organic layer was concentrated under reduced pressure. The residue was purified by column chromatography (silica gel, hexane/ethyl acetate=80/20→0/100) to give the title compound (830 mg, 96%) as a pale-yellow oil.

¹H-NMR (CDCl₃, 300 MHz) δ 7.19-7.34 (4H, m), 7.40-7.48 (2H, m), 8.37 (1H, d, J=6.9 Hz), 8.51 (1H, d, J=2.4 Hz), 9.77 (1H, br s).

### Reference Example 48

### Phenyl[5-(trifluoromethyl)pyridin-3-yl]carbamate

Using 3-amino-5-(trifluoromethyl)pyridine (780 mg, 4.8 mmol), pyridine (1557 μL, 19.3 mmol), tetrahydrofuran (10 mL) and phenyl chloroformate (664 μL, 5.3 mmol), and in the same manner as in Reference Example 47-1, the title compound (712 mg, 52%) was obtained as a white solid.

¹H-NMR (CDCl₃, 300 MHz) δ 7.17-7.23 (3H, m), 7.28-7.32 (1H, m), 7.39-7.47 (2H, m), 8.39 (1H, br s), 8.63 (1H, s), 8.72 (1H, d, J=2.4 Hz).

### Reference Example 49

### Phenyl[2-(trifluoromethyl)pyridin-4-yl]carbamate

Using 4-amino-2-(trifluoromethyl)pyridine (1630 mg, 10 mmol), pyridine (3.25 mL, 40 mmol), tetrahydrofuran (20 mL) and phenyl chloroformate (1.39 mL, 11 mmol), and in the same manner as in Reference Example 47-1, the title compound (1303 mg, 46%) was obtained as a white solid.

¹H-NMR (CDCl₃, 300 MHz) δ 7.16-7.22 (2H, m), 7.26-7.33 (1H, m), 7.33 (1H, br s), 7.39-7.47 (2H, m), 7.58 (1H, dd, J=5.7, 2.1 Hz), 7.85 (1H, d, J=2.1 Hz), 8.62 (1H, d, J=5.7 Hz).

### Reference Example 50

### Phenyl[1-oxide-5-(trifluoromethyl)pyridin-3-yl]carbamate

Using phenyl[5-(trifluoromethyl)pyridin-3-yl]carbamate (427 mg, 1.5 mmol), dichloromethane (20 mL) and m-chloroperbenzoic acid (70%, 410 mg, 1.7 mmol), and in the same manner as in Reference Example 47-2, the title compound (340 mg, 75%) was obtained as a white solid.

¹H-NMR (CDCl₃, 300 MHz) δ 7.14-7.19 (2H, m), 7.26-7.33 (1H, m), 7.39-7.46 (2H, m), 8.05 (1H, br s), 8.27 (1H, s), 8.76 (1H, t, J=1.5 Hz), 8.99 (1H, br s).

### Reference Example 51

### Phenyl[6-(trifluoromethyl)pyridin-3-yl]carbamate

Using 3-amino-6-(trifluoromethyl)pyridine (3.24 g, 20 mmol), pyridine (6.47 mL, 80 mmol), tetrahydrofuran (20 mL) and phenyl chloroformate (2.76 mL, 22 mmol), and in the same manner as in Reference Example 47-1, the title compound (3.11 g, 55%) was obtained as a white solid.

¹H-NMR (CDCl₃, 300 MHz) δ 7.17-7.32 (4H, m), 7.39-7.47 (2H, m), 7.69 (1H, d, J=8.6 Hz), 8.25 (1H, dd, J=8.6, 2.2 Hz), 8.64 (1H, d, J=2.2 Hz).

### Reference Example 52-1

### 4-nitro-1-(2,2,2-trifluoroethyl)pyrazole

To a solution of 4-nitropyrazole (1.13 g, 10 mmol) and 2,2,2-trifluoroethyltriflate (3.48 g, 15 mmol) in N,N-dimethylformamide (10 mL) was added potassium carbonate (2.76 g, 20 mmol), and the mixture was stirred at room temperature for 3 hr. The reaction mixture was concentrated under reduced pressure, diluted with water and extracted with ethyl acetate. The organic layer was concentrated under reduced pressure, and the residue was purified by column chromatography (silica gel, hexane/ethyl acetate=100/0→60/40) to give the title compound (1.87 g, 96%) as a white solid.

¹H-NMR (CDCl₃, 300 MHz) δ 4.77 (2H, q, J=8.1 Hz), 8.16 (1H, s), 8.29 (1H, s).

### Reference Example 52-2

### 4-amino-1-(2,2,2-trifluoroethyl)pyrazole

To a solution of 4-nitro-1-(2,2,2-trifluoroethyl)pyrazole

(1.8-5 g, 9.5 mmol) in methanol (20 mL) was added 10% palladium carbon (containing 50% water, 742 mg), and the mixture was stirred under a hydrogen atmosphere at room temperature for 3 hr. The catalyst was filtered off and the filtrate was concentrated under reduced pressure. The residue was purified by column chromatography (silica gel, hexane/ethyl acetate=70/30→0/100) to give the title compound (1.40 g, 89%) as a pale-brown oil.

¹H-NMR (CDCl₃, 300 MHz) δ 2.98 (2H, br s), 4.57 (2H, q, J=8.4 Hz), 7.09 (1H, s), 7.25 (1H, s).

### Reference Example 53-1

### 2-cyano-1-(2-furyl)vinyl 4-methylbenzenesulfonate

To a mixture of 3-(2-furyl)-3-oxopropanenitrile (5.29 g, 39.2 mmol), p-toluenesulfonyl chloride (9.00 g, 47.2 mmol) and dichloromethane (60 mL) was added dropwise triethylamine (5.99 g, 59.2 mmol) under ice-cooling. After stirring under ice-cooling for 1.5 hr, the mixture was diluted with dichloromethane (100 mL). The mixture was washed with water (150 mL), dried over anhydrous magnesium sulfate, and filtrated. The filtrate was concentrated under reduced pressure and the residue was purified by column chromatography (silica gel, ethyl acetate/hexane=10/90→25/75) to give the title compound (10.48 g, 93%) as a mixture of (E)-form and (Z)-form (3:1).

¹H-NMR (CDCl₃, 300 MHz) δ 2.47 (3/4H, s), 2.49 (9/4H, s), 5.27 (1/4H, s), 5.63 (3/4H, s), 6.47 (1/4H, m), 6.53 (3/4H, m), 6.86 (1/4H, d, J=3.6 Hz), 6.95 (3/4H, d, J=3.6 Hz), 7.38 (1/2H, d, J=7.8 Hz), 7.42 (3/2H, d, J=7.8 Hz), 7.51 (3/4H, m), 7.55 (1/4H, m), 7.83 (1/2H, d, J=7.8 Hz), 7.97 (3/2H, d, J=7.8 Hz).

### Reference Example 53-2

### Ethyl 3-amino-5-(2-furyl)-1H-pyrrole-2-carboxylate

To a solution of 2-cyano-1-(2-furyl)vinyl 4-methylbenzenesulfonate (10.48 g, 36.2 mmol) and diethyl aminomalonate hydrochloride (7.67 g, 36.2 mmol) in a mixed solvent of ethanol (120 mL)-tetrahydrofuran (64 mL) was added dropwise 20% sodium ethoxide ethanol solution (36.9 mL) under ice-cooling. After stirring at room temperature for 12 hr, the reaction mixture was poured into ice water (350 mL), and adjusted to pH 7 with 1N hydrochloric acid. The organic solvent was evaporated under reduced pressure, and extracted with ethyl acetate (150 mL×3). The organic layers were combined, washed with saturated brine (100 mL), dried over anhydrous magnesium sulfate, and filtrated. The filtrate was concentrated under reduced pressure, and the residue was purified by column chromatography (silica gel, ethyl acetate/hexane=25/75→50/50), and the obtained solid was recrystallized from ethyl acetate-hexane to give the title compound (2.66 g, 33%).

¹H-NMR (CDCl₃, 300 MHz) δ 1.37 (3H, t, J=7.0 Hz), 4.34 (2H, q, J=7.0 Hz), 4.37 (2H, br s), 5.93 (1H, d, J=2.7 Hz), 6.45 (1H, dd, J=3.6, 1.8 Hz), 6.49 (1H, d, J=3.6 Hz), 7.41 (1H, d, J=1.8 Hz), 8.35 (1H, br s).

### Reference Example 53-3

### 6-(2-furyl)-4,5-dihydro-3H-pyrrolo[3,2-d]pyrimidin-4-one

To a solution of ethyl 3-amino-5-(2-furyl)-1H-pyrrole-2-carboxylate (2.58 g, 11.7 mmol) in ethanol (35 mL) was added formamidine acetate (1.83 g, 17.6 mmol), and the mixture was heated under reflux for 18 hr. After cooling to room temperature, the precipitated solid was collected by filtration, washed with ethanol, and dried under reduced pressure at 60° C. to give the title compound (2.26 g, 96%).

¹H-NMR (DMSO-d₆, 300 MHz) δ 6.58 (1H, d, J=2.1 Hz), 6.61 (1H, dd, J=3.5, 2.1 Hz), 7.08 (1H, m), 7.76 (1H, m), 7.80 (1H, d, J=3.5 Hz), 11.91 (1H, br s), 12.50 (1H, br s).

### Reference Example 53-4

### 4-chloro-6-(2-furyl)-5H-pyrrolo[3,2-d]pyrimidine

After stirring a mixture of 6-(2-furyl)-4,5-dihydro-3H′ pyrrolo[3,2-d]pyrimidin-4-one (2.20 g, 10.9 mmol) and phosphoryl chloride (10.7 g, 69.7 mmol) at 100° C. for 20 min, dioxane (30 mL) was added and the mixture was stirred at 100° C. for 3 hr. After concentration under reduced pressure, saturated aqueous sodium hydrogen carbonate was added to the residue, and the mixture was extracted with ethyl acetate-acetone (155 mL×4). The organic layers were combined, and the mixture was washed with saturated brine (100 mL), dried over anhydrous magnesium sulfate, and filtrated. The filtrate was concentrated under reduced pressure. The residue was washed with ethyl acetate-ethylether, and dried under reduced pressure at 60° C. to give the title compound (2.19 g, 91%).

¹H-NMR (DMSO-d₆, 300 MHz) δ 6.74 (1H, dd, J=3.6, 2.1 Hz), 6.95 (1H, d, J=1.8 Hz), 7.37 (1H, dd, J=3.6, 0.6 Hz), 7.95 (1H, dd, J=2.1, 0.6 Hz), 8.60 (1H, s), 12.71 (1H, br s).

### Reference Example 53-5

### 4-chloro-6-(2-furyl)-5-methyl-5H-pyrrolo[3,2-d]pyrimidine

To a solution of 4-chloro-6-(2-furyl)-5H-pyrrolo[3,2-d]pyrimidine (440 mg, 2.00 mmol) in N,N-dimethylformamide solution (5 mL) was added 60% sodium hydride (160 mg, 4.00 mmol) under ice-cooling, and the mixture was stirred for 10 min. Methyl iodide (570 mg, 4.02 mmol) was added, and the mixture was stirred under ice-cooling for 1 hr. The mixture was poured into aqueous ammonium chloride (30 mL), and the mixture was extracted with ethyl acetate (40 mL×2). The organic layers were combined, dried over anhydrous magnesium sulfate, and filtrated. The filtrate was concentrated under reduced pressure, and the residue was purified by column chromatography (silica gel, ethyl acetate/hexane=20/80→60/40) to give the title compound (318 mg, 68%).

¹H-NMR (CDCl₃, 300 MHz) δ 4.29 (3H, s), 6.62 (1H, m), 6.86 (1H, d, J=3.3 Hz), 6.94 (1H, s), 7.67 (1H, m), 8.68 (1H, s).

### Reference Example 53-6

### 2-chloro-4-{[6-(2-furyl)-5-methyl-5H-pyrrolo[3,2-d]pyrimidin-4-yl]oxy}aniline

A mixture of 4-chloro-6-(2-furyl)-5-methyl-5H-pyrrolo[3,2-d]pyrimidine (313 mg, 1.34 mmol), 4-amino-3-chlorophenol (250 mg, 1.74 mmol), potassium carbonate (370 mg, 2.68 mmol) and N-methylpyrrolidone (5.4 mL) was stirred at 110° C. for 2 hr. After cooling, the reaction mixture was diluted with ethyl acetate (120 mL), washed with water and saturated brine, dried over anhydrous magnesium sulfate and filtrated. The filtrate was concentrated under reduced pressure and the residue was purified by column chromatography (silica gel, ethyl acetate/hexane=10/90→100/0), and the obtained residue was washed with ethyl acetate-ether and dried to give the title compound (267 mg, 59%).

¹H-NMR (CDCl₃, 300 MHz) 8.4.05 (2H, br s), 4.28 (3H, s), 6.59 (1H, m), 6.80 (1H, m), 6.84 (1H, d, J=8.7 Hz), 6.90 (1H, s), 7.01 (1H, dd, J=8.7, 2.4 Hz), 7.21 (1H, d, J=2.4 Hz), 7.62 (1H, d, J=0.9 Hz), 8.44 (1H, s).

### Reference Example 54

### Tert-butyl {2-[4-(4-amino-3-chlorophenoxy)-5H-pyrrolo[3,2-d]pyrimidin-5-yl]ethyl}carbamate

To a solution of tert-butyl[2-(4-chloro-5H-pyrrolo[3,2-d]pyrimidin-5-yl)ethyl]carbamate (500 mg, 1.68 mmol) in N-methylpyrrolidone (5.0 mL) were added potassium carbonate (700 mg, 5.05 mmol) and 4-amino-3-chlorophenol (290 mg, 2.02 mmol), and the mixture was stirred at 110° C. for 2 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by silica gel column chromatography (ethyl acetate/hexane) to give the title compound (428 mg, 63%) as a white solid.

¹H-NMR (CDCl₃, 300 MHz) δ 1.37 (9H, s), 3.55-3.61 (2H, m), 4.07 (2H, br s), 4.48-4.57 (2H, m), 4.75 (1H, br s), 6.66 (1H, d, J=3.0 Hz), 6.83 (1H, d, J=8.7 Hz), 7.00 (1H, dd, J=8.7, 3.0 Hz), 7.20 (1H, d, J=3.0 Hz), 7.35 (1H, d, J=3.0 Hz), 8.45 (1H, s)

### Reference Example 55

### 4-(4-amino-3-chlorophenoxy)-6-iodopyrimidine-5-amine

To absolution of 4,6-diiodopyrimidine-5-amine (25 g, 72.1 mmol) in N-methylpyrrolidone-(200 mL) were added potassium carbonate (23.9 g, 173 mmol) and 4-amino-3-chlorophenol (11.4 g, 79.3 mmol), and the mixture was stirred at 110° C. for 1 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by silica gel column chromatography (ethyl acetate/hexane) to give the title compound (25.1 g, 96%) as a brown solid.

¹H-NMR (DMSO-d₆, 300 MHz) δ 5.32 (2H, s), 5.43 (2H, s), 6.82 (1H, d, J=8.6 Hz), 6.92 (1H, dd, J=8.6, 2.6 Hz), 7.13 (1H, d, J=2.6 Hz), 7.70 (1H, s).

### Reference Example 56

### Tert-butyl{3-[5-amino-6-(4-amino-3-chlorophenoxy)pyrimidin-4-yl]prop-2-yn-1-yl}carbamate

To a solution of 4-(4-amino-3-chlorophenoxy)-6-iodopyrimidine-5-amine (9.0 g, 24.8 mmol) in acetonitrile (360 mL)/triethylamine (270 mL) were added tert-butyl prop-2-yn-1-ylcarbamate (4.25 g, 27.3 mmol), bis(triphenylphosphine)palladium(II) dichloride (870 mg, 1.24 mmol) and copper iodide (285 mg, 1.49 mmol), and the mixture was stirred at 80° C. for 4 hr. The reaction mixture was concentrated under reduced pressure, and the residue was purified by silica gel column chromatography (ethyl acetate/hexane) to give the title compound (7.6 g, 79%) as a brown solid.

¹H-NMR (DMSO-d₆, 300 MHz) δ 1.42 (9H, s), 4.07 (2H, d, J=5.4 Hz), 5.31 (2H, s), 5.73 (2H, br s), 6.82 (1H, d, J=8.9 Hz), 6.91 (1H, dd, J=8.9, 2.6 Hz), 7.12 (1H, d, J=2.6 Hz), 7.47 (1H, br s), 7.84 (1H, s).

### Reference Example 57

### Tert-butyl {[4-(4-amino-3-chlorophenoxy)-5H-pyrrolo[3,2-d]pyrimidin-6-yl]methyl}carbamate

To a solution of tert-butyl {3-[5-amino-6-(4-amino-3-chlorophenoxy)pyrimidin-4-yl]prop-2-yn-1-yl}carbamate (7.6 g, 19.5 mmol) in N,N-dimethylformamide (152 mL) was added copper iodide (0.37 g, 1.95 mmol), and the mixture was stirred at 80° C. for 4 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by silica gel column chromatography (ethyl acetate/hexane) and recrystallized from ethyl acetate-hexane to give the title compound (2.97 g, 39%) as a brown solid.

¹H-NMR (DMSO-d₆, 300 MHz) δ 1.42 (9H, s), 4.34 (2H, d, J=6.0 Hz), 5.32 (2H, s), 6.38 (1H, s), 6.85 (1H, d, J=8.6 Hz), 6.96 (1H, dd, J=8.6, 2.6 Hz), 7.19 (1H, s), 7.43 (1H, br s), 8.24 (1H, s), 12.10 (1H, s).

### Reference Example 58-1

### 4-(4-amino-3-chlorophenoxy)-6-iodo-N-methylpyrimidine-5-amine

To a solution of 4,6-diiodo-N-methylpyrimidine-5-amine (9.6 g, 55.4 mmol) in N-methylpyrrolidone (200 mL) were added potassium carbonate (18.4 g, 133 mmol) and 4-amino-3-chlorophenol (8.8 g, 61 mmol), and the mixture was stirred at 110° C. for 1 hr. After cooling to room temperature, water was added to the reaction mixture, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (9.6 g, 46%) as a brown solid.

¹H-NMR (DMSO-d₆, 300 MHz) δ 3.02 (3H, d, J=5.3 Hz), 3.34 (3H, s), 4.80 (1H, q, J=5.3 Hz), 5.31 (2H, s), 6.82 (1H, d, J=8.7 Hz), 6.90 (1H, dd, J=8.7, 2.7 Hz), 7.13 (1H, d, J=2.7 Hz), 7.77 (1H, s).

### Reference Example 58-2

### 2-chloro-4-[(5,6-dimethyl-5H-pyrrolo[3,2-d]pyrimidin-4-yl)oxy]aniline

To a solution of 4-(4-amino-3-chlorophenoxy)-6-iodo-N-methylpyrimidine-5-amine (3.0 g, 7.97 mmol) in acetonitrile (120 mL)/triethylamine (60 mL) were added 1-(trimethylsilyl)-1-propyne (2.36 mL, 15.9 mmol), dichlorobis(triphenylphosphine)palladium (280 mg, 0.40 mmol), triphenylphosphine (209 mg, 0.80 mmol), copper iodide (152 mg, 0.80 mmol) and potassium fluoride (1.02 g, 17.5 mmol), and the mixture was stirred at 80° C. for 36 hr. The solvent was evaporated under reduced pressure, saturated aqueous sodium hydrogen carbonate was added to the residue, and the mixture was extracted with ethyl acetate/tetrahydrofuran. The mixture was washed with saturated brine, dried over anhydrous magnesium sulfate, and filtrated. The filtrate was concentrated under reduced pressure, and the residue was purified by column chromatography (NH silica gel, ethyl acetate/hexane) to give the title compound (1.21 g, 52%) as a brown solid.

¹H-NMR (DMSO-d₆, 300 MHz) p 2.47 (3H, s), 3.96 (3H, s), 5.32 (2H, s), 6.41 (1H, s), 6.85 (1H, d, J=8.7 Hz), 6.98 (1H, dd, J=8.7, 2.7 Hz), 7.20 (1H, d, J=2.7 Hz), 8.20 (1H, s).

### Reference Example 59

### 2-(3-amino-4-fluorophenyl)-1,1,1-trifluoropropan-2-ol

To a solution of 4-fluoro-3-nitroacetophenone (1.10 g, 6.01 mmol) in tetrahydrofuran (10 mL) were added trifluoromethyltrimethylsilane (976 μL, 6.61 mmol) and cesium fluoride (9.00 mg, 0.06 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in methanol (20 mL). Palladium carbon (50% water-containing product, 100 mg) was added, and the mixture was stirred under a hydrogen atmosphere at room temperature for 3 hr. The reaction mixture was filtered through celite. The filtrate was concentrated under reduced pressure, and the residue was collected by filtration and washed with ethyl acetate-hexane to give the title compound (1.28 g, 96%) as a yellow solid.

¹H-NMR (DMSO-d₆, 300 MHz) δ 1.61 (3H, s), 5.17 (2H, s), 6.44 (1H, s), 6.65-6.70 (1H, m), 6.92-7.05 (2H, m).

### Reference Example 60-1

### 1-nitro-3-(2,2,2-trifluoro-1-methoxyethyl)benzene

To a solution of 3-nitrobenzaldehyde (1.93 g, 12.8 mmol) in tetrahydrofuran (10 mL) were added trifluoromethyltrimethylsilane (2.07 mL, 14.0 mmol) and cesium fluoride (19.5 mg, 0.128 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was dissolved in tetrahydrofuran (5 mL). The solution was added dropwise to a solution of sodium hydride (508 mg, 12.8 mmol) in tetrahydrofuran (5 mL) at 0° C., and the mixture was stirred at room temperature for 1 hr. Iodomethane (902 μL, 14.5 mmol) was added to the reaction mixture, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure and dried to give the title compound (2.83 g, 95%) as a yellow solid.

¹H-NMR (DMSO-d₆, 300 MHz) δ 3.41 (3H, s), 5.37-5.43 (1H, m), 7.77-7.82 (1H, m), 7.93 (1H, d, J=7.8 Hz), 8.31-8.35 (2H, m).

### Reference Example 60-2

### 3-(2,2,2-trifluoro-1-methoxyethyl)aniline

To a solution of 1-nitro-3-(2,2,2-trifluoro-1-methoxyethyl)benzene (2.80 g, 11.9 mmol) in methanol (10 mL) was added palladium carbon (50% water-containing product, 100 mg), and the mixture was stirred under a hydrogen atmosphere at room temperature for 3 hr. The reaction mixture was filtered through celite. The filtrate was concentrated under reduced pressure and dried to give the title compound (1.23 g, 50%) as a white solid.

¹H-NMR (DMSO-d₆, 300 MHz) δ 3.29 (3H, s), 4.74-4.81 (1H, m), 5.23 (2H, s), 6.52-6.62 (3H, m), 7.04 (1H, t, J=7.7 Hz).

### Reference Example 61-1

### 4-chloro-5-methyl-5H-pyrrolo[3,2-d]pyrimidine-6-carbonitrile

To a solution of diisopropylamine (1518 mg, 15 mmol) in tetrahydrofuran (30 mL) was added n-butyllithium (1.6M hexane solution, 8.1 mL, 13 mmol) with stirring under ice-cooling. After stirring at 0° C. for 30 min, the mixture was cooled to −78° C. A solution of 4-chloro-5-methyl-5H-pyrrolo[3,2-d]pyrimidine (1676 mg, 10 mmol) in tetrahydrofuran (40 mL) was added dropwise to the reaction mixture. After stirring at −78° C. for 1 hr, p-toluenesulfonyl cyanide (3624 mg, 20 mmol) was added and the mixture was stirred for 1 hr while elevating the temperature from −78° C. to −20° C. The reaction mixture was diluted with water (70 mL), and extracted with ethyl acetate. (70 mL, 40 mL). The organic layer was washed with saturated brine (30 mL), and concentrated under reduced pressure. The residue was purified by column chromatography (silica gel, hexane/ethyl acetate=100/0→50/50) and crystallized from diisopropyl ether to give the title compound (881 mg, 46%).

¹H-NMR (CDCl₃, 300 MHz) δ 4.31 (3H, s), 7.34 (1H, s), 8.83 (1H, s).

### Reference Example 61-2

### 4-(4-amino-3-chlorophenoxy)-5-methyl-5H-pyrrolo[3,2-d]pyrimidine-6-carbonitrile

A mixture of 4-chloro-5-methyl-5H-pyrrolo[3,2-d]pyrimidine-6-carbonitrile (828 mg, 4.3 mmol), 4-amino-3-chlorophenol (741 mg, 5.2 mmol), potassium carbonate (1783 mg, 12.9 mmol) and N-methylpyrrolidone (5 mL) was stirred at 110° C. for 2 hr. The reaction mixture was diluted with water (50 mL) and extracted with ethyl acetate (50 mL, 30 mL×2). The organic layer was concentrated under reduced pressure, and the residue was purified by column chromatography (silica gel, hexane/ethyl acetate=10/90→90/10) and recrystallized from diisopropyl ether/ethyl acetate to give the title compound (774 mg, 60%).

¹H-NMR (DMSO-d₆, 300 MHz) δ 4.18 (3H, s), 5.41 (2H, s), 6.86 (1H, d, J=9.0 Hz), 7.04 (1H, dd, J=9.0, 2.5 Hz), 7.27 (1H, d, J=2.5 Hz), 7.61 (1H, s), 8.45 (1H, s).

### Reference Example 62-1

### 2-(4-chloro-5H-pyrrolo[3,2-d]pyrimidin-5-yl)acetamide

A mixture of 4-chloro-5H-pyrrolo[3,2-d]pyrimidine (1.54 g, 10.0 mmol), 2-bromoacetamide (1.51 g, 10.9 mmol), cesium carbonate (3.58 g, 11.0 mmol) and N,N-dimethylformamide (13 mL) was stirred at room temperature for 69 hr. Cesium carbonate (1.30 g, 3.99 mmol) was added, and the reaction mixture was stirred at room temperature for 24 hr. The reaction mixture was diluted with water, and extracted with ethyl acetate (×7). The organic layers were combined, dried over anhydrous magnesium sulfate, and filtrated. The filtrate was concentrated under reduced pressure to give the title compound (1.81 g, 86%) as a yellow solid.

¹H-NMR (DMSO-d₆, 300 MHz) δ 5.14 (2H, s), 6.73 (1H, d, J=3.3 Hz), 7.30 (1H, br s) 7.68 (1H, br s), 7.95 (1H, d, J=3.3 Hz), 8.61 (1H, s).

### Reference Example 62-2

### 2-[4-(4-amino-3-chlorophenoxy)-5H-pyrrolo[3,2-d]pyrimidin-5-yl]acetamide

A mixture of 2-(4-chloro-5H-pyrrolo[3,2-d]pyrimidin-5-yl)acetamide (1.81 g, 8.59 mmol), 4-amino-3-chlorophenol (1.36 g, 9.47 mmol), potassium carbonate (2.37 g, 17.1 mmol) and N-methylpyrrolidone (10 mL) was stirred at 110° C. for 2 hr. The reaction mixture was diluted with water and extracted with ethyl acetate (×3). The organic layer was dried over anhydrous magnesium sulfate, and concentrated under reduced pressure. The residue was purified by column chromatography (silica gel, hexane/ethyl acetate=80/20→0/100), and the obtained solid was washed with ethyl acetate-ether and dried to give the title compound (572 mg, 21%).

¹H-NMR (DMSO-d₆, 300 MHz) δ 5.04 (2H, s), 5.32 (2H, br s), 6.58 (1H, d, J=3.3 Hz), 6.84 (1H, d, J=8.7 Hz), 6.94 (1H, dd, J=8.7, 2.7 Hz), 7.10 (1H, d, J=2.7 Hz), 7.24 (1H, br s), 7.58 (1H, br s), 7.73 (1H, d, J=3.3 Hz), 8.28 (1H, s).

### Reference Example 63-1

### 4-(4-amino-3-chlorophenoxy)-N-methyl-6-{3-methyl-3-[(trimethylsilyl)oxy]but-1-yn-1-yl}pyrimidine-5-amine

To a solution of 4-(4-amino-3-chlorophenoxy)-6-iodo-N-methylpyrimidine-5-amine (1.5 g, 3.98 mmol) in acetonitrile (60 mL)/triethylamine (30 mL) were added 3-methyl-3-trimethylsilyloxy-1-butyne (0.93 mL, 4.78 mmol), dichlorobis(triphenylphosphine)palladium (140 mg, 0.20 mmol) and copper iodide (76 mg, 0.40 mmol), and the mixture was stirred at 80° C. for 4 hr. After cooling to room temperature, the solvent was evaporated under reduced pressure, and the residue was purified by column chromatography (silica gel, ethyl acetate/hexane) to give the title compound (1.65 g, quant.) as a brown liquid.

¹H-NMR (DMSO-d₆, 300 MHz) δ 0.20 (9H, s), 1.54 (6H, s), 3.16 (3H, d, J=5.4 Hz), 5.31 (2H, s), 5.83 (1H, q, J=5.4 Hz), 6.82 (1H, d, J=8.7 Hz), 6.90 (1H, dd, J=8.7, 2.7 Hz), 7.12 (1H, d, J=2.7 Hz), 7.85 (1H, s).

### Reference Example 63-2

### 2-chloro-4-[(5-methyl-6-{1-methyl-1-[(trimethylsilyl)oxy]ethyl}-5H-pyrrolo[3,2-d]pyrimidin-4-yl)oxy]aniline

To a solution of 4-(4-amino-3-chlorophenoxy)-N-methyl-6-{3-methyl-3-[(trimethylsilyl)oxy]but-1-yn-1-yl}pyrimidine-5-amine (1.65 g, 4.20 mmol) in N,N-dimethylformamide (33 mL) was added copper iodide (80 mg, 0.42 mmol), and the mixture was stirred at 80° C. for 1 hr. After cooling to room temperature, water was added to the reaction mixture, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (0.96 g, 60%) as a brown solid.

¹H-NMR (CDCl₃, 300 MHz) δ 0.07 (9H, s), 1.76 (6H, s), 4.05 (2H, s), 4.31 (3H, s), 6.56 (1H, br), 6.83 (1H, d, J=8.7 Hz), 6.99 (1H, dd, J=8.7, 2.7 Hz), 7.20 (1H, d, J=2.7 Hz), 8.45 (1H, br).

### Reference Example 64

### 3-(benzyloxy)-5-(trifluoromethyl)aniline

A mixture of 3-nitro-5-(trifluoromethyl)phenol (4.15 g, 20.0 mmol), benzyl bromide (5.10 g, 29.8 mmol), potassium carbonate (5.53 g, 40.0 mmol) and N,N-dimethylformamide (35 mL) was stirred at 60° C. for 6 hr. The reaction mixture was diluted with ethyl acetate, washed with water (×2), and dried over anhydrous magnesium sulfate. After concentration under reduced pressure, the residue was purified by column chromatography (silica gel, hexane/ethyl acetate=100/0→75/25) to give 1-(benzyloxy)-3-nitro-5-(trifluoromethyl)benzene (6.16 g) containing ethyl acetate. A mixture of the thus-obtained 1-(benzyloxy)-3-nitro-5-(trifluoromethyl)benzene (6.16 g), calcium chloride (1.11 g, 10.0 mmol), ethanol (200 mL) and water (20 mL) was stirred at 90° C. for 5 min. Reduced iron (6.70 g, 120 mmol) was added and the mixture was stirred at 90° C. for 9.5 hr. After cooling to room temperature, the mixture was filtered through celite. The filtrate was concentrated under reduced pressure, and the residue was diluted with ethyl acetate and dried over anhydrous magnesium sulfate. After concentration under reduced pressure, the residue was purified by column chromatography (silica gel, hexane/ethyl acetate=90/10→70/30) to give the title compound (4.73 g, 88%).

¹H-NMR (CDCl₃, 300 MHz) δ 3.82 (2H, br s), 5.04 (2H, s), 6.42 (1H, m), 6.52 (1H, s), 6.62 (1H, s), 7.30-7.43 (5H, m).

### Reference Example 65

### N-[4-hydroxy-2-(hydroxymethyl)phenyl]-N′-[3-(trifluoromethyl)phenyl]urea

To a solution of 4-amino-3-(hydroxymethyl)phenol (241 mg, 1.00 mmol) and triethylamine (415 μL, 3.00 mmol) in tetrahydrofuran, (10 mL) was added 3-(trifluoromethyl)phenylisocyanate (140 μL, 1.00 mmol), and the mixture was stirred at room temperature for 5 hr. The precipitated solid was collected by filtration and washed with ethyl acetate-hexane to give the title compound (283 mg, 87%) as a white solid.

¹H-NMR (DMSO-d₆, 300 MHz) δ 4.42 (2H, d, J=5.6 Hz), 5.28 (1H, t, J=5.6 Hz), 6.62 (1H, dd, J=8.6, 2.8 Hz), 6.78 (1H, d, J=2.8 Hz), 7.25 (1H, d, J=8.0 Hz), 7.36 (1H, d, J=8.6 Hz), 7.47 (1H, t, J=8.0 Hz), 7.56 (1H, d, J=8.0 Hz), 7.93 (1H, s), 7.99 (1H, s), 9.17 (1H, s), 9.39 (1H, s).

### Reference Example 66-1

### 4-nitro-2-(trifluoromethyl)phenol

A mixture of 1-methoxy-4-nitro-2-(trifluoromethyl)benzene (10.29 g, 46.5 mmol), lithium chloride (5.92 g, 140 mmol) and N,N-dimethylformamide (46.5 mL) was heated under reflux for 6.5 hr. After cooling to room temperature, 10% aqueous sodium hydroxide solution (230 mL) was added, and the mixture was washed with ethylether (×2). The aqueous solution was acidified with 10% hydrochloric acid, and extracted with ether (×2). The extract was washed with saturated brine, and dried over anhydrous magnesium sulfate. After concentration under reduced pressure, the residue was purified by column chromatography (silica gel, hexane/ethyl acetate=80/20) to give the title compound (6.20 g, 65%).

¹H-NMR (CDCl₃, 300 MHz) δ 7.08 (1H, d, J=9.0 Hz), 8.32 (1H, dd, J=9.0, 2.7 Hz), 8.48 (1H, d, J=2.7 Hz).

### Reference Example 66-2

### 4-(benzyloxy)-3-(trifluoromethyl)aniline

A mixture of 4-nitro-2-(trifluoromethyl)phenol (6.20 g, 29.9 mmol), benzyl bromide (7.68 g, 44.9 mmol), potassium carbonate (8.26 g, 59.8 mmol) and N,N-dimethylformamide (50 mL) was stirred at 60° C. for 1.5 hr. The reaction mixture was diluted with ethyl acetate, washed with water (×2) and dried over anhydrous magnesium sulfate. After concentration under reduced pressure, the residue was purified by column chromatography (silica gel, hexane/ethyl acetate=100/0→475/25) to give 1-(benzyloxy)-4-nitro-2-(trifluoromethyl)benzene (8.44 g). A mixture of the thus-obtained 1-(benzyloxy)-4-nitro-2-(trifluoromethyl)benzene (8.44 g), calcium chloride (1.58 g, 14.2 mmol), ethanol (280 mL) and water (28 mL) was stirred at 90° C. for 5 min and reduced iron (9.52 g, 170 mmol) was added. The mixture was stirred at 90° C. for 6.5 hr, cooled to room temperature and filtered through celite. The filtrate was concentrated under reduced pressure, and the residue was diluted with ethyl acetate and dried over anhydrous magnesium sulfate. After concentration under reduced pressure, the residue was purified by column chromatography (silica gel, hexane/ethyl acetate=90/10→70/30) to give the title compound (4.80 g, 63%).

¹H-NMR (CDCl₃, 300 MHz) δ 3.54 (2H, br s), 5.08 (2H, s), 6.75 (1H, dd, J=9.0, 2.7 Hz), 6.86 (1H, d, J=9.0 Hz), 6.92 (1H, d, J=2.7 Hz), 7.25-7.44 (5H, m).

### Reference Example 67-1

### 4-(4-amino-3-chlorophenoxy)-N-methyl-6-[3-(tetrahydro-2H-pyran-2-yloxy)prop-1-yn-1-yl]pyrimidine-5-amine

To a solution of 4-(4-amino-3-chlorophenoxy)-6-iodo-N-methylpyrimidine-5-amine (3.0 g, 7.97 mmol) in acetonitrile (90 mL)/triethylamine (90 mL) were added tetrahydro-2-(2-propynyloxy)-2H-pyran (1.34 g, 9.56 mmol), dichlorobis(triphenylphosphine)palladium (0.28 g, 0.40 mmol) and copper iodide (0.15 g, 0.80 mmol), and the mixture was stirred at room temperature for 30 min and then stirred at 60° C. for 1 hr. After cooling to room temperature, the solvent was evaporated under reduced pressure. Water was added to the residue, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (2.11 g, 68%) as a brown liquid.

¹H-NMR (CDCl₃, 300 MHz) δ 1.25-1.86 (6H, m), 3.27 (3H, d, J=5.4 Hz), 3.54-3.61 (1H, m), 3.85-3.94 (1H, m), 4.04 (2H, s), 4.42-4.49 (1H, m), 4.56 (2H, s), 4.87-4.90 (1H, m), 6.83 (1H, d, J=8.7 Hz), 6.89 (1H, dd, J=8.7, 2.7 Hz), 7.10 (1H, d, J=2.7 Hz), 8.05 (1H, s).

### Reference Example 67-2

### 2-chloro-4-({5-methyl-6-[(tetrahydro-2H-pyran-2-yloxy)methyl]-5H-pyrrolo[3,2-d]pyrimidin-4-yl}oxy)aniline

To a solution of 4-(4-amino-3-chlorophenoxy)-N-methyl-6-[3-(tetrahydro-2H-pyran-2-yloxy)prop-1-yn-1-yl]pyrimidine-5-amine (2.1 g, 5.40 mmol) in N,N-dimethylformamide (42 mL) was added copper iodide (103 mg, 0.54 mmol), and the mixture was stirred at 80° C. for 1 hr. After cooling to room temperature, water was added to the reaction mixture, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (1.35 g, 64%) as a brown solid.

¹H-NMR (CDCl₃, 300 MHz) δ 1.48-1.86 (6H, m), 3.56-3.64 (1H, m), 3.85-3.97 (1H, m), 4.06 (2H, s), 4.13 (3H, s), 4.71 (1H, d, J=12.8 Hz), 4.73-4.76 (1H, m), 4.93 (1H, d, J=12.8 Hz), 6.66 (1H, s), 6.83 (1H, d, J=8.7 Hz), 6.99 (1H, dd, J=8.7, 2.6 Hz), 7.19 (1H, d, J=2.6 Hz), 8.43 (1H, s).

### Reference Example 68-1

### 4-(4-amino-3-chlorophenoxy)-6-(3-methoxyprop-1-yn-1-yl)-N-methylpyrimidine-5-amine

To a solution of 4-(4-amino-3-chlorophenoxy)-6-iodo-N-methylpyrimidine-5-amine (3.0 g, 7.97 mmol) in acetonitrile (90 mL)/triethylamine (90 mL) were added methylpropalgyl ether (0.79 mL, 9.56 mmol), dichlorobis(triphenylphosphine)palladium (0.28 g, 0.40 mmol) and copper iodide (0.15 g, 0.80 mmol) and the mixture was stirred at room temperature for 30 min and then stirred at 60° C. for 1 hr. After cooling to room temperature, the solvent was evaporated under reduced pressure. Water was added to the residue, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (1.49 g, 59%) as a brown liquid.

¹H-NMR (CDCl₃, 300 MHz) δ 3.28 (3H, d, J=5.4 Hz), 3.47 (3H, s), 4.04 (2H, s), 4.40 (2H, s), 4.37-4.46 (1H, m), 6.79 (1H, d, J=8.7 Hz), 6.88 (1H, dd, J=8.7, 2.6 Hz), 7.09 (1H, d, J=2.6 Hz), 8.05 (1H, s).

### Reference Example 68-2

### 2-chloro-4-{[6-(methoxymethyl)-5-methyl-5H-pyrrolo[3,2-d]pyrimidin-4-yl]oxy}aniline

To a solution of 4-(4-amino-3-chlorophenoxy)-6-(3-methoxyprop-1-yn-1-yl)-N-methylpyrimidine-5-amine (1.49 g, 4.67 mmol) in N,N-dimethylformamide (14.9 mL) was added copper iodide (89 mg, 0.47 mmol), and the mixture was stirred at 80° C. for 1 hr. After cooling to room temperature, water was added to the reaction mixture, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) to give the title compound (725 mg, 49%) as a brown solid.

¹H-NMR (DMSO-d₆, 300 MHz) δ 2.34 (3H, s), 4.02 (3H, s), 4.66 (2H, s), 5.34 (2H, s), 6.62 (1H, s), 6.84 (1H, d, J=8.7 Hz), 7.00 (1H, dd, J=8.7, 2.7 Hz), 7.22 (1H, d, J=2.7 Hz), 8.25 (1H, s).

### Reference Example 69

### Ethyl {4-[(5-methyl-5H-pyrrolo[3,2-d]pyrimidin-4-yl)oxy]phenyl}acetate

A mixture of 4-chloro-5-methyl-5H-pyrrolo[3,2-d]pyrimidine (670 mg, 4.0 mmol), ethyl (4-hydroxyphenyl)acetate (937 mg, 5.2 mmol), potassium carbonate (1106 mg, 8.0 mmol) and N-methylpyrrolidone (8 mL) was stirred at 90° C. for 8 hr. The reaction mixture was diluted with water and extracted with ethyl acetate. The organic layer was washed with water and saturated brine, and concentrated under reduced pressure. The residue was purified by column chromatography (NH silica gel, hexane/ethyl acetate=90/10→10/90) then by column chromatography (silica gel, hexane/ethyl acetate=80/20→0/100) to give the title compound (1095 mg, 88%) as a white solid.

¹H-NMR (CDCl₃, 300 MHz) δ 1.28 (3H, t, J=7.2 Hz), 3.65 (2H, s), 4.14 (3H, s), 4.17 (2H, q, J=7.2 Hz), 6.65 (1H, d, J=3.0 Hz), 7.22 (2H, d, J=8.4 Hz), 7.32 (1H, d, J=3.0 Hz), 7.39 (2H, d, J=8.4 Hz), 8.44 (1H, s).

### Reference Example 70

### {4-[(5-methyl-5H-pyrrolo[3,2-d]pyrimidin-4-yl)oxy]phenyl}acetic Acid

To a solution of ethyl {4-[(5-methyl-5H-pyrrolo[3,2-d]pyrimidin-4-yl)oxy]phenyl}acetate (990 mg, 3.2 mmol) in methanol (10 mL) was added 8N aqueous sodium hydroxide solution (1.00 mL), and the mixture was stirred at room temperature for 18 hr. 1N Hydrochloric acid (8.00 mL) was added to the reaction mixture and the mixture was concentrated under reduced pressure. The residue was diluted with water, and the precipitate was collected by filtration and washed with water. The precipitate was recrystallized from methanol to give the title compound (669 mg, 74%) as a white solid.

¹H-NMR (DMSO-d₆, 300 MHz) δ 3.62 (2H, s), 4.10 (3H, s), 6.59 (1H, d, J=3.2 Hz), 7.25 (2H, d, J=8.6 Hz), 7.35 (2H, d, J=8.6 Hz), 7.78 (1H, d, J=3.2 Hz), 8.28 (1H, s), 12.37 (1H, br s).

### Reference Example 71

### 3-[(5-methyl-5H-pyrrolo[3,2-d]pyrimidin-4-yl)oxy]aniline

A mixture of 4-chloro-5-methyl-5H-pyrrolo[3,2-d]pyrimidine (2.08 g, 9.83 mmol), 3-aminophenol (1.29 g, 11.8 mmol), potassium carbonate (3.26 g, 23.6 mmol) and N-methylpyrrolidone (15 mL) was stirred at 110° C. for 2 hr. The reaction mixture was diluted with water, and extracted with ethyl acetate (×3). The organic layer was washed with saturated brine, dried over anhydrous magnesium sulfate, and filtrated. The filtrate was concentrated under reduced pressure. The residue was collected by filtration and washed with ethyl acetate-hexane to give the title compound (1.46 g, 62%) as a white solid.

¹H-NMR (DMSO-d₆, 300 MHz) δ 4.07 (3H, s), 5.28 (2H, br s), 6.34-6.50 (3H, m), 6.58 (1H, d, J=3.2 Hz), 7.06 (1H, t, J=7.8 Hz), 7.76 (1H, d, J=3.2 Hz), 8.28 (1H, s).

### Reference Example 72

### 1-tert-butyl-4-nitro-1H-pyrazole

To a solution of 4-nitropyrazole (1.13 g, 10 mmol) and 2-bromo-2-methylpropane (17.81 g, 130 mmol) in N,N-dimethylformamide (50 mL) was added potassium carbonate (21.56 g, 156 mmol) and the mixture was stirred at 80° C. for 3 days. The reaction mixture was diluted with water (200 mL), and extracted with ethyl acetate (100 mL×3). The organic layer was washed with water (50 mL) and saturated brine (50 mL), and concentrated under reduced pressure. The residue was purified by column chromatography (silica gel, hexane/ethyl acetate=100/0→60/40) to give the title compound (1.13 g, 67%) as a white solid.

¹H-NMR (CDCl₃, 300 MHz) δ 1.63 (9H, s), 8.09 (1H, s), 8.24 (1H, s).

### Reference Example 73

### 1-tert-butyl-1H-pyrazole-4-amine

To a solution of 1-tert-butyl-4-nitro-1H-pyrazole (1.10 g, 6.5 mmol) in methanol (20 mL) was added 10% palladium carbon (containing 50% water, 442 mg), and the mixture was stirred under a hydrogen atmosphere at room temperature for 3 hr. The catalyst was filtered off and the filtrate was concentrated under reduced pressure. The residue was purified by column chromatography (silica gel, hexane/ethyl acetate=70/30→0/100) to give the title compound (838 mg, 92%).

¹H-NMR (CDCl₃, 300 MHz) δ 1.53 (9H, s), 2.86 (2H, br s), 7.15 (1H, s), 7.19 (1H, s).

### Reference Example 74

### 4-(4-amino-3-chlorophenoxy)-6-prop-1-yn-1-ylpyrimidine-5-amine

To a mixture of 4-(4-amino-3-chlorophenoxy)-6-iodopyrimidine-5-amine (2.54 g, 7.0 mmol), trimethylsilylacetylene (1.57 g, 14.0 mmol), triphenylphosphine (184 mg, 0.70 mmol), copper iodide (133 mg, 0.70 mmol), triethylamine (70 mL) and acetonitrile (70 mL) were added bis(triphenylphosphine)palladium(II) dichloride (246 mg, 0.35 mmol) and potassium fluoride (895 mg, 15.4 mmol), and the mixture was stirred at 80° C. for 24 hr. The reaction mixture was concentrated under reduced pressure, and the residue was diluted with water and extracted with ethyl acetate. The organic layer was concentrated under reduced pressure, and the residue was purified by column chromatography (silica gel, hexane/ethyl acetate=50/50→0/100) to give the title compound (1.14 g, 59%) as a brown solid.

¹H-NMR (DMSO-d₆, 300 MHz) δ 2.15 (3H, s), 5.31 (2H, s), 5.58 (2H, s), 6.81 (1H, d, J=8.7 Hz), 6.91 (1H, dd, J=8.7, 2.6 Hz), 7.12 (1H, d, J=2.6 Hz), 7.83 (1H, s).

### Reference Example 75

### 2-chloro-4-[(6-methyl-5H-pyrrolo[3,2-d]pyrimidin-4-yl)oxy]aniline

To a solution of 4-(4-amino-3-chlorophenoxy)-6-prop-1-yn-1-ylpyrimidine-5-amine (940 mg, 3.4 mmol) in tetrahydrofuran (70 mL) was added potassium t-butoxide (576 mg, 5.1 mmol), and the mixture was stirred at room temperature for 18 hr. The reaction mixture was diluted with water and extracted with ethyl acetate. The organic layer was concentrated under reduced pressure, and the residue was purified by column chromatography (silica gel, hexane/ethyl acetate=50/50→0/100) and recrystallized from ethyl acetate to give the title compound (617 mg, 66%) as a yellow solid.

¹H-NMR (DMSO-d₆, 300 MHz) δ 2.46 (3H, s), 5.32 (2H, s), 6.33 (1H, s), 6.85 (1H, d, J=8.7 Hz), 6.96 (1H, dd, J=8.7, 2.6 Hz), 7.18 (1H, d, J=2.6 Hz), 8.22 (1H, s), 12.04 (1H, s).

### Reference Example 76

### 3-{[(5-methyl-5H-pyrrolo[3,2-d]pyrimidin-4-yl)oxy]methyl}aniline

To a solution of sodium hydride (792 mg, 33.0 mmol) in N,N-dimethylformamide (15 mL) added dropwise a solution of (3-aminophenyl)methanol (4.19 g, 30.0 mmol) in N,N-dimethylformamide (15 mL) at 0° C., and the mixture was stirred at room temperature for 1 hr. 4-Chloro-5-methyl-5H-pyrrolo[3,2-d]pyrimidine (3.69 g, 25.0 mmol) was added to the reaction mixture, and the mixture was stirred at 100° C. for 6 hr. The reaction mixture was diluted with water, and extracted with ethyl acetate (×3). The organic layer was washed with saturated brine, dried over anhydrous magnesium sulfate, and filtrated. The filtrate was concentrated under reduced pressure. The residue was collected by filtration and washed with ethyl acetate-hexane to give the title compound (3.90 g, 61%) as a white solid.

¹H-NMR (DMSO-d₆, 300 MHz) δ 4.01 (3H, s), 5.13 (2H, br s), 5.44 (2H, s), 6.49-6.52 (2H, m), 6.52-6.69 (2H, m), 7.02 (1H, t, J=7.8 Hz), 7.64 (1H, d, J=3.0 Hz), 8.36 (1H, s).

### Reference Example 77

### 4-[(5-methyl-5H-pyrrolo[3,2-d]pyrimidin-4-yl)oxy]benzoic Acid

A mixture of 4-chloro-5-methyl-5H-pyrrolo[3,2-d]pyrimidine (2.08 g, 12.4 mmol), 4-hydroxybenzoic acid (1.88 g, 13.7 mmol), cesium carbonate (12.1 g, 37.2 mmol) and dimethyl sulfoxide (10 mL) was stirred at 100° C. for 4 hr. 1N Hydrochloric acid was added to adjust the reaction solution to pH=4. The precipitated solid was collected by filtration and washed with water to give the title compound (3.08 g, 72%) as a white solid.

¹H-NMR (DMSO-d₆, 300 MHz) δ 4.10 (3H, s), 6.62 (1H, d, J=3.3 Hz), 7.44 (2H, d, J=8.9 Hz), 7.81 (1H, d, J=3.3 Hz), 8.04 (2H, d, J=8.9 Hz), 8.31 (1H, s), 13.02 (1H, br s).

### Reference Example 78

### 3-[(5-methyl-5H-pyrrolo[3,2-d]pyrimidin-4-yl)oxy]benzoic Acid

Using 4-chloro-5-methyl-5H-pyrrolo[3,2-d]pyrimidine (2.23 g, 13.3 mmol), 3-hydroxybenzoic-acid (2.02 g, 14.6 mmol), cesium carbonate (13.0 g, 39.9 mmol) and dimethyl sulfoxide (20 mL) as starting materials, and in the same manner as in Reference Example 77, the title compound (3.48 g, 76%) was obtained as a white solid.

¹H-NMR (DMSO-d₆, 300 MHz) δ 4.12 (3H, s), 6.62 (1H, d, J=3.0 Hz), 7.58-7.64 (2H, m), 7.80-7.91 (3H, m), 8.30 (1H, s), 13.21 (1H, br s).

### Reference Example 79

### 5-methyl-5H-pyrrolo[3,2-d]pyrimidin-4-ol

A mixture of 4-chloro-5-methyl-5H-pyrrolo[3,2-d]pyrimidine (3.30 g, 19.7 mmol) and aqueous 6N hydrochloric acid solution (20 mL) was stirred at 120° C. for 1 hr. The reaction solution was cooled to 0° C., and 8N aqueous sodium hydroxide solution was added to adjust to pH 7. The precipitated solid was collected by filtration, washed with water and dried to give the title compound (2.60 g, 88%) as a yellow solid.

¹H-NMR (DMSO-d₆, 300 MHz) δ 3.99 (3H, s), 6.31 (1H, d, J=2.7 Hz), 7.35 (1H, d, J=2.7 Hz), 7.73 (1H, d, J=3.0 Hz), 11.81 (1H, br s).

### Reference Example 80

### 5-methyl-4-[(4-nitrobenzyl)oxy]-5H-pyrrolo[3,2-d]pyrimidine

A mixture of 5-methyl-5H-pyrrolo[3,2-d]pyrimidin-4-ol (489 mg, 3.28 mmol), 1-(bromomethyl)-4-nitrobenzene (1.06 g, 4.92 mmol), potassium carbonate (1.36 g, 9.84 mmol), sodium iodide (98.3 mg, 0.656 mmol) and N,N-dimethylformamide (7 mL) was stirred at 50° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (ethyl acetate/hexane=30/70→100/0) to give the title compound (489 mg, 52%) as a yellow solid.

¹H-NMR (DMSO-d₆, 300 MHz) δ 3.98 (3H, s), 5.30 (2H, s), 6.35-6.36 (1H, m), 7.41 (1H, d, J=2.7 Hz), 7.54 (2H, d, J=8.9 Hz), 8.20 (2H, d, J=8.9 Hz), 8.29 (1H, s).

### Reference Example 81

### 4-{[(5-methyl-5H-pyrrolo[3,2-d]pyrimidin-4-yl)oxy]methyl}aniline

To a solution of 5-methyl-4-[(4-nitrobenzyl)oxy]-5H-pyrrolo[3,2-d]pyrimidine (489 mg, 1.72 mmol) in methanol (10 mL) was added palladium carbon (50% water-containing product, 50 mg), and the mixture was stirred under a hydrogen atmosphere at room temperature for 4 hr. The reaction mixture was filtered through celite. The filtrate was concentrated under reduced pressure, and the residue was collected by filtration and washed with ethyl acetate-hexane to give the title compound (315 mg, 72%) as a white solid.

¹H-NMR (DMSO-d₆, 300 MHz) δ 3.99 (3H, s), 4.95 (2H, s), 5.08 (2H, s), 6.29 (1H, d, J=2.9 Hz), 6.48 (2H, d, J=8.4 Hz), 7.04 (2H, d, J=8.4 Hz), 7.36 (1H, d, J=2.9 Hz), 8.17 (1H, s).

### Reference Example 82

### 6-[(5-methyl-5H-pyrrolo[3,2-d]pyrimidin-4-yl)oxy]-1-naphthoic Acid

Using 4-chloro-5-methyl-5H-pyrrolo[3,2-d]pyrimidine (2.63 g, 15.7 mmol), 6-hydroxy-1-naphthoic acid (3.25 g, 17.3 mmol), cesium carbonate (15.3 g, 47.1 mmol) and dimethyl sulfoxide (15 mL) as starting materials, and in the same manner as in Reference Example 77, the title compound (5.22 g, 99%) was obtained as a white solid.

¹H-NMR (DMSO-d₆, 300 MHz) δ 4.17 (3H, s), 6.65 (1H, d, J=2.9 Hz), 7.61-7.66 (2H, m), 7.86 (1H, d, J=2.9 Hz), 7.97 (1H, d, J=2.4 Hz), 8.15-8.18 (2H, m), 8.34 (1H, br s), 8.97 (1H, d, J=8.7 Hz), 12.29 (1H, br s).

### Example 1

### N-methyl-N′-{4-[(5-methyl-5H-pyrrolo[3,2-d]pyrimidin-4-yl)oxy]phenyl}urea

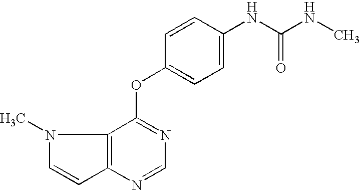

A mixture of 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol) and N,N-dimethylformamide (3 mL) was stirred at room temperature for 1 hr. 2M-Methylamine tetrahydrofuran solution (330 μL, 0.655 mmol) was added to the reaction mixture, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=50/50→100/0) and recrystallized from ethyl acetate/hexane to give the title compound (47.5 mg, 26%) as a white solid.

¹H-NMR (DMSO-d₆, 200 MHz) δ 2.59 (3H, d, J=4.8 Hz), 4.03 (3H, s), 5.99 (1H, d, J=4.8 Hz), 6.51 (1H, d, J=2.4 Hz), 7.08 (2H, d, J=8.8 Hz), 7.39 (2H, d, J=8.8 Hz), 7.70 (1H, d, J=2.4 Hz), 8.19 (1H, s), 8.54 (1H, br s).

### Example 2

### N-{4-[(5-methyl-5H-pyrrolo[3,2-d]pyrimidin-4-yl)oxy]phenyl}-N′-phenylurea

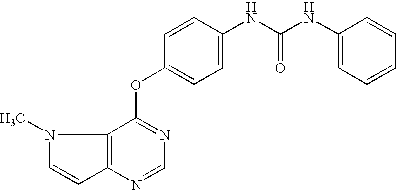

A mixture of 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol) and N,N-dimethylformamide (3 mL) was stirred at room temperature for 1 hr. Aniline (61.0 mg, 0.655 mmol) was added to the reaction mixture, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=20/80→100/0) and recrystallized from ethyl acetate-hexane to give the title compound (75.1 mg, 34%) as a white solid.

¹H-NMR (DMSO-d₆, 200 MHz) δ 4.04 (3H, s), 6.53 (1H, d, J=2.9 Hz), 6.90 (1H, t, J=7.4 Hz), 7.14-7.26 (4H, m), 7.39-7.49 (4H, m), 7.71 (1H, d, J=2.9 Hz), 8.21 (1H, s), 8.68 (1H, br s), 8.75 (1H, br s).

### Example 3

### N-{4-[(5-methyl-5H-pyrrolo[3,2-d]pyrimidin-4-yl)oxy]phenyl}-N′-[3-(trifluoromethyl)phenyl]urea

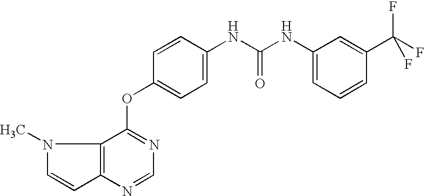

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), 3-(trifluoromethyl)aniline (105 mg, 0.655 mmol) and N,N-dimethylformamide (3 mL) as starting materials, and in the same manner as in Example 2, the title compound (36.0 mg, 14%) was obtained as a white solid.

¹H-NMR (DMSO-d₆, 200 MHz) δ 4.04 (3H, s), 6.53 (1H, d, J=2.8 Hz), 7.15-7.27 (3H, m), 7.41-7.51 (4H, m), 7.71 (1H, d, J=2.8 Hz), 7.98 (1H, s), 8.21 (1H, s), 8.94 (1H, br s), 9.14 (1H, br s).

### Example 4

### N-(3-chlorophenyl)-N′-{4-[(5-methyl-5H-pyrrolo[3,2-d]pyrimidin-4-yl)oxy]phenyl}urea

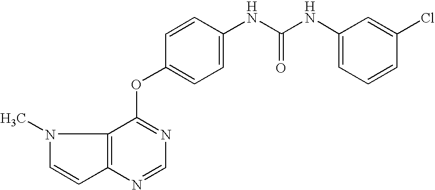

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), 3-chloroaniline (83.6 mg, 0.655 mmol) and N,N-dimethylformamide (3 mL) as starting materials, and in the same manner as in Example 2, the title compound (34.8 mg, 14%) was obtained as a white solid melting point 184-186° C.

¹H-NMR (DMSO-d₆, 200 MHz) δ 4.04 (3H, s), 6.53 (1H, d, J=2.8 Hz), 6.94-6.98 (1H, m), 7.15-7.25 (4H, m), 7.46 (2H, d, J=8.8 Hz), 7.66 (1H, s), 7.71 (1H, d, J=2.8 Hz), 8.21 (1H, s), 8.78 (1H, br s), 8.85 (1H, br s).

### Example 5

### N-(4-chlorophenyl)-N′-{4-[(5-methyl-5H-pyrrolo[3,2-d]pyrimidin-4-yl)oxy]phenyl}urea

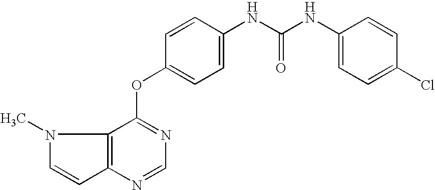

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), 4-chloroaniline (83.6 mg, 0.655 mmol) and N,N-dimethylformamide (3 mL) as starting materials, and in the same manner as in Example 2, the title compound (45.1 mg, 18%) was obtained as a white solid.

¹H-NMR (DMSO-d₆, 200 MHz) δ 4.04 (3H, s), 6.53 (1H, d, J=3.1 Hz), 7.16 (2H, d, J=8.8 Hz), 7.26 (2H, d, J=8.6 Hz), 7.24-7.48 (4H, m), 7.71 (1H, d, J=3.1 Hz), 8.21 (1H, s), 8.73 (1H, br s), 8.78 (1H, br s).

### Example 6

### N-(4-methoxyphenyl)-N′-{4-[(5-methyl-5H-pyrrolo[3,2-d]pyrimidin-4-yl)oxy]phenyl}urea

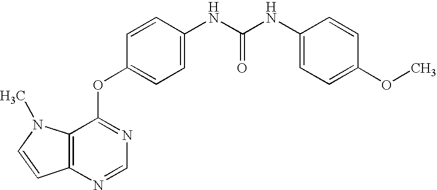

A mixture of 4-chloro-5-methyl-5H-pyrrolo[3,2-d]pyrimidine (84 mg, 0.5 mmol), 4-amino-phenol (110 mg, 1.0 mmol), cesium carbonate (326 mg, 1.0 mmol) and N-methylpyrrolidone (5 mL) was stirred at 120° C. for 6 hr. The reaction mixture was diluted with water, and extracted with ethyl acetate (×2). The organic layer was washed with water and concentrated under reduced pressure to give 4-[(5-methyl-5H-pyrrolo[3,2-d]pyrimidin-4-yl)oxy]aniline (120 mg, quant.). This was dissolved in N,N-dimethylformamide (5 mL), N,N′-carbonyldiimidazole (324 mg, 2.0 mmol) and diisopropylethylamine (650 mg, 5.0 mmol) were added under ice-cooling, and the mixture was stirred at room temperature for 18 hr. 4-Methoxyaniline (123 mg, 1.0 mmol) was added thereto under ice-cooling, and the mixture was further stirred at room temperature for 18 hr. The reaction mixture was diluted with water and extracted with ethyl acetate (×3). The organic layer was concentrated under reduced pressure, and the residue was purified by column chromatography (silica gel) to give the title compound (100 mg, 51%) as a white powder.

¹H-NMR (CDCl₃, 300 MHz) δ 3.82 (3H, s), 4.14 (3H, s), 6.33 (1H, br s), 6.49 (1H, br s), 6.65 (1H, d, J=2.9 Hz), 6.92 (2H, d, J=8.7 Hz), 7.20 (2H, d, J=9.2 Hz), 7.27 (2H, d, J=8.7 Hz), 7.32 (1H, d, J=2.9 Hz), 7.44 (2H, d, J=9.2 Hz), 8.43 (1H, s).

### Example 7

### N-(5-methylisoxazol-3-yl)-N′-{4-[(5-methyl-5H-pyrrolo[3,2-d]pyrimidin-4-yl)oxy]phenyl}urea

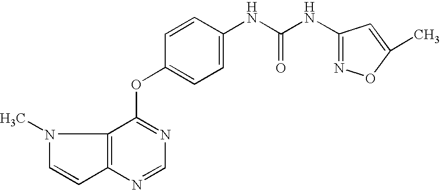

A mixture of 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol) and N,N-dimethylformamide (3 mL) was stirred at room temperature for 1 hr. 3-Amino-5-methylisoxazole (61.2 mg, 0.624 mmol) was added to the reaction mixture, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=50/50→100/0) and recrystallized from ethyl acetate to give the title compound (43.1 mg, 19%) as a white solid.

¹H-NMR (DMSO-d₆, 200 MHz) δ 2.30 (3H, s), 4.04 (3H, s), 6.94 (1H, s), 6.53 (1H, d, J=3.0 Hz), 7.17 (2H, d, J=9.0 Hz), 7.47 (2H, d, J=9.0 Hz), 7.71 (1H, d, J=3.0 Hz), 8.20 (1H, s), 9.12 (1H, br s), 9.64 (1H, br s).

### Example 8

### N-{2-chloro-4-[(5-methyl-5H-pyrrolo[3,2-d]pyrimidin-4-yl)oxy]phenyl}-N′-(5-methylisoxazol-3-yl)urea

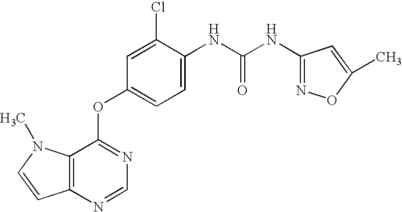

To a solution of 3-amino-5-methylisoxazole (65 mg, 0.66 mmol) and pyridine (210 mg, 2.65 mmol) in N,N-dimethylacetamide (1 mL) was added phenyl chloroformate (83 μL, 0.66 mmol) with stirring under ice-cooling, and the reaction mixture was stirred at room temperature for 2 hr. 2-Chloro-4-[(5-methyl-5H-pyrrolo[3,2-d]pyrimidin-4-yl)oxy]aniline (91 mg, 0.33 mmol) was added to the reaction mixture, and the mixture was stirred at 90° C. for 7 hr. The reaction mixture was diluted with water (20 mL), basified with 1N sodium hydroxide solution, and extracted with ethyl acetate (20 mL, 10 mL×2). The organic layer was washed with saturated brine (10 mL), and concentrated under reduced pressure. The residue was purified by column chromatography (NH silica gel, ethyl acetate/methanol=100/0→90/10) and recrystallized from ethyl acetate to give the title compound (71 mg, 54%) as a white solid.

¹H-NMR (DMSO-d₆, 300 MHz) δ 2.38 (3H, s), 4.10 (3H, s), 6.51 (1H, s), 6.61 (1H, d, J=3.0 Hz), 7.32 (1H, dd, J=9.0, 2.6 Hz), 7.58 (1H, d, J=2.6 Hz), 7.80 (1H, d, J=3.0 Hz), 8.17 (1H, d, J=9.0 Hz), 8.30 (1H, s), 8.77 (1H, br s), 10.17 (1H, br s).

### Example 9

### N-{2-chloro-4-[(5-ethyl-5H-pyrrolo[3,2-d]pyrimidin-4-yl)oxy]phenyl}-N′-(5-methylisoxazol-3-yl)urea

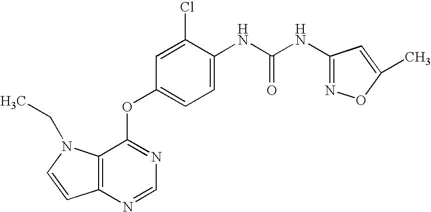

Using 3-amino-5-methylisoxazole (196 mg, 2.0 mmol), pyridine (633 mg, 8.0 mmol), N,N-dimethylacetamide (2 mL), phenyl chloroformate (251 μL, 2.0 mmol) and 2-chloro-4-[(5-ethyl-5H-pyrrolo[3,2-d]pyrimidin-4-yl)oxy]aniline (289 mg, 1.0 mmol) as starting materials, and in the same manner as in Example 8, the title compound (286 mg, 69%) was obtained as a white solid.

¹H-NMR (CDCl₃, 300 MHz) δ 1.54 (3H, t, J=7.1 Hz), 2.43 (3H, s), 4.48 (2H, q, J=7.1 Hz), 6.04 (1H, s), 6.67 (1H, d, J=3.0 Hz), 7.20 (1H, dd, J=9.0, 2.7 Hz), 7.36 (1H, d, J=2.7 Hz), 7.41 (1H, d, J=3.0 Hz), 8.40 (1H, d, J=9.0 Hz), 8.46 (1H, s), 8.64 (1H, br s), 9.48 (1H, br s).

### Example 10

### N-{4-[(5-methyl-5H-pyrrolo[3,2-d]pyrimidin-4-yl)oxy]phenyl}-N′-propylurea

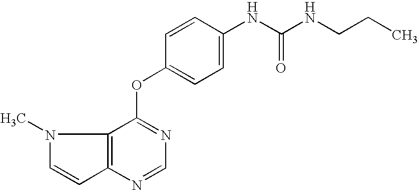

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), n-propylamine (38.7 mg, 0.655 mmol) and N,N-dimethylformamide (3 mL) as starting materials, and in the same manner as in Example 2, the title compound (34.5 mg, 17%) was obtained as a white solid.

¹H-NMR (DMSO-d₆, 300 MHz) δ 0.86 (3H, t, J=7.5 Hz), 1.37-1.46 (2H, m), 2.99-3.06 (2H, m), 4.07 (3H, s), 6.15 (1H, t, J=6.0 Hz), 6.55 (1H, d, J=3.0 Hz), 7.13 (2H, d, J=8.7 Hz), 7.43 (2H, d, J=8.7 Hz), 7.74 (1H, d, J=3.0 Hz), 8.23 (1H, s), 8.48 (1H, br s).

### Example 11

### N-(2-chlorophenyl)-N′-{4-[(5-methyl-5H-pyrrolo[3,2-d]pyrimidin-4-yl)oxy]phenyl}urea

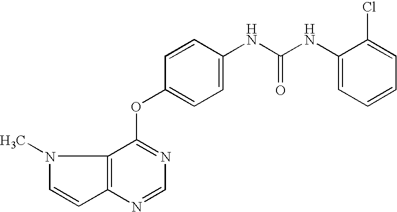

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), 2-chloroaniline (63.6 mg, 0.655 mmol) and N,N-dimethylformamide (3 mL) as starting materials, and in the same manner as in Example 2, the title compound (16.5 mg, 7.0%) was obtained as a white solid.

¹H-NMR (DMSO-d₆, 200 MHz) δ 4.05 (3H, s), 6.53 (1H, d, J=2.9 Hz), 6.94-7.01 (1H, m), 7.16-7.28 (3H, m), 7.38-7.50 (3H, m), 7.72 (1H, d, J=2.9 Hz), 8.09-8.13 (1H, m), 8.21 (1H, s), 8.32 (1H, br s), 9.56 (1H, br s).

### Example 12

### N-(3-bromophenyl)-N′-{4-[(5-methyl-5H-pyrrolo[3,2-d]pyrimidin-4-yl)oxy]phenyl}urea

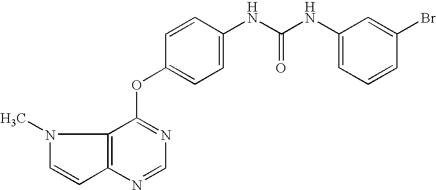

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), 3-bromoaniline (113 mg, 0.655 mmol) and N,N-dimethylformamide (3 mL) as starting materials, and in the same manner as in Example 2, the title compound (42.6 mg, 16%) was obtained as a white solid.

¹H-NMR (DMSO-d₆, 300 MHz) δ 4.08 (3H, s), 6.57 (1H, d, J=3.0 Hz), 7.13 (1H, d, J=8.0 Hz), 7.20-7.24 (3H, m), 7.31 (1H, d, J=8.0 Hz), 7.51 (2H, d, J=9.0 Hz), 7.75 (1H, d, J=3.0 Hz), 7.85 (1H, t, J=2.1 Hz), 8.25 (1H, s), 8.88 (1H, br s), 8.95 (1H, br s).

### Example 13

### N-benzyl-N′-{4-[(5-methyl-5H-pyrrolo[3,2-d]pyrimidin-4-yl)oxy]phenyl}urea

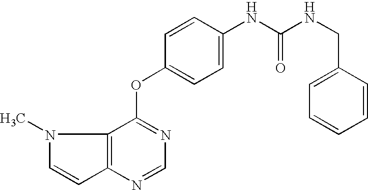

Using 4-[(5-methyl-5H-pyrrolo[3,2-d]pyrimidin-4-yl)oxy]aniline (150 mg, 0.624 mmol), N,N′-carbonyldiimidazole (101 mg, 0.624 mmol), benzylamine (70.2 mg, 0.655 mmol) and N,N-dimethylformamide (3 mL) as starting materials, and in the same manner as in Example 2, the title compound (34.3 mg, 15%) was obtained as a white solid.

¹H-NMR (DMSO-d₆, 300 MHz) δ 4.07 (3H, s), 4.29 (2H, d, J=6.0 Hz), 6.56 (1H, d, J=3.2 Hz), 6.64 (1H, t, J=6.0 Hz), 7.14 (2H, d, J=9.2 Hz), 7.22-7.35 (5H, m), 7.45 (2H, d, J=9.2 Hz), 7.74 (1H, d, J=3.2 Hz), 8.23 (1H, s), 8.65 (1H, br s).

### Example 14

### N-[2-chloro-4-({5-[2-(2-hydroxyethoxy)ethyl]-5H-pyrrolo[3,2-d]pyrimidin-4-yl}oxy)phenyl]-N′-[3-(trifluoromethyl)phenyl]urea

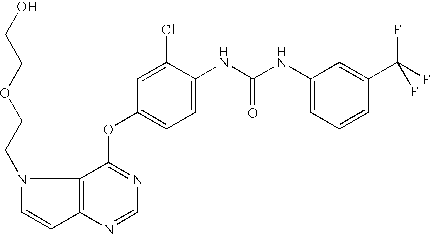

To a solution of 2-{2-[4-(4-amino-3-chlorophenoxy)-5H-pyrrolo[3,2-d]pyrimidin-5-yl]ethoxy}ethyl benzoate (260 mg, 0.463 mmol) and pyridine (83.0 μL, 1.02 mmol) in N,N-dimethylacetamide (3 mL) was added phenyl chloroformate (65.0 μL, 0.510 mmol) with stirring under ice-cooling, and the mixture was stirred at room temperature for 1 hr. 3-(Trifluoromethyl)aniline (89.7 mg, 0.556 mmol) was added to the reaction mixture, and the mixture was stirred at 90° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was dissolved in 0.5N-sodium hydroxide methanol solution (2 mL), and the mixture was stirred at room temperature for 30 min. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=100/0) and recrystallized from ethyl acetate to give the title compound (41.7 mg, 17%) as a white solid melting point 170-174° C.

¹H-NMR (DMSO-d₆, 300 MHz) δ 3.40-3.41 (4H, m), 3.81-3.85 (2H, m), 4.56-4.59 (3H, m), 6.60 (1H, d, J=3.0 Hz), 7.27-7.34 (2H, m), 7.49-7.56 (3H, m), 7.83 (1H, d, J=3.0 Hz), 8.03 (1H, br s), 8.14 (1H, d, J=8.7 Hz), 8.29 (1H, s), 8.44 (1H, s), 9.71 (1H, br s).

### Example 15

### N-{2-chloro-4-[(5-methyl-5H-pyrrolo[3,2-d]pyrimidin-4-yl)oxy]phenyl}-N′-[3-(trifluoromethyl)phenyl]urea

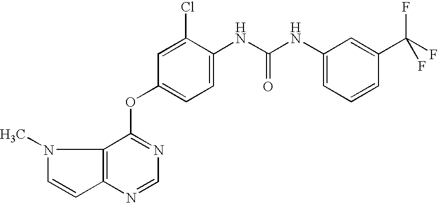

To a solution of 2-chloro-4-[(5-methyl-5H-pyrrolo[3,2-d]pyrimidin-4-yl)oxy]aniline (200 mg, 0.728 mmol), triethylamine (454 μL, 3.28 mmol) in tetrahydrofuran (5 mL) was added 3-(trifluoromethyl)phenylisocyanate (204 mg, 1.09 mmol), and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=30/70→100/0) and recrystallized from ethyl acetate-hexane to give the title compound (206 mg, 61%) as a white solid melting point 192-194° C.

¹H-NMR (DMSO-d₆, 300 MHz) δ 4.11 (3H, s), 6.61 (1H, d, J=3.0 Hz), 7.30-7.36 (2H, m), 7.52-7.58 (3H, m), 7.80 (1H, d, J=3.0 Hz), 8.06 (1H, br s), 8.18 (1H, d, J=8.7 Hz), 8.31 (1H, s), 8.45 (1H, br s), 9.73 (1H, br s).

### Example 16

### N-[2-chloro-4-({5-[2-(2-methoxyethoxy)ethyl]-5H-pyrrolo[3,2-d]pyrimidin-4-yl}oxy)phenyl]-N′-(5-methylisoxazol-3-yl)urea

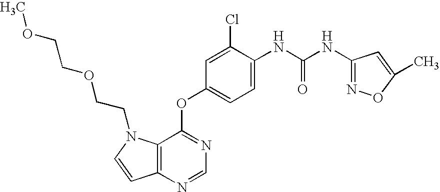

To a solution of 3-amino-5-methylisoxazole (196 mg, 2.0 mmol) and pyridine (633 mg, 8.0 mmol) in N,N-dimethylacetamide (2 mL) was added phenyl chloroformate (0.251 mL, 2.0 mmol) with stirring under ice-cooling, and the reaction mixture was stirred at room temperature for 2 hr. 2-Chloro-4-({5-[2-(2-methoxyethoxy)ethyl]-5H-pyrrolo[3,2-d]pyrimidin-4-yl}oxy)aniline (290 mg, 0.80 mmol) was added to the reaction mixture, and the mixture was stirred at 90° C. for 18 hr. The reaction mixture was diluted with 1N aqueous sodium hydroxide solution (15 mL), and extracted with ethyl acetate (20 mL, 10 mLx²). The organic layer was washed with saturated brine, and concentrated under reduced pressure. The residue was purified by column chromatography (NH silica gel, ethyl acetate/methanol=100/0→90/10) and recrystallized from ethyl acetate to give the title compound (190 mg, 49%) as a white solid.

¹H-NMR (DMSO-d₆, 300 MHz) δ 2.38 (3H, s), 3.15 (3H, s), 3.30-3.40 (2H, m), 3.45-3.55 (2H, m), 3.84 (2H, t, J=5.3 Hz), 4.59 (2H, t, J=5.3 Hz), 6.52 (1H, s), 6.63 (1H, d, J=3.2 Hz), 7.32 (1H, dd, J=9.0, 2.8 Hz), 7.57 (1H, d, J=2.8 Hz), 7.83 (1H, d, J=3.2 Hz), 8.17 (1H, d, J=9.0 Hz), 8.32 (1H, s), 8.74 (1H, br s), 10.14 (1H, br s).

### Example 17

### N-(3-fluorophenyl)-N′-{4-[(5-methyl-5H-pyrrolo[3,2-d]pyrimidin-4-yl)oxy]phenyl}urea

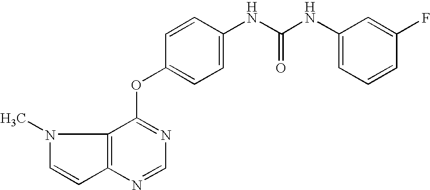

Using 4-[(5-methyl-5H-pyrrolo[3,2-d]pyrimidin-4-yl)oxy]aniline (100 mg, 0.416 mmol), triethylamine (288 μL, 2.08 mmol), 3-fluorophenylisocyanate (69.0 mg, 0.499 mmol), tetrahydrofuran (3 mL) as starting materials, and in the same manner as in Example 15, the title compound (92.1 mg, 59%) was obtained as a white solid.

¹H-NMR (DMSO-d₆, 300 MHz) δ 4.08 (3H, s), 6.56-6.57 (1H, m), 6.73-6.57 (1H, m), 7.10-7.13 (1H, m), 7.20-7.32 (3H, m), 7.46-7.52 (3H, m), 7.75 (1H, d, J=3.3 Hz), 8.25 (1H, s), 8.81 (1H, s), 8.91 (1H, br s).

### Example 18

### N-(3-methylphenyl)-N′-{4-[(5-methyl-5H-pyrrolo[3,2-d]pyrimidin-4-yl)oxy]phenyl}urea

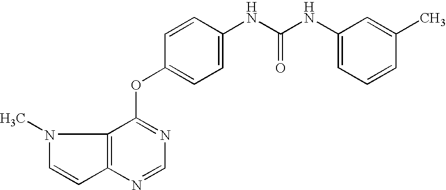

Using 4-[(5-methyl-5H-pyrrolo[3,2-d]pyrimidin-4-yl)oxy]aniline (100 mg, 0.416 mmol), triethylamine (288 μL, 2.08 mmol), 3-tolylisocyanate (65.2 mg, 0.499 mmol), tetrahydrofuran (3 mL) as starting materials, and in the same manner as in Example 15, the title compound (118 mg, 76%) was obtained as a white compound melting point 174-176° C.

¹H-NMR (DMSO-d₆, 300 MHz) δ 2.26 (3H, s), 4.09 (3H, s), 6.57 (1H, d, J=3.2 Hz), 6.77 (1H, d, J=7.2 Hz), 7.11-7.29 (5H, m), 7.48-7.51 (2H, m), 7.75 (1H, d, J=3.2 Hz), 8.25 (1H, s), 8.59 (1H, s), 8.71 (1H, br s).

### Example 19

### N-{4-[(5-methyl-5H-pyrrolo[3,2-d]pyrimidin-4-yl) oxy]phenyl}-N′-[3-(trifluoromethoxy)phenyl]urea

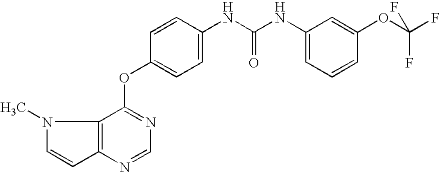

To a solution of 3-(trifluoromethoxy)aniline (110 mg, 0.624 mmol), pyridine (151 μL, 1.87 mmol) in N,N-dimethylacetamide (3 mL) was added phenyl chloroformate (83.0 μL, 0.625 mmol) with stirring under ice-cooling, and the mixture was stirred at room temperature for 1 hr. 4-[(5-Methyl-5H-pyrrolo[3,2-d]pyrimidin-4-yl)oxy]aniline (100 mg, 0.416 mmol) was added to the reaction mixture, and the mixture was stirred at 90° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=10/90→100/0) and recrystallized from ethyl acetate-hexane to give the title compound (74.6 mg, 40%) as a white solid.

¹H-NMR (DMSO-d₆, 300 MHz) δ 4.09 (3H, s), 6.57 (1H, d, J=3.0 Hz), 6.93 (1H, d, J=8.4 Hz), 7.22 (2H, d, J=8.8 Hz), 7.29 (1H, d, J=8.4 Hz), 7.38 (1H, t, J=8.4 Hz), 7.51 (2H, d, J=8.8 Hz), 7.69 (1H, s), 7.75 (1H, d, J=3.0 Hz), 8.25 (1H, s), 8.83 (1H, s), 9.01 (1H, br s).

### Example 20

### N-{2-chloro-4-[(5-methyl-5H-pyrrolo[3,2-d]pyrimidin-4-yl)oxy]phenyl}-N′-[3-(trifluoromethoxy)phenyl]urea

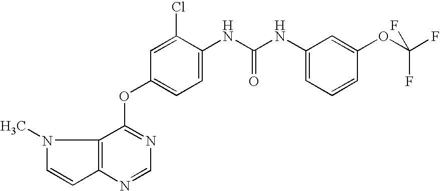

To a solution of 2-chloro-4-[(5-methyl-5H-pyrrolo[3,2-d]pyrimidin-4-yl)oxy]aniline (275 mg, 1.00 mmol) and pyridine (170 μL, 2.10 mmol) in N,N-dimethylacetamide (5 mL) was added phenyl chloroformate (133 μL, 1.05 mmol) with stirring under ice-cooling, and the mixture was stirred at room temperature for 1 hr. 3-(Trifluoromethoxy)aniline (186 mg, 1.05 mmol) was added to the reaction mixture, and the mixture was stirred at 70° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=30/70→100/0) and recrystallized from ethyl acetate-hexane to give the title compound (265 mg, 55%) as a white solid melting point 171-174° C.

¹H-NMR (DMSO-d₆, 300 MHz) δ 4.08 (3H, s), 6.57 (1H, d, J=3.0 Hz), 6.95-6.97 (1H, m), 7.25-7.31 (2H, m), 7.41 (1H, t, J=8.4 Hz), 7.55 (1H, d, J=2.7 Hz), 7.71 (1H, s), 7.78 (1H, d, J=3.0 Hz), 8.14 (1H, d, J=9.0 Hz), 8.28 (1H, s), 8.41 (1H, s), 9.66 (1H, br s).

### Example 21

### N-{2-chloro-4-[(5-methyl-5H-pyrrolo[3,2-d]pyrimidin-4-yl)oxy]phenyl}-N′-(2,2,4,4-tetrafluoro-4H-1,3-benzodioxin-6-yl)urea

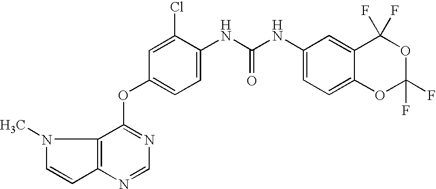

Using 2-chloro-4-[(5-methyl-5H-pyrrolo[3,2-d]pyrimidin-4-yl)oxy]aniline (100 mg, 0.364), triethylamine (252 μL, 1.82 mmol), 2,2,4,4-tetrafluoro-6-isocyanato-4H-1,3-benzodioxin (95.0 mg, 0.382 mmol), tetrahydrofuran (5 mL) as starting materials, and in the same manner as in Example 15, the title compound (78.5 mg, 25%) was obtained as a white solid.

¹H-NMR (DMSO-d₆, 300 MHz) δ 4.08 (3H, s), 6.58 (1H, d, J=3.0 Hz), 7.30 (1H, dd, J=9.0, 2.7 Hz), 7.43 (1H, d, J=9.0 Hz), 7.55 (1H, d, J=2.4 Hz), 7.61 (1H, dd, J=9.0, 2.4 Hz), 7.78 (1H, d, J=3.0 Hz), 8.11-8.14 (2H, m), 8.28 (1H, s), 8.47 (1H, s), 9.75 (1H, br. s).

### Example 22

### N-{3-chloro-4-[(5-methyl-5H-pyrrolo[3,2-d]pyrimidin-4-yl)oxy]phenyl}-N′-[3-(trifluoromethyl)phenyl]urea

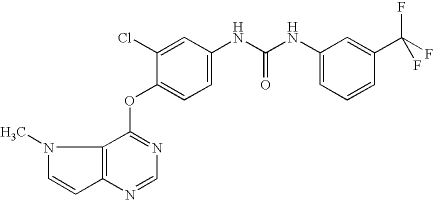

Using 3-chloro-4-[(5-methyl-5H-pyrrolo[3,2-d]pyrimidin-4-yl)oxy]aniline (100 mg, 0.364 mmol), triethylamine (504 μL, 3.64 mmol), 3-(trifluoromethyl)phenylisocyanate (88.4 mg, 0.546 mmol), tetrahydrofuran (3 mL) as starting materials, and in the same manner as in Example 15, the title compound (144 mg, 85%) was obtained as a white solid.

¹H-NMR (DMSO-d₆, 300 MHz) δ 4.10 (3H, s), 6.59-6.60 (1H, m), 7.31 (1H, d, J=8.0 Hz), 7.37-7.43 (2H, m), 7.51 (1H, t, J=8.0 Hz), 7.59 (1H, d, J=8.7 Hz), 7.79 (1H, d, J=3.3 Hz), 7.84-7.85 (1H, m), 8.01 (1H, s), 8.25 (1H, s), 9.06 (1H, s), 9.16 (1H, br s).

### Example 23

### N-(2-chloro-4-{[5-(2-methoxyethyl)-5H-pyrrolo[3,2-d]pyrimidin-4-yl]oxy}phenyl)-N′-[3-(trifluoromethyl)phenyl]urea

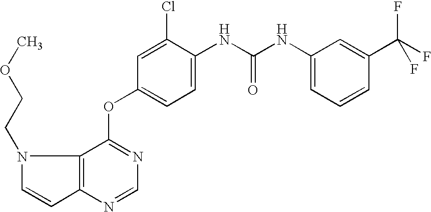

Using 2-chloro-4-{[5-(2-methoxyethyl)-5H-pyrrolo[3,2-d]pyrimidin-4-yl]oxy}aniline (100 mg, 0.314 mmol), triethylamine (998 μL, 7.21 mmol), 3-(trifluoromethyl)phenylisocyanate (127 mg, 0.793 mmol), tetrahydrofuran (3 mL) as starting materials, and in the same manner as in Example 15, the title compound (74.5 mg, 47%) was obtained as a white solid.

¹H-NMR (DMSO-d₆, 300 MHz) δ 3.22 (3H, s), 3.74 (2H, t, J=5.3 Hz), 4.57 (2H, t, J=5.3 Hz), 6.60 (1H, d, J=3.2 Hz), 7.27-7.34 (2H, m), 7.49-7.55 (3H, m), 7.80 (1H, d, J=3.2 Hz), 8.03 (1H, br s), 8.15 (1H, d, J=9.0 Hz), 8.30 (1H, s), 8.43 (1H, s), 9.71 (1H, br s).

### Example 24

### N-(2-chloro-4-{[5-(2-hydroxyethyl)-5H-pyrrolo[3,2-d]pyrimidin-4-yl]oxy}phenyl)-N′-[3-(trifluoromethyl)phenyl]urea

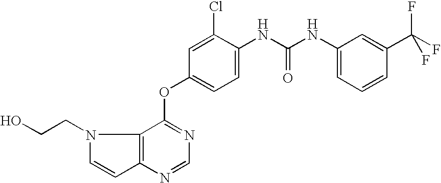

To a solution of 2-[4-(4-amino-3-chlorophenoxy)-5H-pyrrolo[3,2-d]pyrimidin-5-yl]ethyl benzoate (300 mg, 0.734 mmol) and triethylamine (1.00 mL, 7.34 mmol) in tetrahydrofuran (5 mL) was added 3-(trifluoromethyl)phenylisocyanate (130 mg, 0.807 mmol), and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was dissolved in 0.5N-sodium hydroxide methanol solution (2 mL). The mixture was stirred at room temperature for 30 min. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=20/80→100/0) and recrystallized from ethyl acetate to give the title compound (93.4 mg, 26%) as a white solid.

¹H-NMR (DMSO-d₆, 300 MHz) δ 3.76-3.81 (2H, m), 4.45 (2H, t, J=5.4 Hz), 4.91 (1H, t, J=5.4 Hz), 6.59-6.60 (1H, m), 7.27 (1H, dd, J=9.2, 2.7 Hz), 7.32-7.33 (1H, m), 7.49-7.57 (3H, m), 7.79 (1H, d, J=3.0 Hz), 8.03 (1H, br s), 8.15 (1H, d, J=9.2 Hz), 8.29 (1H, s), 8.43 (1H, s), 9.71 (1H, br s).

### Example 25

### N-[2-chloro-4-({5-[2-(2-methoxyethoxy)ethyl]-5H-pyrrolo[3,2-d]pyrimidin-4-yl}oxy)phenyl]-N′-[3-(trifluoromethyl)phenyl]urea

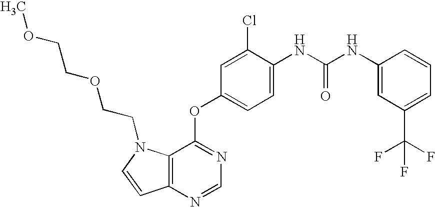

To a solution of 2-chloro-4-({5-[2-(2-methoxyethoxy)ethyl]-5H-pyrrolo[3,2-d]pyrimidin-4-yl}oxy)aniline (181 mg, 0.50 mmol) and triethylamine (0.014 mL, 0.10 mmol) in tetrahydrofuran (10 mL) was added 3-(trifluoromethyl)phenylisocyanate (0.083 mL, 0.60 mmol) with stirring, and the reaction mixture was stirred at room temperature for 18 hr. The reaction mixture was concentrated under reduced pressure, and the residue was purified by column chromatography (NH silica gel, ethyl acetate/methanol=100/0→90/10) and recrystallized from ethyl acetate/diisopropyl ether to give the title compound (159 mg, 58%) as a white solid.

¹H-NMR (DMSO-d₆, 300 MHz) δ 3.15 (3H, s), 3.30-3.40 (2H, m), 3.45-3.55 (2H, m), 3.84 (2H, t, J=5.4 Hz), 4.59 (2H, t, J=5.4 Hz), 6.63 (1H, d, J=3.2 Hz), 7.31 (1H, dd, J=9.1, 2.6 Hz), 7.35 (1H, br d, J=6.9 Hz), 7.50-7.65 (3H, m), 7.83 (1H, d, J=3.2 Hz), 8.06 (1H, br s), 8.17 (1H, d, J=9.1 Hz), 8.32 (1H, s), 8.46 (1H, br s), 9.74 (1H, br s).

### Example 26

### N-{3-fluoro-4-[(5-methyl-5H-pyrrolo[3,2-d]pyrimidin-4-yl)oxy]phenyl}-N′-[3-(trifluoromethyl)phenyl]urea

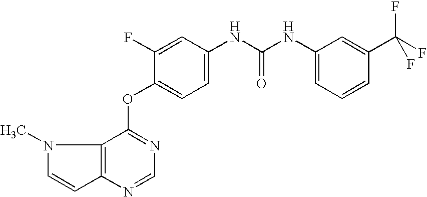

Using 3-fluoro-4-[(5-methyl-5H-pyrrolo[3,2-d]pyrimidin-4-yl)oxy]aniline (130 mg, 0.503 mmol), triethylamine (348 μL, 2.52 mmol), 3-(trifluoromethyl)phenylisocyanate (113 mg, 0.604 mmol), tetrahydrofuran (3 mL) as starting materials, and in the same manner as in Example 15, the title compound (90.0 mg, 52%) was obtained as a white solid.

¹H-NMR (DMSO-d₆, 300 MHz) δ 4.09 (3H, s), 6.59-6.61 (1H, m), 7.22-7.41 (3H, m), 7.48-7.68 (3H, m), 7.80 (1H, d, J=3.3 Hz), 8.00 (1H, s), 8.27 (1H, s), 9.07 (1H, s), 9.15 (1H, s).

### Example 27

### N-{4-[(5-methyl-5H-pyrrolo[3,2-d]pyrimidin-4-yl)oxy]phenyl}-N′-[5-(trifluoromethyl)-1,3,4-thiadiazol-2-yl]urea

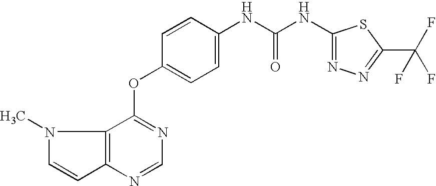

Using 5-(trifluoromethyl)-1,3,4-thiadiazole-2-amine (127 mg, 0.749 mmol), pyridine (151 μL, 1.87 mmol), phenyl chloroformate (97.0 μL, 0.772 mmol), 4-[(5-methyl-5H-pyrrolo[3,2-d]pyrimidin-4-yl)oxy]aniline (150 mg, 0.624 mmol) and N,N-dimethylacetamide (3 mL) as starting materials, and in the same manner as in Example 19, the title compound (130 mg, 48%) was obtained as a white solid.

¹H-NMR (DMSO-d₆, 300 MHz) δ 4.09 (3H, s), 6.57-6.58 (1H, m), 7.29 (2H, d, J=8.3 Hz), 7.56 (2H, d, J=8.3 Hz), 7.76 (1H, d, J=3.0 Hz), 8.25 (1H, d, J=1.2 Hz), 9.32 (1H, s), 11.82 (1H, br s).

### Example 28

### N-{4-[(5-methyl-5H-pyrrolo[3,2-d]pyrimidin-4-yl)oxy]phenyl}-N′-[4-(trifluoromethyl)-1,3-oxazol-2-yl]urea

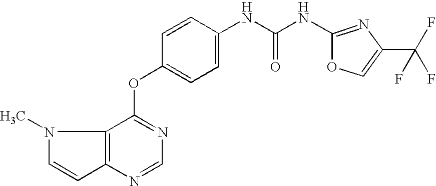

Using 4-(trifluoromethyl)-1,3-oxazole-2-amine (114 mg, 0.749 mmol), pyridine (151 μL, 1.87 mmol), phenyl chloroformate (97.0 μL, 0.772 mmol), 4-[(5-methyl-5H-pyrrolo[3,2-d]pyrimidin-4-yl)oxy]aniline (150 mg, 0.624 mmol) and N,N-dimethylacetamide (3 mL) as starting materials, and in the same manner as in Example 19, the title compound (75.0 mg, 29%) was obtained as a white solid.

¹H-NMR (DMSO-d₆, 300 MHz) δ 4.09 (3H, s), 6.57-6.58 (1H, m), 7.27 (2H, d, J=8.4 Hz), 7.53 (2H, d, J=8.4 Hz), 7.76-7.77 (1H, m), 8.25 (1H, d, J=0.9 Hz), 8.61 (1H, s), 9.57 (1H, s), 10.94 (1H, br. s).

### Example 29

### N-{2-fluoro-4-[(5-methyl-5H-pyrrolo[3,2-d]pyrimidin-4-yl)oxy]phenyl}-N′-[3-(trifluoromethyl)phenyl]urea

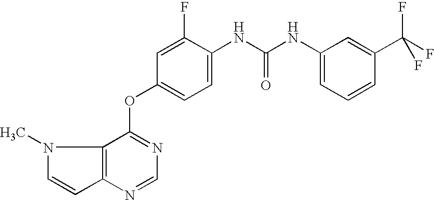

Using 2-fluoro-4-[(5-methyl-5H-pyrrolo[3,2-d]pyrimidin-4-yl)oxy]aniline (100 mg, 0.387 mmol), triethylamine (268 μL, 1.94 mmol), 3-(trifluoromethyl)phenylisocyanate (134 mg, 0.465-mmol) and tetrahydrofuran (3 mL) as starting materials, and in the same manner as in Example 15, the title compound (150 mg, 87%) was obtained as a white solid melting point 196-198° C.

¹H-NMR (DMSO-d₆, 300 MHz) δ 4.10 (3H, s), 6.61 (1H, d, J=3.0 Hz), 7.12-7.19 (1H, m), 7.30-7.44 (2H, m), 7.50-7.60 (2H, m), 7.79 (1H, d, J=3.0 Hz), 8.05 (1H, s), 8.13 (1H, t, J=9.3 Hz), 8.30 (1H, s), 8.67 (1H, s), 9.41 (1H, s).

### Example 30

### N-{2-chloro-4-[(5-methyl-5H-pyrrolo[3,2-d]pyrimidin-4-yl)oxy]phenyl}-N′-[4-(trifluoromethyl)pyridin-2-yl]urea

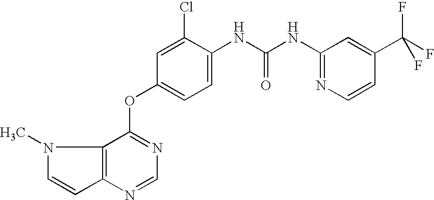

Using 2-chloro-4-[(5-methyl-5H-pyrrolo[3,2-d]pyrimidin-4-yl)oxy]aniline (130 mg, 0.473 mmol), pyridine (138 μL, 1.70 mmol), phenyl chloroformate (75.0 μL, 0.596 mmol), 2-amino-4-(trifluoromethyl)pyridine (92.2 mg, 0.568 mmol) and N,N-dimethylacetamide (3 mL) as starting materials, and in the same manner as in Example 20, the title compound (45.0 mg, 21%) was obtained as a white solid melting point 259-260° C.

¹H-NMR (DMSO-d₆, 300 MHz) δ 4.09 (3H, s), 6.58-6.59 (1H, m), 7.31-7.38 (2H, m), 7.59 (1H, d, J=2.1 Hz), 7.77-7.80 (2H, m), 8.28-8.31 (2H, m), 8.57 (1H, d, J=5.7 Hz), 10.36 (1H, s), 10.61 (1H, br s).

### Example 31

### N-{2-chloro-4-[(5-methyl-5H-pyrrolo[3,2-d]pyrimidin-4-yl)oxy]phenyl}-N′-(3-phenoxyphenyl)urea

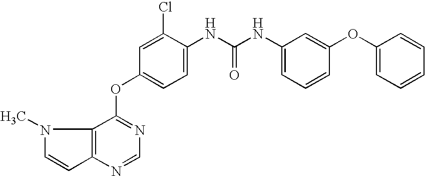

Using 2-chloro-4-[(5-methyl-5H-pyrrolo[3,2-d]pyrimidin-4-yl)oxy]aniline (100 mg, 0.364 mmol), triethylamine (252 μL, 1.82 mmol), 3-phenoxyphenylisocyanate (92.4 mg, 0.437 mmol) and tetrahydrofuran (3 mL) as starting materials, and in the same manner as in Example 15, the title compound (154 mg, 87%) was obtained as a white solid.

¹H-NMR (DMSO-d₆, 300 MHz) δ 4.08 (3H, s), 6.58-6.59 (1H, m), 6.62-6.65 (1H, m), 7.02-7.05 (2H, m), 7.12-7.16 (2H, m), 7.22-7.31 (3H, m), 7.39 (2H, t, J=7.5 Hz), 7.52 (1H, d, J=1.8 Hz), 7.77 (1H, d, J=2.7 Hz), 8.12 (1H, d, J=8.7 Hz), 8.27 (1H, s), 8.31 (1H, s), 9.46 (1H, s).

### Example 32

### N-(3-tert-butylphenyl)-N′-{2-chloro-4-[(5-methyl-5H-pyrrolo[3,2-d]pyrimidin-4-yl)oxy]phenyl}urea

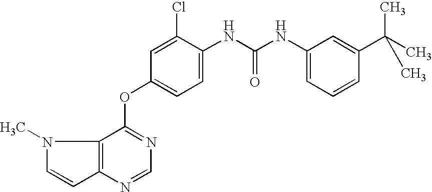

Using 2-chloro-4-[(5-methyl-5H-pyrrolo[3,2-d]pyrimidin-4-yl)oxy]aniline (150 mg, 0.546 mmol), pyridine (132 μL, 1.64 mmol), phenyl chloroformate (752 μL, 0.573 mmol), 3-tert-butylaniline (98.0 mg, 0.655 mmol) and N,N-dimethylacetamide (3 mL) as starting materials, and in the same manner as in Example 20, the title compound (152 mg, 52%) was obtained as a white solid.

¹H-NMR (DMSO-d₆, 300 MHz) δ 1.26 (9H, s), 4.08 (3H, s), 6.58 (1H, d, J=3.0 Hz), 7.02 (1H, d, J=8.9 Hz), 7.20 (1H, t, J=7.5 Hz), 7.25-7.33 (2H, m), 7.44 (1H, s), 7.52 (1H, d, J=3.0 Hz), 7.77 (1H, d, J=3.0 Hz), 8.20 (1H, d, J=8.9 Hz), 8.28-8.31 (2H, m), 9.36 (1H, s).

### Example 33

### N-{2-chloro-4-[(5-methyl-5H-pyrrolo[3,2-d]pyrimidin-4-yl)oxy]phenyl}-N′-[4-chloro-3-(trifluoromethyl)phenyl]urea

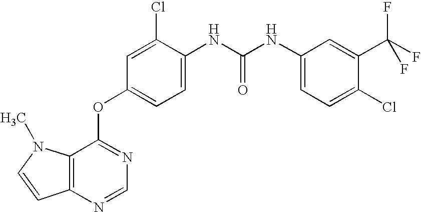

Using 2-chloro-4-[(5-methyl-5H-pyrrolo[3,2-d]pyrimidin-4-yl)oxy]aniline (100 mg, 0.364 mmol), triethylamine (252 μL, 1.82 mmol), 4-chloro-3-(trifluoromethyl)phenylisocyanate (97.4 mg, 0.437 mmol) and tetrahydrofuran (3 mL) as starting materials, and in the same manner as in Example 15, the title compound (107 mg, 59%) was obtained as a white solid.

¹H-NMR (DMSO-d₆, 300 MHz) δ 4.07 (3H, s), 6.57-6.58 (1H, m), 7.27-7.31 (1H, m), 7.54-7.61 (3H, m), 7.76-7.78 (1H, m), 8.10-8.13 (2H, m), 8.27 (1H, d, J=1.5 Hz), 8.45 (1H, s), 9.79 (1H, s).

### Example 34

### N-{4-[(5-methyl-5H-pyrrolo[3,2-d]pyrimidin-4-yl)oxy]-1-naphthyl}-N′-[3-(trifluoromethyl)phenyl]urea

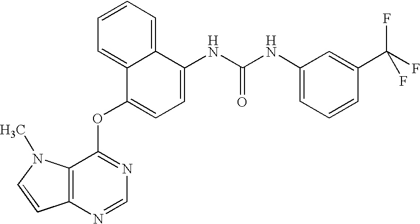

Using 4-[(5-methyl-5H-pyrrolo[3,2-d]pyrimidin-4-yl)oxy]naphthalene-1-amine (29.0 mg, 0.100 mmol), triethylamine (42.2 μL, 0.300 mmol), 3-(trifluoromethyl)phenylisocyanate (22.4 mg, 0.120 mmol) and tetrahydrofuran (2 mL) as starting materials, and in the same: manner as in Example 15, the title compound (11.0 mg, 27%) was obtained as a white solid.

¹H-NMR (DMSO-d₆, 300 MHz) δ 4.21 (3H, s), 6.61 (1H, d, J=3.2 Hz), 7.31 (1H, d, J=7.5 Hz), 7.43 (1H, d, J=8.1 Hz), 7.49-7.55 (2H, m), 7.60-7.66 (2H, m), 7.83 (1H, d, J=3.2 Hz), 7.90-7.95 (2H, m), 8.07 (1H, br s), 8.15-8.17 (2H, m), 8.92 (1H, s), 9.40 (1H, s).

### Example 35

### N-{8-[(5-methyl-5H-pyrrolo[3,2-d]pyrimidin-4-yl)oxy]quinolin-5-yl}-N′-[3-(trifluoromethyl)phenyl]urea

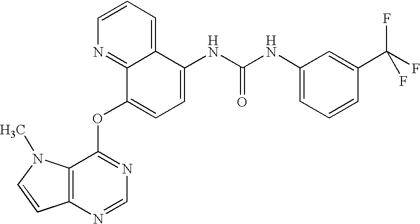

Using 8-[(5-methyl-5H-pyrrolo[3,2-d]pyrimidin-4-yl)oxy]quinoline-5-amine (100 mg, 0.364 mmol), triethylamine (252 μL, 1.82 mmol), 3-(trifluoromethyl)phenylisocyanate (97.4 mg, 0.437 mmol) and tetrahydrofuran (3 mL) as starting materials, and in the same manner as in Example 15, the title compound (107 mg, 59%) was obtained as a white solid.

¹H-NMR (DMSO-d₆, 300 MHz) δ 4.18 (3H, s), 6.59 (1H, d, J=3.0 Hz), 7.32 (1H, d, J=7.7 Hz), 7.52 (1H, t, J=7.7 Hz), 7.58-7.63 (2H, m), 7.72 (1H, d, J=8.3 Hz), 7.79 (1H, d, J=3.0 Hz), 7.98 (1H, d, J=8.3 Hz), 8.06-8.07 (2H, m), 8.50-8.53 (1H, m), 8.75-8.76 (1H, m), 9.02 (1H, s), 9.37 (1H, s).

### Example 36

### N-{2-chloro-4-[(5-methyl-5H-pyrrolo[3,2-d]pyrimidin-4-yl)oxy]phenyl}-N′-[3-(1,1,2,2-tetrafluoroethoxy)phenyl]urea

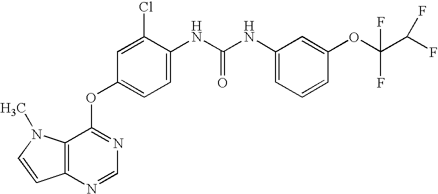

Using 3-(1,1,2,2-tetrafluoroethoxy)aniline (129 mg, 0.617 mmol), phenyl chloroformate (81.0 μL, 0.648 mmol), pyridine (150 μL, 1.85 mmol), 2-chloro-4-[(5-methyl-5H-pyrrolo[3,2-d]pyrimidin-4-yl)oxy]aniline (141 mg, 0.514 mmol) and N,N-dimethylacetamide (3 mL) as starting materials, and in the same manner as in Example 19, the title compound (110 mg, 42%) was obtained as a white solid.

¹H-NMR (DMSO-d₆, 300 MHz) δ 4.08 (3H, s), 6.58-6.59 (1H, m), 6.78-6.80 (1H, m), 6.86-6.89 (1H, m), 7.22-7.24 (1H, m), 7.29 (1H, dd, J=8.9, 3.0 Hz), 7.38 (1H, t, J=8.1 Hz), 7.54-7.55 (1H, m), 7.66 (1H, s), 7.77 (1H, d, J=3.0 Hz), 8.15 (1H, d, J=8.9 Hz), 8.28 (1H, s), 8.39 (1H, s), 9.62 (1H, s).

### Example 37

### N-{2-chloro-4-[(5-methyl-5H-pyrrolo[3,2-d]pyrimidin-4-yl)oxy]phenyl}-N′-[6-(trifluoromethyl)pyridin-2-yl]urea

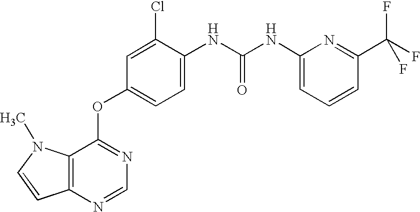

Using 2-amino-6-(trifluoromethyl)pyridine (71.0 mg, 0.437 mmol), phenyl chloroformate (58.0 μL, 0.459 mmol), pyridine (106 μL, 1.31 mmol), 2-chloro-4-[(5-methyl-5H-pyrrolo[3,2-d]pyrimidin-4-yl)oxy]aniline (100 mg, 0.364 mmol) and N,N-dimethylacetamide (3 mL) as starting materials, and in the same manner as in Example 19, the title compound (74.0 mg, 44%) was obtained as a white solid.

¹H-NMR (DMSO-d₆, 300 MHz) δ 4.09 (3H, s), 6.58-6.59 (1H, m), 7.33 (1H, dd, J=9.3, 2.4 Hz), 7.51 (1H, d, J=8.0 Hz), 7.57-7.58 (1H, m), 7.77 (1H, d, J=2.7 Hz), 7.86 (1H, d, J=8.0 Hz), 8.03 (1H, t, J=8.0 Hz), 8.23 (1H, d, J=9.3 Hz), 8.28 (1H, d, J=0.9 Hz), 9.78 (1H, br s), 10.45 (1H, s).

### Example 38

### N-{2-chloro-4-[(5-methyl-5H-pyrrolo[3,2-d]pyrimidin-4-yl)oxy]phenyl}-N′-[3-(1H-imidazol-1-yl)phenyl]urea

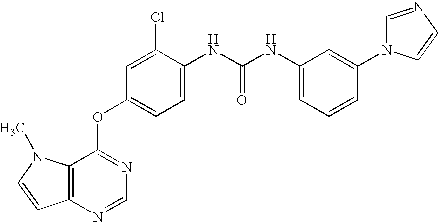

Using 2-chloro-4-[(5-methyl-5H-pyrrolo[3,2-d]pyrimidin-4-yl)oxy]aniline (150 mg, 0.606 mmol), pyridine (147 μL, 1.82 mmol), phenyl chloroformate (80.0 μL, 0.636 mmol), 3-(1H-imidazol-1-yl)aniline (144 mg, 0.909 mmol) and N,N-dimethylacetamide (3 mL) as starting materials, and in the same manner as in Example 20, the title compound (90.0 mg, 33%) was obtained as a white solid.

¹H-NMR (DMSO-d₆, 300 MHz) δ 4.08 (3H, s), 6.58-6.59 (1H, m), 7.10 (1H, d, J=0.9 Hz), 7.23-7.31 (2H, m), 7.37-7.46 (2H, m), 7.54-7.55 (1H, m), 7.65 (1H, d, J=0.9 Hz), 7.74-7.78 (2H, m), 8.15-8.18 (2H, m), 8.28 (1H, d, J=0.9 Hz), 8.45 (1H, s), 9.59 (1H, s).

### Example 39

### N-[2-chloro-4-(5H-pyrrolo[3,2-d]pyrimidin-4-yloxy)phenyl]-N′-[3-(trifluoromethyl)phenyl]urea

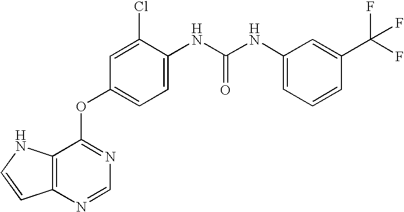

To a solution of 2-chloro-4-(5H-pyrrolo[3,2-d]pyrimidin-4-yloxy)aniline (1043 mg, 4.0 mmol) and triethylamine (320 μL, 2.3 mmol) in tetrahydrofuran (160 mL) was added 3-(trifluoromethyl)phenylisocyanate (661 μL, 4.8 mmol), and the mixture was stirred at room temperature for 18 hr. The reaction mixture was concentrated under reduced pressure, and the residue was purified by column chromatography (silica gel, hexane/ethyl acetate=70/40→0/100) and recrystallized from ethyl acetate to give the title compound (1190 mg, 66%). melting point 316-319° C.

¹H-NMR (DMSO-d₆, 300 MHz) δ 6.65 (1H, d, J=3.0 Hz), 7.30 (1H, dd, J=9.2, 2.7 Hz), 7.35 (1H, br d, J=6.6 Hz), 7.52-7.60 (3H, m), 7.82 (1H, d, J=3.0 Hz), 8.06 (1H, br s), 8.16 (1H, d, J=9.2 Hz), 8.34 (1H, s), 8.47 (1H, br s), 9.75 (1H, br s), 12.35 (1H, br s).

### Example 40

### N-{2-chloro-4-[(5-methyl-5H-pyrrolo[3,2-d]pyrimidin-4-yl)oxy]phenyl}-N′-[2-fluoro-5-(trifluoromethyl)phenyl]urea

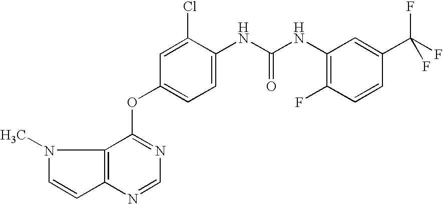

Using 2-fluoro-5-(trifluoromethyl)aniline (260 mg, 1.45 mmol), pyridine (363 μL, 4.50 mmol), phenyl chloroformate (193 μL, 1.53 mmol), 2-chloro-4-[(5-methyl-5H-pyrrolo[3,2-d]pyrimidin-4-yl)oxy]aniline (300 mg, 1.21 mmol) and N-methylpyrrolidone (3 mL) as starting materials, and in the same manner as in Example 19, the title compound (195 mg, 34%) was obtained as a white solid. melting point 193-197° C.

¹H-NMR (DMSO-d₆, 300 MHz) δ 4.11 (3H, s), 6.60-6.61 (1H, m), 7.32 (1H, dd, J=9.0, 2.7 Hz), 7.38-7.46 (1H, m), 7.49-7.55 (1H, m), 7.58 (1H, d, J=2.7 Hz), 7.80 (1H, d, J=3.0 Hz), 8.19 (1H, d, J=9.0 Hz), 8.30 (1H, s), 8.62-8.68 (1H, m), 8.98 (1H, br s), 9.66 (1H, br s).

### Example 41

### N-{2-methyl-4-[(5-methyl-5H-pyrrolo[3,2-d]pyrimidin-4-yl)oxy]phenyl}-N′-[3-(trifluoromethyl)phenyl]urea

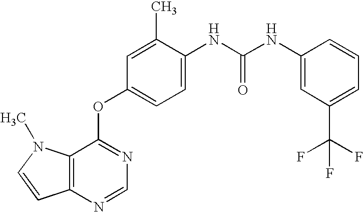

To a solution of 2-methyl-4-[(5-methyl-5H-pyrrolo[3,2-d]pyrimidin-4-yl)oxy]aniline (254 mg, 1.0 mmol) and triethylamine (4.0 μL, 0.3 mmol) in tetrahydrofuran (20 mL) was added 3-(trifluoromethyl)phenylisocyanate (165 μL, 1.2 mmol), and the mixture was stirred at room temperature for 18 hr. The reaction mixture was concentrated under reduced pressure, and the residue was purified by column chromatography (silica gel, ethyl acetate/methanol=100/0→80/20) and recrystallized from ethyl acetate to give the title compound (323 mg, 73%) as a white solid. melting point 213-215° C.

¹H-NMR (DMSO-d₆, 300 MHz) δ 2.28 (3H, s), 4.11 (3H, s), 6.59 (1H, d, J=3.2 Hz), 7.11 (1H, dd, J=8.8, 2.6 Hz), 7.17 (1H, d, J=2.6 Hz), 7.31 (1H, br d, J=7.2 Hz), 7.50-7.60 (2H, m), 7.78 (1H, d, J=3.2 Hz), 7.81 (1H, d, J=8.8 Hz), 8.05 (1H, br s), 8.11 (1H, br s), 8.28 (1H, s), 9.37 (1H, br s).

### Example 42

### N-{2-chloro-4-[(5-methyl-5H-pyrrolo[3,2-d]pyrimidin-4-yl)oxy]phenyl}-N′-[3-(difluoromethoxy)phenyl]urea

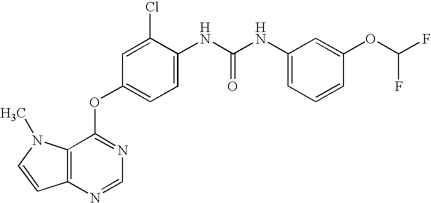

To a solution of 3-(difluoromethoxy)aniline (318 mg, 2.0 mmol) and pyridine (633 mg, 8.0 mmol) in N-methylpyrrolidone (2 mL) was added phenyl chloroformate (251 μL, 2.0 mmol) under ice-cooling, and the mixture was stirred at room temperature for 2 hr. 2-chloro-4-[(5-methyl-5H-pyrrolo[3,2-d]pyrimidin-4-yl)oxy]aniline (275 mg, 1.0 mmol) was added to the reaction mixture, and the mixture was stirred at 80° C. for 8 hr. The reaction mixture was diluted with water, basified with 1N aqueous sodium hydroxide solution and extracted with ethyl acetate. The organic layer was washed with water and saturated brine, and concentrated under reduced pressure. The residue was purified by NH silica gel column chromatography (hexane/ethyl acetate=80/20→0/100), and then silica gel column chromatography (hexane/ethyl acetate=80/20→0/100) and recrystallized from diisopropyl ether to give the title compound (229 mg, 50%) as a white solid.

¹H-NMR (DMSO-d₆, 300 MHz) δ 4.11 (3H, s), 6.61 (1H, d, J=3.0 Hz), 6.81 (1H, dd, J=8.0, 2.1 Hz), 7.19-7.23 (1H, m), 7.22 (1H, t, J=74.1 Hz), 7.31 (1H, dd, J=9.0, 2.7 Hz), 7.35 (1H, t, J=8.0 Hz), 7.49 (1H, t, J=2.1 Hz), 7.56 (1H, d, J=2.7 Hz), 7.80 (1H, d, J=3.0 Hz), 8.17 (1H, d, J=9.0 Hz), 8.30 (1H, s), 8.43 (1H, br s), 9.60 (1H, br s).

### Example 43

### N-{2-chloro-4-[(5-methyl-5H-pyrrolo[3,2-d]pyrimidin-4-yl)oxy]phenyl}-N′-[3-(trifluoromethyl)phenyl]urea hydrochloride

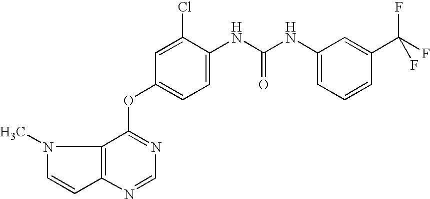

N-{2-Chloro-4-[(5-methyl-5H-pyrrolo[3,2-d]pyrimidin-4-yl)oxy]phenyl}-N′-[3-(trifluoromethyl)phenyl]urea (2.76 g, 5.98 mmol) was dissolved in a mixed solvent of ethyl acetate (55 mL) and ethanol (2.5 mL) at 70° C., and 1N hydrogen chloride-ethyl acetate solution (6.0 mL) was added dropwise. The solvent was evaporated under reduced pressure, and the residue was recrystallized from methanol to give the title compound (1.64 g, 55%) as a white solid.

¹H-NMR (DMSO-d₆, 300 MHz) δ 4.18 (3H, s), 6.77-6.79 (1H, m), 7.32-7.39 (2H, m), 7.54 (1H, t, J=8.1 Hz), 7.61-7.64 (2H, m), 8.06 (1H, s), 8.11 (1H, d, J=3.0 Hz), 8.22 (1H, d, J=9.0 Hz), 8.70-8.80 (2H, m), 10.37 (1H, br s).

### Example 44

### N-{2-chloro-4-[(5-methyl-5H-pyrrolo[3,2-d]pyrimidin-4-yl)oxy]phenyl}-N′-[3-(trifluoromethyl)phenyl]urea p-toluenesulfonic Acid Salt

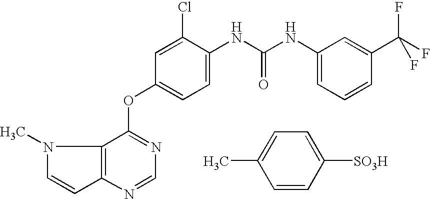

N-{2-Chloro-4-[(5-methyl-5H-pyrrolo[3,2-d]pyrimidin-4-yl)oxy]phenyl}-N′-[3-(trifluoromethyl)phenyl]urea (500 mg, 1.08 mmol) was dissolved in ethyl acetate (10 mL) at 70° C., and 0.5N p-toluenesulfonic acid acetone solution (2.16 mL) was added dropwise. The solvent was evaporated under reduced pressure, and the residue was recrystallized from methanol to give the title compound (445 mg, 65%) as a white solid.

¹H-NMR (DMSO-d₆, 300 MHz) δ 2.29 (3H, s), 4.17 (3H, s), 6.75-6.76 (1H, m), 7.11 (2H, d, J=8.1 Hz), 7.34-7.39 (2H, m), 7.48 (2H, d, J=8.1 Hz), 7.52-7.60 (2H, m), 7.64 (1H, d, J=2.1 Hz), 8.06-8.07 (2H, m), 8.22 (1H, d, J=9.0 Hz), 8.51 (1H, s), 8.68 (1H, br s), 9.76 (1H, br s).

### Example 45

### N-{2-chloro-4-[(5-methyl-5H-pyrrolo[3,2-d]pyrimidin-4-yl)oxy]phenyl}-N′-[4-fluoro-3-(trifluoromethyl)phenyl]urea

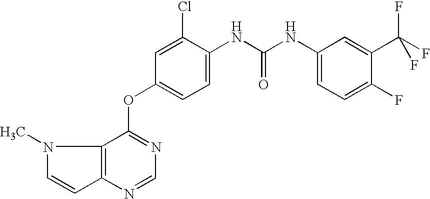

Using 4-fluoro-3-(trifluoromethyl)aniline (183 mg, 1.02 mmol), pyridine (247 μL, 3.06 mmol), phenyl chloroformate (135 μL, 1.07 mmol), 2-chloro-4-[(5-methyl-5H-pyrrolo[3,2-d]pyrimidin-4-yl)oxy]aniline (280 mg, 1.02 mmol) and N-methylpyrrolidone (3 mL) as starting materials, and in the same manner as in Example 19, the title compound (195 mg, 40%) was obtained as a white solid.

¹H-NMR (DMSO-d₆, 200 MHz) δ 4.10 (3H, s), 6.61 (1H, d, J=3.2 Hz), 7.32 (1H, dd, J=9.1, 2.8 Hz), 7.42-7.51 (1H, m), 7.56-7.69 (2H, m), 7.80 (1H, d, J=3.2 Hz), 8.03 (1H, dd, J=6.4, 2.6 Hz), 8.15 (1H, d, J=9.1 Hz), 8.30 (1H, s), 8.44 (1H, br s), 9.71 (1H, br s).

### Example 46

### N-{2-chloro-4-[(5-methyl-5H-pyrrolo[3,2-d]pyrimidin-4-yl)oxy]phenyl}-N′-{3-[(trifluoromethyl)thio]phenyl}urea

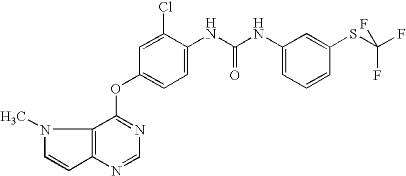

Using 2-chloro-4-[(5-methyl-5H-pyrrolo[3,2-d]pyrimidin-4-yl)oxy]aniline (712 mg, 2.59 mmol), 3-[(trifluoromethyl)thio]phenylisocyanate (500 zL, 3.11 mmol), triethylamine (1.08 mL, 7.77 mmol) and tetrahydrofuran (10 mL) as starting materials, and in the same manner as in Example 15, the title compound (803 mg, 63%) was obtained as a white solid.

¹H-NMR (DMSO-d₆, 300 MHz) δ 4.10 (3H, s), 6.61 (1H, d, J=3.0 Hz), 7.30-7.35 (2H, m), 7.48 (1H, t, J=7.8 Hz), 7.54-7.57 (2H, m), 7.80 (1H, d, J=3.0 Hz), 8.01 (1H, s), 8.17 (1H, d, J=9.0 Hz), 8.30 (1H, s), 8.44 (1H, br s), 9.68 (1H, br s).

### Example 47

### N-{2-chloro-4-[(5-ethyl-5H-pyrrolo[3,2-d]pyrimidin-4-yl)oxy]phenyl}-N′-[3-(trifluoromethyl)phenyl]urea

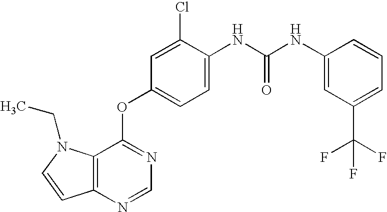

To a solution of 2-chloro-4-[(5-ethyl-5H-pyrrolo[3,2-d]pyrimidin-4-yl)oxy]aniline (289 mg, 1.0 mmol) and triethylamine (40 μL, 0.3 mmol) in tetrahydrofuran (20 mL) was added 3-(trifluoromethyl)phenylisocyanate (165 μL, 1.2 mmol), and the mixture was stirred at room temperature for 18 hr. The reaction mixture was concentrated under reduced pressure, and the residue was purified by column chromatography (silica gel, hexane/ethyl acetate=80/20→0/100) and recrystallized from ethyl acetate/diisopropyl ether to give the title compound (354 mg, 74%) as a white solid. melting point 172-176° C.

¹H-NMR (DMSO-d₆, 300 MHz) δ 1.47 (3H, t, J=7.1 Hz), 4.47 (2H, q, J=7.1 Hz), 6.63 (1H, d, J=3.0 Hz), 7.32 (1H, dd, J=9.0, 2.6 Hz), 7.35 (1H, br d, J=7.2 Hz), 7.16-7.60 (2H, m), 7.58 (1H, d, J=2.6 Hz), 7.89 (1H, d, J=3.0 Hz), 8.06 (1H, br s), 8.17 (1H, d, J=9.0 Hz), 8.32 (1H, s), 8.48 (1H, br s), 9.75 (1H, br s).

### Example 48

### N-{2-chloro-4-[(5-isopropyl-5H-pyrrolo[3,2-d]pyrimidin-4-yl)oxy]phenyl}-N′-[3-(trifluoromethyl)phenyl]urea

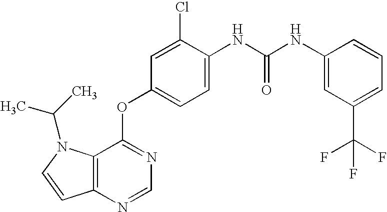

Using 2-chloro-4-[(5-isopropyl-5H-pyrrolo[3,2-d]pyrimidin-4-yl)oxy]aniline (303 mg, 1.0 mmol), triethylamine (40 μL, 0.3 mmol), tetrahydrofuran (20 mL) and 3-(trifluoromethyl)phenylisocyanate (165 μL, 1.2 mmol), and in the same manner as in Example 47, the title compound (368 mg, 75%) was obtained as a white solid.

¹H-NMR (DMSO-d₆, 300 MHz) δ 1.56 (6H, d, J=6.7 Hz), 5.19 (1H, sept, J=6.7 Hz), 6.67 (1H, d, J=3.0 Hz), 7.31 (1H, dd, J=9.0, 2.7 Hz), 7.35 (1H, br d, J=8.4 Hz), 7.52-7.60 (2H, m), 7.58 (1H, d, J=2.7 Hz), 8.02 (1H, d, J=3.0 Hz), 8.06 (1H, br s), 8.17 (1H, d, J=9.0 Hz), 8.32 (1H, s), 8.48 (1H, br s), 9.75 (1H, br s).

### Example 49

### N-{2-fluoro-4-[(5-methyl-5H-pyrrolo[3,2-d]pyrimidin-4-yl)oxy]phenyl}-N′-[3-(trifluoromethyl)phenyl]urea

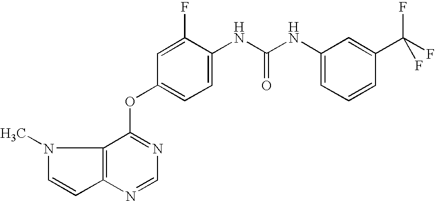

To a solution of 2-fluoro-4-[(5-methyl-5H-pyrrolo[3,2-d]pyrimidin-4-yl)oxy]aniline (37.5 g, 145 mmol) and triethylamine (60.2 mL, 435 mmol) in tetrahydrofuran (500 mL) was added 3-(trifluoromethyl)phenylisocyanate (32.6 g, 174 mmol), and the mixture was stirred at room temperature for 15 hr. The reaction mixture was diluted with ethyl acetate, washed with water and saturated brine, dried over anhydrous magnesium sulfate, and filtrated. The filtrate was concentrated under reduced pressure, and the residue was purified by silica gel column chromatography (ethyl acetate/hexane=30/70→100/0) and recrystallized from ethyl acetate to give the title compound (42.3 g, 66%) as a white solid.

¹H-NMR (DMSO-d₆, 300 MHz) δ 4.10 (3H, s), 6.60 (1H, d, J=3.2 Hz), 7.12-7.18 (1H, m), 7.30-7.44 (2H, m), 7.50-7.60 (2H, m), 7.79 (1H, d, J=3.2 Hz), 8.05 (1H, s), 8.13 (1H, t, J=9.0 Hz), 8.30 (1H, s), 8.69 (1H, s), 9.42 (1H, s).

### Example 50

### N-{2-fluoro-4-[(5-methyl-5H-pyrrolo[3,2-d]pyrimidin-4-yl)oxy]phenyl}-N′-[3-(trifluoromethoxy)phenyl]urea

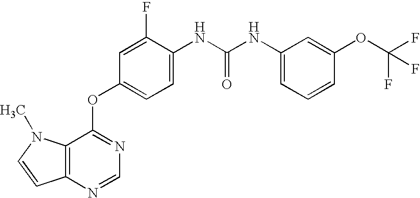

To a solution of 2-fluoro-4-[(5-methyl-5H-pyrrolo[3,2-d]pyrimidin-4-yl)oxy]aniline (350 mg, 1.36 mmol) and triethylamine (1.75 mL) in chloroform (14 mL) was added triphosgene (422 mg, 1.42 mmol), and the mixture was stirred at room temperature for 30 min. 3-(Trifluoromethoxy) aniline (199 μL, 1.49 mmol) was added, and the mixture was stirred at room temperature for 4 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and recrystallized from ethyl acetate-hexane to give the title compound (164 mg, 26%) as a white solid.

¹H-NMR (DMSO-d₆, 300 MHz) δ 4.10 (3H, s), 6.61 (1H, d, J=3.0 Hz), 6.95-6.99 (1H, m), 7.13-7.16 (1H, m), 7.27-7.30 (1H, m), 7.36-7.45 (2H, m), 7.73 (1H, s), 7.80 (1H, d, J=3.0 Hz), 8.12 (1H, t, J=9.2 Hz), 8.30 (1H, s), 8.65 (1H, br s), 9.37 (1H, s).

### Example 51

### N-{2-fluoro-4-[(5-methyl-5H-pyrrolo[3,2-d]pyrimidin-4-yl)oxy]phenyl}-N′-[2-fluoro-5-(trifluoromethyl)phenyl]urea

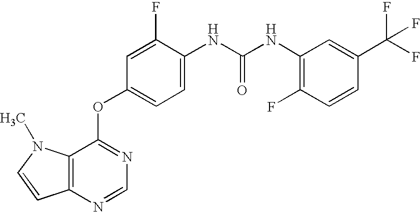

To a solution of 2-fluoro-4-[(5-methyl-5H-pyrrolo[3,2-d]pyrimidin-4-yl)oxy]aniline (350 mg, 1.36 mmol) and triethylamine (910 μL, 6.51 mmol) in tetrahydrofuran (10.5 mL) was added 2-fluoro-5-(trifluoromethyl)phenylisocyanate (235 μL, 1.63 mmol), and the mixture was stirred at room temperature for 5 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by silica gel column chromatography (ethyl acetate/hexane) and recrystallized from ethyl acetate-hexane to give the title compound (314 mg, 50%) as a white solid.

¹H-NMR (DMSO-d₆, 300 MHz) δ 4.11 (3H, s), 6.61 (1H, d, J=3.0 Hz), 7.14-7.19 (1H, m), 7.39-7.44 (2H, m), 7.52-7.56 (1H, m), 7.80 (1H, d, J=3.0 Hz), 8.21 (1H, t, J=9.2 Hz), 8.31 (1H, s), 8.67 (1H, d, J=7.2 Hz), 9.21 (1H, br s), 9.38 (1H, br s).

### Example 52

### N-{2-fluoro-4-[(5-methyl-5H-pyrrolo[3,2-d]pyrimidin-4-yl)oxy]phenyl}-N′-[4-(trifluoromethyl)pyridin-2-yl]urea

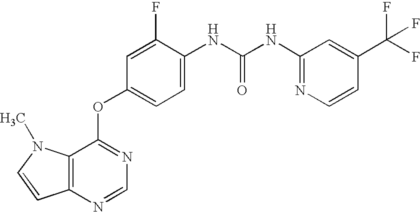

To a solution of 2-fluoro-4-[(5-methyl-5H-pyrrolo[3,2-d]pyrimidin-4-yl)oxy]aniline (350 mg, 1.36 mmol) and triethylamine (1.75 mL) in chloroform (10.5 mL) was added triphosgene (422 mg, 1.42 mmol), and the mixture was stirred at room temperature for 20 min. 2-Amino-4-trifluoromethylpyridine (242 μL, 1.49 mmol) was added, and the mixture was stirred at room temperature for 16 hr. The reaction mixture was diluted with water, and extracted with ethyl acetate/tetrahydrofuran (×2). The organic layer was washed with saturated brine, dried over anhydrous magnesium sulfate, and filtrated. The filtrate was concentrated under reduced pressure, and the residue was purified by silica gel column chromatography (ethyl acetate/hexane) and recrystallized from methanol to give the title compound (105 mg, 17%) as a white solid.

¹H-NMR (DMSO-d₆, 300 MHz) δ 4.11 (3H, s), 6.61 (1H, d, J=3.2 Hz), 7.15-7.20 (1H, m), 7.36-7.46 (2H, m), 7.80 (1H, d, J=3.2 Hz), 8.02 (1H, s), 8.21 (1H, t, J=9.2 Hz), 8.31 (1H, s), 8.55 (1H, d, J=5.1 Hz), 9.97 (1H, br s), 10.10 (1H, s).

### Example 53

### N-{2-chloro-4-[(5-methyl-5H-pyrrolo[3,2-d]pyrimidin-4-yl)oxy]phenyl}-N′-[2-(trifluoroacetyl)-1,2,3,4-tetrahydroisoquinolin-7-yl]urea

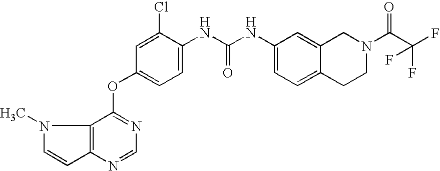

Using 2-(trifluoroacetyl)-1,2,3,4-tetrahydroisoquinoline-7-amine (330 mg, 2.03 mmol), pyridine (346 μL, 4.30 mmol), phenyl chloroformate (181 μL, 2.15 mmol), 2-chloro-4-[(5-methyl-5H-pyrrolo[3,2-d]pyrimidin-4-yl)oxy]aniline (200 mg, 0.660 mmol) and N-methylpyrrolidone (3 mL) as starting materials, and in the same manner as in Example 19, the title compound (265 mg, 82%) was obtained as a white solid.

¹H-NMR (DMSO-d₆, 300 MHz) δ 2.82-2.89 (2H, m), 3.76-3.83 (2H, m), 4.10 (3H, m), 4.73 (2H, s), 6.60 (1H, d, J=3.2 Hz), 7.12-7.15 (1H, m), 7.26-7.35 (3H, m), 7.54 (1H, d, J=2.7 Hz), 7.78 (1H, d, J=3.2 Hz), 8.18 (1H, d, J=8.7 Hz), 8.29 (1H, s), 8.38 (1H, s), 9.37 (1H, s).

### Example 54

### N-{2-chloro-4-[(5-methyl-5H-pyrrolo[3,2-d]pyrimidin-4-yl)oxy]phenyl}-N′-(1,2,3,4-tetrahydroisoquinolin-7-yl)urea

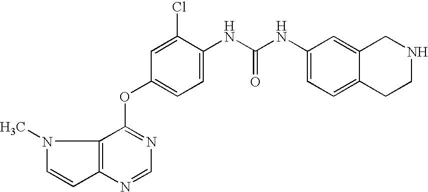

To a solution of N-{2-chloro-4-[(5-methyl-5H-pyrrolo[3,2-d]pyrimidin-4-yl)oxy]phenyl}-N′-[2-(trifluoroacetyl)-1,2,3,4-tetrahydroisoquinolin-7-yl]urea (250 mg, 0.460 mmol) in methanol (2 mL) was added potassium hydroxide (100 mg, 1.78 mmol), and the mixture was stirred at room temperature for 3 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was filtrated and washed with ethyl acetate-hexane to give the title compound (125 mg, 61%) as a white solid.

¹H-NMR (DMSO-d₆, 300 MHz) δ 2.60-2.73 (2H, m), 2.90-2.94 (2H, m), 3.81 (2H, s), 4.10 (3H, s), 6.61 (1H, d, J=3.2 Hz), 6.99 (1H, d, J=8.1 Hz), 7.13-7.18 (2H, m), 7.29 (1H, dd, J=9.1, 2.8 Hz), 7.55 (1H, d, J=2.8 Hz), 7.80 (1H, d, J=3.2 Hz), 8.20 (1H, d, J=9.1 Hz), 8.30-8.34 (2H, m), 9.27 (1H, s).

### Example 55

### N-{2-chloro-4-[(5-methyl-5H-pyrrolo[3,2-d]pyrimidin-4-yl)oxy]phenyl}-N′-(2-methyl-1,2,3,4-tetrahydroisoquinolin-7-yl)urea

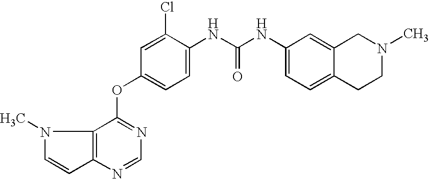

To a solution of N-{2-chloro-4-[(5-methyl-5H-pyrrolo[3,2-d]pyrimidin-4-yl)oxy]phenyl}-N′-(1,2,3,4-tetrahydroisoquinolin-7-yl)urea (100 mg, 0.223 mmol) and cesium carbonate (218 mg, 0.669 mmol) in N,N-dimethylformamide (5 mL) was added methyl methanesulfonate (21.0 μL, 0.245 mmol), and the mixture was stirred at room temperature for 10 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=5/95→90/10) and recrystallized from ethyl acetate-hexane to give the title compound (7.0 mg, 7%) as a white solid.

¹H-NMR (DMSO-d₆, 300 MHz) δ 2.09 (3H, s), 2.50-2.60 (2H, m), 2.70-2.80 (2H, m), 3.43 (2H, s), 4.10 (3H, s), 6.60 (1H, d, J=3.0 Hz), 7.03 (1H, d, J=9.0 Hz), 7.18-7.31 (3H, m), 7.54 (1H, d, J=2.7 Hz), 7.79 (1H, d, J=3.0 Hz), 8.19 (1H, d, J=9.0 Hz), 8.30 (1H, s), 8.31 (1H, s), 9.26 (1H, s).

### Example 56

### N-{2-chloro-4-[(5-methyl-5H-pyrrolo[3,2-d]pyrimidin-4-yl)oxy]phenyl}-N′-[4-(1H-imidazol-1-yl)-3-(trifluoromethyl)phenyl]urea

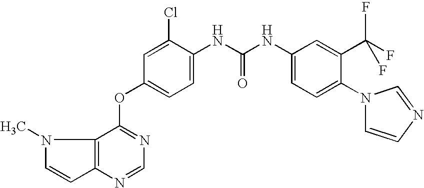

To a solution of 1-[4-nitro-2-(trifluoromethyl)phenyl]-1H-imidazole (147 mg, 0.570 mmol) in methanol (20 mL) was added palladium carbon (50% water-containing product, 15 mg), and the mixture was stirred under a hydrogen atmosphere at room temperature for 3 hr. The reaction mixture was filtered through celite. The filtrate was concentrated under reduced pressure and dried, and the residue was dissolved in N-methylpyrrolidone (3 mL). Pyridine (92.0 μL, 1.14 mmol) and phenyl {2-chloro-4-[(5-methyl-5H-pyrrolo[3,2-d]pyrimidin-4-yl)oxy]phenyl}carbamate (150 mg, 0.380 mmol) were added, and the mixture was stirred at 110° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=20/80→100/0) and recrystallized from ethyl acetate-hexane to give the title compound (63.0 mg, 31%) as a white solid.

¹H-NMR (DMSO-d₆, 300 MHz) δ 4.11 (3H, s), 6.60-6.61 (1H, m), 7.07 (1H, s), 7.31-7.36 (2H, m), 7.51 (1H, d, J=8.7 Hz), 7.57-7.59 (1H, m), 7.72-7.78 (1H, m), 7.79-7.82 (2H, m), 8.12-8.20 (2H, m), 8.30 (1H, s), 8.53 (1H, s), 9.96 (1H, s).

### Example 57

### 3-{[({2-chloro-4-[(5-methyl-5H-pyrrolo[3,2-d]pyrimidin-4-yl)oxy]phenyl}amino)carbonyl]amino}-N-methyl-5-(trifluoromethyl)benzamide

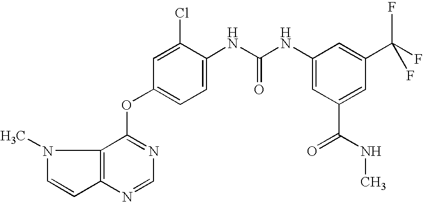

3-Amino-N-methyl-5-(trifluoromethyl)benzamide (100 mg, 0.456 mmol) was dissolved in N-methylpyrrolidone (3 mL), and pyridine (92.0 μL, 1.14 mmol) and phenyl {2-chloro-4-[(5-methyl-5H-pyrrolo[3,2-d]pyrimidin-4-yl)oxy]phenyl}carbamate (150 mg, 0.380 mmol) were added. The mixture was stirred at 110° C. for 15 hr and the reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=20/80→100/0) and recrystallized from ethyl acetate-hexane to give the title compound (19.0 mg, 10%) as a white solid.

¹H-NMR (DMSO-d₆, 300 MHz) δ 2.81 (3H, d, J=4.8 Hz), 4.11 (3H, s), 6.61 (1H, d, J=3.0 Hz), 7.32 (1H, dd, J=9.0, 2.6 Hz), 7.58 (1H, d, J=2.6 Hz), 7.79-7.80 (2H, m), 8.03 (1H, s), 8.15-8.18 (2H, m), 8.30 (1H, s), 8.46 (1H, s), 8.71 (1H, d, J=4.8 Hz), 9.89 (1H, s).

### Example 58

### N-{2-chloro-4-[(5-methyl-5H-pyrrolo[3,2-d]pyrimidin-4-yl)oxy]phenyl}-N′-(4-methylpyridin-2-yl)urea

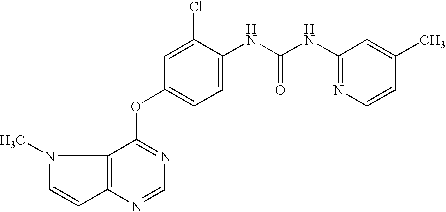

Using 2-amino-4-methylpyridine (49.3 mg, 0.456 mmol), pyridine (92.0 μL, 1.14 mmol), phenyl {2-chloro-4-[(5-methyl-5H-pyrrolo[3,2-d]pyrimidin-4-yl)oxy]phenyl}carbamate (150 mg, 0.380 mmol) and N-methylpyrrolidone (3 mL) as starting materials, and in the same manner as in Example 57, the title compound (23.0 mg, 15%) was obtained as a white solid.

¹H-NMR (DMSO-d₆, 300 MHz) δ 2.30 (3H, s), 4.11 (3H, s), 6.60 (1H, d, J=3.2 Hz), 6.89-6.90 (1H, m), 7.03 (1H, s), 7.32 (1H, dd, J=8.9, 2.4 Hz), 7.58 (1H, d, J=2.4 Hz), 7.79 (1H, d, J=3.2 Hz), 8.18 (1H, d, J=5.1 Hz), 8.30 (1H, s), 8.40 (1H, d, J=8.9 Hz), 9.96 (1H, s), 12.04 (1H, br s).

### Example 59

### N-{2-chloro-4-[(5-methyl-5H-pyrrolo[3,2-d]pyrimidin-4-yl)oxy]phenyl}-N′-(6-methoxypyrimidin-4-yl)urea

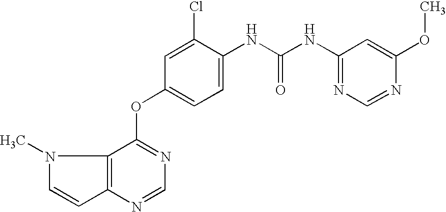

Using 4-amino-6-methoxypyrimidine (237 mg, 1.90 mmol), pyridine (92.0 μL, 1.14 mmol), phenyl {2-chloro-4-[(5-methyl-5H-pyrrolo[3,2-d]pyrimidin-4-yl)oxy]phenyl}carbamate (150 mg, 0.380 mmol) and N-methylpyrrolidone (3 mL) as starting materials, and in the same manner as in Example 57, the title compound (20.0 mg, 12%) was obtained as a white solid.

¹H-NMR (DMSO-d₆, 300 MHz) δ 3.91 (3H, s), 4.11 (3H, s), 6.61 (1H, d, J=2.6 Hz), 6.86 (1H, s), 7.34 (1H, dd, J=8.9, 2.5 Hz), 7.60 (1H, d, J=2.5 Hz), 7.80 (1H, d, J=2.6 Hz), 8.26 (1H, d, J=8.9 Hz), 8.31 (1H, s), 8.36 (1H, s), 8.58 (1H, s), 9.08 (1H, s).

### Example 60

### N-{2-chloro-4-[(5-methyl-5H-pyrrolo[3,2-d]pyrimidin-4-yl)oxy]phenyl}-N′-[4-(trifluoromethyl)pyridin-2-yl]urea Methanesulfonic Acid Salt

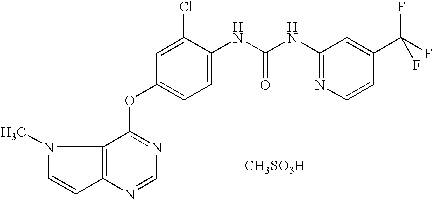

To a solution of N-{2-chloro-4-[(5-methyl-5H-pyrrolo[3,2-d]pyrimidin-4-yl)oxy]phenyl}-N′-[4-(trifluoromethyl)pyridin-2-yl]urea (300 mg, 0.648 mmol) in a mixture of ethanol (10 mL) and N,N-dimethylformamide (7 mL) was added methanesulfonic acid (42.0 μL, 0.648 mmol), and the mixture was stood for 3 days. The precipitated crystals were collected by filtration, and washed with ethyl acetate to give the title compound (129 mg, 40%) as a white solid. melting point 222-230° C.

¹H-NMR (DMSO-d₆, 300 MHz) δ 2.40 (3H, s), 4.15 (3H, s), 6.70-6.71 (1H, m), 7.36-7.41 (2H, m), 7.65 (1H, d, J=2.7 Hz), 7.83 (1H, s), 7.97 (1H, s), 8.35 (1H, d, J=9.0 Hz), 8.54-8.61 (2H, m), 10.64 (1H, s), 10.67 (1H, br s).

### Example 61

### N-{2-chloro-4-[(5-methyl-5H-pyrrolo[3,2-d]pyrimidin-4-yl)oxy]phenyl}-N′-(4,6-dimethylpyridin-2-yl)urea

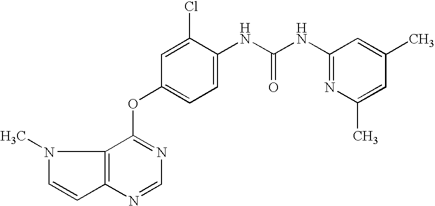

Using 2-amino-4,6-dimethylpyridine (117 mg, 0.962 mmol), pyridine (233 μL, 2.89 mmol), phenyl chloroformate (127 μL, 1.01 mmol), 2-chloro-4-[(5-methyl-5H-pyrrolo[3,2-d]pyrimidin-4-yl)oxy]aniline (200 mg, 0.728 mmol) and N-methylpyrrolidone (4 mL) as starting materials, and in the same manner as in Example 19, the title compound (121 mg, 39%) was obtained as a white solid.

¹H-NMR (DMSO-d₆, 300 MHz) δ 2.26 (3H, s), 2.44 (3H, s), 4.11 (3H, s), 6.58-6.62 (1H, m), 6.74-6.78 (2H, m), 7.33 (1H, dd, J=9.0, 2.6 Hz), 7.60 (1H, d, J=2.6 Hz), 7.81 (1H, d, J=3.0 Hz), 8.32 (1H, s), 8.42 (1H, d, J=9.0 Hz), 9.93 (1H, s), 11.99 (1H, br s).

### Example 62

### N-{2-chloro-4-[(5-methyl-5H-pyrrolo[3,2-d]pyrimidin-4-yl)oxy]phenyl}-N′-(3,4-dimethoxyphenyl)urea

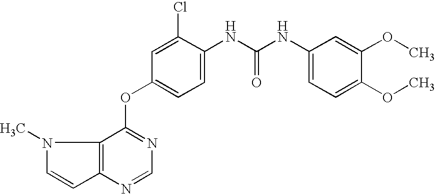

Using 3,4-dimethoxyaniline (64.7 mg, 0.422 mmol), pyridine (85.2 μL, 1.06 mmol), phenyl {2-chloro-4-[(5-methyl-5H-pyrrolo[3,2-d]pyrimidin-4-yl)oxy]phenyl}carbamate (139 mg, 0.352 mmol) and N-methylpyrrolidone (3 mL) as starting materials, and in the same manner as in Example 57, the title compound (130 mg, 81%) was obtained as a white solid.

¹H-NMR (DMSO-d₆, 300 MHz) δ 3.71 (3H, s), 3.75 (3H, s), 4.10 (3H, s), 6.60 (1H, d, J=3.0 Hz), 6.86-6.93 (2H, m), 7.20 (1H, s), 7.28 (1H, dd, J=9.0, 2.7 Hz), 7.53 (1H, d, J=2.7 Hz), 7.79 (1H, d, J=3.0 Hz), 8.20 (1H, d, J=9.0 Hz), 8.20 (1H, s), 8.29 (1H, s), 9.25 (1H, s).

### Example 63

### N-{2-chloro-4-[(5-methyl-5H-pyrrolo[3,2-d]pyrimidin-4-yl)oxy]phenyl}-N′-[3-morpholin-4-yl-5-(trifluoromethyl)phenyl]urea

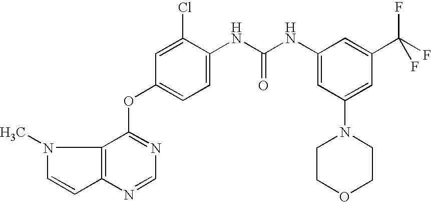

To a solution of 3-morpholin-4-yl-5-(trifluoromethyl)benzoic acid (1.00 g, 3.63 mmol) and triethylamine (2.50 mL, 18.2 mmol) in toluene (30 mL) was added diphenylphosphoryl azide (860 μL, 3.99 mmol), and the mixture was stirred under reflux for 4 hr. After cooling the reaction mixture to room temperature, 2-chloro-4-[(5-methyl-5H-pyrrolo[3,2-d]pyrimidin-4-yl)oxy]aniline (907 mg, 3.30 mmol) was added, and the mixture was stirred at room temperature for 2 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=10/90→100/0) and recrystallized from ethyl acetate-hexane to give the title compound (572 mg, 32%) as a white solid.

¹H-NMR (DMSO-d₆, 300 MHz) δ 3.16-3.19 (4H, m), 3.74-3.77 (4H, m), 4.10 (3H, s), 6.60 (1H, d, J=3.0 Hz), 6.88 (1H, s), 7.18 (1H, s), 7.29-7.35 (2H, m), 7.56 (1H, d, J=2.7 Hz), 7.79 (1H, d, J=3.0 Hz), 8.17 (1H, d, J=8.7 Hz), 8.30 (1H, s), 8.39 (1H, s), 9.60 (1H, s).

### Example 64

### N-{2-chloro-4-[(5-methyl-5H-pyrrolo[3,2-d]pyrimidin-4-yl)oxy]phenyl}-N′-[3-(trifluoromethyl)phenyl]urea Benzenesulfonic Acid Salt

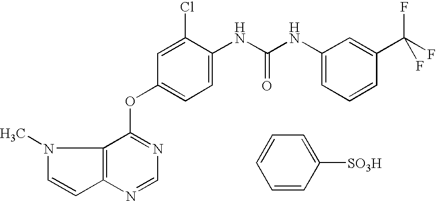

N-{2-Chloro-4-[(5-methyl-5H-pyrrolo[3,2-d]pyrimidin-4-yl)oxy]phenyl}-N′-[3-(trifluoromethyl)phenyl]urea (500 mg, 1.08 mmol) was dissolved in ethyl acetate (10 mL) at 70° C., and 0.5N benzenesulfonic acid ethyl acetate solution (2.16 mL, 1.08 mmol) was added dropwise. The solvent was evaporated under reduced pressure, and the residue was recrystallized from acetone to give the title compound (423 mg, 63%) as a white solid.

¹H-NMR (DMSO-d₆, 300 MHz) δ 4.16 (3H, s), 6.73-6.74 (1H, m), 7.29-7.38 (5H, m), 7.55-7.63 (5H, m), 8.02-8.06 (2H, m), 8.22 (1H, d, J=8.7 Hz), 8.49 (1H, s), 8.63 (1H, s), 9.75 (1H, s).

### Example 65

### Methyl 3-{[({2-chloro-4-[(5-methyl-5H-pyrrolo[3,2-d]pyrimidin-4-yl)oxy]phenyl}amino)carbonyl]amino}-5-(trifluoromethyl)benzoate

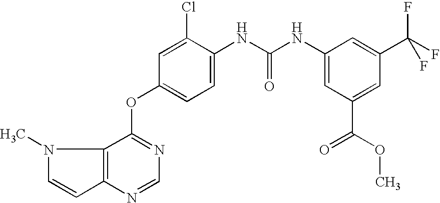

Using 2-chloro-4-[(5-methyl-5H-pyrrolo[3,2-d]pyrimidin-4-yl)oxy]aniline (1.35 g, 4.91 mmol), pyridine (1.20 mL, 14.7 mmol), phenyl chloroformate (652 μL, 5.16 mmol), methyl 3-amino-5-(trifluoromethyl)benzoate (1.18 g, 5.40 mmol) and N-methylpyrrolidone (3 mL) as starting materials, and in the same manner as in Example 20, the title compound (1.02 g, 40%) was obtained as a white solid.

¹H-NMR (DMSO-d₆, 300 MHz) δ 3.91 (3H, s), 4.11 (3H, s), 6.61 (1H, d, J=3.0 Hz), 7.33 (1H, dd, J=8.8, 2.6 Hz), 7.59 (1H, d, J=2.6 Hz), 7.79-7.81 (2H, m), 8.13-8.17 (2H, m), 8.29-8.31 (2H, m), 8.46 (1H, s), 9.48 (1H, s).

### Example 66

### N-{2-chloro-4-[(5-methyl-5H-pyrrolo[3,2-d]pyrimidin-4-yl)oxy]phenyl}-N′-[4-morpholin-4-yl-3-(trifluoromethyl)phenyl]urea

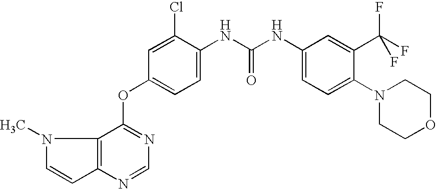

Using 4-morpholin-4-yl-3-(trifluoromethyl)aniline (204 mg, 0.828 mmol), pyridine (200 μL, 2.48 mmol), phenyl chloroformate (105 μL, 0.828 mmol), 2-chloro-4-[(5-methyl-5H-pyrrolo[3,2-d]pyrimidin-4-yl)oxy]aniline (190 mg, 0.690 mmol) and N-methylpyrrolidone (3 mL) as starting materials, and in the same manner as in Example 19, the title compound (127 mg, 34%) was obtained as a white solid.

¹H-NMR (DMSO-d₆, 300 MHz) δ 2.80-2.83 (4H, s), 3.68-3.71 (4H, s), 4.10 (3H, s), 6.60 (1H, d, J=3.0 Hz), 7.30 (1H, dd, J=9.1, 2.7 Hz), 7.54-7.63 (3H, m), 7.79 (1H, d, J=3.0 Hz), 7.94 (1H, J=2.1 Hz), 8.16 (1H, d, J=9.1 Hz), 8.29 (1H, s), 8.42 (1H, s), 9.67 (1H, s).

### Example 67

### N-{2-chloro-4-[(5-methyl-5H-pyrrolo[3,2-d]pyrimidin-4-yl)oxy]phenyl}-N′-[3-(morpholin-4-ylcarbonyl)-5-(trifluoromethyl)phenyl]urea

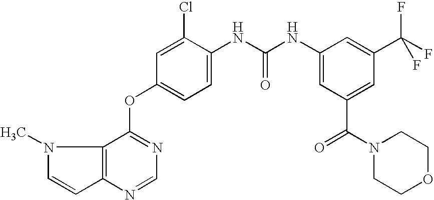

Using 3-(morpholin-4-ylcarbonyl)-5-(trifluoromethyl)aniline (589 mg, 2.14 mmol), pyridine (518 μL, 6.42 mmol), phenyl chloroformate (273 μL, 2.17 mmol), 2-chloro-4-[(5-methyl-5H-pyrrolo[3,2-d]pyrimidin-4-yl)oxy]aniline (588 mg, 2.14 mmol) and N-methylpyrrolidone (3 mL) as starting materials, and in the same manner as in Example 19, the title compound (101 mg, 8%) was obtained as a white solid.

¹H-NMR (DMSO-d₆, 300 MHz) δ 3.45-3.75 (8H, m), 4.10 (3H, s), 6.60 (1H, d, J=3.0 Hz), 7.30-7.36 (2H, m), 7.58 (1H, d, J=2.7 Hz), 7.67 (1H, s), 7.79 (1H, d, J=3.0 Hz), 8.00 (1H, s), 8.12 (1H, d, J=9.0 Hz), 8.30 (1H, s), 8.52 (1H, s), 9.85 (1H, s).

### Example 68

### N-{2-chloro-4-[(5-methyl-5H-pyrrolo[3,2-d]pyrimidin-4-yl)oxy]phenyl}-N′-[3-(morpholin-4-ylmethyl)-5-(trifluoromethyl)phenyl]urea

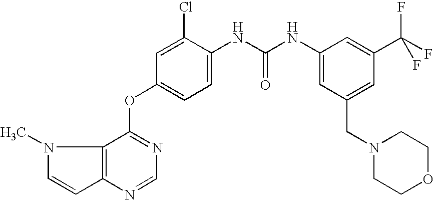

Using 3-(morpholin-4-ylmethyl)-5-(trifluoromethyl)aniline (228 mg, 0.877 mmol), pyridine (212 μL, 2.63 mmol), phenyl chloroformate (111 mL, 0.877 mmol), 2-chloro-4-[(5-methyl-5H-pyrrolo[3,2-d]pyrimidin-4-yl)oxy]aniline (240 mg, 0.877 mmol) and N-methylpyrrolidone (3 mL) as starting materials, and in the same manner as in Example 19, the title compound (109 mg, 23%) was obtained as a white solid.

¹H-NMR (DMSO-d₆, 300 MHz) δ 2.30-2.45 (4H, m), 3.30-3.61 (6H, m), 4.10 (3H, s), 6.60 (1H, d, J=3.3 Hz), 7.26 (1H, s), 7.28 (1H, dd, J=8.8, 2.6 Hz), 7.54 (1H, s), 7.56 (1H, d, J=2.6 Hz), 7.78 (1H, d, J=3.3 Hz), 7.94 (1H, s), 8.16 (1H, d, J=8.8 Hz), 8.30 (1H, s), 8.40 (1H, s), 9.74 (1H, s).

### Example 69

### N-{2-chloro-4-[(5-methyl-5H-pyrrolo[3,2-d]pyrimidin-4-yl)oxy]phenyl}-N′-[3-[(4-methylpiperazin-1-yl)carbonyl]-5-(trifluoromethyl)phenyl]urea

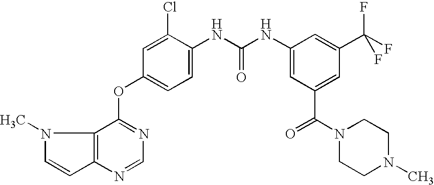

Using 3-[(4-methylpiperazin-1-yl)carbonyl]-5-(trifluoromethyl)aniline (635 mg, 2.21 mmol), pyridine (535 μL, 6.63 mmol), phenyl chloroformate (279 μL, 2.21 mmol), 2-chloro-4-[(5-methyl-5H-pyrrolo[3,2-d]pyrimidin-4-yl)oxy]aniline (607 mg, 2.21 mmol) and N-methylpyrrolidone (3 mL) as starting materials, and in the same manner as in Example 19, the title compound (49.0 mg, 4%) was obtained as a white solid.

¹H-NMR (DMSO-d₆, 300 MHz) δ 2.20 (3H, s), 2.21-2.40 (6H, m), 3.50-3.70 (2H, m), 4.10 (3H, s), 6.60 (1H, d, J=3.0 Hz), 7.30-7.34 (2H, m), 7.58 (1H, d, J=3.0 Hz), 7.65 (1H, s), 7.79 (1H, d, J=3.0 Hz), 7.99 (1H, s), 8.12 (1H, d, J=9.0 Hz), 8.30 (1H, s), 8.51 (1H, s), 9.84 (1H, s).

### Example 70

### N-{2-chloro-4-[(5-methyl-5H-pyrrolo[3,2-d]pyrimidin-4-yl)oxy]phenyl}-N′-[4-[(1-methylpiperidin-4-yl)oxy]-3-(trifluoromethyl)phenyl]urea

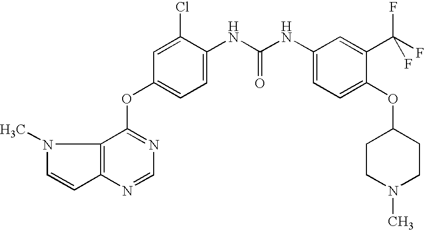

Using 4-[(1-methylpiperidin-4-yl)oxy]-3-(trifluoromethyl)aniline (314 mg, 1.15 mmol), pyridine (280 μL, 3.45 mmol), phenyl chloroformate (147 μL, 1.17 mmol), 2-chloro-4-[(5-methyl-5H-pyrrolo[3,2-d]pyrimidin-4-yl)oxy]aniline (316 mg, 1.15 mmol) and N-methylpyrrolidone (3 mL) as starting materials, and in the same manner as in Example 19, the title compound (75.0 mg, 11%) was obtained as a white solid.

¹H-NMR (DMSO-d₆, 300 MHz) δ 1.65-1.75 (2H, m), 1.85-1.95 (2H, m), 2.19-2.35 (5H, m), 2.50-2.60 (2H, m), 4.10 (3H, s), 4.45-4.51 (1H, m), 6.60 (1H, d, J=3.0 Hz), 7.26-7.31 (2H, m), 7.52-7.55 (2H, m), 7.79 (1H, d, J⁼3.0 Hz), 7.85 (1H, d, J=2.4 Hz), 8.17 (1H, d, J=9.6 Hz), 8.30 (1H, s), 8.33 (1H, s), 9.47 (1H, s).

### Example 71

### N-{2-chloro-4-[(5-methyl-5H-pyrrolo[3,2-d]pyrimidin-4-yl)oxy]phenyl}-N′-[4-[(4-methylpiperazin-1-yl)methyl]-3-(trifluoromethyl)phenyl]urea

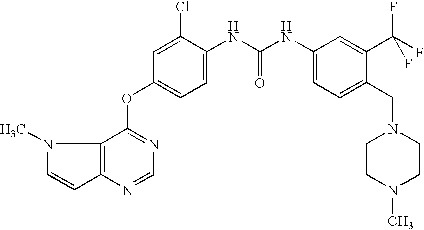

Using 2-chloro-4-[(5-methyl-5H-pyrrolo[3,2-d]pyrimidin-4-yl)oxy]aniline (284 mg, 1.03 mmol), pyridine (250 μL, 3.09 mmol), phenyl chloroformate (137 μL, 1.09 mmol), 4-[(4-methylpiperazin-1-yl)methyl]-3-(trifluoromethyl)aniline (338 mg, 1.24 mmol) and N-methylpyrrolidone (3 mL) as starting materials, and in the same manner as in Example 20, the title compound (133 mg, 23%) was obtained as a white solid.

¹H-NMR (DMSO-d₆, 300 MHz) δ 2.16 (3H, s), 2.20-2.45 (8H, m), 3.54 (2H, s), 4.10 (3H, s), 6.61 (1H, d, J=2.9 Hz), 7.31 (1H, dd, J=8.8, 2.6 Hz), 7.56-7.67 (3H, m), 7.80 (1H, d, J=5=2.9 Hz), 8.00 (1H, s), 8.18 (1H, d, J=8.8 Hz), 8.30 (1H, s), 8.43 (1H, s), 9.70 (1H, s).

### Example 72

### N-{2-chloro-4-[(5-methyl-5H-pyrrolo[3,2-d]pyrimidin-4-yl)oxy]phenyl}-N′-[4-(morpholin-4-ylmethyl)-3-(trifluoromethyl)phenyl]urea

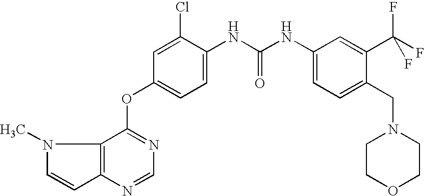

Using 2-chloro-4-[(5-methyl-5H-pyrrolo[3,2-d]pyrimidin-4-yl)oxy]aniline (284 mg, 1.03 mmol), pyridine (250 μL, 3.09 mmol), phenyl chloroformate (137 μL, 1.09 mmol), 4-(morpholin-4-ylmethyl)-3-(trifluoromethyl)aniline (322 mg, 1.24 mmol) and N-methylpyrrolidone (3 mL) as starting materials, and in the same manner as in Example 20, the title compound (173 mg, 29%) was obtained as a white solid.

¹H-NMR (DMSO-d₆, 300 MHz) δ 2.30-2.40 (4H, m), 3.55-3.60 (6H, m), 4.10 (3H, s), 6.60 (1H, d, J=2.9 Hz), 7.31 (1H, dd, J=8.9, 2.6 Hz), 7.56-7.59 (2H, m), 7.68 (1H, d, J=8.7 Hz), 7.79 (1H, d, J=2.9 Hz), 8.00 (1H, d, J=2.1 Hz), 8.18 (1H, d, J=8.9 Hz), 8.30 (1H, s), 8.42 (1H, s), 9.70 (1H, s).

### Example 73

### N-{2-chloro-4-[(5-methyl-5H-pyrrolo[3,2-d]pyrimidin-4-yl)oxy]phenyl}-N′-[4-[(4-methylpiperazin-1-yl)carbonyl]-3-(trifluoromethyl)phenyl]urea

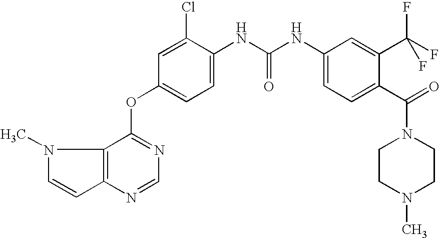

Using 2-chloro-4-[(5-methyl-5H-pyrrolo[3,2-d]pyrimidin-4-yl)oxy]aniline (275 mg, 1.00 mmol), pyridine (242 μL, 3.00 mmol), phenyl chloroformate (132 μL, 1.05 mmol), 4-[(4-methylpiperazin-1-yl)carbonyl]-3-(trifluoromethyl)aniline (345 mg, 1.20 mmol) and N-methylpyrrolidone (3 mL) as starting materials, and in the same manner as in Example 20, the title compound (158 mg, 28%) was obtained as a white solid.

¹H-NMR (DMSO-d₆, 300 MHz) δ 2.19-2.45 (7H, m), 3.00-3.20 (2H, m), 3.50-3.70 (2H, m), 4.11 (3H, s), 6.65 (1H, d, J=3.2 Hz), 7.30-7.38 (2H, m), 7.57-7.65 (2H, m), 7.79 (1H, d, J=3.2 Hz), 8.07 (1H, s), 8.15 (1H, d, J=9.3 Hz), 8.30 (1H, s), 8.49 (1H, s), 9.84 (1H, s).

### Example 74

### N-{2-chloro-4-[(5-methyl-5H-pyrrolo[3,2-d]pyrimidin-4-yl)oxy]phenyl}-N′-[4-(morpholin-4-ylcarbonyl)-3-(trifluoromethyl)phenyl]urea

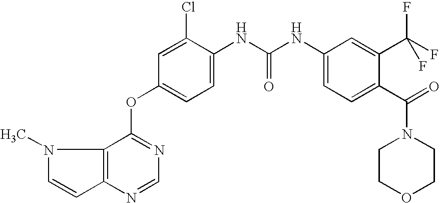

Using 2-chloro-4-[(5-methyl-5H-pyrrolo[3,2-d]pyrimidin-4-yl)oxy]aniline (278 mg, 1.01 mmol), pyridine (245 μL, 3.03 mmol), phenyl chloroformate (134 μL, 1.06 mmol), 4-(morpholin-4-ylcarbonyl)-3-(trifluoromethyl)aniline (332 mg, 1.21 mmol) and N-methylpyrrolidone (3 mL) as starting materials, and in the same manner as in Example 20, the title compound (181 mg, 31%) was obtained as a white solid.

¹H-NMR (DMSO-d₆, 300 MHz) δ 3.05-3.25 (2H, m), 3.40-3.75 (6H, m), 4.10 (3H, s), 6.60 (1H, d, J=3.2 Hz), 7.32° (1H, dd, J=9.1, 2.8 Hz), 7.41 (1H, d, J=8.4 Hz), 7.57 (1H, d, J=2.8 Hz), 7.64 (1H, dd, J=8.4, 1.9 Hz), 7.79 (1H, d, J=3.2 Hz), 8.07 (1H, d, J=1.9 Hz), 8.15 (1H, d, J=9.1 Hz), 8.30 (1H, s), 8.49 (1H, s), 9.84 (1H, s).

### Example 75

### N-{2-chloro-4-[(5-methyl-5H-pyrrolo[3,2-d]pyrimidin-4-yl)oxy]phenyl}-N′-[3-[(4-methylpiperazin-1-yl)methyl]-5-(trifluoromethyl)phenyl]urea

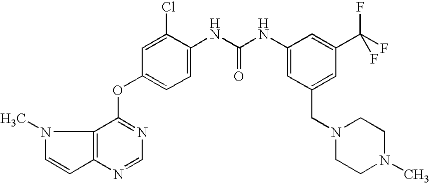

Using 2-chloro-4-[(5-methyl-5H-pyrrolo[3,2-d]pyrimidin-4-yl)oxy]aniline (404 mg, 1.47 mmol), pyridine (357 μL, 4.42 mmol), phenyl chloroformate (195 μL, 1.54 mmol), 3-[(4-methylpiperazin-1-yl)methyl]-5-(trifluoromethyl)aniline (402 mg, 1.47 mmol) and N-methylpyrrolidone (3 mL) as starting materials, and in the same manner as in Example 20, the title compound (58.0 mg, 7%) was obtained as a white solid.

¹H-NMR (DMSO-d₆, 300 MHz) δ 2.15 (3H, s), 2.20-2.50 (8H, m), 3.52 (2H, s), 4.10 (3H, s), 6.60 (1H, d, J=3.0 Hz), 7.23 (1H, s), 7.30 (1H, dd, J=9.0, 2.7 Hz), 7.50 (1H, s), 7.56 (1H, d, J=2.7 Hz), 7.79 (1H, d, J=3.0 Hz), 7.94 (1H, s), 8.17 (1H, d, J=9.0 Hz), 8.29 (1H, s), 8.40 (1H, s), 9.75 (1H, s).

### Example 76

### Tert-butyl 3-{[({2-chloro-4-[(5-methyl-5H-pyrrolo[3,2-d]pyrimidin-4-yl)oxy]phenyl}amino)carbonyl]amino}piperidine-1-carboxylate

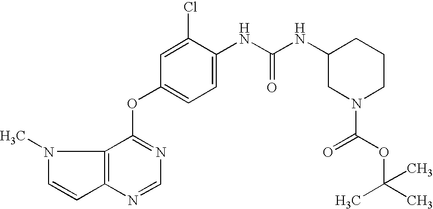

Using 2-chloro-4-[(5-methyl-5H-pyrrolo[3,2-d]pyrimidin-4-yl)oxy]aniline (500 mg, 1.82 mmol), pyridine (441 μL, 5.46 mmol), phenyl chloroformate (241 μL, 1.91 mmol), tert-butyl 3-aminopiperidine-1-carboxylate (437 mg, 2.18 mmol) and N-methylpyrrolidone (3 mL) as starting materials, and in the same manner as in Example 20, the title compound (542 mg, 59%) was obtained as a white solid.

¹H-NMR (DMSO-d₆, 300 MHz) δ 1.38-1.50 (13H, m), 1.60-1.70 (1H, m), 1.75-1.85 (1H, m), 3.20-3.40 (2H, m), 3.50-3.70 (1H, m), 4.09 (3H, s), 6.59 (1H, d, J=2.9 Hz), 7.08 (1H, d, J=6.6 Hz), 7.24 (1H, dd, J=8.8, 2.6 Hz), 7.47 (1H, d, J=2.6 Hz), 7.77 (1H, d, J=2.9 Hz), 8.11 (1H, s), 8.22 (1H, d, J=8.8 Hz), 8.28 (1H, s).

### Example 77

### N-{2-methoxy-4-[(5-methyl-5H-pyrrolo[3,2-d]pyrimidin-4-yl)oxy]phenyl}-N′-[3-(trifluoromethyl)phenyl]urea

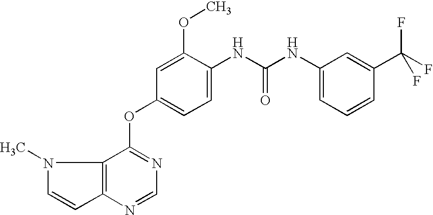

Using 2-methoxy-4-[(5-methyl-5H-pyrrolo[3,2-d]pyrimidin-4-yl)oxy]aniline (268 mg, 0.992 mmol), triethylamine (412 μL, 2.98 mmol), 3-(trifluoromethyl)phenylisocyanate (166 μL, 1.19 mmol) and tetrahydrofuran (5 mL) as starting materials, and in the same manner as in Example 15, the title compound (245 mg, 54%) was obtained as a white solid.

¹H-NMR (DMSO-d₆, 300 MHz) δ 3.38 (3H, s), 4.12 (3H, s), 6.60 (1H, d, J=3.3 Hz), 6.86 (1H, dd, J=8.9, 2.3 Hz), 7.07 (1H, d, J=2.3 Hz), 7.30-7.34 (1H, m), 7.52-7.54 (2H, m), 7.78 (1H, d, J=3.3 Hz), 8.06 (1H, s), 8.15 (1H, d, J=8.9 Hz), 8.29 (1H, s), 8.33 (1H, s), 9.67 (1H, s).

### Example 78

### N-{2-chloro-4-[(5-methyl-5H-pyrrolo[3,2-d]pyrimidin-4-yl)oxy]phenyl}-N′-[3-(2,2,2-trifluoro-1-hydroxyethyl)phenyl]urea

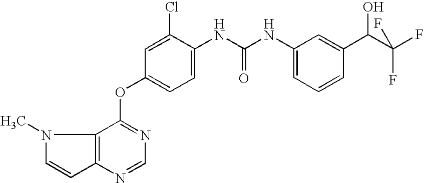

Using 1-(3-aminophenyl)-2,2,2-trifluoroethanol (687 mg, 3.63 mmol), pyridine (880 μL, 10.9 mmol), phenyl chloroformate (481 μL, 3.81 mmol), 2-chloro-4-[(5-methyl-5H-pyrrolo[3,2-d]pyrimidin-4-yl)oxy]aniline (997 mg, 3.63 mmol) and N-methylpyrrolidone (10 mL) as starting materials, and in the same manner as in Example 19, the title compound (161 mg, 9%) was obtained as a white solid.

¹H-NMR (DMSO-d₆, 300 MHz) δ 4.10 (3H, s), 5.12-5.16 (1H, m), 6.60 (1H, d, J=3.0 Hz), 6.83 (1H, d, J=5.4 Hz), 7.12 (1H, d, J=7.5 Hz), 7.29-7.36 (2H, m), 7.50-7.56 (2H, m), 7.63 (1H, s), 7.79 (1H, d, J=3.0 Hz), 8.21 (1H, d, J=9.0 Hz), 8.30 (1H, s), 8.34 (1H, s), 9.51 (1H, s).

### Example 79

### N-{2-chloro-4-[(5-methyl-5H-pyrrolo[3,2-d]pyrimidin-4-yl)oxy]phenyl}-N′-[3-[(4-hydroxypiperidin-1-yl)methyl]-5-(trifluoromethyl)phenyl]urea

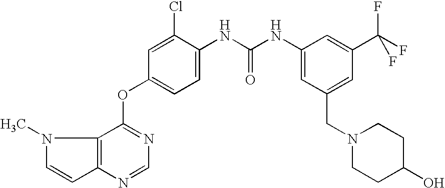

Using 2-chloro-4-[(5-methyl-5H-pyrrolo[3,2-d]pyrimidin-4-yl)oxy]aniline (307 mg, 1.12 mmol), pyridine (271 μL, 3.36 mmol), phenyl chloroformate (148 μL, 1.17 mmol), 1-[3-amino-5-(trifluoromethyl)benzyl]piperidin-4-ol (338 mg, 1.23 mmol) and N-methylpyrrolidone (5 mL) as starting materials, and in the same manner as in Example 20, the title compound (143 mg, 22%) was obtained as a white solid.

¹H-NMR (DMSO-d₆, 300 MHz) δ 1.37-1.52 (2H, m), 1.77-1.92 (2H, m), 2.05-2.11 (2H, m), 2.62-2.77 (2H, m), 3.40-3.51 (3H, m), 4.11 (3H, s), 4.56 (1H, d, J=4.5 Hz), 6.61 (1H, d, J=3.0 Hz), 7.23 (1H, s), 7.32 (1H, dd, J=9.0, 2.7 Hz), 7.51 (1H, s), 7.57 (1H, d, J=2.7 Hz), 7.80 (1H, d, J=3.0 Hz), 7.94 (1H, s), 8.18 (1H, d, J=9.0 Hz), 8.30 (1H, s), 8.39 (1H, s), 9.75 (1H, s).

### Example 80

### N-{4-[(5-methyl-5H-pyrrolo[3,2-d]pyrimidin-4-yl)oxy]cyclohexyl}-N′-[3-(trifluoromethyl)phenyl]urea

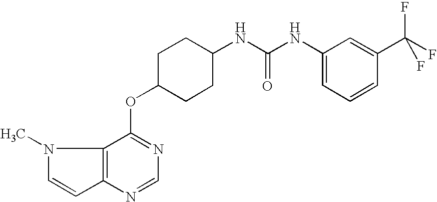

To a solution of sodium hydride (70.0 mg, 1.74 mmol) in N,N-dimethylformamide (5 mL) was added dropwise a solution of N-(trans-4-hydroxycyclohexyl)-N′-[3-(trifluoromethyl)phenyl]urea (500 mg, 1.65 mmol) in N,N-dimethylformamide (5 mL) at 0° C., and the mixture was stirred at room temperature for 1 hr. 4-Chloro-5-methyl-5H-pyrrolo[3,2-d]pyrimidine (291 mg, 1.74 mmol) was added to the reaction mixture, and the mixture was stirred at 70° C. for 5 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=10/90→100/0) and recrystallized from ethyl acetate-hexane to give the title compound (343 mg, 48%) as a white solid.

¹H-NMR (DMSO-d₆, 300 MHz) δ 1.40-1.51 (2H, m), 1.62-1.74 (2H, m), 1.98-2.02 (2H, m), 2.12-2.20 (2H, m), 3.55-3.70 (1H, m), 4.00 (3H, s), 5.26-5.32 (1H, m), 6.33 (1H, d, J=7.5 Hz), 6.48-6.49 (1H, m), 7.22-7.23 (1H, m), 7.42-7.46 (2H, m), 7.62 (1H, d, J=3.0 Hz), 8.00 (1H, s), 8.34 (1H, s), 8.73 (1H, s).

### Example 81

### N-{2-chloro-4-[(5-methyl-5H-pyrrolo[3,2-d]pyrimidin-4-yl)oxy]phenyl}-N′-[3-(2,2,2-trifluoro-1-hydroxy-1-methylethyl)phenyl]urea

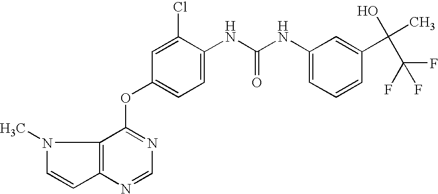

Using 2-chloro-4-[(5-methyl-5H-pyrrolo[3,2-d]pyrimidin-4-yl)oxy]aniline (310 mg, 1.13 mmol), pyridine (275 μL, 3.39 mmol), phenyl chloroformate (150 μL, 1.18 mmol), 2-(3-aminophenyl)-1,1,1-trifluoropropan-2-ol (278 mg, 1.36 mmol) and N-methylpyrrolidone (6 mL) as starting materials, and in the same manner as in Example 20, the title compound (192 mg, 34%) was obtained as a white solid.

¹H-NMR (DMSO-d₆, 300 MHz) δ 1.68 (3H, s), 4.10 (3H, s), 6.58-6.61 (2H, m), 7.20 (1H, d, J=8.1 Hz), 7.28-7.35 (2H, m), 7.51-7.56 (2H, m), 7.70 (1H, s), 7.79 (1H, d, J=3.3 Hz), 8.21 (1H, d, J=9.0 Hz), 8.30 (1H, s), 8.33 (1H, s), 9.52 (1H, s).

### Example 82

### N-[2-methoxy-4-(5H-pyrrolo[3,2-d]pyrimidin-4-yloxy)phenyl]-N′-[3-(trifluoromethyl)phenyl]urea

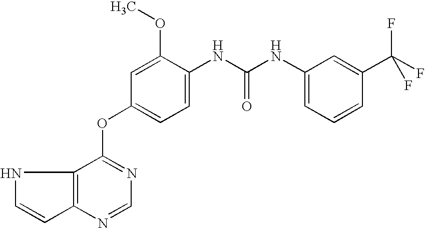

To a solution of 3-methoxy-4-nitrophenol (250 mg, 1.48 mmol) in methanol (10 mL) was added palladium carbon (50% water-containing product, 25 mg), and the mixture was stirred under a hydrogen atmosphere at room temperature for 3 hr. The reaction mixture was filtered through celite. The filtrate was concentrated under reduced pressure and dried, and the residue was dissolved in N-methylpyrrolidone (3 mL). Potassium carbonate (613 mg, 4.44 mmol) and 4-chloro-5H-pyrrolo[3,2-d]pyrimidine (216 mg, 1.40 mmol) were added, and the mixture was stirred at 110° C. for 1 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=10/90→100/0). The residue was dissolved in tetrahydrofuran (7 mL), and triethylamine (77.0 μL, 0.555 mmol) and 3-(trifluoromethyl)phenylisocyanate (31.0 μL, 0.222 mmol) were added, and the mixture was stirred at room temperature for 3 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=10/90→100/0) and recrystallized from ethyl acetate-hexane to give the title compound (11.0 mg, 2%) as a white solid.

¹H-NMR (DMSO-d₆, 300 MHz) δ 3.88 (3H, s), 6.63 (1H, d, J=3.2 Hz), 6.84 (1H, dd, J=8.9, 2.2 Hz), 7.05 (1H, d, J=2.2 Hz), 7.28-7.37 (1H, m), 7.50-7.58 (2H, m), 7.79 (1H, d, J=3.2 Hz), 8.05 (1H, s), 8.14 (1H, d, J=8.9 Hz), 8.30-8.34 (2H, m), 9.67 (1H, s), 12.29 (1H, s).

### Example 83

### N-{2-chloro-4-[(5-methyl-5H-pyrrolo[3,2-d]pyrimidin-4-yl)oxy]phenyl}-N′-{3-[(trifluoromethyl)sulfinyl]phenyl}urea

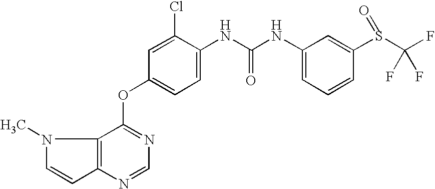

Using 2-chloro-4-[(5-methyl-5H-pyrrolo[3,2-d]pyrimidin-4-yl)oxy]aniline (442 mg, 1.61 mmol), pyridine (390 μL, 4.83 mmol), phenyl chloroformate (213 μL, 1.69 mmol), 3-[(trifluoromethyl)sulfinyl]aniline (380 mg, 1.82 mmol) and N-methylpyrrolidone (5 mL) as starting materials, and in the same manner as in Example 20, the title compound (307 mg, 37%) was obtained as a white solid.

¹H-NMR (DMSO-d₆, 200 MHz) δ 4.11 (3H, s), 6.61 (1H, d, J=2.8 Hz), 7.32 (1H, dd, J=8.9, 2.7 Hz), 7.46-7.52 (1H, m), 7.58 (1H, d, J=2.7 Hz), 7.64-7.67 (2H, m), 7.80 (1H, d, J=2.8 Hz), 8.18 (1H, d, J=8.9 Hz), 8.20 (1H, s), 8.30 (1H, s), 8.47 (1H, s), 9.86 (1H, s).

### Example 84

### N-(3-tert-butyl-1-methyl-1H-pyrazol-5-yl)-N′-{2-chloro-4-[(5-methyl-5H-pyrrolo[3,2-d]pyrimidin-4-yl)oxy]phenyl}urea

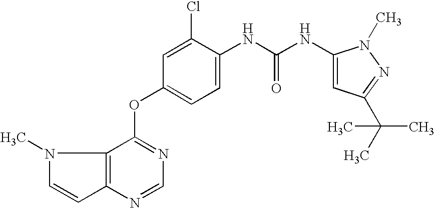

Using 2-chloro-4-[(5-methyl-5H-pyrrolo[3,2-d]pyrimidin-4-yl)oxy]aniline (268 mg, 0.976 mmol), pyridine (236 μL, 2.93 mmol), phenyl chloroformate (129 μL, 1.02 mmol), 3-tert-butyl-1-methyl-1H-pyrazole-5-amine (179 mg, 1.17 mmol) and N-methylpyrrolidone (5 mL) as starting materials, and in the same manner as in Example 20, the title compound (27.0 mg, 6%) was obtained as a white solid.

¹H-NMR (DMSO-d₆, 200 MHz) δ 1.22 (9H, s), 3.65 (3H, s), 4.10 (3H, s), 6.11 (1H, s), 6.60 (1H, d, J=3.0 Hz), 7.30 (1H, dd, J=9.3, 2.7 Hz), 7.56 (1H, d, J=2.7 Hz), 7.79 (1H, d, J=3.0 Hz), 8.16 (1H, d, J=9.3 Hz), 8.30 (1H, s), 8.61 (1H, br s), 9.22 (1H, br s).

### Example 85

### N-{2-chloro-4-[(5-methyl-5H-pyrrolo[3,2-d]pyrimidin-4-yl)oxy]phenyl}-N′-[8-hydroxy-8-(trifluoromethyl)-5,6,7,8-tetrahydronaphthalen-2-yl]urea

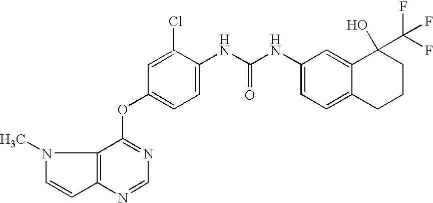

Using 2-chloro-4-[(5-methyl-5H-pyrrolo[3,2-d]pyrimidin-4-yl)oxy]aniline (283 mg, 1.03 mmol), pyridine (250 μL, 3.09 mmol), phenyl chloroformate (137 μL, 1.08 mmol), 7-amino-1-(trifluoromethyl)-1,2,3,4-tetrahydronaphthalen-1-ol (262 mg, 1.13 mmol) and N-methylpyrrolidone (5 mL) as starting materials, and in the same manner as in Example 20, the title compound (97.0 mg, 18%) was obtained as a white solid.

¹H-NMR (DMSO-d₆, 200 MHz) δ 1.75-2.20 (4H, m), 2.65-2.75 (2H, m), 4.10 (3H, s), 6.41 (1H, s), 6.60 (1H, d, J=3.2 Hz), 7.10 (1H, d, J=8.4 Hz), 7.29 (1H, dd, J=9.0, 2.8 Hz), 7.49-7.55 (2H, m), 7.68 (1H, s), 7.79 (1H, d, J=3.2 Hz), 8.19-8.30 (3H, m), 9.46 (1H, s).

### Example 86

### N-{2-chloro-4-[(5-methyl-5H-pyrrolo[3,2-d]pyrimidin-4-yl)oxy]phenyl}-N′-[1-methyl-3-(2,2,2-trifluoro-1-hydroxyethyl)-1H-pyrazol-5-yl]urea

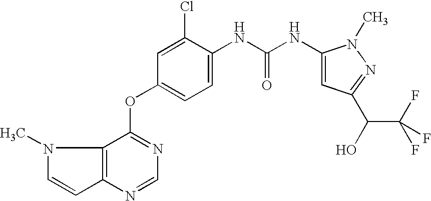

Using 2-chloro-4-[(5-methyl-5H-pyrrolo[3,2-d]pyrimidin-4-yl)oxy]aniline (302 mg, 1.10 mmol), pyridine (266 μL, 3.30 mmol), phenyl chloroformate (146 μL, 1.15 mmol), 1-(5-amino-1-methyl-1H-pyrazol-3-yl)-2,2,2-trifluoroethanol (258 mg, 1.32 mmol) and N-methylpyrrolidone (3 mL) as starting materials, and in the same manner as in Example 20, the title compound (66.4 mg, 12%) was obtained as a white solid.

¹H-NMR (DMSO-d₆, 200 MHz) δ 3.72 (3H, s), 4.10 (3H, s), 4.87-4.95 (1H, m), 6.35 (1H, s), 6.60-6.67 (2H, m), 7.31 (1H, dd, J=8.9, 2.6 Hz), 7.57 (1H, d, J=2.6 Hz), 7.79 (1H, d, J=2.8 Hz), 8.15 (1H, d, J=8.9 Hz), 8.30 (1H, s), 8.67 (1H, s), 9.40 (1H, s).

### Example 87

### N-(3-acetylphenyl)-N′-{2-chloro-4-[(5-methyl-5H-pyrrolo[3,2-d]pyrimidin-4-yl)oxy]phenyl}urea

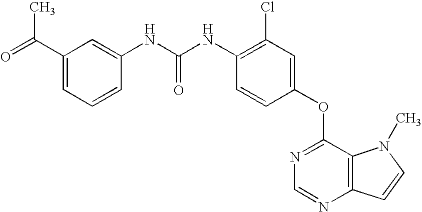

To a solution of 2-chloro-4-[(5-methyl-5H-pyrrolo[3,2-d]pyrimidin-4-yl)oxy]aniline (200 mg, 0.73 mmol) and triethylamine (304 μL, 2.2 mmol) in tetrahydrofuran (4 mL) was added 3-acetylphenylisocyanate (120 μL, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate) and recrystallized from ethyl acetate-hexane to give the title compound (205 mg, 65%) as a colorless solid.

¹H-NMR (DMSO-d₆, 300 MHz) δ 2.58 (3H, s), 4.11 (3H, s), 6.60-6.62 (1H, m), 7.30-8.45 (10H, m), 9.62 (1H, s).

### Example 88

### Methyl 3-{[({2-chloro-4-[(5-methyl-5H-pyrrolo[3,2-d]pyrimidin-4-yl)oxy]phenyl}amino)carbonyl]amino}benzoate

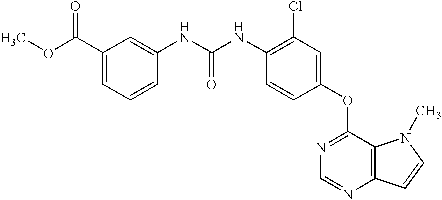

To a solution of 2-chloro-4-[(5-methyl-5H-pyrrolo[3,2-d]pyrimidin-4-yl)oxy]aniline (200 mg, 0.73 mmol) and triethylamine (304 μL, 2.2 mmol) in tetrahydrofuran (4 mL) was added methyl 3-isocyanatobenzoate (155 mg, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate) and recrystallized from ethyl acetate-hexane to give the title compound (258 mg, 78%) as a colorless solid.

¹H-NMR (DMSO-d₆, 300 MHz) δ 3.87 (3H, s), 4.11 (3H, s), 6.61 (1H, d, J=3.0 Hz), 7.30-7.90 (6H, m), 8.10-8.50 (4H, m), 9.65 (1H, s).

### Example 89

### N-{2-chloro-4-[(5-methyl-5H-pyrrolo[3,2-d]pyrimidin-4-yl)oxy]phenyl}-N′-[3-(trifluoromethyl)phenyl]urea methanesulfonic acid salt

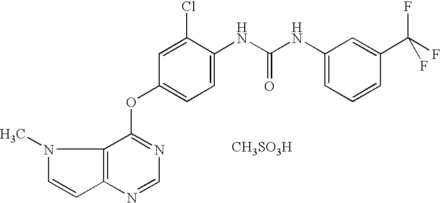

To a solution of N-{2-chloro-4-[(5-methyl-5H-pyrrolo[3,2-d]pyrimidin-4-yl)oxy]phenyl}-N′-[3-(trifluoromethyl)phenyl]urea (10.4 mg, 0.023 mmol) in acetonitrile (0.5 mL) was added methanesulfonic acid (22 μL, 0.02 mmol). The precipitated crystals were collected by filtration, and vacuum dried to give the title compound (8.1 mg, 64%) as colorless crystals.

¹H-NMR (DMSO-d₆, 300 MHz) δ 2.31 (3H, s), 4.14 (3H, s), 6.68 (1H, d, J=3.0 Hz), 7.30-7.40 (2H, m), 7.50-7.65 (3H, m), 7.93 (1H, d, J=3.0 Hz), 8.06 (1H, s), 8.20 (1H, d, J=9.0 Hz), 8.45-8.55 (2H, m), 9.75 (1H, s).

### Example 90

### N-{2-chloro-4-[(5-methyl-5H-pyrrolo[3,2-d]pyrimidin-4-yl)oxy]phenyl}-N′-isoquinolin-3-ylurea

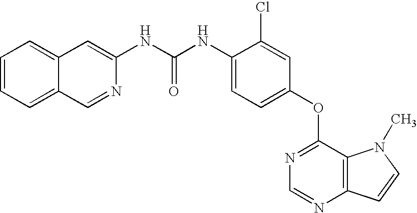

To a solution of 3-aminoisoquinoline (210 mg, 1.5 mmol), pyridine (236 μL, 2.9 mmol) and 4-nitrophenyl chlorocarbonate (294 mg, 1.5 mmol) in N,N-dimethylacetamide (6 mL) was added 2-chloro-4-[(5-methyl-5H-pyrrolo[3,2-d]pyrimidin-4-yl)oxy]aniline (200 mg, 0.73 mmol), and the mixture was stirred at 90° C. for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate) and recrystallized from ethyl acetate-hexane to give the title compound (53 mg, 16%) as a colorless solid.

¹H-NMR (DMSO-d₆, 300 MHz) δ 4.11 (3H, s), 6.61 (1H, d, J=3.0 Hz), 7.30-8.40 (10H, m), 9.19 (1H, s), 10.08 (1H, s).

### Example 91

### N-{2-chloro-4-[(5-methyl-5H-pyrrolo[3,2-d]pyrimidin-4-yl)oxy]phenyl}-N′-(2-phenylethyl)urea

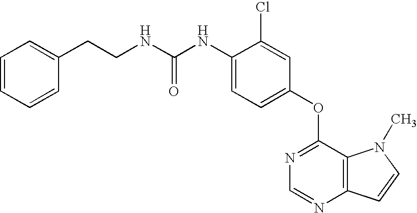

To a solution of 2-chloro-4-[(5-methyl-5H-pyrrolo[3,2-d]pyrimidin-4-yl)oxy]aniline (200 mg, 0.73 mmol) and triethylamine (304 μL, 2.2 mmol) in tetrahydrofuran (4 mL) was added phenethylisocyanate (121 μL, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate:hexane=1:1) and recrystallized from ethyl acetate-hexane to'give the title compound (101 mg, 33%) as a colorless solid.

¹H-NMR (DMSO-d₆, 300 MHz) δ 2.78 (2H, t, J=7.2 Hz), 3.38 (2H, dt, 7.2, 7.2 Hz), 4.09 (3H, s), 6.55-6.65 (1H, m), 6.90-7.05 (1H, m), 7.15-7.40 (6H, m), 7.40-7.50 (1H, m), 7.70-7.85 (1H, m), 8.10 (1H, s), 8.17 (1H, d, J=9.0 Hz), 8.29 (1H, s).

### Example 92

### N-1,3-benzothiazol-6-yl-N′-{2-chloro-4-[(5-methyl-5H-pyrrolo[3,2-d]pyrimidin-4-yl)oxy]phenyl}urea

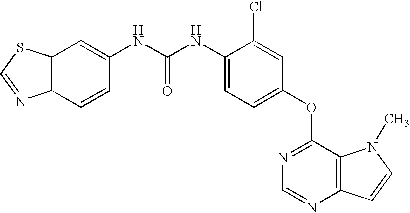

To a solution of 6-aminobenzothiazole (218 mg, 1.5 mmol), pyridine (236 μL, 2.9 mmol) and 4-nitrophenyl chlorocarbonate (294 mg, 1.5 mmol) in N,N-dimethylacetamide (6 mL) was added 2-chloro-4-[(5-methyl-5H-pyrrolo[3,2-d]pyrimidin-4-yl)oxy]aniline (200 mg, 0.73 mmol), and the mixture was stirred at 90° C. for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate) and recrystallized from ethyl acetate-hexane to give the title compound (31 mg, 9%) as a colorless solid.

¹H-NMR (DMSO-d₆; 300 MHz) δ 4.11 (3H, s), 6.55-6.65 (1H, m), 7.30-7.65 (3H, m), 7.75-7.85 (1H, m), 8.02 (1H, d, J=8.1 Hz), 8.21 (1H, d, J=9.0 Hz), 8.31 (1H, s), 8.41 (1H, s), 8.47 (1H, s), 9.23 (1H, s), 9.66 (1H, s).

### Example 93

### N-{2-chloro-4-[(5-methyl-5H-pyrrolo[3,2-d]pyrimidin-4-yl)oxy]phenyl}-N′-2-naphthylurea

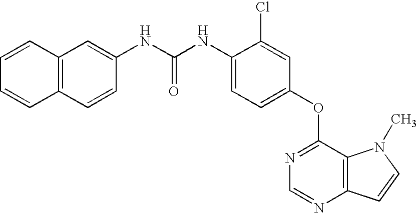

To a solution of 2-chloro-4-[(5-methyl-5H-pyrrolo[3,2-d]pyrimidin-4-yl)oxy]aniline (200 mg, 0.73 mmol) and triethylamine (304 mL, 2.2 mmol) in tetrahydrofuran (4 mL) was added 2-naphthylisocyanate (148 mg, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate:hexane=1:1) and recrystallized from ethyl acetate-hexane to give the title compound (22 mg, 7%) as a colorless solid.

¹H-NMR (DMSO-d₆, 300 MHz) δ 4.11 (3H, s), 6.60-6.65 (1H, m), 7.30-8.40 (12H, m), 8.47 (1H, s), 9.62 (1H, s).

### Example 94

### N-{2-chloro-4-[(5-methyl-5H-pyrrolo[3,2-d]pyrimidin-4-yl)oxy]phenyl}-N′-(5-methyl-3-phenylisoxazol-4-yl)urea

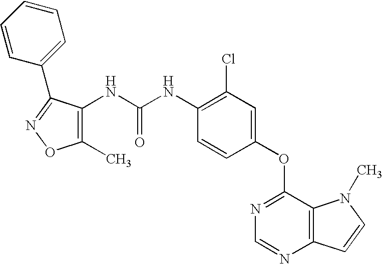

To a solution of 2-chloro-4-[(5-methyl-5H-pyrrolo[3,2-d]pyrimidin-4-yl)oxy]aniline (200 mg, 0.73 mmol) and triethylamine (304 μL, 2.2 mmol) in tetrahydrofuran (4 mL) was added 5-methyl-3-phenylisoxazol-4-ylisocyanate (175 mg, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate:hexane=67:33-100:0) and recrystallized from ethyl acetate-hexane to give the title compound (106 mg, 31%) as a colorless solid.

¹H-NMR (DMSO-d₆, 300 MHz) δ 2.41 (3H, s), 4.09 (3H, s), 6.59 (1H, d, J=3.2 Hz), 7.27 (1H, dd, J=8.8, 3.2 Hz), 7.40-8.60 (11H, m).

### Example 95

### N-{2-chloro-4-[(5-methyl-5H-pyrrolo[3,2-d]pyrimidin-4-yl)oxy]phenyl}-N′-quinolin-3-ylurea

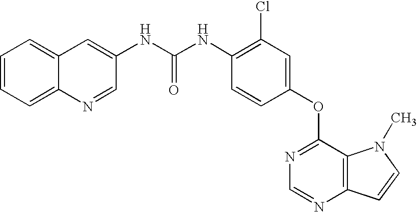

To a solution of 3-aminoquinoline (210 mg, 1.5 mmol), pyridine (236 μL, 2.9 mmol) and 4-nitrophenyl chlorocarbonate (294 mg, 1.5 mmol) in N,N-dimethylacetamide (6 mL) was added 2-chloro-4-[(5-methyl-5H-pyrrolo[3,2-d]pyrimidin-4-yl)oxy]aniline (200 mg, 0.73 mmol), and the mixture was stirred at 90° C. for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate) and recrystallized from ethyl acetate-hexane to give the title compound (44 mg, 7%) as a colorless solid.

¹H-NMR (DMSO-d₆, 300 MHz) δ 4.11 (3H, s), 6.61 (1H, d, J=3.0 Hz), 7.34 (1H, dd, J=9.0, 3.0 Hz), 7.50-7.70 (3H, m), 7.80-8.00 (3H, m), 8.23 (1H, d, J=9.0 Hz), 8.31 (1H, s), 8.55-8.65 (2H, m), 8.83 (1H, d, J=2.4 Hz), 9.85 (1H, s).

### Example 96

### N-biphenyl-2-yl-N′-{2-chloro-4-[(5-methyl-5H-pyrrolo[3,2-d]pyrimidin-4-yl)oxy]phehyl}urea

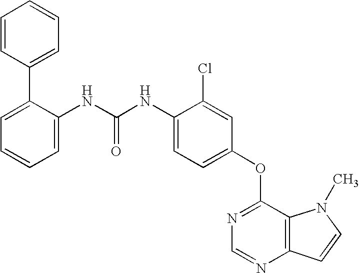

To a solution of 2-chloro-4-[(5-methyl-5H-pyrrolo[3,2-d]pyrimidin-4-yl)oxy]aniline (200 mg, 0.73 mmol) and triethylamine (304 μL, 2.2 mmol) in tetrahydrofuran (4 mL) was added 2-biphenylisocyanate (150 μL, 0.87 mmol), and the mixture was stirred at room temperature for 18 hr. The reaction mixture was diluted with water, and extracted with ethyl acetate (×1). The organic layer was washed with saturated brine, dried over anhydrous sodium sulfate, and filtrated. The filtrate was concentrated under reduced pressure, and the residue was purified by column chromatography (NH silica gel, ethyl acetate:hexane=1:1) and recrystallized from ethyl acetate-hexane to give the title compound (254 mg, 74%) as a colorless solid.

¹H-NMR (DMSO-d₆, 300 MHz) δ 4.10 (3H, s), 6.55-6.65 (1H, m), 7.15-7.60 (10H, m), 7.75-7.85 (2H, m), 8.05-8.20 (1H, m), 8.30 (1H, d, J=1.8 Hz), 8.46 (1H, s), 8.60 (1H, s).

### Example 97

### N-{2-chloro-4-[(5-methyl-5H-pyrrolo[3,2-d]pyrimidin-4-yl)oxy]phenyl}-N′-[2-methoxy-5-(trifluoromethyl)phenyl]urea

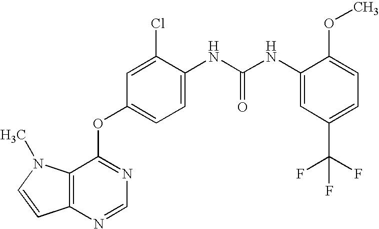

To a solution of 2-methoxy-5-(trifluoromethyl)aniline (382 mg, 2.0 mmol) and pyridine (633 mg, 8.0 mmol) in N-methylpyrrolidone (2 mL) was added phenyl chloroformate (251 μL, 2.0 mmol) under ice-cooling, and the mixture was stirred at room temperature for 3 hr. 2-Chloro-4-[(5-methyl-5H-pyrrolo[3,2-d]pyrimidin-4-yl)oxy]aniline (275 mg, 1.0 mmol) was added to the reaction mixture and the mixture was stirred at 110° C. for 6 hr. The reaction mixture was diluted with 1N aqueous sodium hydroxide solution and extracted with ethyl acetate. The organic layer was washed with water, and concentrated under reduced pressure. The residue was purified by column chromatography (silica gel, hexane/ethyl acetate=70/30→40/100) and recrystallized from ethyl acetate/methanol to give the title compound (350 mg, 71%).

¹H-NMR (DMSO-d₆, 300 MHz) δ 3.99 (3H, s), 4.11 (3H, s), 6.61 (1H, d, J=3.2 Hz), 7.23 (1H, br d, J=8.5 Hz), 7.30 (1H, dd, J=9.0, 2.7 Hz), 7.35 (1H, br dd, J=8.5, 1.9 Hz), 7.56 (1H, d, J=2.7 Hz), 7.80 (1H, d, J=3.2 Hz), 8.13 (1H, d, J=9.0 Hz), 8.31 (1H, s), 8.55 (1H, br d, J=1.9 Hz), 9.14 (1H, br s), 9.25 (1H, br s).

### Example 98

### N-{2-chloro-4-[(5-methyl-5H-pyrrolo[3,2-d]pyrimidin-4-yl)oxy]phenyl}-N′-[4-methoxy-3-(trifluoromethyl)phenyl]urea

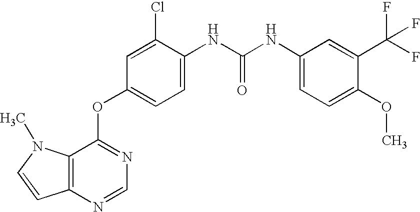

Using 4-methoxy-3-(trifluoromethyl)aniline (382 mg, 2.0 mmol), pyridine (633 mg, 8.0 mmol) and N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (162 mg, 33%) was obtained.

¹H-NMR (DMSO-d₆, 300 MHz) δ 3.86 (3H, s), 4.10 (3H, s), 6.61 (1H, d, J=3.0 Hz), 7.24 (1H, d, J=9.2 Hz), 7.30 (1H, dd, J=9.0, 2.7 Hz), 7.56 (1H, d, J=2.7 Hz), 7.59 (1H, dd, J=9.2, 2.7 Hz), 7.80 (1H, d, J=3.0 Hz), 7.88 (1H, d, J=2.7 Hz), 8.17 (1H, d, J=9.0 Hz), 8.30 (1H, s), 8.37 (1H, br s), 9.51 (1H, br s).

### Example 99

### N-{2-chloro-4-[(5-methyl-5H-pyrrolo[3,2-d]pyrimidin-4-yl)oxy]phenyl}-N′-[4-cyano-3-(trifluoromethyl)phenyl]urea

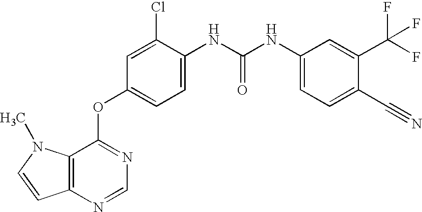

Using 4-amino-2-(trifluoromethyl)benzonitrile (372 mg, 2.0 mmol), pyridine (633 mg, 8.0 mmol) and N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (179 mg, 37%) was obtained.

¹H-NMR (DMSO-d₆, 300 MHz) δ 4.11 (3H, s), 6.61 (1H, d, J=3.0 Hz), 7.34 (1H, dd, J=8.9, 2.7 Hz), 7.60 (1H, d, J=2.7 Hz), 7.76-7.80 (1H, m), 7.80 (1H, d, J=3.0 Hz), 8.07 (1H, d, J=8.7 Hz), 8.11 (1H, d, J=8.9 Hz), 8.23 (1H, d, J=2.1 Hz), 8.31 (1H, s), 8.64 (1H, br s), 10.22 (1H, br s).

### Example 100

### N-{2-chloro-4-[(5-methyl-5H-pyrrolo[3,2-d]pyrimidin-4-yl)oxy]phenyl}-N′-[3-methoxy-5-(trifluoromethyl)phenyl]urea

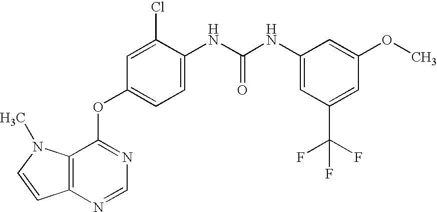

Using 3-methoxy-5-(trifluoromethyl)aniline (382 mg, 2.0 mmol), pyridine (633 mg, 8.0 mmol) and N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (103 mg, 21%) was obtained.

¹H-NMR (DMSO-d₆, 300 MHz) δ 3.83 (3H, s), 4.11 (3H, s), 6.61 (1H, d, J=3.0 Hz), 6.88 (1H, br s), 7.26 (1H, br s), 7.32 (1H, dd, J=9.0, 2.7 Hz), 7.51 (1H, br s), 7.57 (1H, d, J=2.7 Hz), 7.80 (1H, d, J=3.0 Hz), 8.15 (1H, d, J=9.0 Hz), 8.30 (1H, s), 8.47 (1H, br s), 9.76 (1H, br s).

### Example 101

### N-{2-chloro-4-[(5-methyl-5H-pyrrolo[3,2-d]pyrimidin-4-yl)oxy]phenyl}-N′-pyridin-2-ylurea

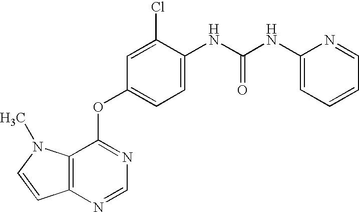

Using 2-aminopyridine (188 mg, 2.0 mmol), pyridine (633 mg, 8.0 mmol), N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (299 mg, 76%) was obtained.

¹H-NMR (DMSO-d₆, 300 MHz) δ 4.11 (3H, s), 6.61 (1H, d, J=3.0 Hz) 7.03-7.07 (1H, m), 7.25 (1H, br d, J=8.4 Hz), 7.33 (1H, dd, J=9.2, 2.6 Hz), 7.60 (1H, d, J=2.6 Hz), 7.77-7.82 (1H, m), 7.80 (1H, d, J=3.0 Hz), 8.31 (1H, s), 8.31-8.34 (1H, m), 8.40 (1H, d, J=9.2 Hz), 10.03 (1H, s), 11.83 (1H, br s).

### Example 102

### N-{2-chloro-4-[(5-methyl-5H-pyrrolo[3,2-d]pyrimidin-4-yl)oxy]phenyl}-N′-pyridin-3-ylurea

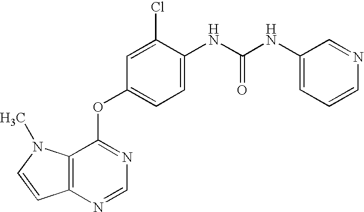

Using 3-aminopyridine (188 mg, 2.0 mmol), pyridine (633 mg, 8.0 mmol), N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (90 mg, 23%) was obtained.

¹H-NMR (DMSO-d₆, 300 MHz) δ 4.11 (3H, s), 6.61 (1H, d, J=3.0 Hz), 7.32 (1H, dd, J=9.0, 2.7 Hz), 7.32-7.37 (1H, m), 7.57 (1H, d, J=2.7 Hz), 7.80 (1H, d, J=3.0 Hz), 7.98 (1H, ddd, J=8.2, 2.5, 1.5 Hz), 8.17 (1H, d, J=9.0 Hz), 8.22 (1H, dd, J=4.5, 1.5 Hz), 8.31 (1H, s), 8.49 (1H, br s), 8.63 (1H, d, J=2.5 Hz), 9.56 (1H, br s).

### Example 103

### N-{2-chloro-4-[(5-methyl-5H-pyrrolo[3,2-d]pyrimidin-4-yl)oxy]phenyl}-N′-pyridin-4-ylurea

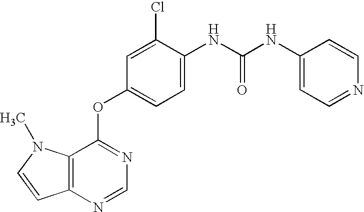

Using 4-aminopyridine (188 mg, 2.0 mmol), pyridine (633 mg, 8.0 mmol), N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (88 mg, 22%) was obtained.

¹H-NMR (DMSO-d₆, 300 MHz) δ 4.11 (3H, s), 6.61 (1H, d, J=3.2 Hz), 7.33 (1H, dd, J=9.0, 2.6 Hz), 7.46 (2H, d, J=6.3 Hz), 7.58 (1H, d, J=2.6 Hz), 7.80 (1H, d, J=3.2 Hz), 8.15 (1H, d, J=9.0 Hz), 8.31 (1H, s), 8.39 (2H, d, J=6.3 Hz), 8.56 (1H, br s), 9.78 (1H, br s).

### Example 104

### N-{2-chloro-4-[(5-methyl-5H-pyrrolo[3,2-d]pyrimidin-4-yl)oxy]phenyl}-N′-(2-chloropyridin-4-yl)urea

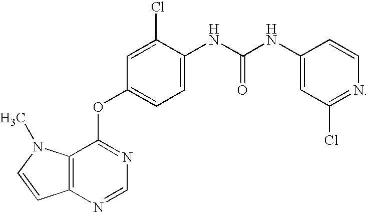

Using 4-amino-2-chloropyridine (257 mg, 2.0 mmol), pyridine (633 mg, 8.0 mmol), N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (348 mg, 81%) was obtained.

¹H-NMR (DMSO-d₆, 300 MHz) δ 4.11 (3H, s), 6.61 (1H, d, J=3.0 Hz), 7.31 (1H, dd, J=5.5, 1.8 Hz), 7.34 (1H, dd, J=8.9, 2.6 Hz), 7.60 (1H, d, J=2.6 Hz), 7.70 (1H, d, J=1.8 Hz), 7.80 (1H, d, J=3.0 Hz), 8.11 (1H, d, J=8.9 Hz), 8.22 (1H, d, J=5.5 Hz), 8.31 (1H, s), 8.64 (1H, br s), 9.99 (1H, br s).

### Example 105

### N-{2-chloro-4-[(5-methyl-5H-pyrrolo[3,2-d]pyrimidin-4-yl)oxy]phenyl}-N′-(cyclopropylmethyl)urea

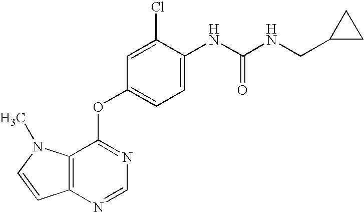

To a solution of 2-chloro-4-[(5-methyl-5H-pyrrolo[3,2-d]pyrimidin-4-yl)oxy]aniline (275 mg, 1.0 mmol) and triethylamine (2.79 mL, 20 mmol) in dichloromethane (10 mL) was added triphosgene (297 mg, 1.0 mmol) under ice-cooling, and the mixture was stirred at 0° C. for 15 min. Cyclopropylmethylamine (260 μL, 3.0 mmol) was added to the reaction mixture, and the mixture was stirred at room temperature for 2 hr. The reaction mixture was concentrated under reduced pressure, and the residue was diluted with ethyl acetate and washed with water and saturated brine. The organic layer was concentrated under reduced pressure, and the residue was purified by column chromatography (silica gel, hexane/ethyl acetate=60/40→0/100) and recrystallized from ethyl acetate/diisopropyl ether to give the title compound (214 mg, 58%).

¹H-NMR (DMSO-d₆, 300 MHz) δ 0.17-0.22 (2H, m), 0.43-0.49 (2H, m), 0.89-1.02 (1H, m), 3.00 (2H, dd, J=6.6, 5.5 Hz), 4.09 (3H, s), 6.60 (1H, d, J=3.0 Hz), 7.08 (1H, br t, J=5.5 Hz), 7.23 (1H, dd, J=9.1, 2.8 Hz), 7.48 (1H, d, J=2.8 Hz), 7.78 (1H, d, J=3.0 Hz), 8.08 (1H, br s), 8.18 (1H, d, J=9.1 Hz), 8.29 (1H, s).

### Example 106

### N-{2-chloro-4-[(5-methyl-5H-pyrrolo[3,2-d]pyrimidin-4-yl)oxy]phenyl}-N′-[1-oxide-4-(trifluoromethyl)pyridin-2-yl]urea

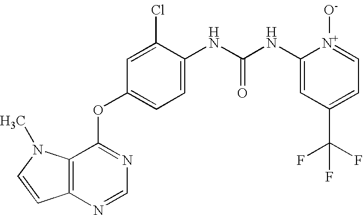

A mixture of phenyl[1-oxide-4-(trifluoromethyl)pyridin-2-yl]carbamate (420 mg, 1.4 mmol), 2-chloro-4-[(5-methyl-5H-pyrrolo[3,2-d]pyrimidin-4-yl)oxy]aniline (275 mg, 1.0 mmol), pyridine (633 mg, 8.0 mmol) and N-methylpyrrolidone (2 mL) was stirred at 90° C. for 7 hr. The reaction mixture was diluted with water and extracted with ethyl acetate. The organic layer was washed with water and saturated brine, and concentrated under reduced pressure. The residue was purified by column chromatography (silica gel, ethyl acetate/methanol=100/0→70/30) and recrystallized from ethyl acetate to give the title compound (263 mg, 55%) as a white solid.

¹H-NMR (DMSO-d₆, 300 MHz) δ 4.11 (3H, s), 6.61 (1H, d, J=3.2 Hz), 7.35 (1H, dd, J=9.0, 2.7 Hz), 7.44 (1H, dd, J=6.7, 2.3 Hz), 7.60 (1H, d, J=2.7 Hz), 7.81 (1H, d, J=3.2 Hz), 8.07 (1H, d, J=9.0 Hz), 8.31 (1H, s), 8.58 (1H, d, J=6.7 Hz), 8.61 (1H, d, J=2.3 Hz), 9.80 (1H, br s), 10.94 (1H, br s)

### Example 107

### N-{2-chloro-4-[(5-methyl-5H-pyrrolo[3,2-d]pyrimidin-4-yl)oxy]phenyl}-N′-(3,3,3-trifluoropropyl)urea

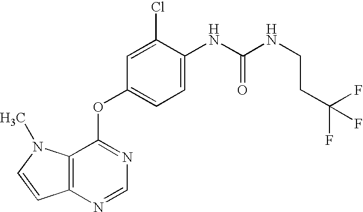

Using 2-chloro-4-[(5-methyl-5H-pyrrolo[3,2-d]pyrimidin-4-yl)oxy]aniline (275 mg, 1.0 mmol), triethylamine (4.18 mL, 30 mmol), dichloromethane (10 mL), triphosgene (297 mg, 1.0 mmol) and 3,3,3-trifluoropropylamine hydrochloride (449 mg, 3.0 mmol), and in the same manner as in Example 105, the title compound (236 mg, 57%) was obtained.

¹H-NMR (DMSO-d₆, 300 MHz) δ 2.41-2.57 (2H, m), 3.38 (2H, q, J=6.4 Hz), 4.09 (3H, s), 6.60 (1H, d, J=3.0 Hz), 7.15 (1H, br t, J=5.8 Hz), 7.25 (1H, dd, J=9.1, 2.8 Hz), 7.49 (1H, d, J=2.8 Hz), 7.79 (1H, d, J=3.0 Hz), 8.14 (1H, d, J=9.1 Hz), 8.22 (1H, br s), 8.29 (1H, s).

### Example 108

### N-{2-chloro-4-[(5-methyl-5H-pyrrolo[3,2-d]pyrimidin-4-yl)oxy]phenyl}-N′-cyclopropylurea

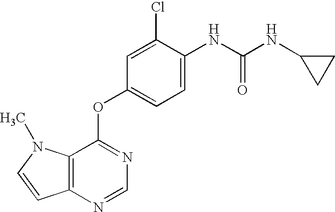

Using 2-chloro-4-[(5-methyl-5H-pyrrolo[3,2-d]pyrimidin-4-yl)oxy]aniline (275 mg, 1.0 mmol), triethylamine (2.79 mL, 20 mmol), dichloromethane (10 mL), triphosgene (297 mg, 1.0 mmol) and cyclopropylamine (208 μL, 3.0 mmol), and in the same manner as in Example 105, the title compound (183 mg, 51%) was obtained.

¹H-NMR (DMSO-d₆, 300 MHz) δ 0.41-0.46 (2H, m), 0.64-0.70 (2H, m), 2.54-2.62 (1H, m), 4.09 (3H, s), 6.60 (1H, d, J=3.0 Hz), 7.16 (1H, br d, J=2.7 Hz), 7.25 (1H, dd, J=9.0, 2.7 Hz), 7.49 (1H, d, J=2.7 Hz), 7.78 (1H, d, J=3.0 Hz), 7.94 (1H, br s), 8.18 (1H, d, J=9.0 Hz), 8.29 (1H, s).

### Example 109

### N-{2-chloro-4-[(5-methyl-5H-pyrrolo[3,2-d]pyrimidin-4-yl)oxy]phenyl}-N′-[5-(trifluoromethyl)pyridin-3-yl]urea

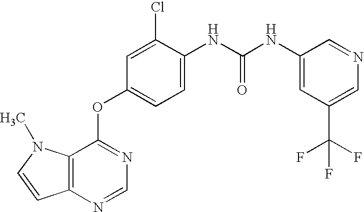

Using phenyl[5-(trifluoromethyl)pyridin-3-yl]carbamate (282 mg, 1.0 mmol), 2-chloro-4-[(5-methyl-5H-pyrrolo[3,2-d]pyrimidin-4-yl)oxy]aniline (275 mg, 1.0 mmol), pyridine (633 mg, 8.0 mmol) and N-methylpyrrolidone (2 mL), and in the same manner as in Example 106, the title compound (228 mg, 49%) was obtained as a white solid.

¹H-NMR (DMSO-d₆, 300 MHz) δ 4.11 (3H, s), 6.61 (1H, d, J=3.0 Hz), 7.34 (1H, dd, J=9.0, 2.7 Hz), 7.59 (1H, d, J=2.7 Hz), 7.80 (1H, d, J=3.0 Hz), 8.14 (1H, d, J=9.0 Hz), 8.31 (1H, s), 8.48 (1H, m), 8.60 (1H, m), 8.65 (1H, br s), 8.77 (1H, m), 9.94 (1H, br s).

### Example 110

### N-{2-chloro-4-[(5-methyl-5H-pyrrolo[3,2-d]pyrimidin-4-yl)oxy]phenyl}-N′-[2-(trifluoromethyl)pyridin-4-yl]urea

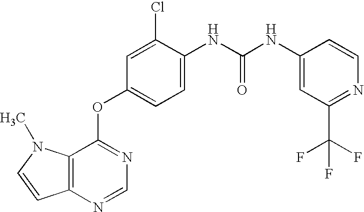

Using phenyl[2-(trifluoromethyl)pyridin-4-yl]carbamate (282 mg, 1.0 mmol), 2-chloro-4-[(5-methyl-5H-pyrrolo[3,2-d]pyrimidin-4-yl)oxy]aniline (275 mg, 1.0 mmol), pyridine (633 mg, 8.0 mmol) and N-methylpyrrolidone (2 mL), and in the same manner as in Example 106, the title compound (251 mg, 54%) was obtained as a white solid.

¹H-NMR (DMSO-d₆, 300 MHz) δ 4.11 (3H, s), 6.61 (1H, d, J=3.0 Hz), 7.35 (1H, dd, J=9.0, 2.7 Hz), 7.57 (1H, dd, J=5.7, 1.7 Hz), 7.60 (1H, d, J=2.7 Hz), 7.80 (1H, d, J=3.0 Hz), 8.09 (1H, d, J=1.7 Hz), 8.12 (1H, d, J=9.0 Hz), 8.31 (1H, s), 8.57 (1H, d, J=5.7 Hz), 8.68 (1H, br s), 10.16 (1H, br s)

### Example 111

### N-{2-chloro-4-[(5-methyl-5H-pyrrolo[3,2-d]pyrimidin-4-yl)oxy]phenyl}-N′-[1-oxide-5-(trifluoromethyl)pyridin-3-yl]urea

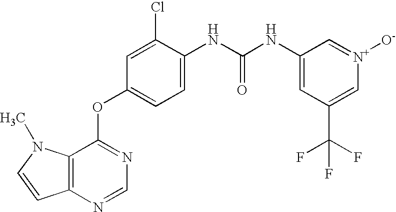

Using phenyl[1-oxide-5-(trifluoromethyl)pyridin-3-yl]carbamate (298 mg, 1.0 mmol), 2-chloro-4-[(5-methyl-5H-pyrrolo[3,2-d]pyrimidin-4-yl)oxy]aniline (275 mg, 1.0 mmol), pyridine (633 mg, 8.0 mmol) and N-methylpyrrolidone (2 mL), and in the same manner as in Example 106, the title compound (269 mg, 56%) was obtained as a white solid.

¹H-NMR (DMSO-d₆, 300 MHz) δ 4.11 (3H, s), 6.61 (1H, d, J=3.0 Hz), 7.35 (1H, dd, J=9.0, 2.7 Hz), 7.60 (1H, d, J=2.7 Hz), 7.73 (1H, br s), 7.80 (1H, d, J=3.0 Hz), 8.05 (1H, d, J=9.0 Hz), 8.31 (1H, s), 8.43 (1H, br s), 8.68 (1H, br s), 8.71 (1H, br s), 9.93 (1H, br s).

### Example 112

### N-{2-chloro-4-[(5-methyl-5H-pyrrolo[3,2-d]pyrimidin-4-yl)oxy]phenyl}-N′-(3-ethynylphenyl)urea

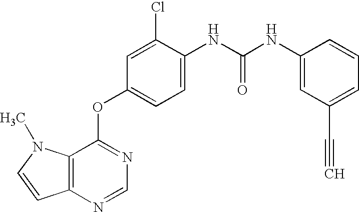

Using 3-ethynylaniline (234 mg, 2.0 mmol), pyridine (633 mg, 8.0 mmol), N-methylpyrrolidone (2 mL), phenyl chloroformate (251 μL, 2.0 mmol) and 2-chloro-4-[(5-methyl-5H-pyrrolo[3,2-d]pyrimidin-4-yl)oxy]aniline (275 mg, 1.0 mmol), and in the same manner as in Example 97, the title compound (87 mg, 21%) was obtained.

¹H-NMR (DMSO-d₆, 300 MHz) δ 4.11 (3H, s), 4.18 (1H, s), 6.61 (1H, d, J=3.2 Hz), 7.11 (1H, dt, J=7.7, 1.3 Hz), 7.32 (1H, dd, J=9.0, 2.8 Hz), 7.33 (1H, t, J=7.7 Hz), 7.39-7.43 (1H, m), 7.56 (1H, d, J=2.8 Hz), 7.72 (1H, t, J=1.8 Hz), 7.80 (1H, d, J=3.2 Hz), 8.17 (1H, d, J=9.0 Hz), 8.31 (1H, s), 8.42 (1H, br s), 9.51 (1H, br s).

### Example 113

### N-(5-tert-butylisoxazol-3-yl)-N′-{2-chloro-4-[(5-methyl-5H-pyrrolo[3,2-d]pyrimidin-4-yl)oxy]phenyl}urea

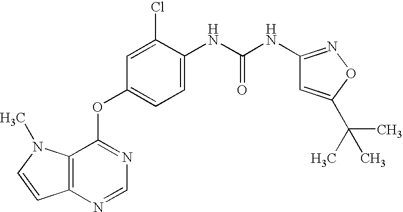

To a solution of 2-chloro-4-[(5-methyl-5H-pyrrolo[3,2-d]pyrimidin-4-yl)oxy]aniline (275 mg, 1.0 mmol) and triethylamine (2.79 mL, 20 mmol) in dichloromethane (10 mL) was added triphosgene (297 mg, 1.0 mmol) under ice-cooling, and the mixture was stirred at 0° C. for 15 min. 5-tert-Butyl-3-aminoisoxazole (280 mg, 2.0 mmol) was added to the reaction mixture and the mixture was stirred at room temperature for 18 hr. The reaction mixture was diluted with water, and extracted with dichloromethane. The organic layer was concentrated under reduced pressure, and the residue was purified by NH silica gel column chromatography (hexane/ethyl acetate=80/20→0/100) and then silica gel column chromatography (hexane/ethyl acetate=95/5→20/80) and recrystallized from ethyl acetate/diisopropyl ether to give the title compound (159 mg, 36%) as a white solid.

¹H-NMR (DMSO-d₆, 300 MHz) δ 1.30 (9H, s), 4.10 (3H, s), 6.47 (1H, s), 6.61 (1H, d, J=3.0 Hz), 7.32 (1H, dd, J=9.2, 2.7 Hz), 7.58 (1H, d, J=2.7 Hz), 7.80 (1H, d, J=3.0 Hz), 8.19 (1H, d, J=9.2 Hz) 8.30 (1H, s), 8.74 (1H, br s), 10.21 (1H, br s).

### Example 114

### N-{2-chloro-4-[(5-methyl-5H-pyrrolo[3,2-d]pyrimidin-4-yl)oxy]phenyl}-N′-[6-(trifluoromethyl)pyridin-3-yl]urea

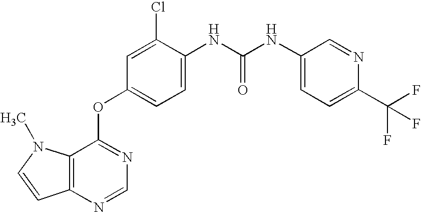

Using phenyl[6-(trifluoromethyl)pyridin-3-yl]carbamate (282 mg, 1.0 mmol), 2-chloro-4-[(5-methyl-5H-pyrrolo[3,2-d]pyrimidin-4-yl)oxy]aniline (275 mg, 1.0 mmol), pyridine (633 mg, 8.0 mmol) and N-methylpyrrolidone (2 mL), and in the same manner as in Example 106, the title compound (248 mg, 54%) was obtained as a white solid.

¹H-NMR (DMSO-d₆, 300 MHz) δ 4.11 (3H, s), 6.61 (1H, d, J=3.2 Hz), 7.34 (1H, dd, J=9.0, 2.6 Hz), 7.59 (1H, d, J=2.6 Hz), 7.80 (1H, d, J=3.2 Hz), 7.85 (1H, d, J=8.7 Hz), 8.15 (1H, d, J=9.0 Hz), 8.26 (1H, dd, J=8.7, 2.3 Hz), 8.31 (1H, s), 8.65 (1H, br s), 8.75 (1H, d, J=2.3 Hz), 9.99 (1H, br s).

### Example 115

### N-{2-chloro-4-[(5-methyl-5H-pyrrolo[3,2-d]pyrimidin-4-yl)oxy]phenyl}-N′-[1-(2,2,2-trifluoroethyl)-1H-pyrazol-4-yl]urea

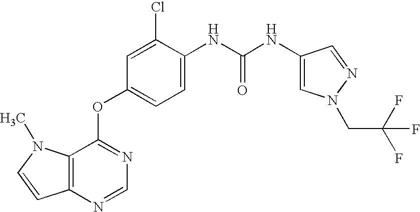

Using 2-chloro-4-[(5-methyl-5H-pyrrolo[3,2-d]pyrimidin-4-yl)oxy]aniline (275 mg, 1.0 mmol), triethylamine (2.79 mL, 20 mmol), dichloromethane (10 mL), triphosgene (297 mg, 1.0 mmol) and 4-amino-1-(2,2,2-trifluoroethyl)pyrazole (330 mg, 2.0 mmol), and in the same manner as in Example 113, the title compound (227 mg, 49%) was obtained.

¹H-NMR (DMSO-d₆, 300 MHz) δ 4.10 (3H, s), 5.09 (2H, q, J=9.2 Hz), 6.61 (1H, d, J=3.2 Hz), 7.29 (1H, dd, J=9.0, 2.6 Hz), 7.54 (1H, d, J=2.6 Hz), 7.56 (1H, s), 7.79 (1H, d, J=3.2 Hz), 7.97 (1H, s), 8.18 (1H, d, J=9.0 Hz), 8.30 (1H, s), 8.32 (1H, br s), 9.25 (1H, br s).

### Example 116

### N-[2-chloro-4-(5H-pyrrolo[3,2-d]pyrimidin-4-yloxy)phenyl]-N′-[3-(trifluoromethyl)phenyl]urea p-toluenesulfonic Acid Salt

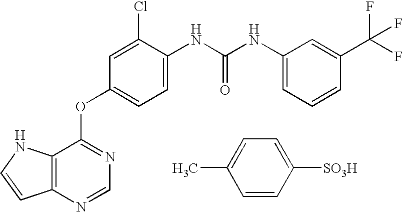

N-[2-Chloro-4-(5H-pyrrolo[3,2-d]pyrimidin-4-yloxy)phenyl]-N′-[3-(trifluoromethyl)phenyl]urea (224 mg, 0.50 mmol) was dissolved in ethanol (20 mL) at 70° C., and p-toluenesulfonic acid monohydrate (95 mg, 0.50 mmol) was added. Ethanol was evaporated under reduced pressure, and the residue was dissolved in ethanol (5 mL) at 70° C. The mixture was cooled to room temperature, and the precipitated solid was collected by filtration, washed with ethanol, and dried under reduced pressure at 90° C. to give the title compound (179 mg, 58%) as a white solid.

¹H-NMR (DMSO-d₆, 300 MHz) δ 2.29 (3H, s), 6.78 (1H, dd, J=2.8, 2.0 Hz), 7.11 (2H, d, J=7.7 Hz), 7.33-7.37 (1H, m), 7.35 (1H, dd, J=9.0, 2.7 Hz), 7.47 (2H, d, J=7.7 Hz), 7.52-7.60 (2H, m), 7.63 (1H, d, J=2.7 Hz), 8.05-8.07 (2H, m), 8.21 (1H, d, J=9.0 Hz), 8.49 (1H, br s), 8.66 (1H, s), 9.76 (1H, br s), 13.01 (1H, br s).

### Example 117

### N-(2-chloro-4-{[6-(2-furyl)-5-methyl-5H-pyrrolo[3,2-d]pyrimidin-4-yl]oxy}phenyl)-N′-[3-(trifluoromethyl)phenyl]urea

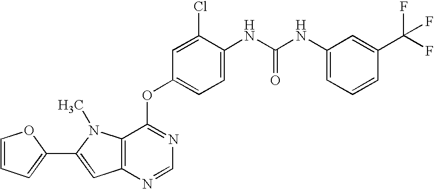

To a solution of 2-chloro-4-{[6-(2-furyl)-5-methyl-5H-pyrrolo[3,2-d]pyrimidin-4-yl]oxy}aniline (265 mg, 0.778 mmol) and triethylamine (162 μL, 1.16 mmol) in tetrahydrofuran (30 mL) was added 3-(trifluoromethyl)phenylisocyanate (655 mg, 3.50 mmol), and the mixture was stirred at room temperature for 16.5 hr. The reaction mixture was concentrated under reduced pressure, and the residue was dissolved in ethyl acetate (80 mL), washed with water, dried over anhydrous magnesium sulfate and filtrated. The filtrate was concentrated under reduced pressure, and the residue was purified by column chromatography (silica gel, ethyl acetate/hexane=10/90→90/10), and purified again by column chromatography (NH silica gel, ethyl acetate/hexane=10/90→60/40). Recrystallization from ethyl acetate-hexane and further recrystallization from ethanol gave the title compound (15.7 mg, 4%).

¹H-NMR (DMSO-d₆, 300 MHz) δ 4.25 (3H, s), 6.78 (1H, m), 6.95 (1H, s), 7.18 (1H, d, J=2.7 Hz), 7.31-7.36 (2H, m), 7.51-7.61 (3H, m), 7.99 (1H, s), 8.06 (1H, br s), 8.18 (1H, d, J=9.0 Hz), 8.33 (1H, s), 8.46 (1H, br s), 9.73 (1H, br s).

### Example 118

### Tert-butyl[2-(4-{3-chloro-4-[({[3-(trifluoromethyl)phenyl]amino}carbonyl)amino]phenoxy}-5H-pyrrolo[3,2-d]pyrimidin-5-yl)ethyl]carbamate

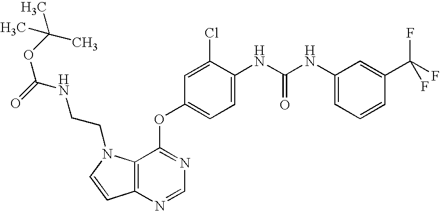

To a solution of tert-butyl {2-[4-(4-amino-3-chlorophenoxy)-5H-pyrrolo[3,2-d]pyrimidin-5-yl]ethyl}carbamate (420 mg, 1.04 mmol) and triethylamine (523 μL, 3.75 mmol) in tetrahydrofuran (12.6 mL) was added 3-(trifluoromethyl)phenylisocyanate (175 μL, 1.25 mmol), and the mixture was stirred at room temperature for 8 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and recrystallized from ethyl acetate-hexane to give the title compound (216 mg, 35%) as a white solid.

¹H-NMR (DMSO-d₆, 300 MHz) δ 1.25 (9H, s), 3.34-3.46 (2H, m), 4.38-4.52 (2H, m), 6.62 (1H, s), 6.88-6.98 (1H, m), 7.29-7.37 (2H, m), 7.52-7.58 (3H, m), 7.72 (1H, d, J=3.0 Hz), 8.05 (1H, s), 8.17 (1H, d, J=9.3 Hz), 8.30 (1H, s), 8.45 (1H, s), 9.72 (1H, s).

### Example 119

### N-[2-(4-{3-chloro-4-[({[3-(trifluoromethyl)phenyl]amino}carbonyl)amino]phenoxy}-5H-pyrrolo[3,2-d]pyrimidin-5-yl)ethyl]-3-hydroxy-3-methylbutanamide

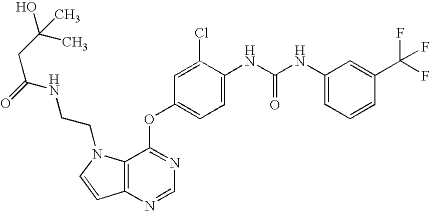

To a solution of tert-butyl[2-(4-{3-chloro-4-[({[3-(trifluoromethyl)phenyl]amino}carbonyl)amino]phenoxy}-5H-pyrrolo[3,2-d]pyrimidin-5-yl)ethyl]carbamate (3.65 g, 6.18 mmol) in methanol (14.6 mL) was added 4N hydrogen chloride/ethyl acetate solution (29.2 mL), and the mixture was stirred at room temperature for 3 hr. The reaction mixture was concentrated under reduced pressure, and ethyl acetate/diethyl ether was added to the residue. The precipitate was collected by filtration to give N-(4-{[5-(2-aminoethyl)-5H-pyrrolo[3,2-d]pyrimidin-4-yl]oxy}-2-chlorophenyl)-N′-[3-(trifluoromethyl)phenyl]urea dihydrochloride (3.4 g, quant.) as a white solid.

¹H-NMR (DMSO-d₆, 300 MHz) δ 3.34-3.48 (2H, m), 4.77-4.86 (2H, m), 6.86 (1H, d, J=2.7 Hz), 7.33 (1H, d, J=7.5 Hz), 7.40 (1H, dd, J=8.6, 2.6 Hz), 7.51-7.57 (1H, m), 7.64 (1H, s), 7.67 (1H, d, J=2.7 Hz), 8.07 (1H, s), 8.20-8.24 (2H, m), 8.32-8.46 (2H, m), 8.78 (1H, s), 8.84 (1H, s), 10.57 (1H, s).

To a solution of N-(4-{[5-(2-aminoethyl)-5H-pyrrolo[3,2-d]pyrimidin-4-yl]oxy}-2-chlorophenyl)-N′-[3-(trifluoromethyl)phenyl]urea dihydrochloride (500 mg, 0.89 mmol) and 3-hydroxy-3-methylbutanoic acid (157 mg, 1.33 mmol) in N,N-dimethylformamide (6.0 mL) were added triethylamine (618 μL, 4.43 mmol), 1-ethyl-3-(3-dimethylaminopropyl)carbodiimide hydrochloride (255 mg, 1.33 mmol) and 1-hydroxybenzotriazole (180 mg, 1.33 mmol), and the mixture was stirred at room temperature for 4 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/ethanol) and recrystallized from ethyl acetate-hexane to give the title compound (286 mg, 55%) as a white solid.

¹H-NMR (DMSO-d₆, 300 MHz) δ 1.06 (6H, s), 2.13 (2H, s), 3.54-3.64 (2H, m), 4.45-4.56 (2H, m), 4.70 (1H, s), 6.61 (1H, d, J=3.0 Hz), 7.30-7.35 (2H, m), 7.54-7.58 (3H, m), 7.76 (1H, d, J=3.0 Hz), 7.88-7.97 (1H, m), 8.06 (1H, s), 8.18 (1H, d, J=9.0 Hz), 8.32 (1H, s), 8.46 (1H, br s), 9.73 (1H, br s).

### Example 120

### N-[2-(4-{3-chloro-4-[({[3-(trifluoromethyl)phenyl]amino}carbonyl)amino]phenoxy}-5H-pyrrolo[3,2-d]pyrimidin-5-yl)ethyl]-2-methyl-2-(methylsulfonyl)propanamide

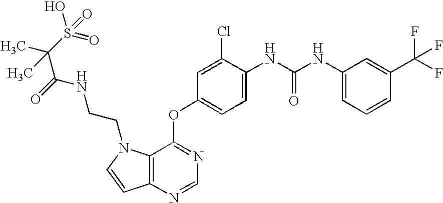

To a solution of 2-methyl-2-(methylsulfonyl)propanoic acid (221 mg, 1.33 mmol) in tetrahydrofuran (5.0 mL) were added N,N-dimethylformamide (0.1 mL) and thionyl chloride (97 μL, 1.33 mmol), and the mixture was stirred at room temperature for 2 hr. To a solution of N-(4-{[5-(2-aminoethyl)-5H-pyrrolo[3,2-d]pyrimidin-4-yl]oxy}-2-chlorophenyl)-N′-[3-(trifluoromethyl)phenyl]urea dihydrochloride (500 mg, 0.89 mmol) and triethylamine (990 μL, 7.09 mmol) in tetrahydrofuran (10.0 mL) was added dropwise the above-mentioned mixture, and the mixture was stirred at room temperature for 3 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/ethanol) and recrystallized from ethyl acetate-hexane to give the title compound (258 mg, 46%) as a white solid.

¹H-NMR (DMSO-d₆, 300 MHz) δ 1.40 (6H, s), 2.93 (3H, s), 3.60-3.69 (2H, m), 4.46-4.53 (2H, m), 6.60 (1H, d, J=3.0 Hz), 7.31-7.36 (2H, m), 7.54-7.59 (3H, m), 7.68 (1H, d, J=3.0 Hz), 8.07 (2H, m), 8.19 (1H, d, J=9.0 Hz), 8.33 (1H, s), 8.47 (1H, s), 9.73 (1H, s).

### Example 121

### N-{2-chloro-4-[(5-methyl-5H-pyrrolo[3,2-d]pyrimidin-4-yl)oxy]phenyl}-N′-[2-methyl-5-(trifluoromethyl)phenyl]urea

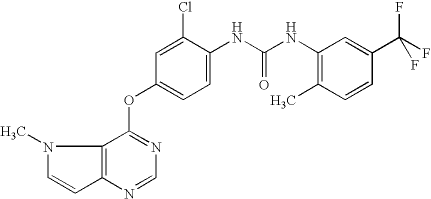

To a solution of 2-chloro-4-[(5-methyl-5H-pyrrolo[3,2-d]pyrimidin-4-yl)oxy]aniline (500 mg, 1.82 mmol) and triethylamine (2.5 mL) in chloroform (15 mL) was added triphosgene (567 mg, 1.91 mmol), and the mixture was stirred at room temperature for 30 min. 2-Methyl-5-(trifluoromethyl)aniline (351 mg, 2.00 mmol) was added, and the mixture was stirred at room temperature for 4 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and recrystallized from ethyl acetate-hexane to give the title compound (153 mg, 18%) as a white solid.

¹H-NMR (DMSO-d₆, 300 MHz) δ 2.37 (3H, s), 4.11 (3H, s), 6.61 (1H, d, J=3.2 Hz), 7.28-7.33 (2H, m), 7.41-7.45 (1H, m), 7.57 (1H, d, J=2.7 Hz), 7.80 (1H, d, J=3.2 Hz), 8.15 (1H, d, J=9.0 Hz), 8.31 (1H, s), 8.35 (1H, s), 8.82 (1H, s), 8.95 (1H, s).

### Example 122

### Tert-butyl[(4-{3-chloro-4-[({[3-(trifluoromethyl)phenyl]amino}carbonyl)amino]phenoxy}-5H-pyrrolo[3,2-d]pyrimidin-6-yl)methyl]carbamate

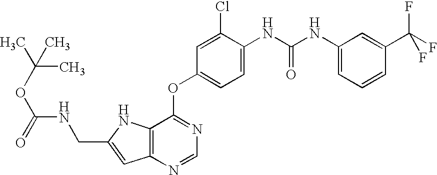

To a solution of tert-butyl {[4-(4-amino-3-chlorophenoxy)-5H-pyrrolo[3,2-d]pyrimidin-6-yl]methyl}carbamate (500 mg, 1.28 mmol) and triethylamine (715 μL, 5.13 mmol) in tetrahydrofuran (10 mL) was added 3-(trifluoromethyl)phenylisocyanate (215 μL, 1.54 mmol), and the mixture was stirred at room temperature for 5 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by silica gel column chromatography (ethyl acetate/hexane) and recrystallized from ethyl acetate-hexane to give the title compound (105 mg, 14%) as a white solid.

¹H-NMR (DMSO-d₆, 300 MHz) δ 1.42 (9H, s), 4.36 (2H, d, J=5.4 Hz), 6.42 (1H, s), 7.28 (1H, dd, J=9.0, 2.7 Hz), 7.33-7.36 (1H, m), 7.42-7.49 (1H, m), 7.53-7.57 (3H, m), 8.05 (1H, s), 8.15 (1H, d, J=9.0 Hz), 8.29 (1H, s), 8.46 (1H, s), 9.73 (1H, s), 12.20 (1H, br s).

### Example 123

### N-[(4-{3-chloro-4-[({[3-(trifluoromethyl)phenyl]amino}carbonyl)amino]phenoxy}-5H-pyrrolo[3,2-d]pyrimidin-6-yl)methyl]-3-hydroxy-3-methylbutanamide

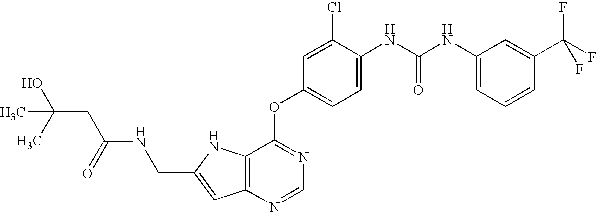

To a solution of tert-butyl[(4-{3-chloro-4-[({[3-(trifluoromethyl)phenyl]amino}carbonyl)amino]phenoxy}-5H-pyrrolo[3,2-d]pyrimidin-6-yl)methyl]carbamate (2.15 g, 3.73 mmol) in methanol (8.6 mL) was added 4N hydrogen chloride/ethyl acetate solution (17.2 mL), and the mixture was stirred at room temperature for 1 hr. The reaction mixture was concentrated under reduced pressure, and ethyl acetate/hexane was added to the residue. The precipitate was collected by filtration to give N-(4-{[6-(aminomethyl)-5H-pyrrolo[3,2-d]pyrimidin-4-yl]oxy}-2-chlorophenyl)-N′-[3-(trifluoromethyl)phenyl]urea dihydrochloride (2.05 g, quant.) as a white solid.

¹H-NMR (DMSO-d₆, 300 MHz) δ 4.38 (2H, d, J=5.4 Hz), 7.02 (1H, s), 7.32-7.37 (2H, m), 7.49-7.58 (1H, m), 7.61-7.66 (2H, m), 8.06 (1H, s), 8.21 (1H, d, J=9.3 Hz), 8.78 (2H, d, J=5.7 Hz), 8.90 (2H, br s), 10.42 (1H, s), 13.44 (1H, s).

To a solution of N-(4-{[6-(aminomethyl)-5H-pyrrolo[3,2-d]pyrimidin-4-yl]oxy}-2-chlorophenyl)-N′-[3-(trifluoromethyl)phenyl]urea dihydrochloride (500 mg, 0.91 mmol) and 3-hydroxy-3-methylbutanoic acid (161 mg, 1.36 mmol) in N,N-dimethylformamide (6.0 mL) were added triethylamine (634 μL, 4.55 mmol), 1-ethyl-3-(3-dimethylaminopropyl)carbodiimide hydrochloride (262 mg, 1.36 mmol) and 1-hydroxybenzotriazole (184 mg, 1.36 mmol), and the mixture was stirred at room temperature for 4 hr. The reaction mixture was diluted with water, and extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/ethanol) and recrystallized from ethyl acetate-hexane to give the title compound (245 mg, 47%) as a white solid.

¹H-NMR (DMSO-d₆, 300 MHz) δ 1.20 (6H, s), 2.33 (2H, s), 4.52 (2H, d, J=6.0 Hz), 4.81 (1H, s), 6.47 (1H, s), 7.28 (1H, dd, J=8.9, 2.9 Hz), 7.35 (1H, d, J=7.2 Hz), 7.53-7.57 (3H, m), 8.05 (1H, s), 8.15 (1H, d, J=8.9 Hz), 8.29 (1H, s), 8.40-78.47 (2H, m), 9.74 (1H, s), 12.23 (1H, s).

### Example 124

### N-{2-chloro-4-[(5,6-dimethyl-5H-pyrrolo[3,2-d]pytimidin-4-yl)oxy]phenyl}-N′-[3-(trifluoromethyl)phenyl]urea

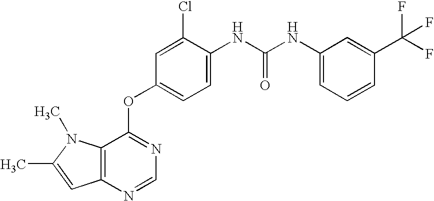

To a solution of 2-chloro-4-[(5,6-dimethyl-5H-pyrrolo[3,2-d]pyrimidin-4-yl)oxy]aniline (400 mg, 1.39 mmol) and triethylamine (0.56 mL, 5.54 mmol) in tetrahydrofuran (20 mL) was added 3-(trifluoromethyl)phenylisocyanate (232 μL, 1.66 mmol), and the mixture was stirred at room temperature for 4 hr. Water was added to the reaction mixture, and the mixture was extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and further by column chromatography (silica gel, ethyl acetate/hexane) and recrystallized from ethyl acetate/hexane to give the title compound (202 mg, 31%) as a white solid. melting point 190-192° C.

¹H-NMR (DMSO-d₆, 300 MHz) δ 2.50 (3H, s), 3.99 (3H, s), 6.45 (1H, s), 7.29 (1H, dd, J=9.0, 2.7 Hz), 7.33-7.37 (1H, m), 7.53-7.57 (3H, m), 8.06 (1H, s), 8.17 (1H, d, J=9.0 Hz), 8.24 (1H, s), 8.46 (1H, s), 9.74 (1H, s).

### Example 125

### N-{2-chloro-4-[(5-methyl-5H-pyrrolo[3,2-d]pyrimidin-4-yl)oxy]phenyl}-N′-[2-fluoro-5-(2,2,2-trifluoro-1-hydroxy-1-methylethyl)phenyl]urea

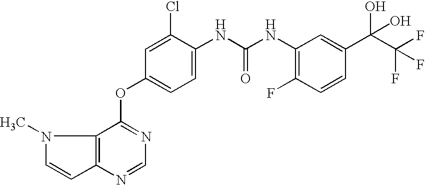

Using 2-chloro-4-[(5-methyl-5H-pyrrolo[3,2-d]pyrimidin-4-yl)oxy]aniline (362 mg, 1.10 mmol), pyridine (266 μL, 3.30 mmol), phenyl chloroformate (146 μL, 1.15 mmol), 2-(3-amino-4-fluorophenyl)-1,1,1-trifluoropropan-2-ol (245 mg, 1.10 mmol) and N-methylpyrrolidone (5 mL) as starting materials, and in the same manner as in Example 20, the title compound (137 mg, 24%) was obtained as a white solid.

¹H-NMR (DMSO-d₆, 200 MHz) δ 1.67 (3H, s), 4.10 (3H, s), 6.60 (1H, d, J=3.0 Hz), 6.66 (1H, s), 7.22-7.32 (3H, m), 7.56 (1H, d, J=2.7 Hz), 7.80 (1H, d, J=3.0 Hz), 8.19 (1H, d, J=9.0 Hz), 8.30 (1H, s), 8.47-8.50 (1H, m), 8.88 (1H, s), 9.39 (1H, s).

### Example 126

### N-{2-chloro-4-[(5-methyl-5H-pyrrolo[3,2-d]pyrimidin-4-yl)oxy]phenyl}-N′-[3-(2,2,2-trifluoro-1-methoxyethyl)phenyl]urea

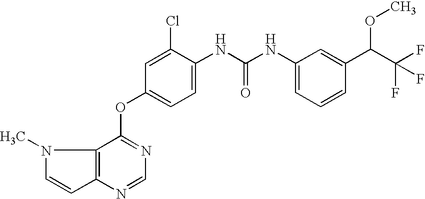

Using 2-chloro-4-[(5-methyl-5H-pyrrolo[3,2-d]pyrimidin-4-yl)oxy]aniline (323 mg, 1.17 mmol), pyridine (283 μL, 3.51 mmol), phenyl chloroformate (178 μL, 1.41 mmol), 3-(2,2,2-trifluoro-1-methoxyethyl)aniline (240 mg, 1.17 mmol) and N-methylpyrrolidone (5 mL) as starting materials, and in the same manner as in Example 20, the title compound (217 mg, 37%) was obtained as a white solid.

¹H-NMR (DMSO-d₆, 200 MHz) δ 3.35 (3H, s), 4.10 (3H, s), 5.01-5.08 (1H, m), 6.60-6.61 (1H, m), 7.08 (1H, d, J=7.9 Hz), 7.30 (1H, dd, J=8.9, 2.7 Hz), 7.38 (1H, t, J=7.9 Hz), 7.51 (1H, d, J=7.9 Hz), 7.56-7.57 (1H, m), 7.65 (1H, s), 7.80 (1H, d, J=3.0 Hz), 8.20 (1H, d, J=8.9 Hz), 8.30 (1H, s), 8.38 (1H, s), 9.58 (1H, s).

### Example 127

### N-{2-chloro-4-[(5-methyl-5H-pyrrolo[3,2-d]pyrimidin-4-yl)oxy]phenyl}-N′-[2-chloro-5-(trifluoromethyl)phenyl]urea

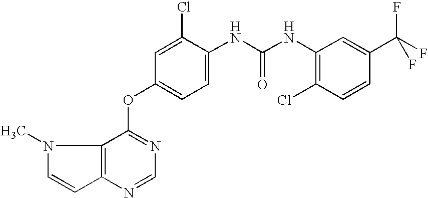

To a solution of 2-chloro-5-(trifluoromethyl)aniline (344 mg, 1.76 mmol) and pyridine (426 μL, 5.28 mmol) in N,N-dimethylacetamide (5 mL) was added phenyl chloroformate (233 μL, 1.85 mmol) with stirring under ice-cooling, and the mixture was stirred at room temperature for 1 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was dissolved in N-methylpyrrolidone (5 mL). Pyridine (383 μL, 4.75 mmol) and 2-chloro-4-[(5-methyl-5H-pyrrolo[3,2-d]pyrimidin-4-yl)oxy]aniline (275 mg, 1.00 mmol) were added, and the mixture was stirred at 80° C. for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=10/90→100/0) and recrystallized from ethyl acetate-hexane to give the title compound (272 mg, 55%) as a white solid.

¹H-NMR (DMSO-d₆, 300 MHz) δ 4.11 (3H, s), 6.61 (1H, d, J=3.0 Hz), 7.32 (1H, dd, J=9.1, 2.8 Hz), 7.41 (1H, dd, J=8.2, 2.3 Hz), 7.58 (1H, d, J=2.8 Hz), 7.74 (1H, d, J=8.2 Hz), 7.80 (1H, d, J=3.0 Hz), 8.10 (1H, d, J=9.1 Hz), 8.30 (1H, s), 8.60 (1H, s), 9.30-9.35 (2H, m).

### Example 128

### N-{2-chloro-4-[(6-cyano-5-methyl-5H-pyrrolo[3,2-d]pyrimidin-4-yl)oxy]phenyl}-N′-[3-(trifluoromethyl)phenyl]urea

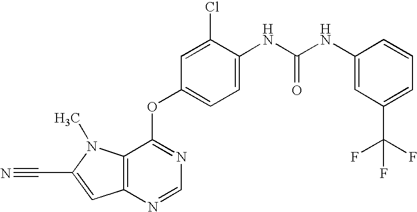

To a solution of 4-(4-amino-3-chlorophenoxy)-5-methyl-5H-pyrrolo[3,2-d]pyrimidine-6-carbonitrile (300 mg, 1.0 mmol) and triethylamine (40 μL, 0.3 mmol) in tetrahydrofuran (30 mL) was added 3-(trifluoromethyl)phenylisocyanate (179 μL, 1.3 mmol), and the mixture was stirred at room temperature for 8 hr. The reaction mixture was concentrated under reduced pressure, and the residue was purified by column chromatography (NH silica gel, hexane/ethyl acetate=90/10→0/100). The object fraction was concentrated under reduced pressure, and the residue was purified by column chromatography (silica gel, hexane/ethyl acetate=90/10→20/80) and recrystallized from ethyl acetate to give the title compound (184 mg, 38%).

¹H-NMR (DMSO-d₆, 300 MHz) δ 4.21 (3H, s), 7.34-7.38 (2H, m), 7.52-7.59 (2H, m), 7.63 (1H, d, J=2.7 Hz), 7.65 (1H, s), 8.07 (1H, br s), 8.21 (1H, d, J=9.0 Hz), 8.49 (1H, s), 8.51 (1H, br s), 9.78 (1H, br s).

### Example 129

### 2-(4-{3-chloro-4-[({[3-(trifluoromethyl)phenyl]amino}carbonyl)amino]phenoxy}-5H-pyrrolo[3,2-d]pyrimidin-5-yl)acetamide

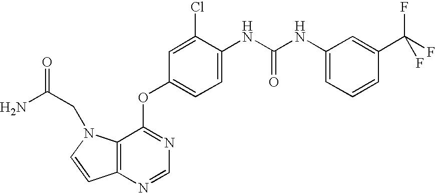

To a solution of 2-[4-(4-amino-3-chlorophenoxy)-5H-pyrrolo[3,2-d]pyrimidin-5-yl]acetamide (317 mg, 1.00 mmol) and triethylamine (279 μL, 2.00 mmol) in tetrahydrofuran (30 mL) was added 3-(trifluoromethyl)phenylisocyanate (374 mg, 2.00 mmol) under ice-cooling, and the mixture was stirred at room temperature for 8 hr. The reaction mixture was concentrated under reduced pressure, and the residue was purified by column chromatography (silica gel, ethyl acetate/methanol=100/0→80/20) and purified again by preparative HPLC. The obtained solid was washed with ether and dried to give the title compound (45.8 mg, 9%).

¹H-NMR (DMSO-d₆, 300 MHz) δ 5.08 (2H, s), 6.60 (1H, d, J=3.0 Hz), 7.21-7.25 (2H, m), 7.31 (1H, m), 7.41 (1H, d, J=2.7 Hz), 7.49-7.59 (3H, m), 7.76 (1H, d, J=3.0 Hz), 8.03 (1H, br s), 8.17 (1H, d, J=9.0 Hz), 8.32 (1H, s), 8.41 (1H, br s), 9.70 (1H, br s).

### Example 130

### N-(2-chloro-4-{[6-(1-hydroxy-1-methylethyl)-5-methyl-5H-pyrrolo[3,2-d]pyrimidin-4-yl]oxy}phenyl)-N′-[3-(trifluoromethyl)phenyl]urea

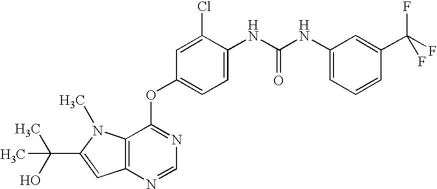

To a solution of 2-chloro-4-[(5-methyl-6-{1-methyl-1-[(trimethylsilyl)oxy]ethyl}-5H-pyrrolo[3,2-d]pyrimidin-4-yl)oxy]aniline (0.95 g, 2.35 mmol) and triethylamine (1.31 mL, 9.38 mmol) in tetrahydrofuran (19.0 mL) was added 3-(trifluoromethyl)phenylisocyanate (0.39 mL, 2.82 mmol), and the mixture was stirred at room temperature for 5 hr. Water was added to the reaction mixture, and the mixture was extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and purified further by column chromatography (silica gel, ethyl, acetate/hexane) to give N-{2-chloro-4-[(5-methyl-6-{1-methyl-1-[(trimethylsilyl)oxy]ethyl}-5H-pyrrolo[3,2-d]pyrimidin-4-yl)oxy]phenyl}-N′-[3-(trifluoromethyl)phenyl]urea (0.81 g, 58%) as a white solid.

¹H-NMR (DMSO-d₆, 300 MHz) δ 0.06 (9H, s), 1.75 (6H, s), 4.28 (3H, s), 6.57 (1H, s), 7.30-7.37 (2H, m), 7.54-7.60 (3H, m), 8.06 (1H, s), 8.17 (1H, d, J=9.3 Hz), 8.28 (1H, s), 8.46 (1H, s), 9.74 (1H, s).

To a solution of N-{2-chloro-4-[(5-methyl-6-{1-methyl-1-[(trimethylsilyl)oxy]ethyl}-5H-pyrrolo[3,2-d]pyrimidin-4-yl)oxy]phenyl}-N′-[3-(trifluoromethyl)phenyl]urea (0.81 g, 1.37 mmol) in ethanol (12.3 mL) was added 1N hydrochloric acid (2.43 mL), and the mixture was stirred at room temperature for 15 min. The solvent was evaporated under reduced pressure, saturated aqueous sodium hydrogen carbonate was added to the residue, and the mixture was extracted with ethyl acetate. The mixture was washed with saturated brine, dried over anhydrous magnesium sulfate, and filtrated. The filtrate was concentrated under reduced pressure, and the residue was purified by column chromatography (silica gel, ethyl acetate/hexane) and recrystallized from ethyl acetate/hexane to give the title compound (550 mg, 77%) as a white solid.

¹H-NMR (DMSO-d₆, 300 MHz) δ 1.64 (6H, s), 4.31 (3H, s), 5.55 (1H, s), 6.51 (1H, s), 7.29 (1H, dd, J=9.0, 2.7 Hz), 7.32-7.35 (1H, m), 7.53-7.56 (3H, m), 8.05 (1H, s), 8.16 (1H, d, J=9.0 Hz), 8.25 (1H, s), 8.45 (1H, s), 9.73 (1H, s).

### Example 131

### N-{2-chloro-4-[(5-methyl-5H-pyrrolo[3,2-d]pyrimidin-4-yl)oxy]phenyl}-N′-[3-hydroxy-5-(trifluoromethyl)phenyl]urea

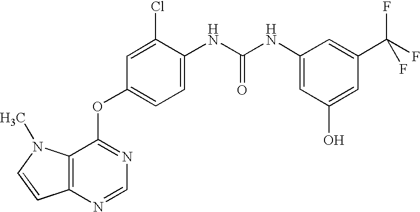

To a mixture of 2-chloro-4-[(5-methyl-5H-pyrrolo[3,2-d]pyrimidin-4-yl)oxy]aniline (824 mg, 3.00 mmol), triethylamine (762 mg, 7.53 mmol) and chloroform (30 mL) was added dropwise a solution (2 mL) of triphosgene (445 mg, 1.50 mmol) in chloroform under ice-cooling. After stirring at room temperature for 30 min, a solution (2 mL) of 3-(benzyloxy)-5-(trifluoromethyl)aniline (962 mg, 3.60 mmol) in chloroform was added dropwise under ice-cooling. The mixture was stirred at room temperature for 20 hr, and concentrated under reduced pressure. Ethyl acetate (200 mL) was added to the residue, and the mixture was washed with water (80 mL) and saturated brine (30 mL) and dried over anhydrous magnesium sulfate. After concentration under reduced pressure, the residue was purified by column chromatography (silica gel, hexane/ethyl acetate=90/10→0/100) and purified further by column chromatography (NH silica gel, hexane/ethyl acetate=90/10→0/100) to give N-[3-(benzyloxy)-5-(trifluoromethyl)phenyl]-N′-{2-chloro-4-[(5-methyl-5H-pyrrolo[3,2-d]pyrimidin-4-yl)oxy]phenyl}urea (73 mg, 4%).

¹H-NMR (CDCl₃, 300 MHz) δ 4.10 (3H, s), 4.99 (2H, s), 6.64 (1H, d, J=3.0 Hz), 6.86 (1H, m), 7.10-7.16 (2H, m), 7.23-7.41 (8H, m), 7.70 (1H, s), 8.24 (1H, d, J=9.0 Hz), 8.45 (1H, s), 8.93 (1H, br s).

A mixture of N-[3-(benzyloxy)-5-(trifluoromethyl)phenyl]-N′-{2-chloro-4-[(5-methyl-5H-pyrrolo[3,2-d]pyrimidin-4-yl)oxy]phenyl}urea (73 mg, 0.13 mmol), 10% palladium carbon (67 mg), 1,4-cyclohexadiene (421 mg, 5.25 mmol) and ethanol (20 mL) was stirred at room temperature for 2 hr and at 40° C. for 12 hr. After celite filtration, the filtrate was concentrated under reduced pressure. The residue was purified by column chromatography (silica gel, hexane/ethyl acetate/methanol=80/20/0→0/100/0→0/90/10) and recrystallized from methanol/ethyl acetate to give the title compound (28 mg, 46%).

¹H-NMR (DMSO-d₆, 300 MHz) δ 4.11 (3H, s), 6.59 (1H, d, J=2.7 Hz), 6.65 (1H, s), 7.12 (1H, s), 7.29 (1H, d, J=8.7 Hz), 7.38 (1H, s), 7.53 (1H, s), 7.77 (1H, d, J=2.7 Hz), 8.12 (1H, d, J=8.7 Hz), 8.29 (1H, s), 8.64 (1H, br s), 9.88 (1H, br s), 10.30 (1H, br s).

### Example 132

### N-{2-(hydroxymethyl)-4-[(5-methyl-5H-pyrrolo[3,2-d]pyrimidin-4-yl)oxy]phenyl}-N′-[3-(trifluoromethyl)phenyl]urea

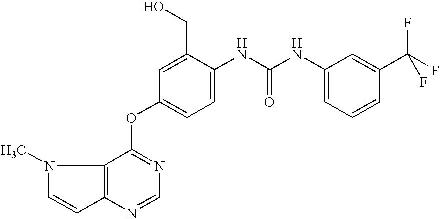

A mixture of 4-chloro-5-methyl-5H-pyrrolo[3,2-d]pyrimidine (115 mg, 0.690 mmol), N-[4-hydroxy-2-(hydroxymethyl)phenyl]-N′-[3-(trifluoromethyl)phenyl]urea (270 mg, 0.828 mmol), potassium carbonate (229 mg, 1.66 mmol) and N-methylpyrrolidone (10 mL) was stirred at 110° C. for 3 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=10/90→100/0) to give the title compound (75.0 mg, 24%) as a white solid.

¹H-NMR (DMSO-d₆, 300 MHz) δ 4.11 (3H, s), 4.85 (2H, d, J=5.5 Hz), 5.57 (1H, t, J=5.5 Hz), 6.59 (1H, d, J=3.0 Hz), 7.18 (1H, dd, J=8.8, 2.7 Hz), 7.27-7.32 (2H, m), 7.51 (1H, t, J=7.8 Hz), 7.59 (1H, d, J=7.8 Hz), 7.79 (1H, d, J=3.0 Hz), 7.85 (1H, d, J=8.8 Hz), 8.04 (1H, s), 8.27 (1H, s), 8.30 (1H, s), 9.68 (1H, s).

### Example 133

### N-{2-fluoro-4-[(5-methyl-5H-pyrrolo[3,2-d]pyrimidin-4-yl)oxy]phenyl}-N′-[3-(trifluoromethyl)phenyl]urea Benzenesulfonic Acid Salt

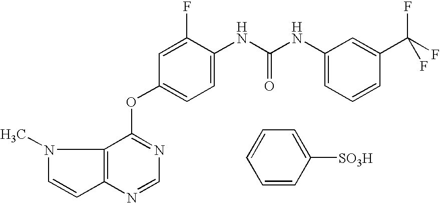

N-{2-fluoro-4-[(5-methyl-5H-pyrrolo[3,2-d]pyrimidin-4-yl)oxy]phenyl}-N′-[3-(trifluoromethyl)phenyl]urea (2.0 g, 4.49 mmol) was dissolved in ethyl acetate (200 mL) at 75° C., and 0.5N benzenesulfonic acid ethyl acetate solution (9.4 mL) was added dropwise. The solvent was evaporated under reduced pressure, and the residue was recrystallized from ethanol to give the title compound (2.33 g, 86%) as a white solid.

¹H-NMR (DMSO-d₆, 300 MHz) δ 4.17 (3H, s), 6.76 (1H, d, J=3.0 Hz), 7.22 (1H, d, J=9.1 Hz), 7.27-7.38 (4H, m), 7.46 (1H, dd, J=11.7, 2.7 Hz), 7.50-7.65 (4H, m), 8.02-8.11 (2H, m), 8.18 (1H, t, J=9.1 Hz), 8.71 (1H, s), 8.75 (1H, d, J=1.9 Hz), 9.45 (1H, s).

### Example 134

### N-{2-chloro-4-[(5,6-dimethyl-5H-pyrrolo[3,2-d]pyrimidin-4-yl)oxy]phenyl}-N′-[3-(trifluoromethyl)phenyl]urea hydrochloride

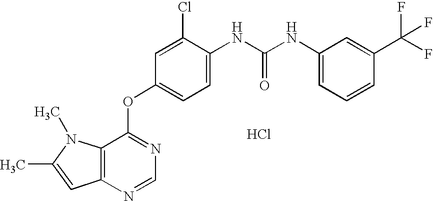

N-{2-Chloro-4-[(5,6-dimethyl-5H-pyrrolo[3,2-d]pyrimidin-4-yl)oxy]phenyl}-N′-[3-(trifluoromethyl)phenyl]urea (207 mg, 0.44 mmol) was dissolved in ethanol (6.2 mL) with heating, 4N hydrogen chloride/ethyl acetate solution (109 μL) was added, and the mixture was stood at room temperature for 18 hr. The precipitated crystals were collected by filtration, and washed with ethanol. The crystals were dried under reduced pressure at 90° C. for 10 hr to give the title compound (140 mg, 63%) as colorless crystals. melting point 226-228° C.

¹H-NMR (DMSO-d₆, 300 MHz) δ 2.59 (3H, s), 4.06 (3H, s), 6.67 (1H, s), 7.32-7.38 (2H, m), 7.54 (1H, t, J=7.8 Hz), 7.59-7.63 (2H, m), 8.06 (1H, s), 8.22 (1H, d, J=9.3 Hz), 8.68 (1H, s), 8.70 (1H, s), 10.20 (1H, s).

### Example 135

### N-{2-fluoro-4-[(5-methyl-5H-pyrrolo[3,2-d]pyrimidin-4-yl)oxy]phenyl}-N′-[3-(trifluoromethyl)phenyl]urea p-toluenesulfonic Acid Salt

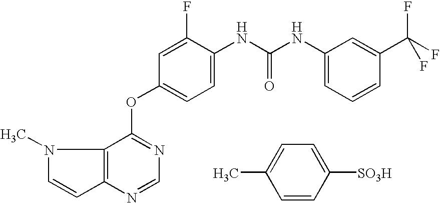

N-{2-Chloro-4-[(5-methyl-5H-pyrrolo[3,2-d]pyrimidin-4-yl)oxy]phenyl}-N′-[3-(trifluoromethyl)phenyl]urea (203 mg, 0.455 mmol) was dissolved in ethyl acetate (6 mL) at 70° C., 0.5N p-toluenesulfonic acid ethyl acetate solution (0.91 mL, 0.455 mmol) was added dropwise. The solvent was evaporated under reduced pressure, and the residue was recrystallized from ethanol to give the title compound (225 mg, 80%) as a white solid.

¹H-NMR (DMSO-d₆, 300 MHz) δ 2.29 (3H, s), 4.18 (3H, s), 6.78 (1H, d, J=3.0 Hz), 7.11 (2H, d, J=8.1 Hz), 7.20-7.24 (1H, m), 7.32-7.35 (1H, m), 7.43-7.58 (5H, m), 8.06 (1H, s), 8.11 (1H, d, J=3.0 Hz), 8.18 (1H, t, J=9.2 Hz), 8.75-8.77 (2H, m), 9.46 (1H, s).

### Example 136

### N-{2-fluoro-4-[(5-methyl-5H-pyrrolo[3,2-d]pyrimidin-4-yl)oxy]phenyl}-N′-[3-(trifluoromethyl)phenyl]urea Methanesulfonic Acid Salt

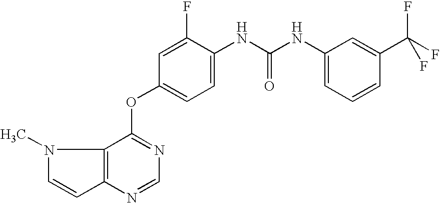

To a suspension of N-{2-fluoro-4-[(5-methyl-5H-pyrrolo[3,2-d]pyrimidin-4-yl)oxy]phenyl}-N′-[3-(trifluoromethyl)phenyl]urea (2.00 g, 4.5 mmol) in ethanol (20 mL) was added methanesulfonic acid (360 μL, 4.9 mmol) at room temperature with stirring. The obtained uniform solution was stirred at room temperature for 15 hr and at 0° C. for 1 hr. The precipitated crystals were collected by filtration, washed with ethanol (5 mLx²), and vacuum dried to give the title compound (2.36 g, 97%).

¹H-NMR (DMSO-d₆, 300 MHz) δ 2.35 (3H, s), 4.16 (3H, s), 6.73 (1H, d, J=3.0 Hz), 7.18-7.22 (1H, m), 7.35 (1H, br d, J=6.6 Hz), 7.44 (1H, dd, J=11.7, 2.4 Hz), 7.51-7.59 (2H, m), 8.01 (1H, d, J=3.0 Hz), 8.06 (1H, br s), 8.17 (1H, t, J=9.1 Hz), 8.60 (1H, s), 8.74 (1H, br d, J=1.8 Hz), 9.45 (1H, br s).

### Example 137

### N-{2-fluoro-4-[(5-methyl-5H-pyrrolo[3,2-d]pyrimidin-4-yl)oxy]phenyl}-N′-[3-(trifluoromethyl)phenyl]urea Hydrochloride

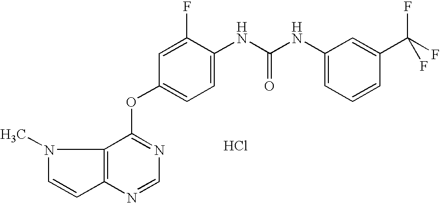

N-{2-fluoro-4-[(5-methyl-5H-pyrrolo[3,2-d]pyrimidin-4-yl)oxy]phenyl}-N′-[3-(trifluoromethyl)phenyl]urea (60.0 g, 135 mmol) was dissolved in ethanol (1300 mL) with heating, 4N hydrogen chloride-ethyl acetate solution (40.0 mL, 160 mmol) was added dropwise, and the mixture was stirred at room temperature for 15 hr. The precipitated solid was collected by filtration, and the solid was suspended in ethanol (1000 mL). After stirring under reflux for 1 hr, the mixture was cooled to room temperature. The solid was collected by filtration, and washed with ethanol and ethyl acetate to give the title compound (56.3 g, 87%) as a white solid. melting point 190-211° C.

¹H-NMR (DMSO-d₆, 300 MHz) δ 4.19 (3H, s), 6.80 (1H, d, J=3.0 Hz), 7.20-7.24 (1H, m), 7.32 (1H, d, J=8.0 Hz), 7.46 (1H, dd, J=11.4, 2.7 Hz), 7.53 (1H, t, J=8.0 Hz), 7.61 (1H, d, J=8.0 Hz), 8.05 (1H, s), 8.14 (1H, d, J=3.0 Hz), 8.20 (1H, t, J=9.0 Hz), 8.80 (1H, s), 9.07 (1H, d, J=1.8 Hz), 10.20 (1H, s).

### Example 138

### N-{2-fluoro-4-[(5-methyl-5H-pyrrolo[3,2-d]pyrimidin-4-yl)oxy]phenyl}-N′-[3-(trifluoromethyl)phenyl]urea hydrobromide

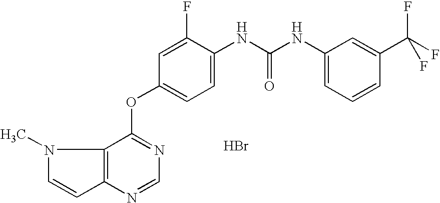

N-{2-Fluoro-4-[(5-methyl-5H-pyrrolo[3,2-d]pyrimidin-4-yl)oxy]phenyl}-N′-[3-(trifluoromethyl)phenyl]urea (2.5 g, 5.61 mmol) was dissolved in ethanol (50 mL) with heating, 20% hydrogen bromide-ethanol solution (2.27 g) was added, and the mixture was stood at room temperature for 3 hr. The precipitated crystals were collected by filtration, washed with ethanol, and dried under reduced pressure at 90° C. for 8 hr to give the title compound (1.78 g, 60%) as colorless crystals.

¹H-NMR (DMSO-d₆, 300 MHz) δ 4.17 (3H, s), 6.75 (1H, d, J=3.0 Hz), 7.19-7.23 (1H, m), 7.34-7.36 (1H, m), 7.46 (1H, dd, J=11.7, 2.7 Hz), 7.51-7.58 (2H, m), 8.05 (1H, d, J=3.0 Hz), 8.07 (1H, s), 8.19 (1H, t, J=9.1 Hz), 8.66 (1H, s), 8.75 (1H, d, J=2.1 Hz), 9.50 (1H, s).

### Example 139

### N-[4-(benzyloxy)-3-(trifluoromethyl)phenyl]-N′-{2-chloro-4-[(5-methyl-5H-pyrrolo[3,2-d]pyrimidin-4-yl)oxy]phenyl}urea

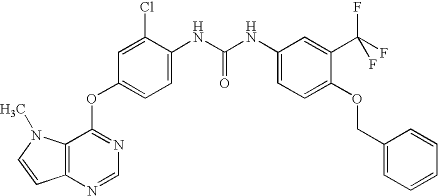

To a mixture of 2-chloro-4-[(5-methyl-5H-pyrrolo[3,2-d]pyrimidin-4-yl)oxy]aniline (1374 mg, 5.00 mmol), triethylamine (2030 mg, 20.0 mmol) and tetrahydrofuran (50 mL) was added dropwise a solution (5 mL) of triphosgene (594 mg, 2.00 mmol) in tetrahydrofuran under ice-cooling, and the mixture was stirred at room temperature for 30 min. After stirring, a solution (10 mL) of 4-(benzyloxy)-3-(trifluoromethyl)aniline (1600 mg, 5.99 mmol) in tetrahydrofuran was added dropwise under ice-cooling. The mixture was stirred at room temperature for 7 hr, and concentrated under reduced pressure. Ethyl acetate (250 mL) was added to the residue, and the mixture was washed with water (100 mL) and dried over anhydrous magnesium sulfate. After concentration under reduced pressure, the residue was purified by column chromatography (silica gel, hexane/ethyl acetate=75/25→0/100) and purified further by column chromatography (NH silica gel, hexane/ethyl acetate=90/10→20/80) and recrystallized from methanol/ethyl acetate to give the title compound (1180 mg, 42%).

¹H-NMR (DMSO-d₆, 300 MHz) δ 4.10 (3H, s), 5.23 (2H, s), 6.59 (1H, d, J=3.0 Hz), 7.26-7.46 (7H, m), 7.52-7.57 (2H, m), 7.77 (1H, d, J=3.0 Hz), 7.89 (1H, d, J=2.7 Hz), 8.17 (1H, d, J=8.7 Hz), 8.29 (1H, s), 8.33 (1H, br s), 9.47 (1H, br s).

### Example 140

### N-{2-chloro-4-[(5-methyl-5H-pyrrolo[3,2-d]pyrimidin-4-yl)oxy]phenyl}-N′-[4-hydroxy-3-(trifluoromethyl)phenyl]urea

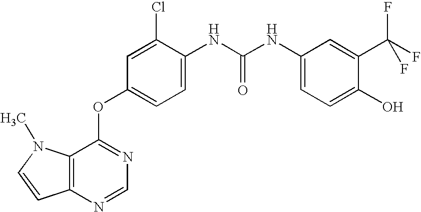

A mixture of N-[4-(benzyloxy)-3-(trifluoromethyl)phenyl]-N′-{2-chloro-4-[(5-methyl-5H-pyrrolo[3,2-d]pyrimidin-4-yl)oxy]phenyl}urea (1050 mg, 18.5 mmol), 10% palladium carbon (800 mg), 1,4-cyclohexadiene (4.21 g, 52.5 mmol) and ethanol (100 mL) was stirred at 40° C. for 3.5 hr. After celite filtration, the filtrate was concentrated under reduced pressure. The residue was purified by column chromatography (silica gel, hexane/ethyl acetate=80/20→0/100) and recrystallized from methanol/ethyl acetate to give the title compound (5.83 mg, 66%).

¹H-NMR (DMSO-d₆, 300 MHz) δ 4.10 (3H, s), 6.59 (1H, d, J=3.0 Hz), 6.98 (1H, d, J=9.0 Hz), 7.27 (1H, dd, J=9.0, 2.7 Hz), 7.38 (1H, dd, J=9.0, 2.1 Hz), 7.52 (1H, d, J=2.1 Hz), 7.76-7.78 (2H, m), 8.18 (1H, d, J=9.0 Hz), 8.27 (1H, br s), 8.29 (1H, s), 9.32 (1H, br s), 10.22 (1H, br s).

### Example 141

### N-{2-fluoro-4-[(5-methyl-5H-pyrrolo[3,2-d]pyrimidin-4-yl)oxy]phenyl}-N′-[3-(trifluoromethyl)phenyl]urea hydrobromide

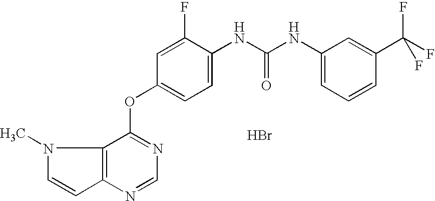

N-{2-Fluoro-4-[(5-methyl-5H-pyrrolo[3,2-d]pyrimidin-4-yl)oxy]phenyl}-N′-[3-(trifluoromethyl)phenyl]urea (0.40 g, 0.90 mmol) was dissolved in ethanol (10 mL) with heating, 48% hydrobromic acid (121 μL) was added, and the mixture was stirred at room temperature for 20 hr. The mixture was concentrated under reduced pressure to about 5 mL. The precipitate was collected by filtration, washed with ethanol, and dried under reduced pressure at 90° C. for 4 hr to give the title compound (438 mg, 93%) as colorless crystals. ¹H-NMR showed similar spectrum as in Example 138.

### Example 142

### N-{2-fluoro-4-[(5-methyl-5H-pyrrolo[3,2-d]pyrimidin-4-yl)oxy]phenyl}-N′-[3-(trifluoromethyl)phenyl]urea 0.5 Fumaric Acid Salt

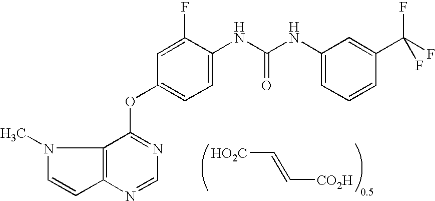

N-{2-Fluoro-4-[(5-methyl-5H-pyrrolo[3,2-d]pyrimidin-4-yl)oxy]phenyl}-N′-[3-(trifluoromethyl)phenyl]urea (2.00 g, 4.49 mmol) was dissolved in ethanol (25 mL) at 75° C., and fumaric acid (261 mg, 2.25 mmol) was added. After cooling to room temperature with stirring, the precipitated solid was collected by filtration, washed with a small amount of ethanol and dried under reduced pressure at 80° C. to give the title compound (1.86 g, 82%) as a white solid.

¹H-NMR (DMSO-d₆, 300 MHz) δ 4.11 (3H, s), 6.59 (1H, d, J=3.0 Hz), 6.62 (1H, s), 7.14 (1H, d, J=9.3 Hz), 7.31-7.40 (2H, m), 7.49-7.56 (2H, m), 7.78 (1H, d, J=3.0 Hz), 8.04 (1H, s), 8.13 (1H, t, J=9.3 Hz), 8.29 (1H, s), 8.66 (1H, br s), 9.39 (1H, br s).

### Example 143

### N-{2-fluoro-4-[(5-methyl-5H-pyrrolo[3,2-d]pyrimidin-4-yl)oxy]phenyl}-N′-[3-(trifluoromethyl)phenyl]urea Maleic Acid Salt

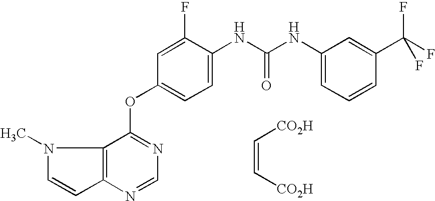

N-{2-Fluoro-4-[(5-methyl-5H-pyrrolo[3,2-d]pyrimidin-4-yl)oxy]phenyl}-N′-[3-(trifluoromethyl)phenyl]urea (445 mg, 0.999 mmol) was dissolved in ethanol (5 mL) at 70° C., and maleic acid (58 mg, 0.500 mmol) was added. After cooling to room temperature with stirring, maleic acid (58 mg, 0.500 mmol) was added. After stirring at room temperature for 7 hr, the precipitated solid was collected by filtration, washed with a small amount of ethanol, and dried under reduced pressure at 80° C. to give the title compound (406 mg, 72%) as a white solid.

¹H-NMR (DMSO-d₆, 300 MHz) δ4.12 (3H, s), 6.26 (2H, s), 6.61 (1H, d, J=3.0 Hz), 7.15 (1H, d, J=9.0 Hz), 7.32-7.41 (2H, m), 7.52-7.59 (2H, m), 7.80 (1H, d, J=3.0 Hz), 8.05 (1H, s), 8.15 (1H, t, J=9.0 Hz), 8.33 (1H, s), 8.67 (1H, br s), 9.40 (1H, br s).

### Example 144

### N-{2-fluoro-4-[(5-methyl-5H-pyrrolo[3,2-d]pyrimidin-4-yl)oxy]phenyl}-N′-[3-(trifluoromethyl)phenyl]urea 0.5 Succinic Acid Salt

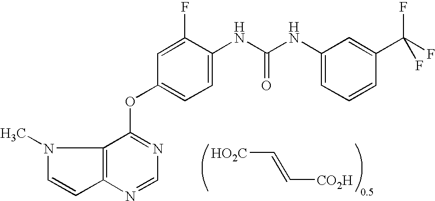

N-{2-Fluoro-4-[(5-methyl-5H-pyrrolo[3,2-d]pyrimidin-4-yl)oxy]phenyl}-N′-[3-(trifluoromethyl)phenyl]urea (445 mg, 0.999 mmol) was dissolved in ethanol (5 mL) at 70° C., and succinic acid (118 mg, 0.999 mmol) was added. After cooling to room temperature with stirring, the precipitated solid was collected by filtration, washed with a small amount of ethanol, and dried under reduced pressure at 80° C. to give the title compound (414 mg, 82%) as a white solid.

¹H-NMR (DMSO-d₆, 300 MHz) δ 2.42 (2H, s), 4.11 (3H, s), 6.60 (1H, d, J=3.0 Hz), 7.15 (1H, d, J=9.0 Hz), 7.31-7.41 (2H, m), 7.52-7.58 (2H, m), 7.78 (1H, d, J=3.0 Hz), 8.05 (1H, s), 8.14 (1H, t, J=9.0 Hz), 8.31 (1H, s), 8.66 (1H, br s), 9.40 (1H, br s), 12.13 (1H, br s).

### Example 145

### N-[2-chloro-4-({5-methyl-6-[(tetrahydro-2H-pyran-2-yloxy)methyl]-5H-pyrrolo[3,2-d]pyrimidin-4-yl}oxy)phenyl]-N′-[3-(trifluoromethyl)phenyl]urea

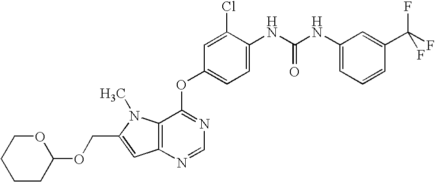

To a solution of 2-chloro-4-({5-methyl-6-[(tetrahydro-2H-pyran-2-yloxy)methyl]-5H-pyrrolo[3,2-d]pyrimidin-4-yl}oxy)aniline (1.35 g, 3.47 mmol) and triethylamine (1.45 mL, 10.4 mmol) in tetrahydrofuran (27 mL) was added 3-(trifluoromethyl)phenylisocyanate (543 μL, 3.89 mmol), and the mixture was stirred at room temperature for 5 hr. Water was added to the reaction mixture, and the mixture was extracted with ethyl acetate. The organic layer was washed with saturated brine, dried over anhydrous magnesium sulfate, and filtrated. The filtrate was concentrated under reduced pressure, and the residue was purified by column chromatography (NH silica gel, ethyl acetate/hexane) and recrystallized from ethyl acetate-hexane to give the title compound (0.91 g, 46%) as a white solid.

¹H-NMR (CDCl₃, 300 MHz) δ 1.45-1.86 (6H, m), 3.54-3.63 (1H, m), 3.86-3.96 (1H, m), 4.14 (3H, s), 4.72 (1H, d, J=12.9 Hz), 4.73-4.77 (1H, m), 4.95 (1H, d, J=12.9 Hz), 6.68 (1H, s), 7.15-7.33 (4H, m), 7.41 (1H, t, J=7.8 Hz), 7.52-7.56 (1H, m), 7.69-7.72 (2H, m), 8.26 (1H, d, J=8.7 Hz), 8.46 (1H, s).

### Example 146

### N-(2-chloro-4-{[6-(hydroxymethyl)-5-methyl-5H-pyrrolo[3,2-d]pyrimidin-4-yl]oxy}phenyl)-N′-[3-(trifluoromethyl)phenyl]urea

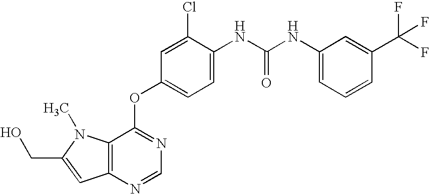

To a solution of N-[2-chloro-4-({5-methyl-6-[(tetrahydro-2H-pyran-2-yloxy)methyl]-5H-pyrrolo[3,2-d]pyrimidin-4-yl}oxy)phenyl]-N′-[3-(trifluoromethyl)phenyl]urea (0.70 g, 1.22 mmol) in ethanol (21 mL) was added p-toluenesulfonic acid monohydrate (509 mg, 2.67 mmol), and the mixture was stirred at room temperature for 3 hr. The solvent was evaporated under reduced pressure, saturated aqueous sodium hydrogen carbonate was added to the residue, and the mixture was extracted with ethyl acetate/tetrahydrofuran. The mixture was washed with saturated brine, dried over anhydrous magnesium sulfate, and filtrated. The filtrate was concentrated under reduced pressure, and the residue was purified by column chromatography (silica gel, ethyl acetate/ethanol) to give the title compound (105 mg, 18%) as a colorless solid.

¹H-NMR (DMSO-d₆, 300 MHz) δ 4.05 (3H, s), 4.73 (2H, d, J=5.4 Hz), 5.55 (1H, t, J=5.4 Hz), 6.57 (1H, s), 7.29-7.37 (2H, m), 7.51-7.58 (3H, m), 8.06 (1H, s), 8.17 (1H, d, J=9.0 Hz), 8.28 (1H, s), 8.47 (1H, s), 9.75 (1H, s).

### Example 147

### N-(2-chloro-4-{[6-(methoxymethyl)-5-methyl-5H-pyrrolo[3,2-d]pyrimidin-4-yl]oxy}phenyl)-N′-[3-(trifluoromethyl)phenyl]urea

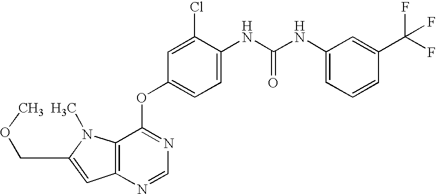

To a solution of 2-chloro-4-{[6-(methoxymethyl)-5-methyl-5H-pyrrolo[3,2-d]pyrimidin-4-yl]oxy}aniline (0.50 g, 1.57 mmol) and triethylamine (0.66 mL, 4.71 mmol) in tetrahydrofuran (10 mL) was added 3-(trifluoromethyl)phenylisocyanate (252 μL, 1.80 mmol), and the mixture was stirred at room temperature for 16 hr. Water was added to the reaction mixture, and the mixture was extracted with ethyl acetate. The organic layer was washed with saturated-brine, dried over anhydrous magnesium sulfate, and filtrated. The filtrate was concentrated under reduced pressure, and the residue was purified by column chromatography (NH silica gel, ethyl acetate/hexane) and recrystallized from ethyl acetate/hexane to give the title compound (296 mg, 37%) as a colorless solid.

¹H-NMR (DMSO-d₆, 300 MHz) δ 3.33 (3H, s), 4.04 (3H, s), 4.69 (2H, s), 6.66 (1H, s), 7.29-7.35 (2H, m), 7.51-7.58 (3H, m), 8.05 (1H, s), 8.16 (1H, d, J=9.0 Hz), 8.29 (1H, s), 8.47 (1H, s), 9.74 (1H, s).

### Example 148

### N-{2-fluoro-4-[(5-methyl-5H-pyrrolo[3,2-d]pyrimidin-4-yl)oxy]phenyl}-N′-[4-hydroxy-3-(trifluoromethyl)phenyl]urea

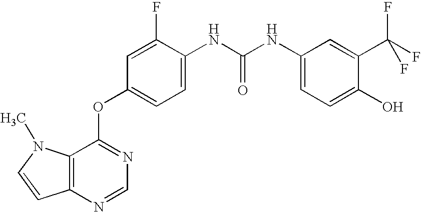

To a mixture of 2-fluoro-4-[(5-methyl-5H-pyrrolo[3,2-d]pyrimidin-4-yl)oxy]aniline (1291 mg, 5.00 mmol), triethylamine (2030 mg, 20.0 mmol) and tetrahydrofuran (50 mL) a solution (5 mL) of triphosgene (594 mg, 2.00 mmol) in tetrahydrofuran was added dropwise under ice-cooling. After stirring at room temperature for 1 hr. a solution (10 mL) of 4-(benzyloxy)-3-(trifluoromethyl)aniline (1600 mg, 5.99 mmol) in tetrahydrofuran was added dropwise under ice-cooling. The mixture was stirred at room temperature for 3.5 hr, and concentrated under reduced pressure. Ethyl acetate (300 mL) and tetrahydrofuran (50 mL) were added to the residue, and the mixture was washed with water (100 mL) and dried over anhydrous magnesium sulfate. After concentration under reduced pressure, the residue was purified by column chromatography (silica gel, hexane/ethyl acetate=75/25→0/100) and purified further by column chromatography (NH silica gel, hexane/ethyl acetate=50/50), and the residue was washed with hexane/ethyl acetate (1/1). The residue was dried under reduced pressure to give N-[4-(benzyloxy)-3-(trifluoromethyl)phenyl]-N′-{2-fluoro-4-[(5-methyl-5H-pyrrolo[3,2-d]pyrimidin-4-yl)oxy]phenyl}urea (1130 mg, 41%). A mixture of the thus-obtained N-[4-(benzyloxy)-3-(trifluoromethyl)phenyl]-N′-{2-fluoro-4-[(5-methyl-5H-pyrrolo[3,2-d]pyrimidin-4-yl)oxy]phenyl}urea (1020 mg, 18.5 mmol), 10% palladium carbon (800 mg), 1,4-cyclohexadiene (841 mg, 10.5 mmol) and ethanol (50 mL) was stirred at 40° C. for 19.5 hr. After celite filtration, the filtrate was concentrated under reduced pressure. The residue was purified by column chromatography (silica gel, hexane/ethyl acetate=80/20→0/100) and purified further by preparative HPLC and recrystallized from ethanol to give the title compound (251 mg, 29%).

¹H-NMR (DMSO-d₆, 300 MHz) δ 4.10 (3H, s), 6.59 (1H, d, J=3.0 Hz), 6.97 (1H, d, J=9.0 Hz), 7.11 (1H, d, J=9.0 Hz), 7.31-7.39 (2H, m), 7.75-7.78 (2H, m), 8.13 (1H, t, J=9.0 Hz), 8.29 (1H, s), 8.51 (1H, br s), 8.99 (1H, br s), 10.18 (1H, br s).

### Example 149

### N-{2-fluoro-4-[(5-methyl-5H-pyrrolo[3,2-d]pyrimidin-4-yl)oxy]phenyl}-N′-[3-(trifluoromethyl)phenyl]urea 0.5 Sulfuric Acid Salt

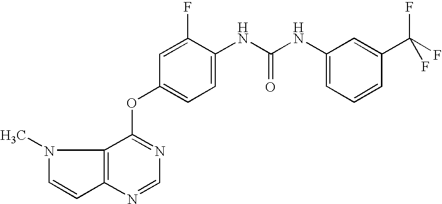

N-{2-Fluoro-4-[(5-methyl-5H-pyrrolo[3,2-d]pyrimidin-4-yl)oxy]phenyl}-N′-[3-(trifluoromethyl)phenyl]urea (200 mg, 0.45 mmol) was dissolved in ethanol (6.0 ml) with heating, 98% sulfuric acid (45 mg) was added, and the mixture was stood at room temperature for 6 hr. The precipitated crystals were collected by filtration, washed with ethanol, and dried under reduced pressure at 90° C. for 8 hr to give the title compound (140 mg, 57%) as colorless crystals.

¹H-NMR (DMSO-d₆, 300 MHz) δ 4.14 (3H, s), 6.69 (1H, s), 7.17-7.22 (1H, d, J=6.3 Hz), 7.35 (1H, dd, J=11.9, 2.9 Hz), 7.53-7.56 (2H, m), 7.95 (1H, s), 8.06 (1H, t, J=8.9 Hz), 8.52 (1H, s), 8.72 (1H, s), 9.43 (1H, s).

### Example 150

### N-{2-chloro-4-[(5-methyl-5H-pyrrolo[3,2-d]pyrimidin-4-yl)oxy]phenyl}-N′-(1-methylpiperidin-3-yl)urea

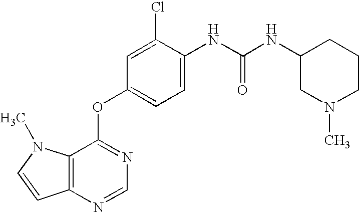

A mixture of tert-butyl 3-{[({2-chloro-4-[(5-methyl-5H-pyrrolo[3,2-d]pyrimidin-4-yl)oxy]phenyl}amino)carbonyl]amino}piperidine-1-carboxylate (200 mg, 0.399 mmol), 37% aqueous formic acid solution (0.3 mL) and acetic acid (3 mL) was stirred at 95° C. for 3 hr. The mixture was neutralized with 8N aqueous sodium hydroxide solution and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=10/90→100/0) and recrystallized from ethyl acetate-hexane to give the title compound (47.0 mg, 28%) as a white solid.

¹H-NMR (DMSO-d₆, 300 MHz) δ 1.20-1.70 (4H, m), 2.05-2.35 (7H, m), 3.65-3.75 (1H, m), 4.07 (3H, s), 6.58 (1H, d, J=3.0 Hz), 7.11-7.23 (2H, m), 7.44-7.46 (1H, m), 7.76 (1H, d, J=3.0 Hz), 8.14-8.20 (2H, m), 8.27 (1H, s).

### Example 151

### N-{4-[(5-methyl-5H-pyrrolo[3,2-d]pyrimidin-4-yl)oxy]phenyl}benzamide

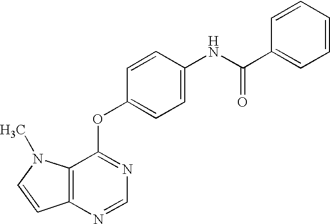

To a solution of 4-[(5-methyl-5H-pyrrolo[3,2-d]pyrimidin-4-yl)oxy]aniline (513 mg, 2.14 mmol) and triethylamine (889 μL, 6.42 mmol) in tetrahydrofuran (5 mL) was added benzoyl chloride (297 μL, 2.56 mmol) with stirring under ice-cooling, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=70/30→100/0) and recrystallized from ethyl acetate to give the title compound (56.0 mg, 7.6%) as a white solid.

¹H-NMR (DMSO-d₆, 300 MHz) δ 4.12 (3H, s), 6.59-6.60 (1H, m), 7.30 (2H, d, J=8.7 Hz), 7.52-7.63 (3H, m), 7.79 (1H, d, J=3.0 Hz), 7.85 (2H, d, J=8.7 Hz), 7.98 (2H, d, J=7.5 Hz), 8.28 (1H, s), 10.36 (1H, s).

### Example 152

### N-{4-[(5-methyl-5H-pyrrolo[3,2-d]pyrimidin-4-yl)oxy]phenyl}-2-phenylacetamide

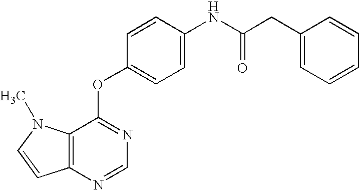

To a solution of 4-[(5-methyl-5H-pyrrolo[3,2-d]pyrimidin-4-yl)oxy]aniline (120 mg, 0.50 mmol) in N-methylpyrrolidone (3 mL) was added phenylacetyl chloride (0.132 mL, 1.00 mmol), and the mixture was stirred at room temperature for 18 hr. The reaction mixture was diluted with water and extracted with ethyl acetate. The organic layer was concentrated under reduced pressure, and the residue was purified by column chromatography (silica gel, ethyl acetate/methanol=100/0→80/20) and recrystallized from diisopropyl ether to give the title compound (151 mg, 84%) as a white solid.

¹H-NMR (DMSO-d₆, 300 MHz) δ 3.66 (2H, s), 4.09 (3H, s), 6.58 (1H, d, J=3.0 Hz), 7.22-7.38 (7H, m), 7.67 (2H, d, J=9.0 Hz), 7.77 (1H, d, J=3.0 Hz), 8.26 (1H, s), 10.26 (1H, br s).

### Example 153

### N-{3-[(5-methyl-5H-pyrrolo[3,2-d]pyrimidin-4-yl)oxy]phenyl}-N′-phenylurea

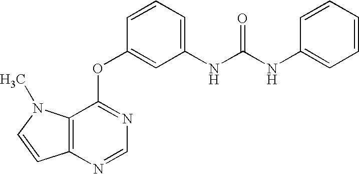

To a solution of 3-[(5-methyl-5H-pyrrolo[3,2-d]pyrimidin-4-yl)oxy]aniline (579 mg, 2.41 mmol) and triethylamine (1.00 mL, 7.22 mmol) in tetrahydrofuran (10 mL) was added phenylisocyanate (314 μL, 2.89 mmol), and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by column chromatography (NH silica gel, ethyl acetate/hexane=30/70→100/0) and recrystallized from ethyl acetate-hexane to give the title compound (690 mg, 80%) as a white solid.

¹H-NMR (DMSO-d₆, 300 MHz) δ 4.10 (3H, s), 6.60-6.61 (1H, m), 6.88-6.98 (2H, m), 7.20-7.30 (3H, m), 7.35 (1H, t, J=8.1 Hz), 7.43 (2H, d, J=8.1 Hz), 7.57 (1H, t, J=2.1 Hz), 7.79 (1H, d, J=3.0 Hz), 8.29 (1H, s), 8.72 (1H, s), 8.86 (1H, s).

### Example 154

### N-{3-[(5-methyl-5H-pyrrolo[3,2-d]pyrimidin-4-yl)oxy]phenyl}benzamide

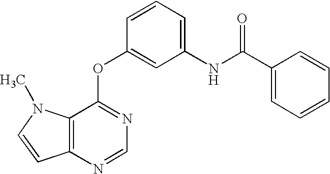

Using 3-[(5-methyl-5H-pyrrolo[3,2-d]pyrimidin-4-yl)oxy]aniline (415 mg, 1.73 mmol), triethylamine (719 μL, 5.19 mmol), benzoyl chloride (221 μL, 1.90 mmol) and tetrahydrofuran (10 mL) as starting materials, and in the same manner as in Example 151, the title compound (262 mg, 44%) was obtained as a white solid.

¹H-NMR (DMSO-d₆, 300 MHz) δ 4.12 (3H, s), 6.61 (1H, d, J=3.0 Hz), 7.04-7.07 (1H, m), 7.44 (1H, t, J=8.1 Hz), 7.50-7.68 (4H, m), 7.80 (1H, d, J=3.0 Hz), 7.84 (1H, t, J=2.3 Hz), 7.93-7.97 (2H, m), 8.30 (1H, s), 10.41 (1H, s).

### Example 155

### N-{2-fluoro-4-[(6-hydroxy-5-methyl-5H-pyrrolo[3,2-d]pyrimidin-4-yl)oxy]phenyl}-N′-[3-(trifluoromethyl)phenyl]urea

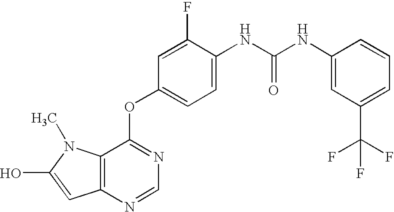

To a solution of N-{2-fluoro-4-[(5-methyl-5H-pyrrolo[3,2-d]pyrimidin-4-yl)oxy]phenyl}-N′-[3-(trifluoromethyl)phenyl]urea (445 mg, 1.0 mmol) in tert-butanol/water (20 mL/10 mL) was added bromine (0.205 mL, 4.0 mmol), and the mixture was stirred at room temperature for 1 hr. 10% sodium thiosulfate (50 mL) was added to the reaction mixture, and the mixture was stirred at room temperature for 15 min. After stirring, the mixture was diluted with water, and extracted with a mixed solvent of ethyl acetate/tetrahydrofuran. The organic layer was concentrated under reduced pressure, and the residue was purified by column chromatography (silica gel, ethyl acetate/methanol=100/0→80/20). The object fractions were collected and, after concentration under reduced pressure, the residue was applied to high performance liquid chromatography (ODS column, 0.1% trifluoroacetic acid-containing water/0.1% trifluoroacetic acid-containing acetonitrile=60/40→0/100) The object fractions were collected. Acetonitrile was evaporated under reduced pressure, and the residue was extracted with a mixed solvent of ethyl acetate/tetrahydrofuran. The organic layer was washed with 5% aqueous sodium hydrogen carbonate solution, dried over anhydrous magnesium sulfate, filtrated, and concentrated under reduced pressure. The residue was recrystallized from a mixed solvent of ethyl acetate/diisopropyl ether to give the title compound (169 mg, 37%).

### Example 156

### N-(1-tert-butyl-1H-pyrazol-4-yl)-N′-{2-chloro-4-[(5-methyl-5H-pyrrolo[3,2-d]pyrimidin-4-yl)oxy]phenyl}urea

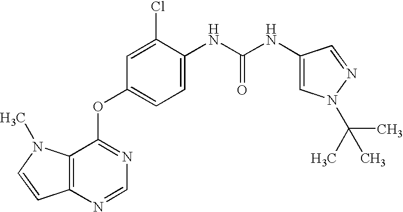

Using 2-chloro-4-[(5-methyl-5H-pyrrolo[3,2-d]pyrimidin-4-yl)oxy]aniline (275 mg, 1.0 mmol), triethylamine (2.79 mL, 20 mmol), dichloromethane (10 mL), triphosgene (297 mg, 1.0 mmol) and 1-tert-butyl-1H-pyrazole-4-amine (278 mg, 2.0 mmol), and in the same manner as in Example 113, the title compound (224 mg, 51%) was obtained.

¹H-NMR (DMSO-d₆, 300 MHz) δ 1.51 (9H, s), 4.10 (3H, s), 6.60 (1H, d, J=3.0 Hz), 7.28 (1H, dd, J=9.0, 2.7 Hz), 7.43 (1H, s), 7.53 (1H, d, J=2.7 Hz), 7.79 (1H, d, J=3.0 Hz), 7.85 (1H, s), 8.21 (1H, d, J=9.0 Hz), 8.28 (1H, s), 8.30 (1H, s), 9.08 (1H, s).

### Example 157

### N-{2-chloro-4-[(6-methyl-5H-pyrrolo[3,2-d]pyrimidin-4-yl)oxy]phenyl}-N′-[3-(trifluoromethyl)phenyl]urea

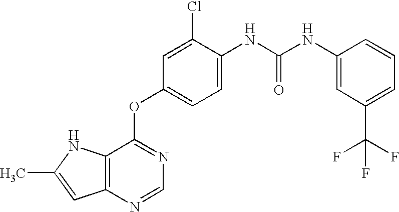

To a solution of 2-chloro-4-[(6-methyl-5H-pyrrolo[3,2-d]pyrimidin-4-yl)oxy]aniline (275 mg, 1.0 mmol) in tetrahydrofuran (40 mL) was added 3-(trifluoromethyl)phenylisocyanate (179 μL, 1.3 mmol), and the mixture was stirred at room temperature for 18 hr. The reaction mixture was concentrated under reduced pressure, and the residue was purified by column chromatography (NH silica gel, ethyl acetate/methanol=100/0→80/20) and recrystallized from methanol to give the title compound (124 mg, 27%) as a white solid.

¹H-NMR (DMSO-d₆, 300 MHz) δ 2.48 (3H, s), 6.37 (1H, s), 7.27 (1H, dd, J=8.9, 2.9 Hz), 7.35 (1H, br d, J=7.2 Hz), 7.52-7.60 (3H, m), 8.06 (1H, br s), 8.14 (1H, d, J=8.9 Hz), 8.26 (1H, s), 8.47 (1H, br s), 9.75 (1H, br s), 12.12 (1H, br s).

### Example 158

### N-(3-{[(5-methyl-5H-pyrrolo[3,2-d]pyrimidin-4-yl)oxy]methyl}phenyl)benzamide Hydrochloride

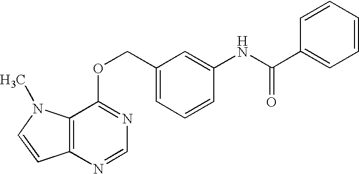

To a solution of 3-{[(5-methyl-5H-pyrrolo[3,2-d]pyrimidin-4-yl)oxy]methyl}aniline (540 mg, 2.12 mmol) and triethylamine (881 μL, 6.36 mmol) in tetrahydrofuran (5 mL) was added benzoyl chloride (271 μL, 2.33 mmol) with stirring under ice-cooling, and the mixture was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was purified by silica gel column chromatography (ethyl acetate/hexane=70/30→100/0). The residue was dissolved in ethanol (2 mL), 4N hydrochloric acid ethyl acetate solution (518 μL, 2.07 mmol) was added, and the mixture was stirred at room temperature for 15 hr. The precipitated solid was collected by filtration and washed with ethyl acetate to give the title compound (603 mg, 72%) as a white solid.

¹H-NMR (DMSO-d₆, 300 MHz) δ 4.11 (3H, s), 5.75 (2H, s), 6.71 (1H, d, J=3.2 Hz), 7.29 (1H, d, J=7.5 Hz), 7.41 (1H, t, J=8.0 Hz), 7.50-7.62 (3H, m), 7.72-7.77 (1H, m), 7.94-7.98 (2H, m), 8.01 (1H, d, J=3.2 Hz), 8.08 (1H, s), 8.89 (1H, s), 10.39 (1H, s).

### Example 159

### N-(3-{[(5-methyl-5H-pyrrolo[3,2-d]pyrimidin-4-yl)oxy]methyl}phenyl)-N′-phenylurea

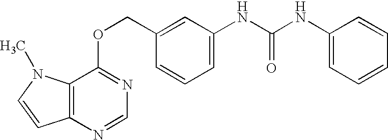

Using 3-{[(5-methyl-5H-pyrrolo[3,2-d]pyrimidin-4-yl)oxy]methyl}aniline (509 mg, 2.00 mmol), triethylamine (831 μL, 6.00 mmol), phenylisocyanate (261 μL, 2.40 mmol) and tetrahydrofuran (10 mL) as starting materials, and in the same manner as in Example 153, the title compound (466 mg, 62%) was obtained as a white solid.

¹H-NMR (DMSO-d₆, 300 MHz) δ 4.04 (3H, s), 5.57 (2H, s), 6.52 (1H, d, J=3.0 Hz), 6.93-6.99 (1H, m), 7.12 (1H, d, J=7.8 Hz), 7.23-7.33 (3H, m), 7.38-7.46 (3H, m), 7.65-7.67 (2H, m), 8.38 (1H, s), 8.66 (1H, s), 8.73 (1H, s).

### Example 160

### N-{4-[(5-methyl-5H-pyrrolo[3,2-d]pyrimidin-4-yl) oxy]phenyl}-N′-phenylthiourea

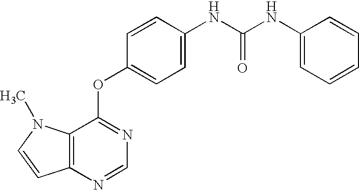

Using 4-[(5-methyl-5H-pyrrolo[3,2-d]pyrimidin-4-yl)oxy]aniline (1.10 g, 4.58 mmol), triethylamine (1.90 mL, 13.7 mmol), phenylisothiocyanate (657 μL, 5.44 mmol) and tetrahydrofuran (10 mL) as starting materials, and in the same manner as in Example 153, the title compound (1.29 g, 75%) was obtained as a white solid.

¹H-NMR (DMSO-d₆, 300 MHz) δ 4.11 (3H, s), 6.60 (1H, d, J=3.2 Hz), 7.10-7.16 (1H, m), 7.24-7.38 (4H, m), 7.47-7.58 (4H, m), 7.78 (1H, d, J=3.2 Hz), 8.29 (1H, s), 9.84 (2H, s).

### Example 161

### 4-[(5-methyl-5H-pyrrolo[3,2-d]pyrimidin-4-yl)oxy]-N-phenylbenzamide

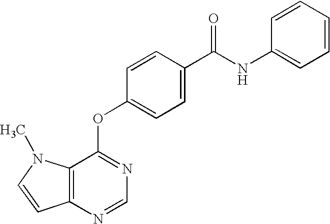

A mixture of 4-[(5-methyl-5H-pyrrolo[3,2-d]pyrimidin-4-yl)oxy]benzoic acid (567 mg, 2.11 mmol), 1-ethyl-3-(3-dimethylaminopropyl)carbodiimide hydrochloride (607 mg, 3.17 mmol), 1-hydroxy-1H-benzotriazole (428 mg, 3.17 mmol), aniline (192 μL, 2.11 mmol) and N,N-dimethylformamide (5 mL) was stirred at room temperature for 15 hr. The reaction mixture was diluted with water, and extracted with ethyl acetate (×3). The organic layer was washed with saturated brine, dried over anhydrous magnesium sulfate, and filtrated. The filtrate was concentrated under reduced pressure, and the residue was collected by filtration and recrystallized from ethyl acetate-hexane to give the title compound (207 mg, 28%) as a yellow solid.

¹H-NMR (DMSO-d₆, 300 MHz) δ 4.12 (3H, s), 6.62 (1H, d, J=3.0 Hz), 7.10 (1H, t, J=7.7 Hz), 7.36 (2H, t, J=7.7 Hz), 7.48 (2H, d, J=8.6 Hz), 7.78-7.83 (3H, m), 8.06 (2H, d, J=8.6 z), 8.31 (1H, s), 10.30 (1H, s).

### Example 162

### 3-[(5-methyl-5H-pyrrolo[3,2-d]pyrimidin-4-yl)oxy]-N-phenylbenzamide

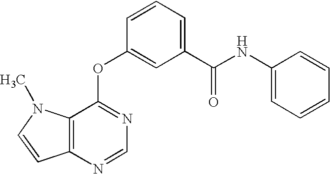

Using 3-[(5-methyl-5H-pyrrolo[3,2-d]pyrimidin-4-yl)oxy]benzoic acid (552 mg, 2.05 mmol), 1-ethyl-3-(3-dimethylaminopropyl)carbodiimide hydrochloride (589 mg, 3.07 mmol), 1-hydroxy-1H-benzotriazole (415 mg, 3.07 mmol), aniline (187 μL, 2.05 mmol) and N,N-dimethylformamide (5 mL) as starting materials, and in the same manner as in Example 161, the title compound (496 mg, 70%) was obtained as a white solid.

¹H-NMR (DMSO-d₆, 300 MHz) δ 4.14 (3H, s), 6.62 (1H, d, J=3.0 Hz), 7.07-7.13 (1H, m), 7.32-7.38 (2H, m), 7.54-7.67 (2H, m), 7.75-7.82 (3H, m), 7.89-7.92 (2H, m), 8.30 (1H, s), 10.31 (1H, s).

### Example 163

### N-(4-{[(5-methyl-5H-pyrrolo[3,2-d]pyrimidin-4-yl) oxy]methyl}phenyl)-N′-phenylurea

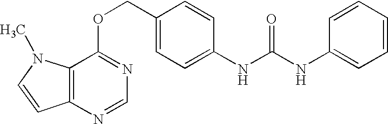

Using 4-{[(5-methyl-5H-pyrrolo[3,2-d]pyrimidin-4-yl)oxy]methyl}aniline (205 mg, 0.806 mmol), triethylamine (335 μL, 2.41 mmol), phenylisocyanate (105 μL, 0.967 mmol) and tetrahydrofuran (5 mL) as starting materials, and in the same manner as in Example 153, the title compound (232 mg, 77%) was obtained as a white solid.

¹H-NMR (DMSO-d₆, 300 MHz) δ 4.00 (3H, s), 5.09 (2H, s), 6.32 (1H, d, J=3.0 Hz), 6.95 (1H, t, J=7.2 Hz), 7.27-7.30 (4H, m), 7.37-7.46 (5H, m), 8.24 (1H, s), 8.63 (1H, s), 8.68 (1H, s).

### Example 164

### N-methyl-6-[(5-methyl-5H-pyrrolo[3,2-d]pyrimidin-4-yl)oxy]-1-naphthamide

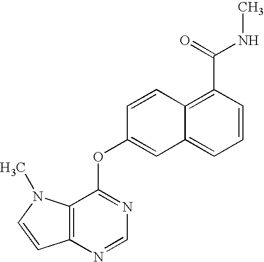

Using 6-[(5-methyl-5H-pyrrolo[3,2-d]pyrimidin-4-yl)oxy]-1-naphthoic acid (500 mg, 1.57 mmol), 1-ethyl-3-(3-dimethylaminopropyl)carbodiimide hydrochloride (360 mg, 1.88 mmol), 1-hydroxy-1H-benzotriazole (254 mg, 1.88 mmol), 2M methyl amine tetrahydrofuran solution (715 μL, 1.43 mmol) and N,N-dimethylformamide (5 mL) as starting materials, and in the same manner as in Example 161, the title compound (361 mg, 76%) was obtained as a white solid.

¹H-NMR (DMSO-d₆, 300 MHz) δ 2.87 (3H, d, J=4.8 Hz), 4.16 (3H, s), 6.63 (1H, d, J=3.0 Hz), 7.54-7.63 (3H, m), 7.83 (1H, d, J=3.0 Hz), 7.92 (1H, d, J=2.3 Hz), 8.02 (1H, dd, J=7.4, 2.3 Hz), 8.28-8.32 (2H, m), 8.51-8.58 (1H, m).

### Example 165

### 2-{4-[(5-methyl-5H-pyrrolo[3,2-d]pyrimidin-4-yl)oxy]phenyl}-N-phenylacetamide

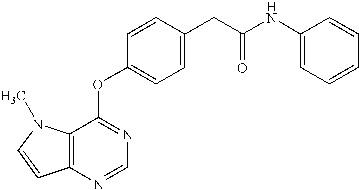

A mixture of {4-[(5-methyl-5H-pyrrolo[3,2-d]pyrimidin-4-yl)oxy]phenyl}acetic acid (142 mg, 0.50 mmol), aniline (56 mg, 0.60 mmol), 1-hydroxy-1H-benzotriazole hydrate (92 mg, 0.60 mmol), 1-ethyl-3-(3-dimethylaminopropyl)carbodiimide hydrochloride (192 mg, 1.00 mmol), triethylamine (0.209 mL, 1.5 mmol) and N,N-dimethylformamide (5 mL) was stirred at room temperature for 18 hr. The reaction mixture was concentrated under reduced pressure, the residue was diluted with water, and the precipitate was collected by filtration and washed with water. The precipitate was recrystallized from methanol to give the title compound (146 mg, 81%) as a white solid.

¹H-NMR (DMSO-d₆, 300 MHz) δ 3.69 (2H, s), 4.10 (3H, s), 6.59 (1H, d, J=3.0 Hz), 7.05 (1H, t, J=7.5 Hz), 7.26 (2H, d, J=8.7 Hz), 7.32 (2H, d, J=7.5 Hz), 7.42 (2H, d, J=8.7 Hz), 7.61 (2H, d, J=7.5 Hz), 7.78 (1H, d, J=3.0 Hz), 8.27 (1H, s), 10.18 (1H, br s).

### Formulation Example 1

A pharmaceutical agent containing the compound of the present invention as an active ingredient can be produced, for example, by the following formulation.

### 1. Capsule

| | | |
|---|---|---|
| (1) compound obtained in Example 1 | 40 | mg |
| (2) lactose | 70 | mg |
| (3) microcrystalline cellulose | 9 | mg |
| (4) magnesium stearate | 1 | mg |
| 1 capsule | 120 | mg |

(1), (2), (3) and ½ of (4) are admixed and granulated. Thereto is added the rest of (4) and the whole is encapsulated in a gelatin capsule.

### 2. Tablet

| | | |
|---|---|---|
| (1) compound obtained in Example 1 | 40 | mg |
| (2) lactose | 58 | mg |
| (3) cornstarch | 18 | mg |
| (4) microcrystalline cellulose | 3.5 | mg |
| (5) magnesium stearate | 0.5 | mg |
| 1 tablet | 120 | mg |

(1), (2), (3), ⅔ of (4) and ½ of (5) are admixed and granulated. To the granules are added the rest of (4) and (5) and the mixture is compression-formed to give a tablet.

### Formulation Example 2

The compound (50 mg) obtained in Example 1 was dissolved in Japanese Pharmacopoeia distilled water for injection (50 ml), Japanese Pharmacopoeia-distilled water for injection was added to 100 ml. This solution was filtered under sterile conditions. The solution (1 ml) was filled in a injection vial under sterile conditions, lyophilized and sealed.

### Experimental Example 1

### Cloning of Human VEGF Receptor 2 (VEGFR2) Gene and Preparation of Recombinant Baculovirus

For cloning of human VEGF receptor 2 (hereinafter to be abbreviated as VEGFR2) gene, PCR was performed using cDNA Libraries Human Placenta (Clontech) as a template. The primer used for PCR was prepared by, based on the information of the base sequence (Genbank Accession AF035121) of VEGFR2 gene, adding a base sequence encoding the flag peptide and a restriction enzyme recognition sequence to the base sequence (2671-4374 of Genbank Accession AF035121) encoding the VEGFR2 intracellular domain, so that a flag tag would be added to the N terminal of the protein. The primer base sequence is shown in the following.

VEGFR2-U:

(SEQ ID NO:1)

5′-AATTAAGTCGACATGGACTACAAGGATGACGATGACAAGAAGCGGGC
CAATGGAGGGGAACTGAAGACA-3′

and

VEGFR2-L:

(SEQ ID NO:2)

5′-AATTAAGCATGCTTAAACAGGAGGAGAGCTCAGTGTGGTCCC-3′

PCR reaction was carried out using a KOD-plus kit (TOYOBO). The obtained PCR product was electrophoresed on an agarose gel (1%), and a DNA fragment amplified by PCR was recovered from the gel and digested with restriction enzymes Sal I and Sph I. The DNA after the restriction enzyme treatment was electrophoresed on an agarose gel (1%), and the obtained DNA fragment was recovered and ligated to the plasmid pFASTBAC1 (Invitrogen) digested with restriction enzymes Sal I and Sph I to give an expression plasmid pFB-VEGFR2. The base sequence of the inserted fragment was confirmed to have matched with the base sequence (2671-4374 of Genbank Accession AF035121) of the VEGFR2 intracellular domain. Furthermore, virus stock BAC-VEGFR2 of the recombinant Baculovirus was prepared using BAC-TO-BAC Baculovirus Expression System (Invitrogen).

### Experimental Example 2

### Preparation of VEGF Receptor 2 (VEGFR2) Intracellular Domain Protein

SF-21 cells were inoculated into 1 L of Sf-900IISFM medium (Invitrogen) containing 10% fetal calf serum (trace), 50 mg/L Gentamicin (Invitrogen) and 0.1% Pluronic F-68 (Invitrogen) at 1×10⁶ cells/ml, and shake culture was performed using a 2 L Erlenmeyer flask at 27° C., 100 rpm. After 24 hr of culture, recombinant Baculovirus BAC-VEGFR2 (13.4 mL) was added, and the culture was continued for 3 days. The culture medium was centrifuged at 2,000 rpm for 5 min to give virus-infected cells. The infected cells were washed with phosphate buffered saline (Invitrogen), centrifuged under the same conditions, and preserved at −80° C. The cryopreserved cells were thawed in ice, suspended in 30 mL of buffer A (Tris buffer, pH 7.4, containing 20% glycerol and 0.15M NaCl (50 mM)) supplemented with Complete Protease Inhibitor (Boehringer), and disrupted three times using a polytron homogenizer (Kinematica) at 20,000 rpm, 30 sec. The disrupt was clarified by centrifugation at 40,000 rpm, 30 min and filtered through a 0.45 μm filter. The filtrate was passed through a column packed with Anti-FLAG M2 Affinity Gel (Sigma Ltd., 4 mL) at a flow rate of about 0.5 mL/min. The column was washed with buffer A and eluted with buffer A containing 100 μg/mL FLAG peptide. The eluate was concentrated by Vivaspin 20 (Viva Science) having a fraction molecular weight of 30K. The concentrate was applied to NAP™ 25 column (Amersham Biosciences) equilibrated with buffer A for buffer exchange. The fractions containing the VEGFR2 intracellular domain protein were collected, glycerol was added to the final concentration of 50% and the fractions cryopreserved at ˜80° C.

### Test Example 1

### Determination of VEGF Receptor 2 Kinase (VEGFR2) Inhibitory Activity

The test compound dissolved in dimethyl sulfoxide (DMSO) was diluted with a buffer (50 mM Tris-HCl, pH 7.5, 5 mM MgCl₂, 5 mM MnCl₂, 2 mM dithiothreitol, 0.01% Tween-20). A buffer (10 μl) containing 50 ng/ml VEGFR2 intracellular domain protein and 250 ng/ml biotin-labeled polypeptide biotinyl-poly-Glu:Tyr (4:1) (CIS Bio International) was added to the compound solution (5 μl). A buffer (10 μl) containing 25 μM ATP was added to the obtained mixture, the mixture was allowed to react at 25° C. for 5 min. Then, 25 μl of a stop solution (100 mM EDTA·2 sodium salt, 62.5 mM HEPES buffer (pH 7.4), 250 mM NaCl, 0.1% bovine serum albumin, 10 μg/ml Streptavidin Donor beads: PerkinElmer for alpha screen assay, 10 μg/ml Anti-phosphotyrosine (P-Tyr-100) Acceptor beads: PerkinElmer for alpha screen assay) was added to quench the reaction. The reaction solution was stood at 25° C. for 16 hr, and the count was measured using a plate reader·Fusion™ (manufactured by PerkinElmer). The kinase inhibitory rate (%) of the test compound was calculated by the following formula.

Inhibitory rate(%)=(1−(count of test compound−blank)÷(control−blank))×100

wherein the count of the solution reacted without addition of the compound is “control” and the count of the solution without addition of the compound and ATP is “blank”.

The inhibitory rates of the compounds of Examples 4, 14, 15, 18, 20, 29, 30, 39, 40, 41, 47, 60, 77, 116 and 137 at 1 μM were not less than 90%.

### Test Example 2

### Determination of PDGFR Alpha Receptor Kinase (PDGFRalpha) Inhibitory Activity

The test compound dissolved in dimethyl sulfoxide (DMSO) was diluted with a buffer (50 mM Tris-HCl, pH 7.5, 5 mM MgCl₂, 5 mM MnCl₂, 2 mM dithiothreitol, 0.01% Tween-20). A buffer (10 μl) containing 125 ng/ml PDGFRalpha intracellular domain protein (UPSTATE) and 250 ng/ml biotin-labeled polypeptide biotinyl-poly-Glu:Tyr (˜4:1) (CIS Bio International) was added to the compound solution (5 μl). At 5 min after mixing a kinase enzyme, the compound and a biotin-labeled polypeptide, a buffer (10 μl) containing 25 μM ATP was added to the obtained mixture, the mixture was allowed to react at 25° C. for 30 min. Then, 25 μl of a stop solution (100 mM EDTA-2 sodium salt, 62.5 mM HEPES buffer (pH 7.4), 250 mM NaCl, 0.1% bovine serum albumin, 10 μg/ml Streptavidin Donor beads: PerkinElmer for alpha screen assay, 10 μg/ml Anti-phosphotyrosine (P-Tyr-66)

Acceptor beads: PerkinElmer for alpha screen assay) was added to quench the reaction. The reaction solution was stood at 25° C. for 16 hr, and the count was measured using a plate reader·Fusion™ (manufactured by PerkinElmer). The kinase inhibitory rate (%) of the test compound was calculated by the following formula.

Inhibitory rate(%)=(1−(count of test compound−blank)÷(control−blank))×100

wherein the count of the solution reacted without addition of the compound is “control” and the count of the solution without addition of the compound and ATP is “blank”.

The kinase inhibitory rate (%) of the test compound at concentrations of 10 μM, 1 μM, 100 nM, 10 nM, 1 nM, 100 μM, 10 μM was calculated, and the concentration (IC₅₀ value) of the test compound necessary for inhibiting the enzyme by 50% was calculated using Graphpad Prism (Graphpad Software) according to the nonlinear regression sigmoidal dose-response (variable slope) analysis method.

The IC₅₀ values of the compounds of Examples 4, 14, 15, 18, 20, 29, 39, 41, 47, 77, 116 and 137 were not more than 500 nM.

### Test Example 3

### Determination of PDGFRbeta Receptor Kinase (PDGFRbeta) Inhibitory Activity

The test compound dissolved in dimethyl sulfoxide (DMSO) was diluted with a buffer (50 mM Tris-HCl, pH 7.5, 5 mM MgCl₂, 5 mM MnCl₂, 2 mM dithiothreitol, 0.01% Tween-20). A buffer (10 μl) containing 125 ng/ml PDGFRbeta intracellular domain protein (UPSTATE) and 250 ng/ml biotin-labeled polypeptide biotinyl-poly-Glu:Tyr (4:1) (CIS Bio International) was added to the compound solution (5 μl). At 5 min after mixing a kinase enzyme, the compound and a biotin-labeled polypeptide, a buffer (10 μl) containing 25 μM ATP was added to the obtained mixture, the mixture was allowed to react at 25° C. for 30 min. Then, 25 μl of a stop solution (100 mM EDTA·2 sodium salt, 62.5 mM HEPES buffer (pH 7.4), 250 mM NaCl, 0.1% bovine serum albumin, 10 μg/ml Streptavidin Donor beads: PerkinElmer for alpha screen assay, 10 μg/ml Anti-phosphotyrosine (P-Tyr-66) Acceptor beads: PerkinElmer for alpha screen assay) was added to quench the reaction. The reaction solution was stood at 25° C. for 16 hr, and the count was measured using a plate reader·Fusion™ (manufactured by PerkinElmer). The kinase inhibitory rate (%) of the test compound was calculated by the following formula.

Inhibitory rate(%)=(1−(count of test compound−blank)÷(control−blank))×100

wherein the count of the solution reacted without addition of the compound is “control” and the count of the solution without addition of the compound and ATP is “blank”.

The kinase inhibitory rate (%) of the test compound at concentrations of 10 μM, 1 μM, 100 nM, 10 nM, 1 nM, 100 pM, 10 pM was calculated, and the concentration (IC₅₀ value) of the test compound necessary for inhibiting the enzyme by 50% was calculated using Graphpad Prism (Graphpad Software) according to the nonlinear regression sigmoidal dose-response (variable slope) analysis method.

The IC₅₀ values of the compounds of Examples 4, 15, 18, 20, 29, 41, 47, 77 and 137 were not more than 500 nM.

### Test Example 4

### Determination of TIE2 Receptor Kinase (TIE2) Inhibitory Activity

The test compound dissolved in dimethyl sulfoxide (DMSO) was diluted with a buffer (50 mM Tris-HCl, pH 7.5, 5 mM MgCl₂, 5 mM MnCl₂, 2 mM dithiothreitol, 0.01% Tween-20). A buffer (10 μl) containing 50 ng/ml TIE2 intracellular domain protein (UPSTATE) and 250 ng/ml biotin-labeled polypeptide biotinyl-poly-Glu:Tyr (4:1) (CIS Bio International) was added to the compound solution (5 μl). At 5 min after mixing a kinase enzyme, the compound and a biotin-labeled polypeptide, a buffer (10 μl) containing 5 μM ATP was added to the obtained mixture, the mixture was allowed to react at 25° C. for 10 min. Then, 25 μl of a stop solution (100 mM EDTA·2 sodium salt, 62.5 mM HEPES buffer (pH 7.4), 250 mM NaCl, 0.1% bovine serum albumin, 10 μg/ml Streptavidin Donor beads: PerkinElmer for alpha screen assay, 10 μg/ml Anti-phosphotyrosine (P-Tyr-66) Acceptor beads: PerkinElmer for alpha screen assay) was added to quench the reaction. The reaction solution was stood at 25° C. for 16 hr, and the count was measured using a plate reader·Fusion™ (manufactured by PerkinElmer). The kinase inhibitory rate (%) of the test compound was calculated by the following formula.

Inhibitory rate(%)=(1−(count of test compound−blank)÷(control−blank))×100

wherein the count of the solution reacted without addition of the compound is “control” and the count of the solution without addition of the compound and ATP is “blank”.

The kinase inhibitory rate (%) of the test compound at concentrations of 10 μM, 1 μM, 100 nM, 10 nM, 1 nM, 100 pM, 10 pM was calculated, and the concentration (IC₅₀ value) of the test compound necessary for inhibiting the enzyme by 50% was calculated using Graphpad. Prism (Graphpad Software) according to the nonlinear-regression sigmoidal dose-response (variable slope) analysis method.

The IC₅₀ values of the compounds of Examples 14, 15, 20, 29, 30, 39, 40, 41, 47, 60, 77, 116 and 137 were not more than 5.00 nM.

### Test Example 5

### Vascular Endothelial Cell Growth Inhibitory Test

Human umbilical vein endothelial cells (HUVEC, purchased from KURABO) were cultured in a gas incubator (37° C., CO₂ 5%) in a vascular endothelial cell medium (Invitrogen) containing 5% fetal calf serum and a basic fibroblast growth factor (2.5 ng/mL). To be specific, HUVEC suspended in the aforementioned vascular endothelial cell medium (Invitrogen) containing 5% fetal calf serum were seeded into a 96 well flat-bottom plate (50 μL, 3000 cells each well). After culturing overnight, various concentrations of test substance and the final concentration (120 ng/mL) of vascular endothelial growth factor (VEGF) were dissolved in a vascular endothelial cell medium (Invitrogen) containing 5% fetal calf serum and added to each well (50 μL). After culturing for 5 days, an XTT reagent (Wako Pure Chemical Industries, Ltd.) was added to each well (10 μL), and reacted in a gas incubator (37° C., CO₂ 5%) for 2-3 hr. The absorbance at 450 nm was measured by a microtiter plate reader, and the cell growth inhibitory activity was determined. Using the absorbance with the addition of the test substance at each concentration and according to the non-linear least-squares method using the logistic curve of the SAS system NLIN procedure, the concentration (IC₅₀ value) of the test substance necessary for showing 50% of the value obtained without addition of the test substance was calculated. The results are shown in the following Table 1.

TABLE 1

| Example | IC₅₀ (nM) |
|---|---|
| 4 | 91 |
| 14 | 33 |
| 15 | 8.2 |
| 18 | 53 |
| 20 | 6.6 |
| 29 | 4.4 |
| 30 | 2.7 |
| 39 | 4.8 |
| 40 | 11 |
| 41 | 19 |
| 47 | 11 |
| 60 | 6.5 |
| 116 | 3.4 |
| 124 | 13 |
| 134 | 5.4 |
| 137 | 3.6 |

### Test Example 6

### Antitumor Test

The cancer cells were cultured in a gas incubator (37° C., CO₂ 5%) in a culture medium containing 10% fetal calf serum. The cells were isolated by a trypsin treatment, washed with HBSS (HANK's Balanced Saline Solution), and adjusted to the cell density of 1×10⁸ cells/mL with HBSS. The cell suspension (0.1 mL, 1×10⁷ cells) was subcutaneously injected to the abdomen of 6-week-old female nude mice (BALB/c nu/nu, CLEA Japan, Inc.) to implant the cells. When the tumor volume reached 100-200 mm³, they were divided into groups, and various doses of the test substance were orally administered for 14 consecutive days from the next day. The tumor volume was obtained by measuring the long diameter and the short diameter of the tumor over time and calculating from tumor volume=long diameter×short diameter×short diameter×0.5.

### INDUSTRIAL APPLICABILITY

The compounds (I)-(III) of the present invention, salts thereof and prodrugs thereof can provide clinically useful agents for the prophylaxis or treatment diseases (e.g., cancer and the like) associated with the action of vascular endothelial growth factors in living organisms, since they show a superior inhibitory action on kinases such as vascular endothelial growth factor receptor and the like. In addition, since the compounds (I)-(III) of the present invention, salts thereof and prodrugs thereof are superior in the efficacy expression, pharmacokinetic, solubility, interaction with other pharmaceutical products, safety and stability, they are useful as pharmaceutical agents.

This application is based on patent application Nos. 2005-196866 and 2006-054102 filed in Japan, the contents of which are incorporated in full herein by this reference.

1. A compound represented by the formula: 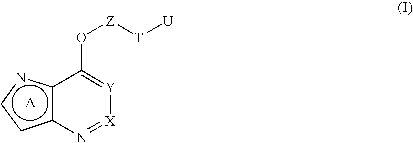
wherein ring A is an optionally substituted pyrrolc ring, X is an optionally substituted CH, Y is an optionally substituted CH or nitrogen atom, Z is an optionally substituted divalent hydrocarbon group or an optionally substituted divalent heterocyclic group, T is a single bond or an optionally substituted C₁₋₃ alkylene group, and U is an optionally substituted amido group, an optionally substituted sulfonamido group, an optionally substituted ureido group, an optionally substituted carbarnoyl group or an optionally substituted thioureido group, or a salt thereof.

2. The compound of claim 1, wherein X is CH, or a salt thereof.

3. The compound of claim 1, wherein Y is a nitrogen atom, or a salt thereof.

4. The compound of claim 1, wherein U is an optionally substituted ureido group, or a salt thereof.

5. The compound of claim 1, wherein T is a single bond, or a salt thereof.

6. The compound of claim 1, wherein ring A is an unsubstituted pyrrole ring or a pyrrole ring having substituent(s) on a ring nitrogen atom, or a salt thereof.

7. The compound of claim 1, which is represented by the formula: 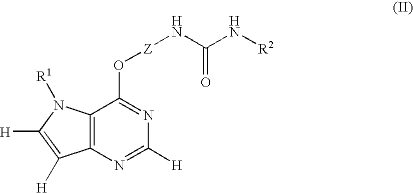
wherein R¹ is a hydrogen atom, an optionally substituted hydrocarbon group, an optionally substituted heterocyclic group or an acyl group, Z is an optionally substituted divalent hydrocarbon group or all optionally substituted divalent heterocyclic group, and R² is a hydrogen atom, an optionally substituted hydrocarbon group, an optionally substituted heterocyclic group, an optionally substituted amino group, an optionally substituted hydroxy group, an optionally substituted sulfanyl group or an acyl group, or a salt tliereof.

8. The compound of claim 7, wherein R¹ is a hydrogen atom or an optionally substituted hydrocarbon group, or a salt thereof.

9. The compound of claim 7, wherein Z is an optionally substituted C₆₋₁₄ arylene group or an optionally substituted divalent heterocyclic group, or a salt thereof.

10. The compound of claim 7, wherein Z is a C₆₋₁₄ arylene group substituted by a halogen atom, or a salt thereof.

11. The compound of claim 7, wherein R² is an optionally substituted C₆₋₁₄ aryl group or an optionally substituted heterocyclic group, or a salt thereof.

12. The compound of claim 1, which is represented by the formula: 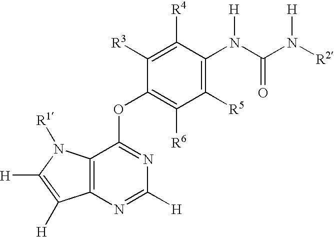
wherein R^{1′} is a hydrogen atom or an optionally substituted hydrocarbon group, R^{2′} is an optionally substituted phenyl group or an optionally substituted heterocyclic group, and R³, R⁴, R⁵ and R⁶ are each independently a hydrogen atom, a halogen atom, a cyano group, an optionally substituted hydrocarbon group, an optionally substituted amino group, an optionally substituted hydroxy group, an optionally substituted sulfanyl group or an acyl group or a salt thereof.

13. The compound of claim 12, wherein R⁴ is a halogen atom and R^{1′} is an optionally substituted hydrocarbon group, or a salt thereof.

14. (i) N-{2-Chloro-4-[(5-methyl-5H-pyrrolo[3,2-d]pyrimidinA-yl)oxy]phenyl}-N′-[3-(trifluorornethyl)phenyl]urea,
(ii) N-{2-chloro-4-[(5-methyl-5H-pyrrolo[3,2-d]pyrimidin-4-yl)oxy]phenyl}-N′-[3-(trifluoromethoxy)phenyl]urea,
(iii) N-{2-fluoro-4-[(5-methyl-5H-pyrrolo[3,2-d]pyrimidinyl)oxy]phenyl}-N′-[3-(trifluoromethyl)phenyl]urea,
(iv) N-{2-chloro-4-[(5-methyl-5H-pyrrolo[3,2-d]pyrimidin-4-yl)oxy]phenyl}-N′-[4-(tri fluoromethyl)pyridin-2-yl]urea,
(v) N-[2-chloro-4-(5H-pyrrolo[3,2-d]pyrinudin-4-yloxy)phenyl]-N′-[3-(trifluoromethyl)phenyl]urea,
(vi) N-{2-chloro-4-[(5-methyl-5H-pyrrolo[3,2-d]pyrimidin-4-yloxy]phenyl}-N′-[2-fluoro-5-(trifluoromethyl)phenyl]urea,
(vii) N-{2-chloro-4-[(5,6-dimethyl-5H-pyrrolo[3,2-d]pyrimidin-4-yl)oxy]phenyl}-N′-[3-(trifluoromethyl)phenyl]urea, or a salt of any thereof.

15. A prodrug of the compound of claim 1.

16. A pharmaceutical agent comprising the compound of claim 1 or a prodrug thereof.

17. The pharmaceutical agent of claim 16, which is a kinase inhibitor.

18. The pharmaceutical agent of claim 17, wherein the kinase is a vascular endothelial growth factor receptor (VEGFR).

19. The pharmaceutical agent of claim 17, wherein the kinase is vascular endothelial growth factor receptor (VEGFR) 2.

20. The pharmaceutical agent of claim 17, wherein the kinase is a platelet-derived growth factor receptor (PDGFR).

21. The pharmaceutical agent of claim 17, wherein the kin ase is a tyrosine kinase with Ig and EGF homology domains2 (TIE2).

22. The pharmaceutical agent of claim 16, which is an angiogenesis inhibitor.

23. The pharmaceutical agent of claim 16, which is an agent for the prophylaxis or treatment of cancer.

24. The pharmaceutical agent of claim 16, which is an agent for inhibiting growth of cancer.

25. The pharmaceutical agent of claim 16, which is an agent for suppressing metastasis of cancer.

26. A method for the prophylaxis or treatment of cancer, which comprises administering an effective amount of the compound of claim 1 or a prodrig thereof to a mammal.

27. (canceled)
